(19) United States
(12) Patent Application Publication
(10) Pub. No.: US 2011/0123568 A1
(43) Pub. Date: May 26, 2011

(54) NOVEL USE

(75) Inventors: Emmanuel Jules Hanon, Rixensart (BE); Jean Stephenne, Rixensart (BE)

(73) Assignee: GlaxoSmithKline Biologicals, s.a.

(21) Appl. No.: 12/951,857

(22) Filed: Nov. 22, 2010

Related U.S. Application Data
(63) Continuation of application No. 12/148,095, filed on Apr. 16, 2008, now abandoned.
Application No. 12/148,095 is a continuation-in-part of application No. 11/909,351, filed on Sep. 21, 2007.

(30) Foreign Application Priority Data
Mar. 23, 2005 (GB) 0505989.4
Mar. 23, 2005 (GB) 0505998.5
Mar. 23, 2005 (GB) 0506000.9
Mar. 23, 2005 (GB) 0506001.7
Mar. 23, 2005 (GB) 0506004.1
May 24, 2005 (GB) 0510589.5
May 24, 2005 (GB) 0510591.1
May 24, 2005 (GB) 0510593.7
May 24, 2005 (GB) 0510596.0
May 24, 2005 (GB) 0510598.6
Feb. 24, 2006 (GB) 0603788.1
Feb. 24, 2006 (GB) 0603789.9
Feb. 24, 2006 (GB) 0603790.7

(51) Int. Cl. A61K 39/145; A61P 31/16; A61P 37/04

(52) U.S. Cl. 424/210.1; 424/209.1

(57) ABSTRACT

The present invention relates to influenza vaccine formulations and vaccination regimes for immunizing against influenza disease. In particular the invention relates to vaccine formulations comprising an oil-in-water emulsion adjuvant and optionally 3D-MPL, their use in medicine, in particular their use in augmenting immune responses to influenza antigens, and to methods of preparation, wherein the oil in water emulsion comprises a sterol, a metabolizable oil and an emulsifying agent. The present invention also provides for new prime-boost vaccination regimes for immunizing humans against influenza disease, and in particular for ensuring and ameliorating the immune response to the booster administration, in which a first influenza virus vaccine is administered in the presence of an adjuvant.

### CROSS-REFERENCE TO RELATED APPLICATIONS

This application is a Continuation of U.S. patent application Ser. No. 12/148,095 (currently pending), which is a Continuation-In-Part (CIP) of U.S. patent application Ser. No. 11/909,351 (currently pending), which is the National Stage of International Application No. PCT/EP2006/002836, filed 21 Mar. 2006, the disclosures of which applications are incorporated herein by reference. This application also claims benefit of the earlier filing dates of Great Britain Applications Nos: 0506001.7, filed 23 Mar. 2005; 0506000.9, filed 23 Mar. 2005; 0505998.5, filed 23 Mar. 2005; 0505989.4, filed 23 Mar. 2005; 0506004.1, filed 23 Mar. 2005; 0510589.5, filed 24 May 2005; 0510591.1, filed 24 May 2005; 0510593.7, filed 24 May 2005; 0510596.0, filed 24 May 2005; 0510598.6, filed 24 May 2005; 0603789.9, filed 24 Feb. 2006; 0603788.1, filed 24 Feb. 2006; and 0603790.7, filed 24 Feb. 2006.

### TECHNICAL FIELD

The present invention relates to influenza vaccine formulations and vaccination regimes for immunizing against influenza disease. In particular the invention relates to vaccine formulations comprising an oil-in-water emulsion adjuvant and optionally 3D-MPL, their use in medicine, in particular their use in augmenting immune responses to influenza antigens, and to methods of preparation, wherein the oil in water emulsion comprises a sterol, a metabolizable oil and an emulsifying agent.

### TECHNICAL BACKGROUND

Influenza viruses are one of the most ubiquitous viruses present in the world, affecting both humans and livestock. Influenza results in an economic burden, morbidity and even mortality, which are significant.

The influenza virus is an RNA enveloped virus with a particle size of about 125 nm in diameter. It consists basically of an internal nucleocapsid or core of ribonucleic acid (RNA) associated with nucleoprotein, surrounded by a viral envelope with a lipid bilayer structure and external glycoproteins. The inner layer of the viral envelope is composed predominantly of matrix proteins and the outer layer mostly of host-derived lipid material.

Influenza virus comprises two surface antigens, glycoproteins neuraminidase (NA) and haemagglutinin (HA), which appear as spikes, 10 to 12 nm long, at the surface of the particles. It is these surface proteins, particularly the haemagglutinin that determine the antigenic specificity of the influenza subtypes.

These surface antigens progressively, sometimes rapidly, undergo some changes leading to the antigenic variations in influenza. These antigenic changes, called ‘drifts’ and ‘shifts’ are unpredictable and may have a dramatic impact from an immunological point of view as they eventually lead to the emergence of new influenza strains and that enable the virus to escape the immune system causing the well known, almost annual, epidemics.

The influenza virus strains to be incorporated into influenza vaccine each season are determined by the World Health Organisation in collaboration with national health authorities and vaccine manufacturers.

HA is the most important antigen in defining the serological specificity of the different influenza strains. This 75-80 kD protein contains numerous antigenic determinants, several of which are in regions that undergo sequence changes in different strains (strain-specific determinants) and others in regions which are common to many HA molecules (common to determinants).

Influenza viruses cause epidemics almost every winter, with infection rates for type A or B virus as high as 40% over a six-week period. Influenza infection results in various disease states, from a sub-clinical infection through mild upper respiratory infection to a severe viral pneumonia. Typical influenza epidemics cause increases in incidence of pneumonia and lower respiratory disease as witnessed by increased rates of hospitalization or mortality. The severity of the disease is primarily determined by the age of the host, his immune status and the site of infection.

Elderly people, 65 years old and over, are especially vulnerable, accounting for 80-90% of all influenza-related deaths in developed countries. Individuals with underlying chronic diseases are also most likely to experience such complications. Young infants also may suffer severe disease. These groups in particular therefore need to be protected. Besides these ‘at risk’-groups, the health authorities are also recommending to vaccinate healthy adults who are in contact with elderly persons.

Vaccination plays a critical role in controlling annual influenza epidemics. Currently available influenza vaccines are either inactivated or live attenuated influenza vaccine. Inactivated flu vaccines are composed of three possible forms of antigen preparation: inactivated whole virus, sub-virions where purified virus particles are disrupted with detergents or other reagents to solubilise the lipid envelope (so-called “split” vaccine) or purified HA and NA (subunit vaccine). These inactivated vaccines are given intramuscularly (i.m.) or intranasaly (i.n.).

Influenza vaccines, of all kinds, are usually trivalent vaccines. They generally contain antigens derived from two influenza A virus strains and one influenza B strain. A standard 0.5 ml injectable dose in most cases contains 15 μg of haemagglutinin antigen component from each strain, as measured by single radial immunodiffusion (SRD) (J. M. Wood et al.: An improved single radial immunodiffusion technique for the assay of influenza haemagglutinin antigen: adaptation for potency determination of inactivated whole virus and subunit vaccines. J. Biol. Stand. 5 (1977) 237-247; J. M. Wood et al., International collaborative study of single radial diffusion and immunoelectrophoresis techniques for the assay of haemagglutinin antigen of influenza virus. J. Biol. Stand. 9 (1981) 317-330).

Influenza vaccines currently available are considered safe in all age groups (De Donato et al. 1999, Vaccine, 17, 3094-3101). However, there is little evidence that current influenza vaccines work in small children under two years of age. Furthermore, reported rates of vaccine efficacy for prevention of typical confirmed influenza illness are 23-72% for the elderly, which are significantly lower than the 60-90% efficacy rates reported for younger adults (Govaert, 1994, J. Am. Med. Assoc., 21, 166-1665; Gross, 1995, Ann Intern. Med. 123, 523-527). The effectiveness of an influenza vaccine has been shown to correlate with serum titres of hemagglutination inhibition (HI) antibodies to the viral strain, and several studies have found that older adults exhibit lower HI titres after influenza immunization than do younger adults (Murasko, 2002, Experimental gerontology, 37, 427-439).

New vaccines with an improved immunogenicity are therefore still needed. Formulation of vaccine antigen with potent adjuvants is a possible approach for enhancing immune responses to subvirion antigens.

A sub-unit influenza vaccine adjuvanted with the adjuvant MF59, in the form of an oil-in-water emulsion is commercially available, and has demonstrated its ability to induce a higher antibody titer than that obtained with the non-adjuvanted sub-unit vaccine (De Donato et al. 1999, Vaccine, 17, 3094-3101). However, in a later publication, the same vaccine has not demonstrated its improved profile compared to a non-adjuvanted split vaccine (Puig-Barbera et al., 2004, Vaccine 23, 283-289).

There is still a need for improved influenza vaccines, especially in the elderly population.

### STATEMENT OF INVENTION

In first aspect of the present invention, there is provided the use of:

(a) an influenza virus or antigenic preparation thereof, and
(b) an oil-in-water emulsion adjuvant in the manufacture of an immunogenic composition for inducing at least one of i) an improved CD4 T-cell immune response, ii) an improved B-memory cell response against said virus or antigenic composition in a human wherein said oil-in-water emulsion comprises a metabolizable oil, a sterol and an emulsifying agent.

Suitably said sterol is alpha-tocopherol. In a particular embodiment, said oil-in-water emulsion adjuvant comprises at least one metabolizable oil in an amount of 0.5% to 20% of the total volume, and has oil droplets of which at least 70% by intensity have diameters of less than 1 μm.

In a specific embodiment, the immunogenic composition is capable of inducing both an improved CD4 T-cell immune response and an improved B-memory cell response compared to that obtained with the un-adjuvanted antigen or antigenic composition.

In a second aspect of the present invention, there is provided the use of:
(a) an influenza virus or antigenic preparation thereof, and
(b) an oil-in-water emulsion adjuvant
   in the manufacture of an immunogenic composition for vaccination of a human immuno-compromised individual or population, such as a high risk adult or a elderly, against influenza, wherein said oil-in-water emulsion comprises a metabolizable oil, a sterol and an emulsifying agent.

In a preferred embodiment, said oil-in-water emulsion adjuvant comprises at least one metabolizable oil in an amount of 0.5% to 20% of the total volume, and has oil droplets of which at least 70% by intensity have diameters of less than 1 μm.

In a third aspect of the present invention, there is provided the use of an influenza virus or antigenic preparation thereof in the manufacture of an immunogenic composition for revaccination of humans previously vaccinated with an influenza virus or antigenic preparation thereof formulated with an oil-in-water emulsion adjuvant comprising a metabolizable oil, a sterol, suitably alpha-tocopherol, and an emulsifying agent. Preferably the revaccination is made in subjects who have been vaccinated the previous season against influenza. Typically revaccination is made at least 6 months after the first vaccination, preferably 8 to 14 months after, more preferably at around 10 to 12 months after.

Preferably, there is provided the use of:
(a) an influenza virus or antigenic preparation thereof, and
(b) an oil-in-water emulsion adjuvant comprising a metabolizable oil, a sterol (such as alpha-tocopherol), and an emulsifying agent
   in the manufacture of an immunogenic composition for revaccination of humans previously vaccinated with an influenza virus or antigenic preparation thereof and an oil-in-water emulsion adjuvant, wherein said oil-in-water emulsion adjuvant comprises at least one metabolizable oil in an amount of 0.5% to 20% of the total volume, and has oil droplets of which at least 70% by intensity have diameters of less than 1 μm.

In another preferred embodiment, the immunogenic composition for revaccination contains a split influenza virus or split virus antigenic preparation thereof which shares either common CD4 T-cell epitopes or common B cell epitopes, or both, with the influenza virus or antigenic preparation thereof used for the first vaccination.

In a fourth aspect of the present invention, there is provided the use of:
(a) an influenza virus or antigenic preparation thereof, from a first influenza strain, and
(b) an oil-in-water emulsion adjuvant comprising a metabolizable oil, a sterol and an emulsifying agent
   in the manufacture of an immunogenic composition for protection against influenza infections caused by a influenza strain which is a variant of said first influenza strain. In a preferred embodiment, said oil-in-water emulsion adjuvant comprises at least one metabolizable oil in an amount of 0.5% to 20% of the total volume, and has oil droplets of which at least 70% by intensity have diameters of less than 1 μm.

In another aspect, there is provided a method of vaccination an immunocompromised human individual or population such as high risk adults or elderly, with an immunogenic composition comprising an influenza virus or antigenic preparation thereof and an oil-in-water emulsion adjuvant, as hereinabove defined.

In still another embodiment, the invention provides a method for revaccinating humans previously vaccinated with an influenza virus or virus antigenic preparation thereof and an oil-in-water emulsion adjuvant comprising a metabolizable oil, a sterol and an emulsifying agent, said method comprising administering to said human an immunogenic composition comprising an influenza virus, either adjuvanted or un-adjuvanted. Suitably said sterol is alpha-tocopherol.

In a further embodiment there is provided a method for vaccinating a human population or individual against one influenza virus strain followed by revaccination of said human or population against a variant influenza virus strain, said method comprising administering to said human (i) a first composition comprising an influenza virus or antigenic preparation thereof from a first influenza virus strain and an oil-in-water emulsion adjuvant comprising a metabolizable oil, a sterol and an emulsifying agent, and (ii) a second immunogenic composition comprising a influenza virus strain variant of said first influenza virus strain. Suitably said sterol is alpha-tocopherol.

In another embodiment, the invention provides the use of an influenza virus or antigenic preparation thereof and an oil-in-water emulsion adjuvant comprising a metabolizable oil, a sterol and an emulsifying agent. Suitably said sterol is alpha-tocopherol.

In a still further aspect, the invention provides a method of designing an influenza vaccine, comprising
1) selecting an influenza antigen containing CD4+ epitopes, and
2) combining said influenza antigen with an oil-in-water emulsion as defined above, wherein said vaccine upon administration in a mammal is capable of inducing an enhanced CD4 response in said mammal.

In yet other aspects, the invention provides methods for priming and boosting an immune response against influenza. In the methods disclosed herein, the priming dose of antigen is formulated with an adjuvant, e.g., as described herein. Surprisingly, administration to subjects of a first dose of adjuvanted influenza vaccine significantly enhances (or prevents impairment of) the boosted response as compared to administration of a first dose of an unadjuvanted vaccine.

Other aspects and advantages of the present invention are described further in the following detailed description and examples.

### BRIEF DESCRIPTION OF THE DRAWINGS

FIG. 1: Oil droplet particle size distribution in SB62 oil-in-water emulsion as measured by PCS. FIG. 1A shows SB62 lot 1023 size measurements with the Malvern Zetasizer 3000HS: A=dilution 1/10000 (Rec22 to Rec24) (Analysis in Contin and adapted optical model 1.5/0.01); B=Dilution 1/20000 (Rec28 to Rec30) (Analysis in Contin and adapted optical model 1.5/0.01). FIG. 1B shows a schematic illustration of record 22 (upper part) and record 23 (lower part) by intensity.

FIG. 2: Schematic illustration of the preparation of MPL bulk.

FIG. 3: Schematic illustration of the preparation of AS03+MPL adjuvant.

FIG. 4: Explo Flu-001 clinical trial. CD4 T cell response to split influenza antigen (Q1=first quartile, Q3=third quartile).

FIG. 5: Explo Flu-001 clinical trial. CD8 T cell response to split influenza antigen (Q1=first quartile, Q3=third quartile).

FIG. 6: Explo Flu-001 clinical trial. Cross-reactive CD4 T-cell response to split influenza virus antigen after vaccination with Fluarix+AS03.

FIG. 7: Explo Flu-001 clinical trial. B cell memory response post vaccination.

FIG. 8: Explo Flu-002 clinical trial. CD4 T cell response against split influenza antigen following revaccination.

FIG. 9: Explo Flu-002 clinical trial. Anti-HI titers following revaccination.

FIG. 10: Ferret study I. Temperature monitoring (priming and challenge). FIG. 10A is priming, FIG. 10B is challenge.

FIG. 11: Ferret study I. Viral shedding.

FIG. 12: Ferret study II. Temperature monitoring (priming and challenge). FIG. 12A is priming, FIG. 12B is challenge.

FIG. 13: Ferret study II. Viral shedding.

FIG. 14: Ferret study II. HI titers to H3N2 A/Panama (vaccine strain) (FIG. 14A) and to H3N2 A/Wyoming (challenge strain) (FIG. 14B).

FIG. 15: Mice study. Frequencies of CD4 T cells in C57BI/6 primed mice using whole inactivated virus as re-stimulating antigen (day 7 post-immunization).

FIG. 16: Mice study. Frequencies of CD8 T cells in C57BI/6 primed mice using whole inactivated virus as re-stimulating antigen (day 7 post-immunization).

FIG. 17: Mice study. Frequencies of CD4 (upper part) and CD8 (lower part) T cells in C57BI/6 mice primed with heterologous strains, using whole inactivated virus as re-stimulating antigen (day 7 post-immunization).

FIG. 18: Human clinical trial. B cell memory response post-vaccination of elderly with Fluarix, Fluarix+AS03, Fluarix+AS03+MPL (difference between pre- and post-).

FIG. 19: Ferret study III. Temperature monitoring before and after challenge.

FIG. 20: Ferret study III. Viral shedding before and after challenge.

FIG. 21: Ferret study III. HI titers to H3N2 A/Woming (vaccine strain).

FIG. 22: Ferret study III. HI titers to H3N2 A/Panama (challenge strain).

FIG. 23: Human clinical trial. HI titers (GMTs) at days 21, 90 and 180 post vaccination (persistence).

FIG. 24: Human clinical trial. CD4 response—all double test—Pool antigen at days 21, 90 and 180 post vaccination (persistence).

FIG. 25: Human clinical trial. HI titers in a revaccination clinical trial with AS03+MPL compared to Fluarix.

FIG. 26: Human clinical trial. CMI for CD4 response—all double test—Pool antigen at days 0 and 21.

FIG. 27: Human clinical trial with AS03+MPL at two concentrations. HI titers at days 0 and 21.

FIG. 28: Human clinical trial with AS03+MPL at two concentrations. Reactogenicity.

FIG. 29: Schematic illustration of study design and vaccination schedule.

FIGS. 30A and B: Bar graphs illustrating GMTs of anti-HA Abs against the VT or IN strains up to Month 6+21 days, for adults who received the booster dose at Month 6. Comparison between one or two doses for the first vaccination.

FIGS. 31A and B: Bar graphs illustrating GMTs of anti-HA Abs against the VT or IN strains up to Month 6+21 days, for adults who received the booster dose at Month 6. Comparison between INDO or VIET boosting in subjects having received one primary dose at D0(A) or two primary doses (B) at D0 and D21.

FIGS. 32A-D: Bar graphs illustrating HI Seroconversion rates (in %) at D42 (A), Month 6 (B), Month 6+7 days (C), Month 6+21 days (C) and all time points (D).

FIG. 33: Bar graph illustrating Booster “seroconversion rate” response against the VIET or INDO strains at Month 6+21 days for adults who received the booster dose at Month 6 (using the Month 6 HI value as the pre-vaccination value, i.e. pre-booster).

FIG. 34: Bar graph illustrating Seroprotection rates at Month 6, Month 6+7 days and Month 6+21 days.

FIG. 35: Bar graph illustrating Seroprotection rates at Month 6, Month 6+7 days and Month 6+21 days.

FIG. 36: Schematic illustration of study design and vaccination schedule.

FIG. 37: Bar graph illustrating GMTs of Anti-HA antibody titres at Days 0, 7, 14, 21, 28, 35, 42 in all groups against A/Indonesia/05/2005 strain.

FIG. 38: Bar graph illustrating SCR for anti-HA antibody titer at PI(D14) and PI(D21) and PI(D7) and PII(D35) and PII(D42) in all groups against A/Indonesia/05/2005 strain.

FIG. 39: Bar graph illustrating SCF for anti-HA antibody titer at each post-vaccination time point in all groups against A/Indonesia/05/2005 strain.

FIG. 40: Bar graph illustrating SPR for anti-HA antibody titer at each post-vaccination time point in all groups against A/Indonesia/05/2005 strain.

FIGS. 41A-D: Graphs illustrating CMI analysis: CD4 cells producing at least two Th1 cytokines; A=split H5N1 INDO; B=split H5N1 VIET; C=peptide pool INDO; D=peptide pool VIET.

FIGS. 42A and B: Graphs illustrating Memory B cells specific to Indonesia H5N1 antigen (upper FIG. A) and Vietnam H5N1 antigen (bottom FIG. B).

### DETAILED DESCRIPTION

The present inventors have discovered that an influenza formulation comprising an influenza virus or antigenic preparation thereof together with an oil-in-water emulsion adjuvant comprising a metabolizable oil, a sterol such as alpha-tocopherol and an emulsifying agent, was capable of improving the CD4 T-cell immune response and/or B cell memory response against said antigen or antigenic composition in a human compared to that obtained with the un-adjuvanted virus or antigenic preparation thereof. The claimed formulations will advantageously be used to induce anti-influenza CD4−T cell response capable of detection of influenza epitopes presented by MHC class II molecules. The present Applicant has now found that it is effective to target the cell-mediated immune system in order to increase responsiveness against homologous and drift influenza strains (upon vaccination and infection).

The adjuvanted influenza compositions according to the invention have several advantages:
1) An improved immunogenicity: they will allow to restore weak immune response in the elderly people (over 50 years of age, typically over 65 years of age) to levels seen in young people (antibody and/or T cell responses);
2) An improved cross-protection profile: increased cross-protection against variant (drifted) influenza strains;
3) They will also allow an reduced antigen dosage to be used for a similar response, thus ensuring an increased capacity in case of emergency (pandemics for example).

In particular, the compositions of the present invention have been able to provide better sero-protection against influenza following revaccination, as assessed by the number of human subjects meeting the influenza correlates of protections. Furthermore, the composition for use in the present invention have also been able to induce a trend for a higher B cell memory response following the first vaccination of a human subject, and a higher humoral response following revaccination, compared to the un-adjuvanted composition.

The Inventors have also been capable of demonstrating that the claimed adjuvanted composition was able to not only induce but also maintain protective levels of antibodies against all three strains present in the vaccine, in more individuals than those obtained with the un-advanted composition (see Table 43 for example).

Thus, in still another embodiment, the claimed composition is capable of ensuring a persistent immune response against influenza related disease. In particular, by persistence it is meant an HI antibody immune response which is capable of meeting regulatory criteria after at least three months, preferably after at least 6 months after the vaccination. In particular, the claimed composition is able to induce protective levels of antibodies in >70% of individuals, suitably in >80% of individuals or suitably in >90% of individuals for at least one influenza strain, preferably for all strains present in the vaccine, after at least three months. In a specific aspect, protective levels of antibodies of >90% are obtained at least 6 months post-vaccination against at least one, suitably two, or all strains present in the vaccine composition.

### Influenza Viral Strains and Antigens

An influenza virus or antigenic preparation thereof for use according to the present invention may be a split influenza virus or split virus antigenic preparation thereof. In an alternative embodiment the influenza preparation may contain another type of inactivated influenza antigen, such as inactivated whole virus or purified HA and NA (subunit vaccine), or an influenza virosome. In a still further embodiment, the influenza virus may be a live attenuated influenza preparation.

A split influenza virus or split virus antigenic preparation thereof for use according to the present invention is suitably an inactivated virus preparation where virus particles are disrupted with detergents or other reagents to solubilise the lipid envelope. Split virus or split virus antigenic preparations thereof are suitably prepared by fragmentation of whole influenza virus, either infectious or inactivated, with solubilising concentrations of organic solvents or detergents and subsequent removal of all or the majority of the solubilising agent and some or most of the viral lipid material. By split virus antigenic preparation thereof is meant a split virus preparation which may have undergone some degree of purification compared to the split virus whilst retaining most of the antigenic properties of the split virus components. For example, when produced in eggs, the split virus may be depleted from egg-contaminating proteins, or when produced in cell culture, the split virus may be depleted from host cell contaminants. A split virus antigenic preparation may comprise split virus antigenic components of more than one viral strain. Vaccines containing split virus (called ‘influenza split vaccine’) or split virus antigenic preparations generally contain residual matrix protein and nucleoprotein and sometimes lipid, as well as the membrane envelope proteins. Such split virus vaccines will usually contain most or all of the virus structural proteins although not necessarily in the same proportions as they occur in the whole virus.

Alternatively, the influenza virus may be in the form of a whole virus vaccine. This may prove to be an advantage over a split virus vaccine for a pandemic situation as it avoids the uncertainty over whether a split virus vaccine can be successfully produced for a new strain of influenza virus. For some strains the conventional detergents used for producing the split virus can damage the virus and render it unusable. Although there is always the possibility to use different detergents and/or to develop a different process for producing a split vaccine, this would take time, which may not be available in a pandemic situation. In addition to the greater degree of certainty with a whole virus approach, there is also a greater vaccine production capacity than for split virus since considerable amounts of antigen are lost during additional purification steps necessary for preparing a suitable split vaccine.

In another embodiment, the influenza virus preparation is in the form of a purified sub-unit influenza vaccine. Sub-unit influenza vaccines generally contain the two major envelope proteins, HA and NA, and may have an additional advantage over whole virion vaccines as they are generally less reactogenic, particularly in young vaccinees. Sub-unit vaccines can produced either recombinantly or purified from disrupted viral particles.

In another embodiment, the influenza virus preparation is in the form of a virosome. Virosomes are spherical, unilamellar vesicles which retain the functional viral envelope glycoproteins HA and NA in authentic conformation, intercalated in the virosomes' phospholipids bilayer membrane.

Said influenza virus or antigenic preparation thereof may be egg-derived or tissue-culture derived.

For example, the influenza virus antigen or antigenic preparations thereof according to the invention may be derived from the conventional embryonated egg method, by growing influenza virus in eggs and purifying the harvested allantoic fluid. Eggs can be accumulated in large numbers at short notice. Alternatively, they may be derived from any of the new generation methods using tissue culture to grow the virus or express recombinant influenza virus surface antigens. Suitable cell substrates for growing the virus include for example dog kidney cells such as MDCK or cells from a clone of MDCK, MDCK-like cells, monkey kidney cells such as AGMK cells including Vero cells, suitable pig cell lines, or any other mammalian cell type suitable for the production of influenza virus for vaccine purposes. Suitable cell substrates also include human cells e.g. MRC-5 cells. Suitable cell substrates are not limited to cell lines; for example primary cells such as chicken embryo fibroblasts and avian cell lines are also included.

The influenza virus antigen or antigenic preparation thereof may be produced by any of a number of commercially applicable processes, for example the split flu process described in patent no. DD 300833 and DD 211444, incorporated herein by reference. Traditionally split flu was produced using a solvent/detergent treatment, such as tri-n-butyl phosphate, or diethylether in combination with Tween™ (known as “Tween-ether” splitting) and this process is still used in some production facilities. Other splitting agents now employed include detergents or proteolytic enzymes or bile salts, for example sodium deoxycholate as described in patent no. DD 155 875, incorporated herein by reference. Detergents that can be used as splitting agents include cationic detergents e.g. cetyl trimethyl ammonium bromide (CTAB), other ionic detergents e.g. laurylsulfate, taurodeoxycholate, or non-ionic detergents such as the ones described above including Triton X-100 (for example in a process described in Lina et al, 2000, Biologicals 28, 95-103) and Triton N-101, or combinations of any two or more detergents.

The preparation process for a split vaccine may include a number of different filtration and/or other separation steps such as ultracentrifugation, ultrafiltration, zonal centrifugation and chromatography (e.g. ion exchange) steps in a variety of combinations, and optionally an inactivation step eg with heat, formaldehyde or β-propiolactone or U.V. which may be carried out before or after splitting. The splitting process may be carried out as a batch, continuous or semi-continuous process. A preferred splitting and purification process for a split immunogenic composition is described in WO 02/097072.

Preferred split flu vaccine antigen preparations according to the invention comprise a residual amount of Tween 80 and/or Triton X-100 remaining from the production process, although these may be added or their concentrations adjusted after preparation of the split antigen. Preferably both Tween 80 and Triton X-100 are present. The preferred ranges for the final concentrations of these non-ionic surfactants in the vaccine dose are:

Tween 80: 0.01 to 1%, more preferably about 0.1% (v/v)
Triton X-100: 0.001 to 0.1 (% w/v), more preferably 0.005 to 0.02% (w/v).

In a specific embodiment, the final concentration for Tween 80 ranges from 0.045%-0.09% w/v. In another specific embodiment, the antigen is provided as a 2 fold concentrated mixture, which has a Tween 80 concentration ranging from 0.045%-0.2% (w/v) and has to be diluted two times upon final formulation with the adjuvanted (or the buffer in the control formulation).

In another specific embodiment, the final concentration for Triton X-100 ranges from 0.005%-0.017% w/v. In another specific embodiment, the antigen is provided as a 2 fold concentrated mixture, which has a Triton X-100 concentration ranging from 0.005%-0.034% (w/v) and has to be diluted two times upon final formulation with the adjuvanted (or the buffer in the control formulation).

Preferably the influenza preparation is prepared in the presence of low level of thiomersal, or preferably in the absence of thiomersal. Preferably the resulting influenza preparation is stable in the absence of organomercurial preservatives, in particular the preparation contains no residual thiomersal. In particular the influenza virus preparation comprises a haemagglutinin antigen stabilised in the absence of thiomersal, or at low levels of thiomersal (generally 5 μg/ml or less). Specifically the stabilization of B influenza strain is performed by a derivative of alpha tocopherol, such as alpha tocopherol succinate (also known as vitamin E succinate, i.e. VES). Such preparations and methods to prepare them are disclosed in WO 02/097072.

A preferred composition contains three inactivated split virion antigens prepared from the WHO recommended strains of the appropriate influenza season.

Preferably the influenza virus or antigenic preparation thereof and the oil-in-water emulsion adjuvant are contained in the same container. It is referred to as ‘one vial approach’. Preferably the vial is a pre-filled syringe. In an alternative embodiment, the influenza virus or antigenic preparation thereof and the oil-in-water emulsion adjuvant are contained in separate containers or vials and admixed shortly before or upon administration into the subject. It is referred to as ‘two vials approach’. By way of example, when the vaccine is a 2 components vaccine for a total dose volume of 0.7 ml, the concentrated antigens (for example the concentrated trivalent inactivated split virion antigens) are presented in one vial (335 μl) (antigen container) and a pre-filled syringe contains the adjuvant (360 μl) (adjuvant container). At the time of injection, the content of the vial containing the concentrated trivalent inactivated split virion antigens is removed from the vial by using the syringe containing the adjuvant followed by gentle mixing of the syringe. Prior to injection, the used needle is replaced by an intramuscular needle and the volume is corrected to 530 μl. One dose of the reconstituted adjuvanted influenza vaccine candidate corresponds to 530 μl.

### Oil-in-Water Emulsion Adjuvant

The adjuvant composition of the invention contains an oil-in-water emulsion adjuvant, preferably said emulsion comprises a metabolizable oil in an amount of 0.5% to 20% of the total volume, and having oil droplets of which at least 70% by intensity have diameters of less than 1 μm.

In order for any oil in water composition to be suitable for human administration, the oil phase of the emulsion system has to comprise a metabolizable oil. The meaning of the term metabolizable oil is well known in the art. Metabolisable can be defined as ‘being capable of being transformed by metabolizm’ (Dorland's Illustrated Medical Dictionary, W.B. Sanders Company, 25th edition (1974)). The oil may be any vegetable oil, fish oil, animal oil or synthetic oil, which is not toxic to the recipient and is capable of being transformed by metabolizm. Nuts, seeds, and grains are common sources of vegetable oils. Synthetic oils are also part of this invention and can include commercially available oils such as NEOBEE® and others. A particularly suitable metabolizable oil is squalene. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly preferred oil for use in this invention. Squalene is a metabolizable oil by virtue of the fact that it is an intermediate in the biosynthesis of cholesterol (Merck index, 10th Edition, entry no. 8619).

Oil in water emulsions per se are well known in the art, and have been suggested to be useful as adjuvant compositions (EP 399843; WO 95/17210).

Suitably the metabolizable oil is present in an amount of 0.5% to 20% (final concentration) of the total volume of the immunogenic composition, preferably an amount of 1.0% to 10% of the total volume, preferably in an amount of 2.0% to 6.0% of the total volume.

In a specific embodiment, the metabolizable oil is present in a final amount of about 0.5%, 1%, 3.5% or 5% of the total volume of the immunogenic composition. In another specific embodiment, the metabolizable oil is present in a final amount of 0.5%, 1%, 3.57% or 5% of the total volume of the immunogenic composition.

Preferably the oil-in-water emulsion systems of the present invention have a small oil droplet size in the sub-micron range. Suitably the droplet sizes will be in the range 120 to 750 nm, more preferably sizes from 120 to 600 nm in diameter. Most preferably the oil-in water emulsion contains oil droplets of which at least 70% by intensity are less than 500 nm in diameter, more preferably at least 80% by intensity are less than 300 nm in diameter, more preferably at least 90% by intensity are in the range of 120 to 200 nm in diameter.

The oil droplet size, i.e. diameter, according to the present invention is given by intensity. There are several ways of determining the diameter of the oil droplet size by intensity. Intensity is measured by use of a sizing instrument, suitably by dynamic light scattering such as the Malvern Zetasizer 4000 or preferably the Malvern Zetasizer 3000HS. A detailed procedure is given in Example II.2. A first possibility is to determine the z average diameter ZAD by dynamic light scattering (PCS-Photon correlation spectroscopy); this method additionally give the polydispersity index (PDI), and both the ZAD and PDI are calculated with the cumulants algorithm. These values do not require the knowledge of the particle refractive index. A second mean is to calculate the diameter of the oil droplet by determining the whole particle size distribution by another algorithm, either the Contin, or NNLS, or the automatic “Malvern” one (the default algorithm provided for by the sizing instrument). Most of the time, as the particle refractive index of a complex composition is unknown, only the intensity distribution is taken into consideration, and if necessary the intensity mean originating from this distribution.

The oil in water emulsion comprises a sterol. Sterols are well known in the art, for example cholesterol is well known and is, for example, disclosed in the Merck Index, 11th Edn., page 341, as a naturally occurring sterol found in animal fat. Other suitable sterols include β-sitosterol, stigmasterol, ergosterol, alpha-tocopherol and ergocalciferol. Said sterol is suitably present in an amount of 0.01% to 20% (w/v) of the total volume of the immunogenic composition, preferably at an amount of 0.1% to 5% (w/v). Preferably, when the sterol is cholesterol, it is present in an amount of between 0.02% and 0.2% (w/v) of the total volume of the immunogenic composition, more preferably at an amount of 0.02% (w/v) in a 0.5 ml vaccine dose volume, or 0.07% (w/v) in 0.5 ml vaccine dose volume or 0.1% (w/v) in 0.7 ml vaccine dose volume.

Suitably the sterol is alpha-tocopherol or a derivative thereof such as alpha-tocopherol succinate. Preferably alpha-tocopherol is present in an amount of between 0.2% and 5.0% (v/v) of the total volume of the immunogenic composition, more preferably at an amount of 2.5% (v/v) in a 0.5 ml vaccine dose volume, or 0.5% (v/v) in 0.5 ml vaccine dose volume or 1.7-1.9% (v/v), preferably 1.8% in 0.7 ml vaccine dose volume. By way of clarification, concentrations given in v/v can be converted into concentration in w/v by applying the following conversion factor: a 5% (v/v) alpha-tocopherol concentration is equivalent to a 4.8% (w/v) alpha-tocopherol concentration.

The oil in water emulsion may further comprise an emulsifying agent. The emulsifying agent may be present at an amount of 0.01 to 5.0% by weight of the immunogenic composition (w/w), preferably present at an amount of 0.1 to 2.0% by weight (w/w). Preferred concentration are 0.5 to 1.5% by weight (w/w) of the total composition.

The emulsifying agent may suitably be polyoxyethylene sorbitan monooleate (Tween 80). In a specific embodiment, a 0.5 ml vaccine dose volume contains 1% (w/w) Tween 80, and a 0.7 ml vaccine dose volume contains 0.7% (w/w) Tween 80. In another specific embodiment the concentration of Tween 80 is 0.2% (w/w).

The oil in water emulsion adjuvant may be utilised with other adjuvants or immuno-stimulants and therefore an important embodiment of the invention is an oil in water formulation comprising squalene or another metabolizable oil, alpha tocopherol, and tween 80. The oil in water emulsion may also contain span 85 and/or Lecithin. Typically the oil in water will comprise from 2 to 10% squalene of the total volume of the immunogenic composition, from 2 to 10% alpha tocopherol and from 0.3 to 3% Tween 80, and may be produced according to the procedure described in WO 95/17210. Preferably the ratio of squalene: alpha tocopherol is equal or less than 1 as this provides a more stable emulsion. Span 85 (polyoxyethylene sorbitan trioleate) may also be present, for example at a level of 1%.

### Immunogenic Properties of the Immunogenic Composition Used for the First Vaccination of the Present Invention

In the present invention the influenza composition is capable of inducing an improved CD4 T-cell immune response against at least one of the component antigen(s) or antigenic composition compared to the CD4 T-cell immune response obtained with the corresponding composition which in un-adjuvanted, i.e. does not contain any exogeneous adjuvant (herein also referred to as ‘plain composition’).

By ‘improved CD4 T-cell immune response is meant that a higher CD4 response is obtained in a human patient after administration of the adjuvanted immunogenic composition than that obtained after administration of the same composition without adjuvant. For example, a higher CD4 T-cell response is obtained in a human patient upon administration of an immunogenic composition comprising an influenza virus or antigenic preparation thereof together with an oil-in-water emulsion adjuvant comprising a metabolizable oil, a sterol such as alpha tocopherol and an emulsifying agent, compared to the response induced after administration of an immunogenic composition comprising an influenza virus or antigenic preparation thereof which is un-adjuvanted. Such formulation will advantageously be used to induce anti-influenza CD4−T cell response capable of detection of influenza epitopes presented by MHC class II molecules.

Preferably said immunological response induced by an adjuvanted split influenza composition for use in the present invention is higher than the immunological response induced by any other un-adjuvanted influenza conventional vaccine, such as sub-unit influenza vaccine or whole influenza virus vaccine.

In particular but not exclusively, said ‘improved CD4 T-cell immune response’ is obtained in an immunologically unprimed patient, i.e. a patient who is seronegative to said influenza virus or antigen. This seronegativity may be the result of said patient having never faced such virus or antigen (so-called ‘naive’ patient) or, alternatively, having failed to respond to said antigen once encountered. Preferably said improved CD4 T-cell immune response is obtained in an immunocompromised subject such as an elderly, typically 65 years of age or above, or an adult younger than 65 years of age with a high risk medical condition (‘high risk’ adult), or a child under the age of two.

The improved CD4 T-cell immune response may be assessed by measuring the number of cells producing any of the following cytokines:
cells producing at least two different cytokines (CD40L, IL-2, IFNγ, TNFα)
cells producing at least CD40L and another cytokine (IL-2, TNFα, IFNγ)
cells producing at least IL-2 and another cytokine (CD40L, TNFα, IFNγ)
cells producing at least IFNγ and another cytokine (IL-2, TNFα, CD40L)
cells producing at least TNFα and another cytokine (IL-2, CD40L, IFNγ)

There will be improved CD4 T-cell immune response when cells producing any of the above cytokines will be in a higher amount following administration of the adjuvanted composition compared to the administration of the un-adjuvanted composition. Typically at least one, preferably two of the five conditions mentioned herein above will be fulfilled. In a particular embodiment, the cells producing all four cytokines will be present at a higher amount in the adjuvanted group compared to the un-adjuvanted group.

The improved CD4 T-cell immune response conferred by the adjuvanted influenza composition of the present invention may be ideally obtained after one single administration. The single dose approach will be extremely relevant for example in a rapidly evolving outbreak situation. In certain circumstances, especially for the elderly population, or in the case of young children (below 9 years of age) who are vaccinated for the first time against influenza, it may be beneficial to administer two doses of the same composition for that season. The second dose of said same composition (still considered as ‘composition for first vaccination’) may be administered during the on-going primary immune response and is adequately spaced. Typically the second dose of the composition is given a few weeks, or about one month, e.g. 2 weeks, 3 weeks, 4 weeks, 5 weeks, or 6 weeks after the first dose, to help prime the immune system in unresponsive or poorly responsive individuals.

In a specific embodiment, the administration of said immunogenic composition alternatively or additionally induces an improved B-memory cell response in patients administered with the adjuvanted immunogenic composition compared to the B-memory cell response induced in individuals immunized with the un-adjuvanted composition. An improved B-memory cell response is intended to mean an increased frequency of peripheral blood B lymphocytes capable of differentiation into antibody-secreting plasma cells upon antigen encounter as measured by stimulation of in-vitro differentiation (see Example sections, e.g. methods of Elispot B cells memory).

In a still further specific embodiment, the vaccination with the composition for the first vaccination, adjuvanted, has no measurable impact on the CD8 response.

The Applicants have surprisingly found that a composition comprising an influenza virus or antigenic preparation thereof formulated with an oil-in-water emulsion adjuvant comprising a metabolizable oil, a sterol such as alpha tocopherol and an emulsifying agent, is effective in promoting T cell responses in an immuno-compromised human population. As the Applicants have demonstrated, the administration of a single dose of the immunogenic composition for first vaccination, as described in the invention is capable of providing better sero-protection, as assessed by the correlates of protection for influenza vaccines, following revaccination against influenza in a human elderly population, than does the vaccination with an un-adjuvanted influenza vaccine. The claimed adjuvanted formulation has also been able to induce an improved CD4 T-cell immune response against influenza virus compared to that obtained with the un-adjuvanted formulation. This finding can be associated with an increased responsiveness upon vaccination or infection vis-à-vis influenza antigenic exposure. Furthermore, this may also be associated with a cross-responsiveness, i.e. a higher ability to respond against variant influenza strains. This improved response may be especially beneficial in an immuno-compromised human population such as the elderly population (65 years of age and above) and in particular the high risk elderly population. This may result in reducing the overall morbidity and mortality rate and preventing emergency admissions to hospital for pneumonia and other influenza-like illness. This may also be of benefit to the infant population (below 5 years, preferably below 2 years of age). Furthermore it allows inducing a CD4 T cell response which is more persistent in time, e.g. still present one year after the first vaccination, compared to the response induced with the un-adjuvanted formulation.

Preferably the CD4 T-cell immune response, such as the improved CD4 T-cell immune response obtained in an unprimed subject, involves the induction of a cross-reactive CD4 T helper response. In particular, the amount of cross-reactive CD4 T cells is increased. By ‘cross-reactive’ CD4 response is meant CD4 T-cell targeting shared epitopes between influenza strains.

Usually, available influenza vaccines are effective only against infecting strains of influenza virus that have haemagglutinins of similar antigenic characteristics. When the infecting (circulating) influenza virus has undergone minor changes (such as a point mutation or an accumulation of point mutations resulting in amino acid changes in the for example) in the surface glycoproteins in particular haemagglutinin (antigenic drift variant virus strain) the vaccine may still provide some protection, although it may only provide limited protection as the newly created variants may escape immunity induced by prior influenza infection or vaccination. Antigenic drift is responsible for annual epidemics that occur during interpandemic periods (Wiley & Skehel, 1987, Ann. Rev. Biochem. 56, 365-394). The induction of cross-reactive CD4 T cells provides an additional advantage to the composition of the invention, in that it may provide also cross-protection, in other words protection against heterologous infections, i.e. infections caused by a circulating influenza strain which is a variant (e.g. a drift) of the influenza strain contained in the immunogenic composition. This may be advantageous when the circulating strain is difficult to propagate in eggs or to produce in tissue culture, rendering the use of a drifted strain a working alternative. This may also be advantageous when the subject received a first and a second vaccination several months or a year apart, and the influenza strain in the immunogenic composition used for a second immunization is a drift variant strain of the strain used in the composition used for the first vaccination.

Detection of Cross-Reactive CD4 T-Cells Following Vaccination with Influenza Vaccine

Following classical trivalent Influenza vaccine administration (3 weeks), there is a substantial increase in the frequency of peripheral blood CD4 T-cells responding to antigenic strain preparation (whole virus or split antigen) that is homologous to the one present in the vaccine (H3N2: A/Panama/2007/99, H1N1: A/New Calcdonia/20/99, B: B/Shangdong/7/97) (see Example III). A comparable increase in frequency can be seen if peripheral blood CD4 T-cells are restimulated with influenza strains classified as drifted strains (H3N2: A/Sydney/5/97, H1N1: A/Beijing/262/95, B: B/Yamanashi/166/98).

In contrast, if peripheral blood CD4 T-cells are restimulated with influenza strains classified as shift strains (H2N2: A/Singapore/1/57, H9N2: A/Hongkong/1073/99) by expert in the field, there is no observable increase following vaccination.

CD4 T-cells that are able to recognize both homologous and drifted Influenza strains have been named in the present document “cross-reactive”. The adjuvanted influenza compositions as claimed for use in the present invention have been capable to show heterosubtypic cross-reactivity since there is observable cross-reactivity against drifted Influenza strains.

Consistently with the above observations, CD4 T-cell epitopes shared by different Influenza strains have been identified in human (Gelder C et al. 1998, Int Immunol. 10(2):211-22; Gelder C M et al. 1996 J Virol. 70(7):4787-90; Gelder C M et al. 1995 J Virol. 1995 69(12):7497-506).

In a specific embodiment, the adjuvanted composition may offer the additional benefit of providing better protection against circulating strains which have undergone a major change (such as gene recombination for example, between two different species) in the haemagglutinin (antigenic shift) against which currently available vaccines have no efficacy.

### Other Adjuvants

The composition may comprise an additional adjuvant, in particular a TRL-4 ligand adjuvant, suitably a non-toxic derivative of lipid A. A suitable TRL-4 ligand is 3 de-O-acylated monophosphoryl lipid A (3D-MPL). Other suitable TLR-4 ligands are lipopolysaccharide (LPS) and derivatives, MDP (muramyl dipeptide) and F protein of RSV.

In one embodiment the composition may additionally include a Toll like receptor (TLR) 4 ligand, such as a non-toxic derivative of lipid A, particularly monophosphoryl lipid A or more particularly 3-Deacylated monophoshoryl lipid A (3D-MPL).

3 D-MPL is sold under the trademark MPL® by Corixa corporation (herein MPL) and primarily promotes CD4+ T cell responses with an IFNγ (Th1) phenotype. It can be produced according to the methods disclosed in GB 2 220 211 A. Chemically it is a mixture of 3-deacylated monophosphoryl lipid A with 3, 4, 5 or 6 acylated chains. Preferably in the compositions of the present invention small particle 3 D-MPL is used. Small particle 3D-MPL has a particle size such that it may be sterile-filtered through a 0.22 μm filter. Such preparations are described in WO94/21292 and in Example II.

3D-MPL can be used, for example, at an amount of 1 to 100 μg (w/v) per composition dose, preferably in an amount of 10 to 50 μg (w/v) per composition dose. A suitable amount of 3D-MPL is for example any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μg (w/v) per composition dose. More preferably, 3D-MPL amount ranges from 25 to 75 μg (w/v) per composition dose. Usually a composition dose will be ranging from about 0.5 ml to about 1 ml. A typical vaccine dose are 0.5 ml, 0.6 ml, 0.7 ml, 0.8 ml, 0.9 ml or 1 ml. In a preferred embodiment, a final concentration of 50 μg of 3D-MPL is contained per ml of vaccine composition, or 25 μg per 0.5 ml vaccine dose. In other preferred embodiments, a final concentration of 35.7 μg or 71.4 μg of 3D-MPL is contained per ml of vaccine composition. Specifically, a 0.5 ml vaccine dose volume contains 25 μg or 50 μg of 3D-MPL per dose.

The dose of MPL is suitably able to enhance an immune response to an antigen in a human. In particular a suitable MPL amount is that which improves the immunological potential of the composition compared to the unadjuvanted composition, or compared to the composition adjuvanted with another MPL amount, whilst being acceptable from a reactogenicity profile.

Synthetic derivatives of lipid A are known, some being described as TLR-4 agonists, and include, but are not limited to:
OM174 (2-deoxy-6-o-[2-deoxy-2-[(R)-3-dodecanoyloxytetra-decanoylamino]-4-O-phosphono-β-D-glucopyranosyl]-2-[(R)-3-hydroxytetradecanoylamino]-α-D-glucopyranosyldihydrogenphosphate), (WO 95/14026)
OM 294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO99/64301 and WO 00/0462)
OM 197 MP-Ac DP (3S-,9R)-3-[(R)-dodecanoyloxytetradecanoylamino]-4-oxo-5-aza-9-[(R)-3-hydroxytetradecanoylamino]decan-1,10-diol, 1-dihydrogenophosphate 10-(6-aminohexanoate) (WO 01/46127)

Other suitable TLR-4 ligands are, for example, lipopolysaccharide and its derivatives, muramyl dipeptide (MDP) or F protein of respiratory syncitial virus.

Another suitable immunostimulant for use in the present invention is Quil A and its derivatives. Quil A is a saponin preparation isolated from the South American tree Quilaja Saponaria Molina and was first described by Dalsgaard et al. in 1974 (“Saponin adjuvants”, Archiv. für die gesamte Virusforschung, Vol. 44, Springer Verlag, Berlin, p243-254) to have adjuvant activity. Purified fragments of Quil A have been isolated by HPLC which retain adjuvant activity without the toxicity associated with Quil A (EP 0 362 278), for example QS7 and QS21 (also known as QA7 and QA21). QS-21 is a natural saponin derived from the bark of Quillaja saponaria Molina, which induces CD8+ cytotoxic T cells (CTLs), Th1 cells and a predominant IgG2a antibody response and is a preferred saponin in the context of the present invention.

Particular formulations of QS21 have been described which are particularly preferred, these formulations further comprise a sterol (WO96/33739). The saponins forming part of the present invention may be in the form of an oil in water emulsion (WO 95/17210).

### Revaccination and Composition Used for Revaccination (Boosting Composition)

An aspect of the present invention provides the use of an influenza antigen in the manufacture of an influenza immunogenic composition for revaccination of humans previously vaccinated with an influenza virus or antigenic preparation thereof or variant thereof formulated with an oil-in-water emulsion adjuvant comprising a metabolizable oil, a sterol such as alpha tocopherol and an emulsifying agent.

Typically revaccination is made at least 6 months after the first vaccination(s), preferably 8 to 14 months after, more preferably at around 10 to 12 months after.

The immunogenic composition for revaccination (the boosting composition) may contain any type of antigen preparation, either inactivated or live attenuated. It may contain the same type of antigen preparation i.e. split influenza virus or split influenza virus antigenic preparation thereof, a whole virion, a purified HA and NA (sub-unit) vaccine or a virosome, as the immunogenic composition used for the first vaccination. Alternatively the boosting composition may contain another type of influenza antigen, i.e. split influenza virus or split influenza virus antigenic preparation thereof, a whole virion, a purified HA and NA (sub-unit) vaccine or a virosome, than that used for the first vaccination. The boosting composition may be adjuvanted or un-adjuvanted. The un-adjuvanted boosting composition may be Fluarix™/α-Rix®/Influsplit® given intramuscularly. The formulation contains three inactivated split virion antigens prepared from the WHO recommended strains of the appropriate influenza season.

Accordingly, in a preferred embodiment, the invention provides for the use of:
(a) an influenza virus or antigenic preparation thereof, and
(b) an oil-in-water emulsion adjuvant
   in the manufacture of an immunogenic composition for revaccination of humans previously vaccinated with an influenza virus or antigenic preparation thereof and an oil-in-water emulsion adjuvant comprising a metabolizable oil, a sterol and an emulsifying agent. Said oil-in-water emulsion adjuvant preferably comprises at least one metabolizable oil in an amount of 0.5% to 20% of the total volume, and has oil droplets of which at least 70% by intensity have diameters of less than 1 μm. Suitably said sterol is alpha tocopherol.

In a specific embodiment, the immunogenic composition for revaccination (also called herein below the ‘boosting composition’) contains an influenza virus or antigenic preparation thereof which shares common CD4 T-cell epitopes with the influenza virus or antigenic preparation thereof used for the first vaccination. A common CD4 T cell epitope is intended to mean peptides/sequences/epitopes from different antigens which can be recognised by the same CD4 cell (see examples of described epitopes in: Gelder C et al. 1998, Int Immunol. 10(2):211-22; Gelder C M et al. 1996 J Virol. 70(7):4787-90; Gelder C M et al. 1995 J Virol. 1995 69(12):7497-506).

In a preferred embodiment, the influenza strain may be associated with a pandemic outbreak or have the potential to be associated with a pandemic outbreak. In particular, when the vaccine is a multivalent vaccine such as a bivalent or a trivalent vaccine, at least one strain is associated with a pandemic outbreak or has the potential to be associated with a pandemic outbreak. Suitable strains are, but not limited to: H5N1, H9N2, H7N7, H2N2 and H1N1.

In another aspect of the present invention, there is provided the use of:
(c) an influenza virus or antigenic preparation thereof, from a first influenza strain, and
(d) an oil-in-water emulsion adjuvant comprising a metabolizable oil, a sterol and an emulsifying agent
   in the manufacture of an immunogenic composition for protection against influenza infections caused by a influenza strain which is a variant of said first influenza strain. Preferably said oil-in-water emulsion adjuvant comprises at least one metabolizable oil in an amount of 0.5% to 20% of the total volume, and has oil droplets of which at least 70% by intensity have diameters of less than 1 μm. Suitably said sterol is alpha-tocopherol.

Typically a boosting composition, where used, is given at the next influenza season, e.g. approximately one year after the first immunogenic composition. The boosting composition may also be given every subsequent year (third, fourth, fifth vaccination and so forth). The boosting composition may be the same as the composition used for the first vaccination. Suitably, the boosting composition contains an influenza virus or antigenic preparation thereof which is a variant strain of the influenza virus used for the first vaccination. In particular, the influenza viral strains or antigenic preparation thereof are selected according to the reference material distributed by the World Health Organisation such that they are adapted to the influenza strain which is circulating on the year of the revaccination.

The influenza antigen or antigenic composition used in revaccination preferably comprises an adjuvant or an oil-in-water emulsion, suitably as described above. The adjuvant may be an oil-in-water emulsion adjuvant as herein above described, which is preferred, optionally containing an additional adjuvant such as TLR-4 ligand such as 3D-MPL or a saponin, or may be another suitable adjuvant such as alum or alum alternatives such as polyphosphazene for example.

Preferably revaccination induces any, preferably two or all, of the following: (i) an improved CD4 response against the influenza virus or antigenic preparation thereof, or (ii) an improved B cell memory response or (iii) an improved humoral response, compared to the equivalent response induced after a first vaccination with the un-adjuvanted influenza virus or antigenic preparation thereof. Preferably the immunological responses induced after revaccination with the adjuvanted influenza virus or antigenic preparation thereof as herein defined, are higher than the corresponding response induced after the revaccination with the un-adjuvanted composition. Preferably the immunological responses induced after revaccination with an un-adjuvanted, preferably split, influenza virus are higher in the population first vaccinated with the adjuvanted, preferably split, influenza composition than the corresponding response in the population first vaccinated with the un-adjuvanted, preferably split, influenza composition.

As the Applicants have demonstrated, the revaccination of the subjects with a boosting composition comprising an influenza virus and an oil-in-water emulsion adjuvant comprising a metabolizable oil, a sterol such as alpha tocopherol and an emulsifying agent, as defined herein above, shows higher antibody titers than the corresponding values in the group of people first vaccinated with the un-adjuvanted composition and boosted with the un-adjuvanted composition. The effect of the adjuvant in enhancing the antibody response to revaccination is especially of importance in the elderly population which is known to have a low response to vaccination or infection by influenza virus. The adjuvanted composition-associated benefit was also marked in terms of improving the CD4 T-cell response following revaccination.

The adjuvanted composition of the invention is capable of inducing a better cross-responsiveness against drifted strain (the influenza strain from the next influenza season) compared to the protection conferred by the control vaccine. Said cross-responsiveness has shown a higher persistence compared to that obtained with the un-adjuvanted formulation.

Preclinical data given in Example 3 for example show the ability of the composition of the invention to protect against heterotypic influenza infection and disease as assessed by body temperature readouts. The same conclusion holds true for the clinical trials data obtained in revaccination studies.

### Influenza Viral Strains and Antigens Thereof.

Said influenza virus or antigenic preparation thereof is suitably monovalent or multivalent such as bivalent or trivalent or quadrivalent. Preferably the influenza virus or antigenic preparation thereof is trivalent or quadrivalent, having an antigen from three different influenza strains.

Optionally at least one strain is associated with a pandemic outbreak or has the potential to be associated with a pandemic outbreak.

By way of background, during inter-pandemic periods, influenza viruses circulate that are related to those from the preceding epidemic. The viruses spread among people with varying levels of immunity from infections earlier in life. Such circulation, over a period of usually 2-3 years, promotes the selection of new strains that have changed enough to cause an epidemic again among the general population; this process is termed ‘antigenic drift’. ‘Drift variants’ may have different impacts in different communities, regions, countries or continents in any one year, although over several years their overall impact is often similar. In other words, an influenza pandemics occurs when a new influenza virus appears against which the human population has no immunity. Typical influenza epidemics cause increases in incidence of pneumonia and lower respiratory disease as witnessed by increased rates of hospitalisation or mortality. The elderly or those with underlying chronic diseases are most likely to experience such complications, but young infants also may suffer severe disease.

At unpredictable intervals, novel influenza viruses emerge with a key surface antigen, the haemagglutinin, of a totally different subtype from strains circulating the season before. Here, the resulting antigens can vary from 20% to 50% from the corresponding protein of strains that were previously circulating in humans. This can result in virus escaping ‘herd immunity’ and establishing pandemics. This phenomenon is called ‘antigenic shift’. It is thought that at least in the past pandemics have occurred when an influenza virus from a different species, such as an avian or a porcine influenza virus, has crossed the species barrier. If such viruses have the potential to spread from person to person, they may spread worldwide within a few months to a year, resulting in a pandemic. For example, in 1957 (Asian Flu pandemic), viruses of the H2N2 subtype replaced H1N1 viruses that had been circulating in the human population since at least 1918 when the virus was first isolated. The H2 HA and N2 NA underwent antigenic drift between 1957 and 1968 until the HA was replaced in 1968 (Hong-Kong Flu pandemic) by the emergence of the H3N2 influenza subtype, after which the N2 NA continued to drift along with the H3 HA (Nakajima et al., 1991, Epidemiol. Infect. 106, 383-395).

The features of an influenza virus strain that give it the potential to cause a pandemic outbreak are: it contains a new haemagglutinin compared to the haemagglutinin in the currently circulating strains, which may or not be accompanied by a change in neuraminidase subtype; it is capable of being transmitted horizontally in the human population; and it is pathogenic for humans. A new haemagglutinin may be one which has not been evident in the human population for an extended period of time, probably a number of decades, such as H2. Or it may be a haemagglutinin that has not been circulating in the human population before, for example H5, H9, H7 or H6 which are found in birds. In either case the majority, or at least a large proportion of, or even the entire population has not previously encountered the antigen and is immunologically naïve to it.

Certain parties are generally at an increased risk of becoming infected with influenza in a pandemic situation. The elderly, the chronically ill and small children are particularly susceptible but many young and apparently healthy people are also at risk. For H2 influenza, the part of the population born after 1968 is at an increased risk. It is important for these groups to be protected effectively as soon as possible and in a simple way.

Another group of people who are at increased risk are travelers. People travel more today than ever before and the regions where most new viruses emerge, China and South East Asia, have become popular travel destinations in recent years. This change in travel patterns enables new viruses to reach around the globe in a matter of weeks rather than months or years.

Thus for these groups of people there is a particular need for vaccination to protect against influenza in a pandemic situation or a potential pandemic situation. Suitable strains are, but not limited to: H5N1, H9N2, H7N7, H2N2 and H1N1.

Optionally the composition may contain more than three valencies, for example two non pandemic strains plus a pandemic strain. Alternatively the composition may contain three pandemic strains.

In a further embodiment the invention relates to a vaccination regime in which the first vaccination is made with a split influenza composition containing at least one influenza strain that could potentially cause a pandemic outbreak and the revaccination is made with a circulating strain, either a pandemic strain or a classical strain.

### CD4 Epitope in HA

This antigenic drift mainly resides in epitope regions of the viral surface proteins haemagglutinin (HA) and neuraminidase (NA). It is known that any difference in CD4 and B cell epitopes between different influenza strains, being used by the virus to evade the adaptive response of the host immune system, will play a major role in influenza vaccination and is.

CD4 T-cell epitopes shared by different Influenza strains have been identified in human (see for example: Gelder C et al. 1998, Int Immunol. 10(2):211-22; Gelder C M et al. 1996 J Virol. 70(7):4787-90; and Gelder C M et al. 1995 J Virol. 1995 69(12):7497-506).

In a specific embodiment, the revaccination is made by using a boosting composition which contains an influenza virus or antigenic preparation thereof which shares common CD4 T-cell epitopes with the influenza virus antigen or antigenic preparation thereof used for the first vaccination. The invention thus relates to the use of the immunogenic composition comprising a influenza virus or antigenic preparation thereof and an oil-in-water emulsion adjuvant comprising a metabolizable oil, a sterol such as alpha tocopherol and an emulsifying agent, in the manufacture of a first vaccination-component of a multi-dose vaccine, the multi-dose vaccine further comprising, as a boosting dose, an influenza virus or antigenic preparation thereof which shares common CD4 T-cell epitopes with the influenza virus antigen or virus antigenic preparation thereof of the dose given at the first vaccination.

### Vaccination Means

The composition of the invention may be administered by any suitable delivery route, such as intradermal, mucosal e.g. intranasal, oral, intramuscular or subcutaneous. Other delivery routes are well known in the art.

The intramuscular delivery route is preferred for the adjuvanted influenza composition.

Intradermal delivery is another suitable route. Any suitable device may be used for intradermal delivery, for example short needle devices such as those described in U.S. Pat. No. 4,886,499, U.S. Pat. No. 5,190,521, U.S. Pat. No. 5,328,483, U.S. Pat. No. 5,527,288, U.S. Pat. No. 4,270,537, U.S. Pat. No. 5,015,235, U.S. Pat. No. 5,141,496, U.S. Pat. No. 5,417,662. Intradermal vaccines may also be administered by devices which limit the effective penetration length of a needle into the skin, such as those described in WO99/34850 and EP1092444, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Pat. No. 5,480,381, U.S. Pat. No. 5,599,302, U.S. Pat. No. 5,334,144, U.S. Pat. No. 5,993,412, U.S. Pat. No. 5,649,912, U.S. Pat. No. 5,569,189, U.S. Pat. No. 5,704,911, U.S. Pat. No. 5,383,851, U.S. Pat. No. 5,893,397, U.S. Pat. No. 5,466,220, U.S. Pat. No. 5,339,163, U.S. Pat. No. 5,312,335, U.S. Pat. No. 5,503,627, U.S. Pat. No. 5,064,413, U.S. Pat. No. 5,520,639, U.S. Pat. No. 4,596,556 U.S. Pat. No. 4,790,824, U.S. Pat. No. 4,941,880, U.S. Pat. No. 4,940,460, WO 97/37705 and WO 97/13537. Also suitable are ballistic powder/particle delivery devices which use compressed gas to accelerate vaccine in powder form through the outer layers of the skin to the dermis. Additionally, conventional syringes may be used in the classical mantoux method of intradermal administration.

Another suitable administration route is the subcutaneous route. Any suitable device may be used for subcutaneous delivery, for example classical needle. Preferably, a needle-free jet injector service is used, such as that published in WO 01/05453, WO 01/05452, WO 01/05451, WO 01/32243, WO 01/41840, WO 01/41839, WO 01/47585, WO 01/56637, WO 01/58512, WO 01/64269, WO 01/78810, WO 01/91835, WO 01/97884, WO 02/09796, WO 02/34317. More preferably said device is pre-filled with the liquid vaccine formulation.

Alternatively the vaccine is administered intranasally. Typically, the vaccine is administered locally to the nasopharyngeal area, preferably without being inhaled into the lungs. It is desirable to use an intranasal delivery device which delivers the vaccine formulation to the nasopharyngeal area, without or substantially without it entering the lungs.

Preferred devices for intranasal administration of the vaccines according to the invention are spray devices. Suitable commercially available nasal spray devices include Accuspray™ (Becton Dickinson). Nebulisers produce a very fine spray which can be easily inhaled into the lungs and therefore does not efficiently reach the nasal mucosa. Nebulisers are therefore not preferred.

Preferred spray devices for intranasal use are devices for which the performance of the device is not dependent upon the pressure applied by the user. These devices are known as pressure threshold devices. Liquid is released from the nozzle only when a threshold pressure is applied. These devices make it easier to achieve a spray with a regular droplet size. Pressure threshold devices suitable for use with the present invention are known in the art and are described for example in WO 91/13281 and EP 311 863 B and EP 516 636, incorporated herein by reference. Such devices are commercially available from Pfeiffer GmbH and are also described in Bommer, R. Pharmaceutical Technology Europe, September 1999.

Preferred intranasal devices produce droplets (measured using water as the liquid) in the range 1 to 200 μm, preferably 10 to 120 μm. Below 10 μm there is a risk of inhalation, therefore it is desirable to have no more than about 5% of droplets below 10 μm. Droplets above 120 μm do not spread as well as smaller droplets, so it is desirable to have no more than about 5% of droplets exceeding 120 μm.

Bi-dose delivery is a further preferred feature of an intranasal delivery system for use with the vaccines according to the invention. Bi-dose devices contain two sub-doses of a single vaccine dose, one sub-dose for administration to each nostril. Generally, the two sub-doses are present in a single chamber and the construction of the device allows the efficient delivery of a single sub-dose at a time. Alternatively, a monodose device may be used for administering the vaccines according to the invention.

Alternatively, the epidermal or transdermal vaccination route is also contempletd in the present invention.

In a specific aspect of the present invention, the adjuvanted immunogenic composition for the first administration may be given intramuscularly, and the boosting composition, either adjuvanted or not, may be administered through a different route, for example intradermal, subcutaneous or intranasal. In another specific embodiment, the composition for the first administration may contain a standard HA content of 15 μg per influenza strain, and the boosting composition may contain a low dose of HA, i.e. below 15 μg, and depending on the administration route, may be given in a smaller volume.

### Populations to Vaccinate

The target population to vaccinate may be immuno-compromised human. Immuno-compromised humans generally are less well able to respond to an antigen, in particular to an influenza antigen, in comparison to healthy adults.

Preferably the target population is a population which is unprimed against influenza, either being naïve (such as vis à vis a pandemic strain), or having failed to respond previously to influenza infection or vaccination. Preferably the target population is elderly persons suitably aged 65 years and over, younger high-risk adults (i.e. between 18 and 64 years of age) such as people working in health institutions, or those young adults with a risk factor such as cardiovascular and pulmonary disease, or diabetes. Another target population is all children 6 months of age and over, especially children 6-23 months of age who experience a relatively high influenza-related hospitalization rate. Preferably the target population is elderly above 65 years of age.

### Vaccination Regimes, Dosing and Additional Efficacy Criteria

Suitably the immunogenic compositions according to the present invention are a standard 0.5 ml injectable dose in most cases, and contains 15 μg of haemagglutinin antigen component from the or each influenza strain, as measured by single radial immunodiffusion (SRD) (J. M. Wood et al.: J. Biol. Stand. 5 (1977) 237-247; J. M. Wood et al., J. Biol. Stand. 9 (1981) 317-330). Suitably the vaccine dose volume will be between 0.5 ml and 1 ml, in particular a standard 0.5 ml, or 0.7 ml vaccine dose volume. Slight adaptation of the dose volume will be made routinely depending on the HA concentration in the original bulk sample.

Suitably said immunogenic composition contains a low dose of HA antigen—e.g any of 1, 2, 3, 4, 5, 6, 7, 8, 9, 10, 11, 12, 13 or 14 μg of HA per influenza strain. A suitable low dose of HA is between 1 to 7.5 μg of HA per influenza strain, suitably between 3.5 to 5 μg such as 3.75 μg of HA per influenza strain, typically about 5 μg of HA per influenza strain.

Advantageously, a vaccine dose according to the invention, in particular a low dose vaccine, may be provided in a smaller volume than the conventional injected split flu vaccines, which are generally around 0.5, 0.7 or 1 ml per dose. The low volume doses according to the invention are preferably below 500 μl, more preferably below 300 μl and most preferably not more than about 200 μl or less per dose.

Thus, a preferred low volume vaccine dose according to one aspect of the invention is a dose with a low antigen dose in a low volume, e.g. about 15 μg or about 7.5 μg HA or about 3.0 μg HA (per strain) in a volume of about 200 μl.

The influenza medicament of the invention preferably meets certain international criteria for vaccines.

Standards are applied internationally to measure the efficacy of influenza vaccines. The European Union official criteria for an effective vaccine against influenza are set out in the Table 1 below. Theoretically, to meet the European Union requirements, an influenza vaccine has to meet only one of the criteria in the table, for all strains of influenza included in the vaccine. The compositions of the present invention suitably meet at least one such criteria.

However in practice, at least two or all three of the criteria will need to be met for all strains, particularly for a new vaccine such as a new vaccine for delivery via a different route. Under some circumstances two criteria may be sufficient. For example, it may be acceptable for two of the three criteria to be met by all strains while the third criterion is met by some but not all strains (e.g. two out of three strains). The requirements are different for adult populations (18-60 years) and elderly populations (>60 years).

TABLE 1

| | 18-60 years | >60 years |
|---|---|---|
| Seroconversion rate* | >40% | >30% |
| Conversion factor** | >2.5 | >2.0 |
| Protection rate*** | >70% | >60% |

*Seroconversion rate is defined as the percentage of vaccinees who have at least a 4-fold increase in serum haemagglutinin inhibition (HI) titres after vaccination, for each vaccine strain.
**Conversion factor is defined as the fold increase in serum HI geometric mean titres (GMTs) after vaccination, for each vaccine strain.
***Protection rate is defined as the percentage of vaccinees with a serum HI titre equal to or greater than 1:40 after vaccination (for each vaccine strain) and is normally accepted as indicating protection.

In a further aspect the invention provides a method of designing a vaccine for diseases known to be cured or treated through a CD4+ T cell activation, comprising
1) selecting an antigen containing CD4+ epitopes, and
2) combining said antigen with an oil-in-water emulsion adjuvant as defined herein above, wherein said vaccine upon administration in said mammal is capable of inducing an enhanced CD4 T cell response in said mammal.

The teaching of all references in the present application, including patent applications and granted patents, are herein fully incorporated by reference.

For the avoidance of doubt the terms ‘comprising’, ‘comprise’ and ‘comprises’ herein is intended by the inventors to be optionally substitutable with the terms ‘consisting of’, ‘consist of’, and ‘consists of’, respectively, in every instance.

### ALTERNATIVE EMBODIMENTS

In an alternative embodiment, any oil-in-water emulsion adjuvant may be used, in particular but not exclusively when used with a split influenza antigen or antigenic preparation thereof. Accordingly, the following specific embodiments are also contemplated I the context of the present invention:

The use of:
(a) a split influenza virus or split virus antigenic preparation thereof, and
(b) an oil-in-water emulsion adjuvant
   in the manufacture of an immunogenic composition for inducing at least one of i) an improved CD4 T-cell response, ii) an improved B cell memory response, against said antigen or split virus antigenic preparation thereof in a human.

The use of:
(c) a split influenza virus or split virus antigenic preparation thereof, and
(a) an oil-in-water emulsion adjuvant
   in the manufacture of an immunogenic composition for vaccination of a human immuno-compromised individual or population, such as a high risk adult or a elderly, against influenza.

The use of an influenza virus or antigenic preparation thereof, either adjuvanted or un-adjuvanted, in the manufacture of an immunogenic composition for revaccination of humans previously vaccinated with split influenza virus or split virus antigenic preparation thereof and an oil-in-water emulsion adjuvant. Preferably the revaccination is made in subjects who have been vaccinated the previous season against influenza. Typically revaccination is made at least 6 months after the first vaccination, preferably 8 to 14 months after, more preferably at around 10 to 12 months after.

Preferably, there is provided the use of:
a split influenza virus or split virus antigenic preparation thereof, and
an oil-in-water emulsion adjuvant
   in the manufacture of an immunogenic composition for revaccination of humans previously vaccinated with split influenza virus or split virus antigenic preparation thereof and an oil-in-water emulsion adjuvant.

The use of:
a split influenza virus or split virus antigenic preparation thereof, from a first influenza strain, and
an oil-in-water emulsion adjuvant
   in the manufacture of an immunogenic composition for protection against influenza infections caused by a influenza strain which is a variant of said first influenza strain.

In another specific embodiment, the immunogenic composition for revaccination contains a split influenza virus or split virus antigenic preparation thereof which shares common CD4 T-cell epitopes with the split influenza virus or split virus antigenic preparation thereof used for the first vaccination.

A method of vaccination an immunocompromised human individual or population such as high risk adults or elderly, with an immunogenic composition comprising a split influenza virus or split virus antigenic preparation thereof and an oil-in-water emulsion adjuvant, as hereinabove defined.

A method for revaccinating humans previously vaccinated with split influenza virus or split influenza virus antigenic preparation thereof and an oil-in-water emulsion adjuvant, comprising administering to said human an immunogenic composition comprising an influenza virus, either adjuvanted or un-adjuvanted.

A method for vaccinating a human population or individual against one influenza virus strain followed by revaccination of said human or population against a variant influenza virus strain, said method comprising administering to said human (i) a first composition comprising a split influenza virus or split influenza virus antigenic preparation thereof from a first influenza virus strain and an oil-in-water emulsion adjuvant, and (ii) a second immunogenic composition comprising a influenza virus strain variant of said first influenza virus strain.

A method of designing an influenza vaccine, comprising
1) selecting an influenza antigen containing CD4+ epitopes, and
2) combining said influenza antigen with an oil-in-water emulsion as defined above, wherein said vaccine upon administration in a mammal is capable of inducing an enhanced CD4 response in said mammal.

A method for preventing the impairment of the immune response against influenza virus to a booster administration of an influenza virus vaccine in human subjects, comprising the steps of (i) administering to said subject a first influenza vaccine in combination with an adjuvant, and (ii) administering to said subject a further booster of a influenza virus vaccine. For example, preventing impairment can be measured as an increased boost response relative to a boost response in subjects having received a first non-adjuvanted vaccine, e.g., where impairment is characterized by at least one quantitative measure of the influenza specific immune response, such as one or more of the following criteria: (i) less than a 20% increase in seroconversion rate, (ii) less than a 20% increase in seroprotection rate, (iii) a less than a 2-fold increase in seroconversion factor; (iv) a less than a 2-fold increase in GMT, in human subjects primed with a non-adjuvanted composition compared to subjects primed with an adjuvanted composition.

A method for boosting the immune response against influenza virus to a protective level of at least 80% to a booster administration in human subjects, comprising (i) administering to said human subject a first influenza vaccine in combination with an adjuvant, and (ii) administering to said subject a further booster dose of a influenza virus vaccine.

A method for improving a boosted immune response against influenza virus to a booster administration in human subjects, comprising (i) administering to said human subjects one single dose of a first influenza vaccine in combination with an adjuvant, and (ii) administering to said subject a further booster of a influenza virus vaccine, wherein said boosted immune response is higher than that obtained in subjects having received two doses of the first adjuvanted vaccine.

A method for preserving the boostability of the immune response against one or several influenza virus strains to a booster administration in human subjects, comprising (i) administering to said human subjects one single dose of a first influenza virus vaccine in combination with an adjuvant, and (ii) administering to said subject one single booster dose of a influenza virus vaccine, wherein at least one of the criteria: (i) GMTs, (ii) booster factors, (iii) seroconversion rates, (iv) booster responses or (v) seroprotection rates observed after one dose of booster vaccination, is not significantly decreased, or is similar, or is augmented in said subjects, as compared to the immune response to a booster dose in subjects having received two doses of primary vaccination.

A method for improving an influenza specific immune response to a plurality of vaccine administrations comprising, administering a first and a second dose of a vaccine composition comprising an influenza virus antigen and an adjuvant at an interval of at least 6 months, without administering an intervening vaccine composition, wherein the influenza specific immune response is higher than that obtained in subjects having an intervening administration (for example, an intervening administration is delivered in an interval not exceeding 6 weeks from the first dose).

A method for improving a boosted immune response against influenza virus to a booster administration in human subjects, comprising (i) administering to said human subject one single dose of a first influenza vaccine in combination with an adjuvant, and (ii) administering to said subject a further booster of an influenza virus vaccine at least 6 months after the first dose, wherein said boosted immune response is higher in subjects having received two doses at a 6-months interval compared to subjects having received two doses in an interval not exceeding 6 weeks.

The use of a first influenza virus vaccine in combination with an adjuvant and of a boosting influenza vaccine in the manufacture of a multi-dose vaccine for the prevention of the impairment of the immune response against influenza virus to the booster administration of said influenza virus vaccine in human subjects.

The use of a first influenza virus vaccine in combination with an adjuvant and of a boosting influenza vaccine in the manufacture of a multi-dose vaccine for boosting the immune response against influenza virus to a protective level of at least 80% to the booster administration in human subjects.

The use of a one dose influenza virus vaccine in combination with an adjuvant and of a boosting influenza vaccine in the manufacture of a multi-dose vaccine for improving a boosted immune response against influenza virus to a further booster administration in human subjects, wherein said boosted immune response is higher than that obtained in subjects having received two doses of the first adjuvanted vaccine.

The use of a first one dose influenza virus vaccine in combination with an adjuvant and of a boosting influenza vaccine in the manufacture of a multi-dose vaccine for preserving the boostability of the immune response against one or several influenza virus strains to a booster administration in human subjects, wherein at least one of the criteria: (i) GMTs, (ii) booster factors, (iii) seroconversion rates, (iv) booster responses or (v) seroprotection rates observed after one dose of booster vaccination, is not significantly decreased, or is similar, or is augmented in said subjects, as compared to the immune response to a booster dose in subjects having received two doses of primary vaccination.

The use of first influenza virus vaccine in combination with an adjuvant and of a boosting influenza vaccine in the manufacture of a multi-dose vaccine for improving an influenza specific immune response to a plurality of vaccine administrations wherein a first and a second dose of a adjuvanted vaccine is administered at an interval of at least 6 months, without administering an intervening influenza vaccine, wherein the influenza specific immune response is higher than that obtained in subjects having an intervening administration.

The use of first influenza virus vaccine in combination with an adjuvant and of a boosting influenza vaccine in the manufacture of a multi-dose vaccine for improving a boosted immune response against influenza virus to a booster administration in human subjects, wherein one single dose of said first adjuvanted influenza vaccine is administered, and a further booster dose of an influenza vaccine is administering to said subject at least 6 months after the first dose, wherein said boosted immune response is higher in subjects having received two doses at a 6-months interval compared to subjects having received two doses in an interval not exceeding 6 weeks.

An adjuvanted influenza virus vaccine for use in preventing the impairment of the immune response against influenza virus to a further booster administration of an influenza virus vaccine in human subjects.

An adjuvanted influenza virus vaccine for use in promoting the boost of the immune response against influenza virus to a protective level of at least 80% to a further booster administration in human subjects.

A one-dose adjuvanted influenza virus vaccine for use in improving a boosted immune response against influenza virus to a further booster administration of an influenza virus vaccine in human subjects, wherein said boosted immune response is higher than that obtained in subjects having received two doses of a first adjuvanted vaccine.

A one-dose adjuvanted influenza virus vaccine for use in preserving the boostability of the immune response against one or several influenza virus strains to a one-dose booster administration in human subjects, wherein at least one of the criteria: (i) GMTs, (ii) booster factors, (iii) seroconversion rates, (iv) booster responses or (v) seroprotection rates observed after one dose of booster vaccination, is not significantly decreased, or is similar, or is augmented in said subjects, as compared to the immune response to a booster dose in subjects having received two doses of primary vaccination. For example, when administered in a prime-boost regiment, the adjuvanted influenza virus vaccine yields a boosted immune response that is characterized by a booster factor at least 1.5-fold higher, or at least 2-fold higher, or at least 2.5-fold higher in subjects having received one dose of primary vaccination compared to subjects having received two doses of primary vaccination.

An adjuvanted influenza virus vaccine for use in improving an influenza specific immune response to a plurality of vaccine administrations comprising, wherein a first and a second dose of a vaccine composition comprising an influenza virus antigen and an adjuvant is administered at an interval of at least 6 months, without administering an intervening vaccine composition, and wherein the influenza specific immune response is higher than that obtained in subjects having an intervening administration.

A one dose adjuvanted influenza virus vaccine for use in improving a boosted immune response against influenza virus to a booster administration in human subjects said further booster influenza virus vaccine is administered at least 6 months after the first dose, and wherein said boosted immune response is higher in subjects having received two doses at a 6-months interval compared to subjects having received two doses in an interval not exceeding 6 weeks.

In certain embodiments, the immunogenic composition is further capable of inducing both an improved CD4 T-cell response and an improved B-memory cell response (e.g., in a naïve or seropositive subject) compared to that obtained with the un-adjuvanted antigen or antigenic composition.

In any of the preceding embodiments, the admuvant can be an oil-in-water emulsion. For example, in all of these embodiment, said oil-in-water emulsion adjuvant suitably comprises at least one metabolizable oil in an amount of 0.5% to 20% of the total volume, and has oil droplets of which at least 70% by intensity have diameters of less than 1 μm.

Optionally, the influenza virus vaccine comprises less than 15 μg of haemagglutining (HA) per strain per dose. Such as about 5 μg, less than 5 μg, less than 4 μg, (such as about 3.8 μg, e.g., 3.75 μg, less than 3 μg, less than about 2 μg (such as about 1.9 μg), or about 1 μg of HA per strain per dose.

In any of the preceding “prime-boost” methods, uses or compositions, the boosting composition can include an influenza virus strain which non-identical, e.g., is a variant of, or heterologous to, the strain present in the first adjuvanted vaccine. Accordingly, the influenza virus vaccines can include an influenza virus antigen or antigenic preparation thereof from any strain or serotype of influenza, such as an H1, H2, H5, H3, H7, H9, H10 influenza virus strain.

The disclosed methods, uses and compositions are all applicable for eliciting an immune response against influenza in human subjects including: children of between 1 months and 6 months, children below the age of 36 months, children of between 6 and 12 years, children below the age of 18, young adults (18-49 years), adults of between 18-64, and elderly adults over the age of 65.

### EXAMPLES

The invention will be further described by reference to the following, non-limiting, examples:

Example I describes immunological read-out methods used in mice, ferret and human studies.

Example II describes the preparation and characterization of the oil in water emulsion and adjuvant formulations used in the studies exemplified.

Example III describes a clinical trial in an elderly population aged over 65 years with a vaccine containing a split influenza antigen preparation and AS03 adjuvant

Example IV describes a second clinical trial—revaccination trial—in an elderly population aged over 65 years with a vaccine containing a split influenza antigen preparation and AS03 adjuvant.

Example V shows a pre-clinical evaluation of adjuvanted and un-adjuvanted influenza vaccines in ferrets (study I and study II). The temperature monitoring, viral shedding and CD4 T-cell response were measured.

Example VI shows a pre-clinical evaluation of adjuvanted and un-adjuvanted influenza vaccines in C57BI/6 naïve and primed mice.

Example VII shows a pre-clinical evaluation of adjuvanted and un-adjuvanted split and sub-unit influenza vaccines in C57BI/6 mice primed with heterologous strains.

Example VIII describes a clinical trial in an elderly population aged over 65 years with a vaccine containing a split influenza antigen preparation containing AS03 adjuvant, AS03+MPL adjuvant, or no exogeneous adjuvant.

Example IX shows a pre-clinical evaluation of adjuvanted and un-adjuvanted influenza vaccines in ferrets (study III). The temperature monitoring, viral shedding and HI titers were measured.

Example X shows a clinical trial in an elderly population aged over 65 years with a vaccine containing a split influenza antigen preparation containing AS03 with or without MPL adjuvant: immunogenicity persistence data at day 90 and day 180.

Example XI shows a clinical trial in an elderly population aged over 65 years with a vaccine containing a split influenza antigen preparation containing AS03 with MPL adjuvant.

Example XII shows a clinical trial in an elderly population aged over 65 years with a vaccine containing a split influenza antigen preparation containing AS03 with MPL adjuvant at two concentrations.

Example XIII describes the preparation of the oil-in-water emulsion and adjuvant formulations used in the studies exemplifying additional antigens.

Example XIV shows a Clinical trial in a population aged 18-60 years with a vaccine containing an adjuvanted split influenza antigen preparation according to different vaccination schedules

Example XV shows a Phase II clinical trial in a population aged 19-61 years with a vaccine containing an adjuvanted split influenza antigen preparation according to different vaccination schedules

### Example I

### Immunological Read-Out Methods

### I.1. Mice Methods

### I.1.1. Hemagglutination Inhibition Test

### Test Procedure

Anti-Hemagglutinin antibody titers to the three influenza virus strains were determined using the hemagglutination inhibition test (HI). The principle of the HI test is based on the ability of specific anti-Influenza antibodies to inhibit hemagglutination of chicken red blood cells (RBC) by influenza virus hemagglutinin (HA). Heat inactivated sera were previously treated by Kaolin and chicken RBC to remove non-specific inhibitors. After pretreatment, two-fold dilutions of sera were incubated with 4 hemagglutination units of each influenza strain. Chicken red blood cells were then added and the inhibition of agglutination was scored. The titers were expressed as the reciprocal of the highest dilution of serum that completely inhibited hemagglutination. As the first dilution of sera was 1:20, an undetectable level was scored as a titer equal to 10.

### Statistical Analysis

Statistical analysis were performed on post vaccination HI titers using UNISTAT. The protocol applied for analysis of variance can be briefly described as follow:
Log transformation of data
Shapiro-Wilk test on each population (group) in order to verify the normality of groups distribution
Cochran test in order to verify the homogenicity of variance between the different populations (groups)
Two-way Analysis of variance performed on groups
Tukey HSD test for multiple comparisons

### I.1.2. Intracellular Cytokine Staining

This technique allows a quantification of antigen specific T lymphocytes on the basis of cytokine production: effector T cells and/or effector-memory T cells produce IFN-γ and/or central memory T cells produce IL-2. PBMCs are harvested at day 7 post-immunization.

Lymphoid cells are re-stimulated in vitro in the presence of secretion inhibitor (Brefeldine). These cells are then processed by conventional immunofluorescent procedure using fluorescent antibodies (CD4, CD8, IFN-γ and IL-2). Results are expressed as a frequency of cytokine positive cell within CD4/CD8 T cells. Intracellular staining of cytokines of T cells was performed on PBMC 7 days after the second immunization. Blood was collected from mice and pooled in heparinated medium RPMI+Add. For blood, RPMI+Add-diluted PBL suspensions were layered onto a Lympholyte-Mammal gradient according to the recommended protocol (centrifuge 20 min at 2500 rpm and R.T.). The mononuclear cells at the interface were removed, washed 2× in RPMI+Add and PBMCs suspensions were adjusted to 2×10⁶ cells/ml in RPMI 5% fetal calf serum.

In vitro antigen stimulation of PBMCs was carried out at a final concentration of 1×10⁷ cells/ml (tube FACS) with Whole F1 (1 μgHA/strain) and then incubated 2 hrs at 37° C. with the addition of anti-CD28 and anti-CD49d (1 μg/ml for both).

Following the antigen restimulation step, PBMC are incubated overnight at 37° C. in presence of Brefeldin (1 μg/ml) at 37° C. to inhibit cytokine secretion.

IFN-γ/IL-2/CD4/CD8 staining was performed as follows: Cell suspensions were washed, resuspended in 50 μl of PBS 1% FCS containing 2% Fc blocking reagent (1/50; 2.4 G2). After 10 min incubation at 4° C., 50 μl of a mixture of anti-CD4-PE (2/50) and anti-CD8 perCp (3/50) was added and incubated 30 min at 4° C. After a washing in PBS 1% FCS, cells were permeabilized by resuspending in 200 μl of Cytofix-Cytoperm (Kit BD) and incubated 20 min at 4° C. Cells were then washed with Perm Wash (Kit BD) and resuspended with 50 μl of a mix of anti-IFN-γ APC (1/50)+anti-IL-2 FITC (1/50) diluted in Perm Wash. After an incubation min 2 h max overnight at 4° C., cells were washed with Perm Wash and resuspended in PBS 1% FCS+1% paraformaldehyde. Sample analysis was performed by FACS. Live cells were gated (FSC/SSC) and acquisition was performed on ˜20,000 events (lymphocytes) or 35,000 events on CD4+T cells. The percentages of IFN-γ+ or IL2+ were calculated on CD4+ and CD8+ gated populations.

### I.2. Ferrets Methods

### I.2.1. Hemagglutination Inhibition Test (HI)

### Test Procedure.

Anti-Hemagglutinin antibody titers to the three influenza virus strains were determined using the hemagglutination inhibition test (HI). The principle of the HI test is based on the ability of specific anti-Influenza antibodies to inhibit hemagglutination of chicken red blood cells (RBC) by influenza virus hemagglutinin (HA). Sera were first treated with a 25% neuraminidase solution (RDE) and were heat-inactivated to remove non-specific inhibitors. After pre-treatment, two-fold dilutions of sera were incubated with 4 hemagglutination units of each influenza strain. Chicken red blood cells were then added and the inhibition of agglutination was scored. The titers were expressed as the reciprocal of the highest dilution of serum that completely inhibited hemagglutination. As the first dilution of sera was 1:10, an undetectable level was scored as a titer equal to 5.

### Statistical Analysis.

Statistical analysis were performed on HI titers (Day 41, before challenge) using UNISTAT. The protocol applied for analysis of variance can be briefly described as followed:
Log transformation of data.
Shapiro-wilk test on each population (group) in order to verify the normality of groups distribution.
Cochran test in order to verify the homogenicity of variance between the different populations (groups).
Test for interaction of one-way ANOVA.
Tuckey-HSD Test for multiple comparisons.

### I.2.2. Body Temperature Monitoring

Individual temperatures were monitored during the challenge period with the transmitters and by the telemetry recording. All implants were checked and refurbished and a new calibration was performed by DSI (Data Sciences International, Centaurusweg 123, 5015 TC Tilburg, The Netherlands) before placement in the intraperitoneal cavity. All animals were individually housed in single cage during these measurements.

Temperatures were recorded every 15 minutes 4 days before challenge until 7 days Post-challenge.

### I.2.3. Nasal Washes

The nasal washes were performed by administration of 5 ml of PBS in both nostrils in awoke animals. The inoculum was collected in a Petri dish and placed into sample containers on dry ice.

### Viral Titration in Nasal Washes

All nasal samples were first sterile filtered through Spin X filters (Costar) to remove any bacterial contamination. 50 μl of serial ten-fold dilutions of nasal washes were transferred to microtiter plates containing 50 μl of medium (10 wells/dilution). 100 μl of MDCK cells (2.4×10⁵ cells/ml) were then added to each well and incubated at 35° C. for 5-7 days.

After 5-7 days of incubation, the culture medium is gently removed and 100 μl of a 1/20 WST-1 containing medium is added and incubated for another 18 hrs.

The intensity of the yellow formazan dye produced upon reduction of WST-1 by viable cells is proportional to the number of viable cells present in the well at the end of the viral titration assay and is quantified by measuring the absorbance of each well at the appropriate wavelength (450 nanometers). The cut-off is defined as the OD average of uninfected control cells—0.3 OD (0.3 OD correspond to +/−3 StDev of OD of uninfected control cells). A positive score is defined when OD is <cut-off and in contrast a negative score is defined when OD is >cut-off. Viral shedding titers were determined by “Reed and Muench” and expressed as Log TCID50/ml.

### I.3. Assays for Assessing the Immune Response in Humans

### I.3.1. Hemagglutination Inhibition Assay

The immune response was determined by measuring HI antibodies using the method described by the WHO Collaborating Centre for influenza, Centres for Disease Control, Atlanta, USA (1991).

Antibody titre measurements were conducted on thawed frozen serum samples with a standardised and comprehensively validated micromethod using 4 hemagglutination-inhibiting units (4 HIU) of the appropriate antigens and a 0.5% fowl erythrocyte suspension. Non-specific serum inhibitors were removed by heat treatment and receptor-destroying enzyme.

The sera obtained were evaluated for HI antibody levels. Starting with an initial dilution of 1:10, a dilution series (by a factor of 2) was prepared up to an end dilution of 1:20480. The titration end-point was taken as the highest dilution step that showed complete inhibition (100%) of hemagglutination. All assays were performed in duplicate.

### I.3.2. Neuraminidase Inhibition Assay

The assay was performed in fetuin-coated microtitre plates. A 2-fold dilution series of the antiserum was prepared and mixed with a standardised amount of influenza A H3N2, H1N1 or influenza B virus. The test was based on the biological activity of the neuraminidase which enzymatically releases neuraminic acid from fetuin. After cleavage of the terminal neuraminic acid β-D-glactose-N-acetyl-galactosamin was unmasked. Horseradish peroxidase (HRP)-labelled peanut agglutinin from Arachis hypogaea, which binds specifically to the galactose structures, was added to the wells. The amount of bound agglutinin can be detected and quantified in a substrate reaction with tetra-methylbenzidine (TMB) The highest antibody dilution that still inhibits the viral neuraminidase activity by at least 50% was indicated is the NI titre.

### I.3.3. Neutralising Antibody Assay

Neutralising antibody measurements were conducted on thawed frozen serum samples. Virus neutralisation by antibodies contained in the serum was determined in a microneutralization assay. The sera were used without further treatment in the assay.

Each serum was tested in triplicate. A standardised amount of virus was mixed with serial dilutions of serum and incubated to allow binding of the antibodies to the virus. A cell suspension, containing a defined amount of MDCK cells was then added to the mixture of virus and antiserum and incubated at 33° C. After the incubation period, virus replication was visualised by hemagglutination of chicken red blood cells. The 50% neutralisation titre of a serum was calculated by the method of Reed and Muench.

### I.3.4. Cell-Mediated Immunity was Evaluated by Cytokine Flow Cytometry (CFC)

Peripheral blood antigen-specific CD4 and CD8 T cells can be restimulated in vitro to produce IL-2, CD40L, TNF-alpha and IFN if incubated with their corresponding antigen. Consequently, antigen-specific CD4 and CD8 T cells can be enumerated by flow cytometry following conventional immunofluorescence labelling of cellular phenotype as well as intracellular cytokines production. In the present study, Influenza vaccine antigen as well as peptides derived from specific influenza protein were used as antigen to restimulate Influenza-specific T cells. Influenza antigens derived from drifted strains could also have been used to restimulate influenza-specific T-cells in order to assess the cross-reactivity of the CMI response. Results were expressed as a frequency of cytokine(s)-positive CD4 or CD8 T cell within the CD4 or CD8 T cell sub-population.

### I.3.5. Statistical Methods

### I.3.5.1. Primary Endpoints

Percentage, intensity and relationship to vaccination of solicited local and general signs and symptoms during a 7 day follow-up period (i.e. day of vaccination and 6 subsequent days) after vaccination and overall.
Percentage, intensity and relationship to vaccination of unsolicited local and general signs and symptoms during a 21 day follow-up period (i.e. day of vaccination and 20 subsequent days) after vaccination and overall.
Occurrence of serious adverse events during the entire study.

### I.3.5.2. Secondary Endpoints

### For the Humoral Immune Response:

### Observed Variables:

At days 0 and 21: serum hemagglutination-inhibition (HI) and NI antibody titres, tested separately against each of the three influenza virus strains represented in the vaccine (anti-H1N1, anti-H3N2 & anti-B-antibodies).
At days 0 and 21: neutralising antibody titres, tested separately against each of the three influenza virus strains represented in the vaccine
   Derived Variables (with 95% Confidence Intervals):
Geometric mean titres (GMTs) of serum HI antibodies with 95% confidence intervals (95% CI) pre and post-vaccination
Seroconversion rates* with 95% CI at day 21
Conversion factors** with 95% CI at day 21
Seroprotection rates*** with 95% CI at day 21
Serum NI antibody GMTs' (with 95% confidence intervals) at all timepoints. * Seroconversion rate defined as the percentage of vaccinees who have at least a 4-fold increase in serum HI titres on day 21 compared to day 0, for each vaccine strain.**Conversion factor defined as the fold increase in serum HI GMTs on day 21 compared to day 0, for each vaccine strain.***Protection rate defined as the percentage of vaccinees with a serum HI titre=40 after vaccination (for each vaccine strain) that usually is accepted as indicating protection.

### For the Cell Mediated Immune (CMI) Response

### Observed Variable

At days 0 and 21: frequency of cytokine-positive CD4/CD8 cells per 10⁶ in different tests. Each test quantifies the response of CD4/CD8 T cell to:
Peptide Influenza (pf) antigen (the precise nature and origin of these antigens needs to be given/explained
Split Influenza (sf) antigen
Whole Influenza (wf) antigen.

### Derived Variables:

cells producing at least two different cytokines (CD40L, IL-2, IFNγ, TNFα)
cells producing at least CD40L and another cytokine (IL-2, TNFα, IFNγ)
cells producing at least IL-2 and another cytokine (CD40L, TNFα, IFNγ)
cells producing at least IFNγ and another cytokine (IL-2, TNFα, CD40L)
cells producing at least TNFα and another cytokine (IL-2, CD40L, IFNγ)

### I.3.5.3. Analysis of Immunogenicity

The immunogenicity analysis was based on the total vaccinated cohort. For each treatment group, the following parameters (with 95% confidence intervals) were calculated:
Geometric mean titres (GMTs) of HI and NI antibody titres at days 0 and 21
Geometric mean titres (GMTs) of neutralising antibody titres at days 0 and 21.
Conversion factors at day 21.
Seroconversion rates (SC) at day 21 defined as the percentage of vaccinees that have at least a 4-fold increase in serum HI titres on day 21 compared to day 0.
Protection rates at day 21 defined as the percentage of vaccinees with a serum HI titre=1:40.
The frequency of CD4/CD8 T-lymphocytes secreting in response was summarised (descriptive statistics) for each vaccination group, at each timepoint (Day 0, Day 21) and for each antigen (Peptide influenza (pf), split influenza (sf) and whole influenza (wf)).
Descriptive statistics in individual difference between timepoint (Post-Pre) responses fore each vaccination group and each antigen (pf, sf, and wf) at each 5 different tests.
A non-parametric test (Kruskall-Wallis test) was used to compare the location differences between the 3 groups and the statistical p-value was calculated for each antigen at each 5 different tests. All significance tests were two-tailed. P-values less than or equal to 0.05 were considered as statistically significant.

### I.3.6 ELISPOT

The ELISPOT technology allows the quantification of memory B-cells specific to a given antigen. Memory B-cells can be induced to differentiate into plasma cells in vitro following cultivation with CpG for 5 days. In vitro generated antigen-specific plasma cells can therefore be enumerated using the ELISPOT assay. Briefly, in vitro generated plasma cells are incubated in culture plates coated with antigen. Antigen-specific plasma cells form antibody/antigen spots, which can be detected by conventional immuno-enzymatic procedure. In the present study, influenza vaccine strains or anti-human immunoglobulins were used to coat culture plates in order to enumerate influenza-specific antibody or IgG secreting plasma cells, respectively. Results were expressed as a frequency of influenza-specific antibody secreting plasma cells within the IgG-producing plasma cells.

### Example II

### Preparation and Characterization of the Oil in Water Emulsion and Adjuvant Formulations

Unless otherwise stated, the oil/water emulsion used in the subsequent examples is composed an organic phase made of 2 oils (alpha-tocopherol and squalene), and an aqueous phase of PBS containing Tween 80 as emulsifying agent. Unless otherwise stated, the oil in water emulsion adjuvant formulations used in the subsequent examples were made comprising the following oil in water emulsion component (final concentrations given): 2.5% squalene (v/v), 2.5% alpha-tocopherol (v/v), 0.9% polyoxyethylene sorbitan monooleate (v/v) (Tween 80), see WO 95/17210. This emulsion, termed AS03 in the subsequent examples, was prepared as followed as a two-fold concentrate.

### II.1. Preparation of Emulsion SB62

### II.1.1. Lab-Scale Preparation

Tween 80 is dissolved in phosphate buffered saline (PBS) to give a 2% solution in the PBS. To provide 100 ml two-fold concentrate emulsion 5 g of DL alpha tocopherol and 5 ml of squalene are vortexed to mix thoroughly. 90 ml of PBS/Tween solution is added and mixed thoroughly. The resulting emulsion is then passed through a syringe and finally microfluidised by using an Ml OS microfluidics machine. The resulting oil droplets have a size of approximately 120-180 nm (expressed as Z average measured by PCS).

The other adjuvants/antigen components are added to the emulsion in simple admixture.

### II.1.2. Scaled-Up Preparation

The preparation of the SB62 emulsion is made by mixing under strong agitation of an oil phase composed of hydrophobic components (α-tocopherol and squalene) and an aqueous phase containing the water soluble components (Tween 80 and PBS mod (modified), pH 6.8). While stirring, the oil phase ( 1/10 total volume) is transferred to the aqueous phase ( 9/10 total volume), and the mixture is stirred for 15 minutes at room temperature. The resulting mixture then subjected to shear, impact and cavitation forces in the interaction chamber of a microfluidizer (15000 PSI—8 cycles) to produce submicron droplets (distribution between 100 and 200 nm). The resulting pH is between 6.8±0.1. The SB62 emulsion is then sterilised by filtration through a 0.22 μm membrane and the sterile bulk emulsion is stored refrigerated in Cupac containers at 2 to 8° C. Sterile inert gas (nitrogen or argon) is flushed into the dead volume of the SB62 emulsion final bulk container for at least 15 seconds.

The final composition of the SB62 emulsion is as follows:

Tween 80:1.8% (v/v) 19.4 mg/ml; Squalene: 5% (v/v) 42.8 mg/ml; α-tocopherol: 5% (v/v) 47.5 mg/ml; PBS-mod: NaCl 121 mM, KCl 2.38 mM, Na2HPO4 7.14 mM, KH2PO4 1.3 mM; pH 6.8±0.1.

### II.2. Measure of Oil Droplet Size Dynamic Light Scattering

### II.2.1. Introduction

The size of the diameter of the oil droplets is determined according to the following procedure and under the following experimental conditions. The droplet size measure is given as an intensity measure and expressed as z average measured by PCS.

### II.2.2. Sample Preparation

Size measurements have been performed on the oil-in-water emulsion adjuvant: SB62 prepared following the scaled-up method, AS03 and AS03+MPL (50 μg/ml), the last two being prepared just before use. The composition of the samples is given below (see section II.2.4). Samples were diluted 4000×-8000× in PBS 7.4.

As a control, PL-Nanocal Particle size standards 100 nm (cat n^{o} 6011-1015) was diluted in 10 mM NaCl.

### II.2.3. Malvern Zetasizer 3000HS Size Measurements

All size measurements were performed with both Malvern Zetasizer 3000HS.

Samples were measured into a plastic cuvette for Malvern analysis at a suitable dilution (usually at a dilution of 4000× to 20000× depending on the sample concentration), and with two optical models:
either real particle refractive index of 0 and imaginary one of 0.
or real particle refractive index of 1.5 and imaginary one of 0.01 (the adapted optical model for the emulsion, according to the values found in literature).

The technical conditions were:
laser wavelength: 532 nm (Zeta3000HS).
laser power: 50 mW (Zeta3000HS).
scattered light detected at 90° (Zeta3000HS).
temperature: 25° C.,
duration: automatic determination by the soft,
number: 3 consecutive measurements,
z-average diameter: by cumulants analysis
size distribution: by the Contin or the Automatic method.

The Automatic Malvern algorithm uses a combination of cumulants, Contin and non negative least squares (NNLS) algorithms.

The intensity distribution may be converted into volume distribution thanks to the Mie theory.

### II.2.4. Results (see Table 2)

### Cumulants Analysis (Z Average Diameter):

TABLE 2

| Sample | Dilution | Record | Count rate | ZAD | Polydispersity |
|---|---|---|---|---|---|
| SB62 | 5000 | 1 | 7987 | 153 | 0.06 |
| | | 2 | 7520 | 153 | 0.06 |
| | | 3 | 6586 | 152 | 0.07 |
| | | average | 7364 | 153 | 0.06 |
| SB62 | 8000 | 1 | 8640 | 151 | 0.03 |
| (Example IV) | | 2 | 8656 | 151 | 0.00 |
| | | 3 | 8634 | 150 | 0.00 |
| | | average | 8643 | 151 | 0.01 |
| SB62 + MPL | 8000 | 1 | 8720 | 154 | 0.03 |
| 25 μg (*) | | 2 | 8659 | 151 | 0.03 |
| | | 3 | 8710 | 152 | 0.02 |
| | | average | 8697 | 152 | 0.02 |

(*) Prepared as follows: Water for injection, PBS 10x concentrated, 250 μl of SB62 emulsion and 25 μg of MPL are mixed together to reach a final volume of 280 μl.

The z-average diameter (ZAD) size is weighed by the amount of light scattered by each size of particles in the sample. This value is related to a monomodal analysis of the sample and is mainly used for reproducibility purposes.

The count rate (CR) is a measure of scattered light: it corresponds to thousands of photons per second.

The polydispersity (Poly) index is the width of the distribution. This is a dimensionless measure of the distribution broadness.

### Contin and Automatic Analysis:

Two other SB62 preparations (2 fold concentrated AS03) have been made and assessed according to the procedure explained above with the following minor modifications:

Samples were measured into a plastic cuvette for Malvern analysis, at two dilutions determined to obtain an optimal count rate values: 10000× and 20000× for the Zetasizer 3000HS, the same optical models as used in the above example.

Results are shown in Table 3.

TABLE 3

| | | | | Analysis | | Analysis | |
|---|---|---|---|---|---|---|---|
| | | IR | | in Contin | | in Automatic | |
| | | | Imag- | (mean in nm) | | (mean in nm) | |
| SB62 | Dilution | Real | inary | Intensity | Volume | Intensity | Volume |
| 1022 | 1/10000 | 0 | 0 | 149 | 167 | 150 | — |
| | | 1.5 | 0.01 | 158 | 139 | 155 | 143 |
| | 1/20000 | 0 | 0 | 159 | 200 | 155 | 196 |
| | | 1.5 | 0.01 | 161 | 141 | 147 | — |
| 1023 | 1/10000 | 0 | 0 | 158 | 198 | 155 | — |
| | | 1.5 | 0.01 | 161 | 140 | 150 | 144 |
| | 1/20000 | 0 | 0 | 154 | 185 | 151 | 182 |
| | | 1.5 | 0.01 | 160 | 133 | 154 | — |

“—” when the obtained values were not coherent.

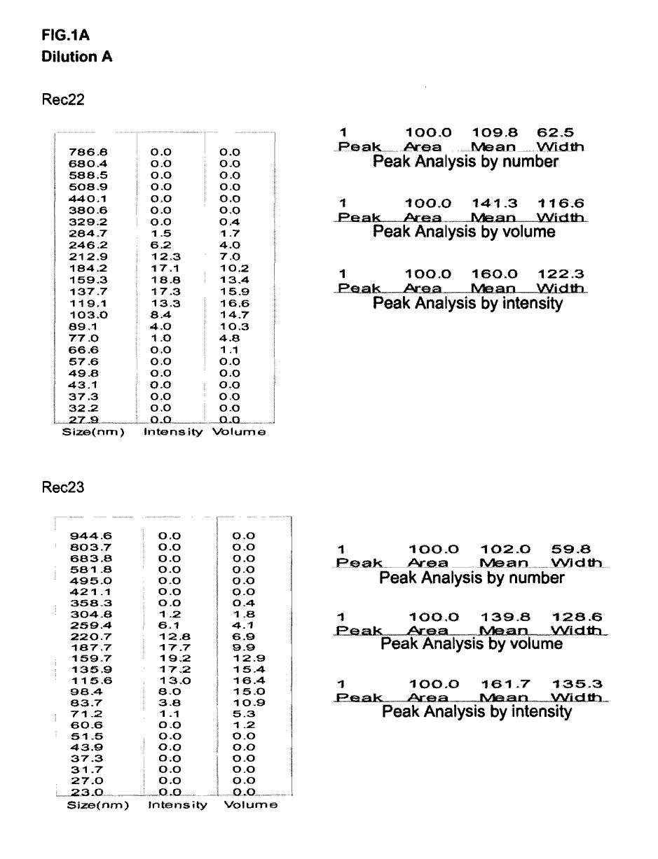

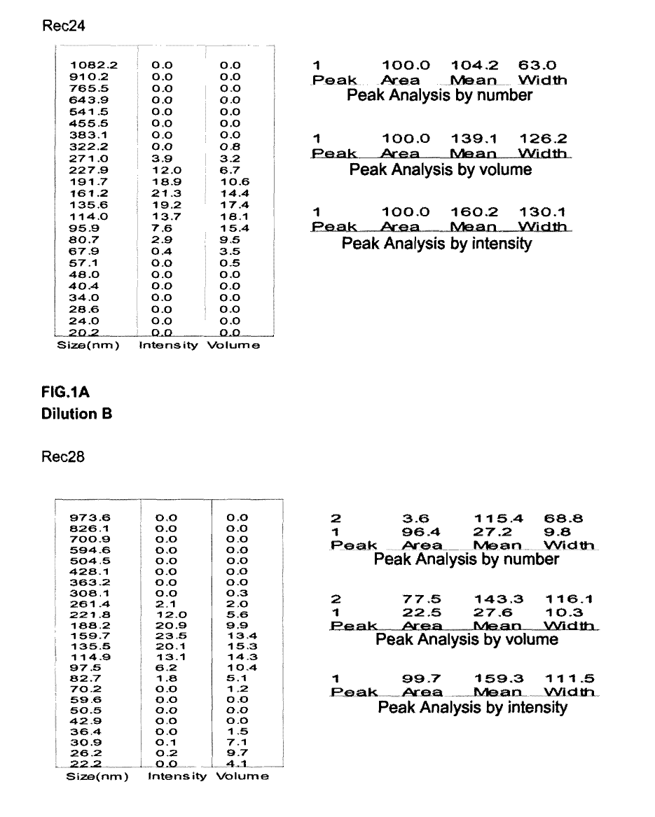

A schematic representation of these results is shown in FIG. 1 for formulation 1023. As can be seen, the great majority of the particles (e.g. at least 80%) have a diameter of less than 300 nm by intensity.

### II.2.5. Overall Conclusion

SB62 formulation was measured at different dilutions with the Malvern Zetasizer 3000HS and two optical models. The particle size ZAD (i.e. intensity mean by cumulant analysis) of the formulations assessed above was around 150-155 nm.

When using the cumulants algorithm, we observed no influence of the dilution on the ZAD and polydispersity.

### II.3. Preparation of AS03 Comprising MPL

### II.3.1. Preparation of MPL Liquid Suspension

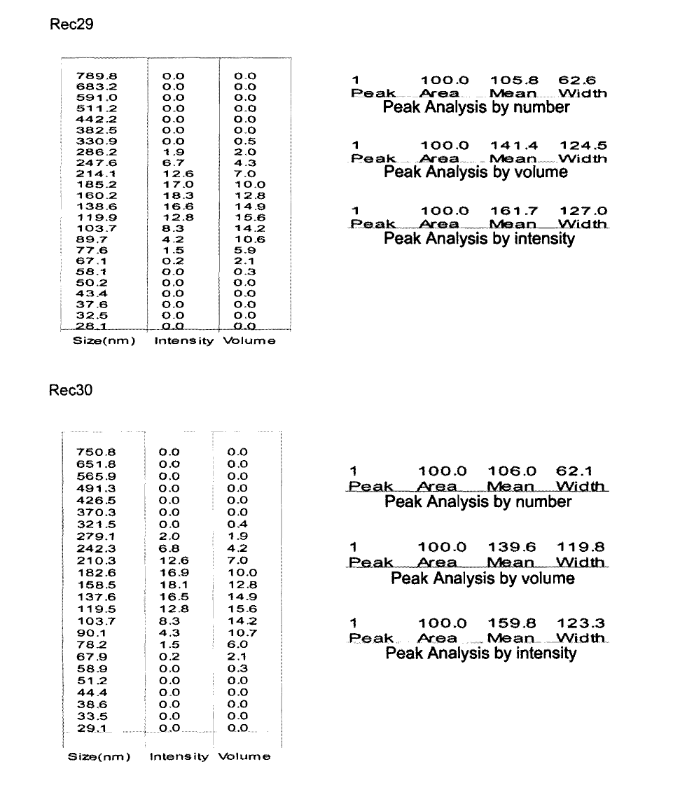

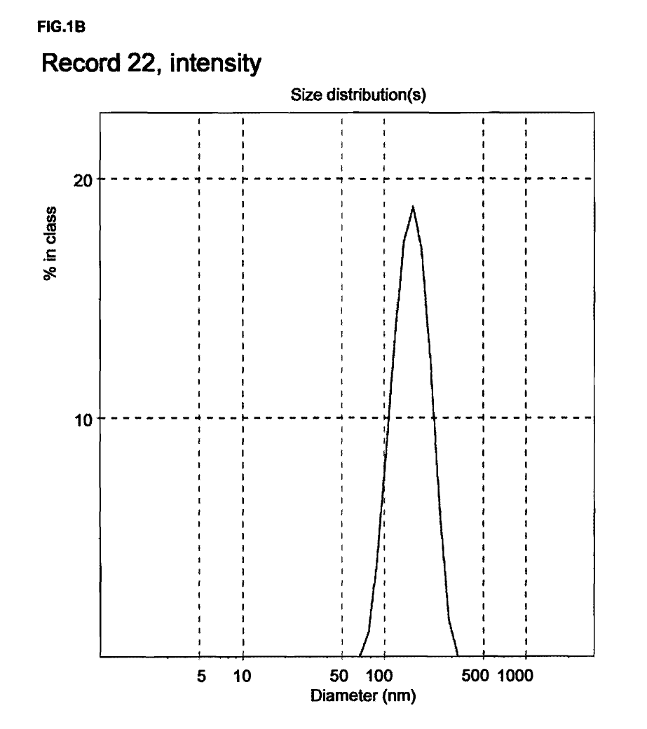

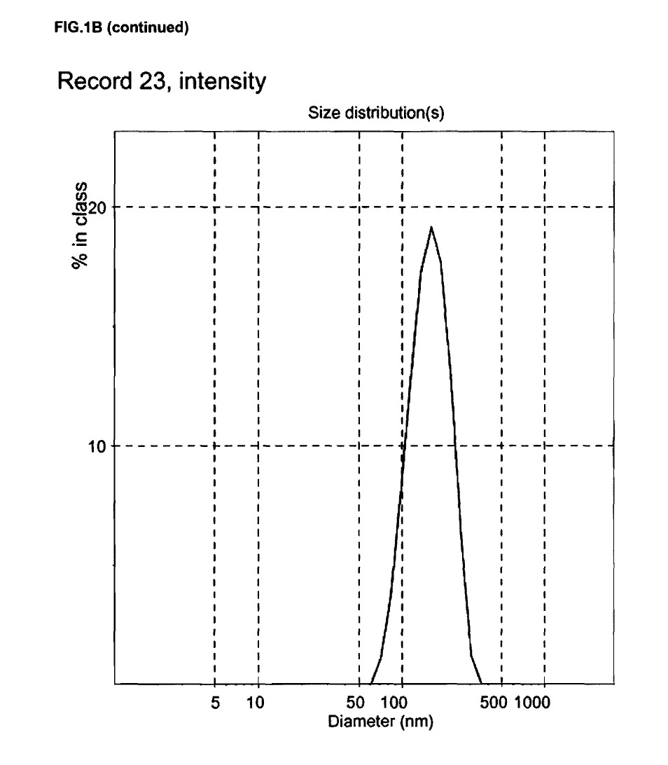

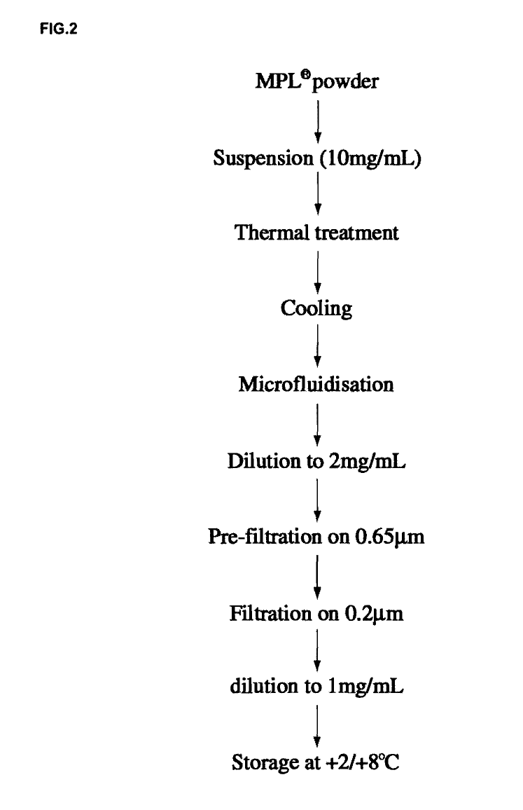

The MPL (as used throughout the document it is an abbreviation for 3D-MPL, i.e. 3-O-deacylated monophosphoryl lipid A) liquid bulk is prepared from MPL® lyophilized powder. MPL liquid bulk is a stable concentrated (around 1 mg/ml) aqueous dispersion of the raw material, which is ready-to-use for vaccine or adjuvant formulation. A schematic representation of the preparation process is given in FIG. 2.

For a maximum batch size of 12 g, MPL liquid bulk preparation is carried over in sterile glass containers. The dispersion of MPL consists of the following steps:
suspend the MPL powder in water for injection
desaggregate any big aggregates by heating (thermal treatment)
reduce the particle size between 100 nm and 200 nm by microfluidization
prefilter the preparation on a Sartoclean Pre-filter unit, 0.8/0.65 μm
sterile filter the preparation at room temperature (Sartobran P unit, 0.22 μm)

MPL powder is lyophilized by microfluidisation resulting in a stable colloidal aqueous dispersion (MPL particle size smaller than 200 nm). The MPL lyophilized powder is dispersed in water for injection in order to obtain a coarse 10 mg/ml suspension. The suspension then undergoes a thermal treatment under stirring. After cooling to room temperature, the microfluidization process is started in order to decrease the particle size. Microfluidization is conducted using Microfluidics apparatus M110EH, by continuously circulating the dispersion through a microfluidization interaction chamber, at a defined pressure for a minimum amount of passages (number of cycles: nₘᵢₙ). The microfluidization duration, representing the number of cycles, is calculated on basis of the measured flow rate and the dispersion volume. On a given equipment at a given pressure, the resulting flow rate may vary from one interaction chamber to another, and throughout the lifecycle of a particular interaction chamber. In the present example the interaction chamber used is of the type F20Y Microfluidics. As the microfluidization efficiency is linked to the couple pressure—flow rate, the processing time may vary from one batch to another. The time required for 1 cycle is calculated on basis of the flow rate. The flow rate to be considered is the flow rate measured with water for injection just before introduction of MPL into the apparatus. One cycle is defined as the time (in minutes) needed for the total volume of MPL to pass once through the apparatus. The time needed to obtain n cycles is calculated as follows:

n×quantity of MPL to treat (ml)/flow rate (ml/min)

The number of cycles is thus adapted accordingly. Minimum amount of cycles to perform (nₘᵢₙ) are described for the preferred equipment and interaction chambers used. The total amount of cycles to run is determined by the result of a particle size measurement performed after nₘᵢₙ cycles. A particle size limit (dₗᵢₘ) is defined, based on historical data. The measurement is realized by photon correlation spectroscopy (PCS) technique, and dₗᵢₘ is expressed as an unimodal result (Z_{average}). Under this limit, the microfluidization can be stopped after nₘᵢₙ cycles. Above this limit, microfluidization is continued until satisfactory size reduction is obtained, for maximum another 50 cycles.

If the filtration does not take place immediately after microfluidization, the dispersed MPL is stored at +2 to +8° C. awaiting transfer to the filtration area.

After microfluidization, the dispersion is diluted with water for injection, and sterile filtered through a 0.22 μm filter under laminal flow. The final MPL concentration is 1 mg/ml (0.80-1.20 mg/ml).

### II.3.2. Preparation of AS03+MPL Adjuvanted Vaccine: 1 Vial Approach

To the AS03 adjuvant formulation, MPL is added at a final concentration of between 10 and 50 μg per vaccine dose.

PBS 10 fold concentrated (pH 7.4 when one fold concentrated) as well as a SB62 mixture containing Tween, Triton X-100 and VES (vitamin E succinate) is added to water for injection. The quantities take into account the detergent present in the influenza strains so as to reach a target final concentration of 750 μg/ml Tween 80, 110 μg/ml Triton X-100 and 100 μg/ml VES. After 5 min stirring, 15 μg of each influenza strain of interest (for example strain H1N1, H3N2 and B in a classical tri-valent vaccine) are added. After 15 min stirring, 250 μl of SB62 emulsion is added and then 25 μg or 50 μg of MPL.

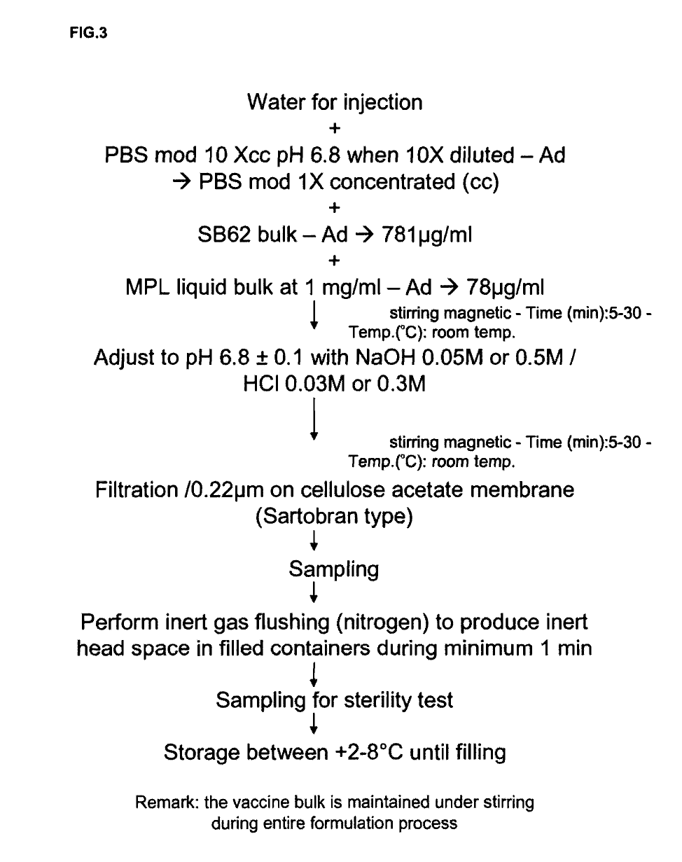

A schematic representation of the preparation process is given in FIG. 3. The final composition of AS03 comprising MPL per human dose is given the Table 4.

TABLE 4

| Ingredients | | | | Per human dose | | |
|---|---|---|---|---|---|---|
| Name | Component | Concentration | | Quantity | | Other |
| SB62 | | 781 | μl/ml | 250 | μl | |
| | Squalene (solution 43 mg/ml) | | | 10.68 | mg | |
| | Tocopherol (solution 48 mg/ml) | | | 11.86 | mg | |
| | Tween 80 (solution 20 mg/ml) | | | 4.85 | mg | |
| MPL** | (solution 1 mg/ml) | 78 | μg/ml or | 25 | μg or | |
| | | 156 | μg/ml | 50 | μg | |
| PBS mod* | NaCl | 137 | mM | 2.56 | mg | |
| | KCl | 2.7 | mM | 0.064 | mg | |
| | Na2HPO4 | 8.1 | mM | 0.368 | mg | |
| | KH2PO4 | 1.47 | mM | 0.064 | mg | |
| Water for | | | | | | Ad 320 μl |
| injection | | | | | | |
| pH | | | | | | 6.8 +/− 0.1 |

*PBS mod 10x concentrated pH 6.8 = KH2PO4, Na2HPO4, NaCl, KCl—HCl
**MPL is either 25 μg or 50 μg per dose

### II.3.3. Preparation of AS03+MPL Adjuvanted Vaccine: 2 Vials Approach

The same formulation can be prepared from a 2 vials approach by mixing 2 fold concentrated antigen or antigenic preparation with the AS03 (SB62 250 μl) or the AS03+MPL (SB62 250 μl+25 μg or 50 μg MPL) adjuvant. In this instance it is proceeded as follows. The manufacturing of the AS25-adjuvanted influenza vaccine consists of three main steps:

1) Formulation of the trivalent final bulk (2× concentrated) without adjuvant and filling in the antigen container
2) Preparation of the AS03+MPL adjuvant
3) Extemporaneous reconstitution of the AS03+MPL adjuvanted split virus vaccine.
1) Formulation of the Trivalent Final Bulk without Adjuvant and Filling in the Antigen Container

The volumes of the three monovalent bulks are based on the HA content measured in each monovalent bulk prior to the formulation and on a target volume of 1100 ml. Concentrated phosphate buffered saline and a pre-mixture of Tween 80, Triton X-100 and α-tocopheryl hydrogen succinate are diluted in water for injection. The three concentrated monobulks (A/New Calcdonia, A/New York, B/Jiangsu) are then successively diluted in the resulting phosphate buffered saline/Tween 80—Triton X-100—α-tocopheryl hydrogen succinate solution (pH 7.4, 137 mM NaCl, 2.7 mM KCl, 8.1 mM Na2HPO4, 1.47 mM KH2PO4, 990 μg/ml Tween 80, 150 μg/ml Triton X-100 and 130 μg/ml α-tocopheryl hydrogen succinate) in order to have a final concentration of 39.47 μg HA of A strains (H1N1, H3N2) per ml of trivalent final bulk (15 μg HA/A strain/380 μl trivalent final bulk) and 46 μg HA of B strain (17.5 μg HA/B strain/380 μl trivalent final bulk). Between addition of each monovalent bulk, the mixture is stirred for 10-30 minutes at room temperature. After addition of the last monovalent bulk and 15-30 minutes of stirring, the pH is checked and adjusted to 7.2±0.2 with HCl or NaOH.

The trivalent final bulk of antigens is aseptically filled into 3-ml sterile Type I (Ph. Eur.) glass vials. Each vial contains a volume of 470 μl (380 μl+90 μl overfill).

### 2) Preparation of AS03/MPL Adjuvant Bulk and Filling in the Adjuvant Container.

The adjuvant AS03/MPL is prepared by mixing of two components: SB62 emulsion (method in section II.1.2) and MPL (method in section II.3.1). One-fold concentrated PBS mod (prepared by diluting 10× concentrated PBS mod in water for injection) with SB62 bulk and MPL liquid bulk at 1 mg/ml. MPL concentration will be determined so as to reach a final content of between 10 to 50 μg, suitably around 25 μg per final human vaccine dose. The mixture is stirred for 5-30 minutes at room temperature, and the pH is adjusted to 6.8±0.1 with NAOH (0.05 or 0.5 M)/HCl (0.03 M or 0.3 M). After another stirring for 5-30 minutes at room temperature the mixture is sterilised by filtration through a 0.22 μm membrane. Sterile inert gas (nitrogen) flushing is performed to produce inert head space in the filled containers during minimum 1 minute. The sterile AS03+MPL adjuvant is stored at +2-8° C. until aseptical filling into 1.25-ml sterile Type I (Ph. Eur.) glass syringes. Each syringe contains a volume overage of 80 μl (320 μl+80 μl overfill).

At the time of injection, the content of the prefilled syringe containing the adjuvant is injected into the vial that contains the concentrated trivalent inactivated split virion antigens. After mixing the content is withdrawn into the syringe and the needle is replaced by an intramuscular needle. One dose of the reconstituted the AS25-adjuvanted influenza candidate vaccine corresponds to 0.7 mL.

### II.4. Preparation of Immunogenic Compositions Comprising an Influenza Antigen and Optionally MPL in an Oil in Water Emulsion Formulation

To the SB62 emulsion of II.1 an equal volume of twice concentrated split influenza antigen (Fluarix™) (15 μg HA per strain) was added and mixed. This was combined, when appropriate, with 50 μg/ml of MPL to give the final formulation.

### Example III

### Clinical Trial in an Elderly Population Aged Over 65 Years with a Vaccine Containing a Split Influenza Antigen Preparation and AS03 Adjuvant (Explo-Flu-001)

A phase I, open, randomised study was conducted in an elderly population aged over 65 years in 2003 in order to evaluate the reactogenicity and the immunogenicity of GlaxoSmithKline Biologicals influenza candidate vaccine containing the adjuvant AS03. The humoral immune response (i.e. anti-hemagglutinin, neutralising and anti-neuraminidase antibody titres) and cell mediated immune response (CD4 and/or CD8 T cell responses) was measured 21 days after intramuscular administration of one dose of an AS03 adjuvanted vaccine or a WV vaccine. Fluarix™ was used as reference.

### III.1. Study Design

Three groups of subjects in parallel received the following vaccine intramuscularly:
one group of 50 subjects receiving one dose of the reconstituted and adjuvanted SV influenza vaccine (FluAS03)
one group of 50 subjects receiving one dose of whole virus influenza vaccine (FluWW)
one group of 50 subjects receiving one dose of Fluarix™ (Fluarix)=control

Vaccination schedule: one injection of influenza vaccine at day 0, blood sample collection, read-out analysis at day 21 (HI antibody determination, NI antibody determination, determination of neutralising antibodies, and CMI analysis) and study conclusion.

The standard trivalent split influenza vaccine—Fluarix™ used in this study, is a commercial vaccine from the year 2003 developed and manufactured by GlaxoSmithKline Biologicals.

### III.2. Vaccine Composition and Administration (Table 5)

### III.2.1. Vaccine Preparation

### AS03 Adjuvanted Influenza Vaccine

The AS03-adjuvanted influenza vaccine candidate is a 2 components vaccine consisting of a concentrated trivalent inactivated split virion antigens presented in a type I glass vial (335 μl) (antigen container) and of a pre-filled type I glass syringe containing the SB62 emulsion (335 μl) (adjuvant container). At the time of injection, the content of the antigen container is removed from the with the help of the SB62 emulsion pre-filled syringe, followed by gently mixing of the syringe. Mixing of the SB62 emulsion with the vaccine antigens reconstitute the AS03 adjuvant. Prior to injection, the used needle is replaced by an intramuscular needle and the volume is corrected to 500 μl.

One dose of the reconstituted AS03-adjuvanted influenza vaccine corresponds to 0.5 ml, contains 15 μg HA of each influenza virus strain as in the registered Fluarix™/α-Rix® vaccine and contains 10.68 mg squalene, 11.86 mg DL-alpha tocopherol, and 4.85 mg polysorbate 80 (Tween 80).

### Preparation

The manufacturing of the AS03-adjuvanted influenza vaccine consists of three main steps:

1) Formulation of the Trivalent Final Bulk without Adjuvant and Filling in the Antigen Container.

The volumes of the three monovalent bulks are based on the HA content measured in each monovalent bulk prior to the formulation and on a target volume of 800 ml.

Concentrated phosphate buffered saline and a pre-mixture of Tween 80, Triton X-100 and α-tocopheryl hydrogen succinate are diluted in water for injection. The three concentrated monobulks (strain A/New Calcdonia -, strain A/Panama - and strain B/Shangdong -) are then successively diluted in the resulting phosphate buffered saline/Tween 80—Triton X-100—α-tocopheryl hydrogen succinate solution (pH 7.4, 137 mM NaCl, 2.7 mM KCl, 8.1 mM Na₂HPO₄, 1.47 mM KH₂PO₄, 1500 μg/ml Tween 80, 220 μg/ml Triton X-100 and 200 μg/ml α-tocopheryl hydrogen succinate) in order to have a final concentration of 60 μg HA of A strains per ml of trivalent final bulk (15 μg HA/A strain/250 μl trivalent final bulk) and 70 μg HA of B strain (17.5 μg HA/B strain/250 μl trivalent final bulk). Between addition of each monovalent bulk, the mixture is stirred for 10 minutes at room temperature. After addition of the last monovalent bulk and 15 minutes of stirring, the pH is checked and adjusted to 7.2±0.1 with HCl or NaOH.

The trivalent final bulk of antigens is aseptically filled into 3-ml sterile Type I glass vials. Each vial contains a 34% volume overage (335 μl total volume).

### 2) Preparation of the SB62 Emulsion Sterile Bulk and Filling in the Adjuvant Container.

Aqueous phase: while stirring, 902 ml of Tween 80 is mixed with 44105 ml of PBS-mod buffer (pH=6.8 after adjustment with HCl).
Oil phase: while stirring, 2550 ml of squalene is added to 2550 ml of α-tocopherol.
Mixing of the aqueous and oil phases: while stirring, 5000 ml of oil phase ( 1/10 total volume) is transferred to 45007 ml of aqueous phase ( 9/10 total volume). The mixture is stirred for 15 minutes at room temperature.
Emulsification: the resulting mixture is subjected to shear, impact and cavitation forces in the interaction chamber of a microfluidizer (15000 PSI—8 cycles) to produce submicron droplets (distribution between 100 and 200 nm). The resulting pH is between 6.8±0.1.
Sterile filtration: the SB62 emulsion is sterilised by filtration through a 0.22 μm membrane and the sterile bulk emulsion is stored refrigerated in Cupac containers at 2 to 8° C. Sterile inert gas (nitrogen or argon) is flushed into the dead volume of the SB62 emulsion final bulk container for at least 15 seconds.

All quantities of ingredients given are for the preparation of 50 L of emulsion and are given in volumes. In practice, amounts are weighed taking into account the densities of the ingredients. Density of PBS is considered equal to 1.

The final composition of the SB62 emulsion is as follows:

TABLE 5

| Tween 80: | 1.8% (v/v) | 19.4 mg/ml |
|---|---|---|
| Squalene: | 5% (v/v) | 42.8 mg/ml |
| alpha-tocopherol: | 5% (v/v) | 47.5 mg/ml |
| PBS-mod: | | |
| NaCl | | 121 mM |
| KCl | | 2.38 mM |
| Na₂HPO₄ | | 7.14 mM |
| KH₂PO₄ | | 1.3 mM |
| pH | | 6.8 ± 0.1 |

The sterile SB62 bulk emulsion is then aseptically filled into 1.25-ml sterile Type I glass syringes. Each syringe contains a 34% volume overage (335 μl total volume).

### 3) Extemporaneous Reconstitution of the AS03 Adjuvanted Split Virus Vaccine.

At the time of injection, the content of the vial containing the concentrated trivalent inactivated split virion antigens is removed from the vial with the help the syringe containing the SB62 emulsion followed by gently mixing of the syringe. Mixing of the SB62 emulsion with the vaccine antigens reconstitutes the AS03 adjuvant.

### III.2.2. Vaccine Composition (Table 6) and Administration

TABLE 6

| Vaccine | Formulation | Group |
|---|---|---|
| Fluarix ™ | HA from 3 influenza strains (total HA = 45 μg) | Fluarix |
| | A/New Caledonia/20/99 (IVR-116): 15 μg | |
| | A/Panama/2007/99 (RESVIR-17): 15 μg | |
| | B/Shangdong/7/97: 15 μg | |
| | Thiomersal content: 5 μg | |
| | In pre-filled syringes of 0.5 ml | |
| WVV | HA from 3 influenza strains (total HA = 45 μg) | FluWVV |
| | A/New Caledonia/20/99 (IVR-116): 15 μg | |
| | A/Panama/2007/99 (RESVIR-17): 15 μg | |
| | B/Shangdong/7/97: 15 μg | |
| | Thiomersal content: 5 μg | |
| | In vials of 0.5 ml | |
| Fluarix + | HA from 3 influenza strains (total HA = 45 μg) | Flu-AS03 |
| AS03 | A/New Caledonia/20/99 (IVR-116): 15 μg | |
| | A/Panama/2007/99 (RESVIR-17): 15 μg | |
| | B/Shangdong/7/97: 15 μg | |
| | Thiomersal content: 5 μg | |
| | In vial of 0.335 ml (2 times concentrated) + | |
| | syringe (0.335 ml) containing oil-in-water SB62 | |
| | emulsion (scaled-up preparation) | |

The vaccines were administered intramuscularly in the deltoid region of the non-dominant arm. The vaccinees were observed closely for at least 30 minutes, with appropriate medical treatment readily available in case of a rare anaphylactic reaction following the administration of vaccine.

### III.3. Study Population Results

A total of 148 subjects were enrolled in this study: 49 subjects in the FluAS03 group, 49 subjects in the Fluarix group and 50 subjects in the FluWVV group. The mean age of the total vaccinated cohort at the time of vaccination was 71.8 years with a standard deviation of 6.0 years. The mean age and gender distribution of the subjects across the three vaccine groups was similar.

### III.4. Safety Conclusions

The administration of the influenza vaccine adjuvanted with AS03 was safe and clinically well tolerated in the study population, i.e. elderly people aged over 65 years.

### III.5. Immunogenicity Results

Analysis of immunogenicity was performed on the total vaccinated cohort.

### III.5.1. Humoral Immune Response

In order to evaluate the humoral immune response induced by the AS03 adjuvanted vaccine, the following parameters (with 95% confidence intervals) were calculated for each treatment group:
Geometric mean titres (GMTs) of HI and NI antibody titres at days 0 and 21
Geometric mean titres (GMTs) of neutralising antibody titres at days 0 and 21.
Seroconversion rates (SC) at day 21 defined as the percentage of vaccinees that have at least a 4-fold increase in serum HI titres on day 21 compared to day 0.
Conversion factors at day 21 defined as the fold increase in serum HI GMTs on day 21 compared to day 0, for each vaccine strain.
Protection rates at day 21 defined as the percentage of vaccinees with a serum HI titre=1:40.

### III.5.1.1 Anti-Hemagglutinin Antibody Response

### a) HI Geometric Mean Titres (GMT)

The GMTs for HI antibodies with 95% CI are shown in Table 7 (GMT for anti-HI antibody). Pre-vaccination GMTs of antibodies for all vaccine strains were within the same range in the three groups. After vaccination, anti-haemagglutinin antibody levels increased significantly. Post vaccination, there was a trend for higher GMTs of HI antibody for all three vaccine strains in the FluAS03 and Fluarix groups although there was some overlap of 95% CI between the Fluarix group and the FluWVV group.

TABLE 7

| | | | | GMT | | |
|---|---|---|---|---|---|---|
| | | | | | 95% CI | |
| Antibody | Group | Timing | N | Value | LL | UL |
| A/ | FluAS03 | Pre | 49 | 25.6 | 17.3 | 37.9 |
| New | Fuarix | PI(day 21) | 49 | 317.7 | 219.1 | 460.7 |
| Caledonia | FluWVV | Pre | 49 | 26.3 | 18.1 | 38.4 |
| | | PI(day 21) | 49 | 358.5 | 244.2 | 526.4 |
| | | Pre | 50 | 19.7 | 13.6 | 28.6 |
| | | PI(day 21) | 50 | 138.2 | 90.3 | 211.7 |
| A/ | FluAS03 | Pre | 49 | 52.3 | 35.4 | 77.4 |
| Panama | Fuarix | PI(day 21) | 49 | 366.1 | 264.5 | 506.6 |
| | FluWVV | Pre | 49 | 40.9 | 28.1 | 59.5 |
| | | PI(day 21) | 49 | 296.0 | 205.4 | 426.6 |
| | | Pre | 50 | 25.8 | 18.0 | 37.1 |
| | | PI(day 21) | 50 | 165.6 | 116.0 | 236.5 |
| B/ | FluAS03 | Pre | 49 | 27.5 | 19.0 | 39.8 |
| Shangdong | Fuarix | PI(day 21) | 49 | 317.7 | 226.9 | 444.9 |
| | FluWVV | Pre | 49 | 26.0 | 17.2 | 39.2 |
| | | PI(day 21) | 49 | 270.0 | 187.0 | 389.7 |
| | | Pre | 50 | 32.0 | 20.8 | 49.3 |
| | | PI(day 21) | 50 | 195.6 | 135.2 | 282.9 |

N = number of subjects with available results
95% CI = 95% confidence interval; LL = Lower Limit; UL = Upper Limit
MIN/MAX = Minimum/Maximum
PRE = Prevaccination at Day 0
PI(D 21) = Post-vaccination at Day 21)

### b) Conversion Factors of Anti-HI Antibody Titres, Seroprotection Rates and Seroconversion Rates (Correlates for Protection in Human)

Results are presented in Table 8.

The conversion factors represent the fold increase in serum HI GMTs for each vaccine strain on day 21 compared to day 0. The conversion factor varies from 6.1 to 13.6 according to the virus strain and the vaccine. This conversion factor is largely superior to the 2.0 fold increase in GMT required by the European Authorities.

The seroprotection rates represent the proportion of subjects with a serum HI titre≧40 on day 21. At the outset of the study, half of the subjects (range 34.0%-69.4%) in all groups had protective levels of antibodies for all strains At day 21, the seroprotection rates in the three groups ranged from 88.0% to 100% for the different virus strains. In terms of protection, this means that more than 88% of the subjects had a serum HI titre≧40 after vaccination and were deemed to be protected against the three strains. This rate is largely superior to the seroprotection rate of 60% required in the ≧60 years old population, by the European Authorities.

The seroconversion rates represent the proportion of subjects with at least a four-fold increase in serum HI titres on day 21 as compared to day 0. Overall response rates for the three strains were essentially equal in the three groups. To be deemed effective and according to European Union, a vaccine should induce a seroconversion rate greater than 30% in the =60 years old population. In this study, the seroconversion rate was greater than 50% for the three groups.

TABLE 8

| | | | Seroprotection | Seroconversion | Conversion |
|---|---|---|---|---|---|
| | | | rate | rate | factor |
| EU standard (>60 years) | | | >60% | >30% | >2.0 |
| Strains | Group | N | % [95% CI] | % [95% CI] | GMR [95% CI] |
| A/New | Flu AS03 | 49 | 98.0 [89.1-99.9] | 69.4 [54.6-81.7] | 12.4 [7.3-21.0] |
| Caledonia | Fluarix | 49 | 98.0 [89.1-99.9] | 69.4 [54.6-81.7] | 13.6 [8.0-23.2] |
| | Flu WVV | 50 | 88.0 [75.7-95.5] | 52.0 [37.4-66.3] | 7.0 [4.0-12.2] |
| A/Panama | Flu AS03 | 49 | 100.0 [92.7-100.0] | 55.1 [40.2-69.3] | 7.0 [4.2-11.6] |
| | Fluarix | 49 | 91.8 [80.4-97.7] | 65.3 [50.4-78.3] | 7.2 [4.7-11.3] |
| | Flu WVV | 50 | 90.0 [78.2-96.7] | 56.0 [41.3-70.0] | 6.4 [3.9-10.4] |
| B/ | Flu AS03 | 49 | 100.0 [92.7-100.0] | 73.5 [58.9-85.1] | 11.6 [7.2-18.6] |
| shangdong | Fluarix | 49 | 95.9 [86.0-99.5] | 69.4 [54.6-81.7] | 10.4 [6.5-16.5] |
| | Flu WVV | 50 | 90.0 [78.2-96.7] | 50.0 [35.5-64.5] | 6.1 [3.6-10.3] |

N = total number of subjects

In Conclusion:
Post vaccination, there was a trend for higher GMTs of HI antibody for all three vaccine strains in the FluAS03 and Fluarix groups although there was some overlap of 95% CI between the Fluarix group and the FluWVV group.
The conversion factor varies from 6.1 to 13.6 according to the virus strain and the vaccine. This conversion factor is largely superior to the 2.0 fold increase in GMT required by the European Authorities.
At day 21, the seroprotection rates in the three groups ranged from 88.0% to 100% for the different virus strains. This rate is largely superior to the seroprotection rate of 60% required in the ≧60 years old population, by the European Authorities.
In this study, the seroconversion rate was greater than 50% for the three groups. Overall response rates for the three strains were essentially equal in the three groups.

### III.5.1.2 Neutralising Antibody Titers

In order to better characterise the immune response to influenza vaccination in the elderly, the serum antibody responses to the neutralising antigens was assessed. Results are shown in Table 9 (Seroprotection rates and geometric mean titres (GMT) for anti-neutralising antibody titres) and Table 10 (Seroconversion rates for anti-neutralising at post vaccination day 21 (fold-increase=4)).

Titres of neutralising antibody against the three influenza strains were measured in pre- and post-immunization sera. The following parameters were determined:
Geometric mean titres (GMTs) of serum neutralising antibodies with 95% confidence intervals (95% CI) pre and post-vaccination
Seroconversion rates with 95% CI at day 21, defined as the percentage of vaccinees with at least a 4-fold increase in HI titres on day 21 compared to day 0, for each vaccine strain.

TABLE 9

| | | | | >=18 1/DIL | | | | GMT | | |
|---|---|---|---|---|---|---|---|---|---|---|
| | | | | | | 95% CI | | | 95% CI | |
| Antibody | Group | Timing | N | n | % | LL | UL | Value | LL | UL |
| A/ | 1 | PRE | 49 | 46 | 93.9 | 83.1 | 98.7 | 106.6 | 77.6 | 146.6 |
| NEW_CALEDONIA | | PI(D 21) | 49 | 49 | 100.0 | 92.7 | 100.0 | 870.2 | 608.5 | 1244.3 |
| | 2 | PRE | 49 | 48 | 98.0 | 89.1 | 99.9 | 115.6 | 89.4 | 149.5 |
| | | PI(D 21) | 49 | 49 | 100.0 | 92.7 | 100.0 | 955.8 | 649.5 | 1406.5 |
| | 3 | PRE | 50 | 46 | 92.0 | 80.8 | 97.8 | 87.7 | 63.6 | 120.8 |
| | | PI(D 21) | 50 | 50 | 100.0 | 92.9 | 100.0 | 375.4 | 271.2 | 519.6 |
| A/ | 1 | PRE | 49 | 49 | 100.0 | 92.7 | 100.0 | 724.7 | 558.0 | 941.1 |
| PANAMA | | PI(D 21) | 49 | 49 | 100.0 | 92.7 | 100.0 | 2012.8 | 1438.4 | 2816.5 |
| | 2 | PRE | 49 | 49 | 100.0 | 92.7 | 100.0 | 727.8 | 556.1 | 952.6 |
| | | PI(D 21) | 49 | 49 | 100.0 | 92.7 | 100.0 | 1597.7 | 1128.8 | 2261.5 |
| | 3 | PRE | 50 | 50 | 100.0 | 92.9 | 100.0 | 512.0 | 409.3 | 640.6 |
| | | PI(D 21) | 50 | 50 | 100.0 | 92.9 | 100.0 | 977.8 | 738.2 | 1295.0 |
| B/ | 1 | PRE | 49 | 29 | 59.2 | 44.2 | 73.0 | 25.6 | 18.8 | 35.0 |
| SHANGDONG | | PI(D 21) | 49 | 48 | 98.0 | 89.1 | 99.9 | 222.5 | 148.1 | 334.2 |
| | 2 | PRE | 49 | 27 | 55.1 | 40.2 | 69.3 | 29.3 | 20.1 | 42.7 |
| | | PI(D 21) | 49 | 49 | 100.0 | 92.7 | 100.0 | 190.4 | 127.6 | 284.3 |
| B/ | 3 | PRE | 50 | 31 | 62.0 | 47.2 | 75.3 | 33.4 | 23.1 | 48.4 |
| SHANGDONG | | PI(D 21) | 50 | 46 | 92.0 | 80.8 | 97.8 | 117.8 | 82.6 | 168.0 |

Group 1: Flu vaccine mix Adjuvant 2× conc Flu vac
Group 2: Flu vaccine Flu vaccine
Group 3: Flu vaccine Flu WVV vaccine
N = number of subjects with available results
n/% = number/percentage of subjects with titre within the specified range
95% CI = 95% confidence interval; LL = Lower Limit; UL = Upper Limit
PRE = Pre-vaccination at Day 0
PI(D 21) = Post-vaccination at Day 21)

TABLE 10

Responders

95% CI

| Antibody | Group | N | n | % | LL | UL |
|---|---|---|---|---|---|---|
| A/ | 1 | 49 | 29 | 59.2 | 44.2 | 73.0 |
| New | 2 | 49 | 30 | 61.2 | 46.2 | 74.8 |
| Caledonia | 3 | 50 | 21 | 42.0 | 28.2 | 56.8 |
| A/ | 1 | 49 | 12 | 24.5 | 13.3 | 38.9 |
| Panama | 2 | 49 | 9 | 18.4 | 8.8 | 32.0 |
| | 3 | 50 | 9 | 18.0 | 8.6 | 31.4 |
| B/ | 1 | 49 | 29 | 59.2 | 44.2 | 73.0 |
| Shangdong | 2 | 49 | 26 | 53.1 | 38.3 | 67.5 |
| | 3 | 50 | 19 | 38.0 | 24.7 | 52.8 |

Group 1: Flu vaccine (DFLU58A16) mix Adjuvant (D621024A8) 2× conc Flu vac
Group 2: Flu vaccine (18854B9) Flu vaccine
Group 3: Flu vaccine (DFLU59A2) Flu WVV vaccine
N = number of subjects with both pre and post vaccination result available.
n = number of responders
% = Proportion of responders (n/N × 100).
95% CI = exact 95% confidence interval; LL = lower limit, UL = upper limit

The main findings are:
For the three vaccines, at day 21, a seroprotection rate of 100% is obtained for both A strains. For the B strain, the seroprotection rates in the three groups ranged from 92% to 100%.
Post vaccination, there was a significant increase of GMT for all strains, in the three groups. However, there was a trend for higher GMTs of neutralising antibody for all three vaccine strains in the FluAS03 and Fluarix groups than in the FluWVV although there was some overlap of 95% CI between the Fluarix group and the FluWVV group.
For the seroconversion rates, overall response rates for the three strains were essentially equal in the three groups.

In all groups, the results were consistent with those obtained from the analysis performed for anti-hemagglutinin antibodies.

### III.5.1.3 Nauraminidase (NA) Antibody Titers

In order to better characterise the immune response to influenza vaccination in the elderly population, the serum antibody responses to the neuraminidase antigens was assessed. Similarly to the HI antibody titre, the following endpoints were determined:
GMT (taking the anti-log of the mean of the log titre transformations)
Seroconversion rate defined as the percentage of vaccinees with at least a 4-fold increase in HI titres on day 21 compared to day 0, for each vaccine strain.

The GMTs and seroconversion rates for NI antibodies with 95% CI are shown in Table 11 (anti-NA antibody GMT) and Table 12 (Seroconversion rates of NA at post-vaccination (day 21) (4-fold-increase)).

TABLE 11

| | | | | | 95% CI | |
|---|---|---|---|---|---|---|
| Antibody | Group | Timing | N | GMT | LL | UL |
| A/ | FluAS03 | PRE | 49 | 77.8 | 61.8 | 97.9 |
| New | | PI(D 21) | 48 | 270.0 | 212.9 | 342.3 |
| Caledonia | Fluarix | PRE | 49 | 77.8 | 64.6 | 93.6 |
| | | PI(D 21) | 49 | 249.1 | 190.0 | 326.5 |
| | FluWVV | PRE | 50 | 66.8 | 53.8 | 83.0 |
| | | PI(D 21) | 50 | 159.2 | 122.8 | 206.4 |
| A/ | FluAS03 | PRE | 49 | 33.3 | 28.5 | 48.7 |
| Panama | | PI(D 21) | 48 | 156.8 | 124.8 | 196.9 |
| | Fluarix | PRE | 49 | 34.2 | 25.6 | 45.8 |
| | | PI(D 21) | 49 | 133.7 | 100.9 | 177.3 |
| | FluWVV | PRE | 50 | 24.6 | 18.7 | 32.4 |
| | | PI(D 21) | 49 | 78.9 | 59.4 | 104.7 |
| B/ | FluAS03 | PRE | 49 | 46.7 | 36.5 | 59.9 |
| Shangdong | | PI(D 21) | 49 | 204.2 | 156.4 | 266.7 |
| | Fluarix | PRE | 49 | 46.1 | 35.3 | 60.1 |
| | | PI(D 21) | 49 | 133.7 | 100.9 | 177.3 |
| | FluWVV | PRE | 50 | 48.6 | 36.4 | 64.7 |
| | | PI(D 21) | 49 | 128.2 | 101.7 | 161.6 |

FluAS03: Flu vaccine (DFLU58A16) mix with AS03 Adjuvant (D621024A8)
Fluarix: Flu vaccine (18854B9)
FluWVV: Flu WVV vaccine (DFLU59A2)
PRE = Pre-vaccination, PI(D 21) = Day 21 post vaccination
95% CI, LL, and UL = 95% confidence interval, lower and upper limit

TABLE 12

| | | | Responders | | | |
|---|---|---|---|---|---|---|
| | | | | | 95% CI | |
| Antibody | Group | N | n | % | LL | UL |
| A/ | FluAS03 | 48 | 25 | 52.1 | 37.2 | 66.7 |
| New | Fluarix | 49 | 24 | 49.0 | 34.4 | 63.7 |
| Caledonia | FluWVV | 49 | 18 | 36.7 | 23.4 | 51.7 |
| A/ | FluAS03 | 48 | 27 | 56.3 | 41.2 | 70.5 |
| Panama | Fluarix | 49 | 23 | 46.9 | 32.5 | 61.7 |
| | FluWVV | 49 | 21 | 42.9 | 28.8 | 57.8 |
| B/ | FluAS03 | 48 | 26 | 54.2 | 39.2 | 68.6 |
| Shangdong | Fluarix | 49 | 23 | 46.9 | 32.5 | 61.7 |
| | FluWVV | 49 | 16 | 32.7 | 19.9 | 47.5 |

FluAS03: Flu vaccine (DFLU58A16) mix with AS03 Adjuvant (D621024A8), Fluarix: Flu vaccine (18854B9), FluWVV: Flu WVV vaccine (DFLU59A2)
N = number of subjects with both pre and post vaccination result available, n = number of responders.
% = Proportion of responders (n/N × 100).
95% CI = exact 95% confidence interval; LL = lower limit, UL = upper limit

The main findings are:
Higher value of the GMT and seroconversion rates were observed for hemagglutinin than for neuraminidase.
Pre-vaccination GMTs of antibodies for all vaccine strains were within the same range in the three groups. After vaccination, anti-neuraminidase antibody levels increased significantly. As for the HI antibody titres, post vaccination, there was a trend for higher GMTs of HI antibody for all three vaccine strains in the FluAS03 and Fluarix groups although there was some overlap of 95% CI between the Fluarix group and the FluWVV group.
Regarding the seroconversion rates, overall response rates for the three strains were essentially equal in the three groups and for the three strains.

Our results show that healthy elderly vaccinated in this study against influenza developed good antibody responses to neuraminidase antigens whatever the influenza vaccine. However, the response to the neuraminidase antigen is lower than the response to the hemagglutinin antigen.

### III.5.2. Cellular Immune Response

Peripheral blood antigen-specific CD4 and CD8 T cells can be restimulated in vitro to produce IL-2, CD40L, TNF-alpha and IFNγ if incubated with their corresponding antigen. Consequently, antigen-specific CD4 and CD8 T cells can be enumerated by flow cytometry following conventional immunofluorescence labelling of cellular phenotype as well as intracellular cytokines production. In the present study, Influenza vaccine antigen as well as peptides derived from specific influenza protein were used as antigen to restimulate Influenza-specific T cells. Results are presented for the CD4 and CD8 T-cell response in Tables 13 to 18.

TABLE 13
Antigen specific CD4 ′T-cell responses expressed into cells producing
at least two different cytokines: Descriptive Statistics on PRE and
POST for CD40L/IL2/TNF-α/IFN-γ (Total vaccinated cohort)

| | | | Time | | | | |
|---|---|---|---|---|---|---|---|
| Secretion | Antigen | Gr | point | N | Mean | SD | Min |
| CD40L/IL2/IFN-γ/ | Peptide | 1 | Day 0 | 44 | 33.50 | 139.026 | 1.00 |
| TNF-α in CD4 | Influenza | 1 | Day 21 | 45 | 58.40 | 132.664 | 1.00 |
| | | 2 | Day 0 | 42 | 92.10 | 368.790 | 1.00 |
| | | 2 | Day 21 | 44 | 88.36 | 272.528 | 1.00 |
| | | 3 | Day 0 | 45 | 80.13 | 284.316 | 1.00 |
| | | 3 | Day 21 | 47 | 91.40 | 382.967 | 1.00 |
| | Split | 1 | Day 0 | 47 | 1901.66 | 1596.203 | 102.00 |
| | Influenza | 1 | Day 21 | 48 | 6163.75 | 4265.900 | 773.00 |
| | | 2 | Day 0 | 45 | 2151.04 | 2622.594 | 265.00 |
| | | 2 | Day 21 | 49 | 4150.73 | 3712.469 | 328.00 |
| | | 3 | Day 0 | 48 | 1678.44 | 916.329 | 142.00 |
| | | 3 | Day 21 | 50 | 3374.60 | 1920.194 | 449.00 |
| | Whole | 1 | Day 0 | 48 | 3134.33 | 2568.369 | 507.00 |
| | Influenza | 1 | Day 21 | 47 | 9332.04 | 6875.403 | 1482.00 |
| | | 2 | Day 0 | 47 | 3050.85 | 2654.936 | 486.00 |
| | | 2 | Day 21 | 49 | 6760.31 | 6788.258 | 1852.00 |
| | | 3 | Day 0 | 48 | 2955.33 | 2019.233 | 473.00 |
| | | 3 | Day 21 | 50 | 5661.40 | 4530.321 | 635.00 |

| | | | | | | | | Kruskall- |
|---|---|---|---|---|---|---|---|---|
| | | | Time | | | | | Wallis test |
| Secretion | Antigen | Gr | point | Q1 | Median | Q3 | Max | (P-value) |
| CD40L/IL2/IFN-γ/ | Peptide | 1 | Day 0 | 1.00 | 1.00 | 4.00 | 915.00 | 0.7631 |
| TNF-α in CD4 | Influenza | 1 | Day 21 | 1.00 | 1.00 | 56.00 | 733.00 | |
| | | 2 | Day 0 | 1.00 | 1.00 | 54.00 | 2393.00 | |
| | | 2 | Day 21 | 1.00 | 1.00 | 69.50 | 1740.00 | |
| | | 3 | Day 0 | 1.00 | 1.00 | 65.00 | 1908.00 | |
| | | 3 | Day 21 | 1.00 | 1.00 | 63.00 | 2615.00 | |
| | Split | 1 | Day 0 | 957.00 | 1560.00 | 2408.00 | 9514.00 | 0.0002 |
| | Influenza | 1 | Day 21 | 3468.00 | 4908.00 | 7624.00 | 21324.00 | |
| | | 2 | Day 0 | 930.00 | 1381.00 | 2274.00 | 16289.00 | |
| | | 2 | Day 21 | 2247.00 | 3036.00 | 4744.00 | 21924.00 | |
| | | 3 | Day 0 | 1086.00 | 1502.00 | 2189.00 | 3899.00 | |
| | | 3 | Day 21 | 2312.00 | 3040.00 | 4437.00 | 10431.00 | |
| | Whole | 1 | Day 0 | 1730.00 | 2298.50 | 3876.00 | 15066.00 | 0.0040 |
| | Influenza | 1 | Day 21 | 4091.00 | 6523.00 | 14045.00 | 29251.00 | |
| | | 2 | Day 0 | 1190.00 | 2031.00 | 4161.00 | 11994.00 | |
| | | 2 | Day 21 | 3573.00 | 4621.00 | 7234.00 | 40173.00 | |
| | | 3 | Day 0 | 1421.50 | 2668.50 | 3411.50 | 10578.00 | |
| | | 3 | Day 21 | 2459.00 | 4315.00 | 7303.00 | 22053.00 | |

Group 1: FluAS03: Flu vaccine Fluarix ™ mixed with AS03 Adjuvant
Group 2: Fluarix: Flu vaccine Fluarix ™
Group 3: FluWVV: Flu WVV vaccine
SD = Standard Deviation; Min, Max = Minimum, Maximum
Q1 = First quartile; Q3 = Third quartile
N = number of subjects with available results
P-value: Kruskall -Wallis Test (Non-parametric procedure) to test location difference (Wilcoxon rank-sum test) between the 3 groups at Day 21.

TABLE 14
Antigen-specific CD4 T-cell responses expressed into cells producing
at least two different cytokines: Descriptive Statistics on difference
between PRE and POST (′Total vaccinated cohort)

| Secretion | Antigen | Group | N | Mean | SD | Min |
|---|---|---|---|---|---|---|
| CD40L/IFN-γ/ | Peptide | 1 | 44 | 9.57 | 159.363 | −860.00 |
| TNF-α in CD4 | Influenza | 2 | 42 | −40.98 | 386.998 | −2392.00 |
| | | 3 | 45 | −50.73 | 256.596 | −1664.00 |
| | Split | 1 | 47 | 4307.02 | 4468.828 | −8161.00 |
| | Influenza | 2 | 45 | 1982.93 | 3802.332 | −14318.0 |
| | | 3 | 48 | 1555.90 | 1596.216 | −526.00 |
| | Whole | 1 | 47 | 6197.98 | 7220.765 | −11763.0 |
| | Influenza | 2 | 47 | 3791.34 | 5820.894 | −2128.00 |
| | | 3 | 48 | 2535.98 | 3966.345 | −4766.00 |
| CD40L/IFN-γ/ | Peptide | 1 | 42 | −15.95 | 215.710 | −451.00 |
| TNF-α in CD8 | Influenza | 2 | 41 | 50.83 | 264.370 | −614.00 |
| | | 3 | 44 | −52.11 | 243.811 | −684.00 |
| | Split | 1 | 42 | 134.71 | 426.699 | −603.00 |
| | Influenza | 2 | 44 | −65.05 | 822.036 | −4938.00 |
| | | 3 | 45 | 2.49 | 330.700 | −1094.00 |
| | Whole | 1 | 39 | 189.38 | 1394.153 | −2641.00 |
| | Influenza | 2 | 44 | −479.75 | 1790.094 | −9455.00 |
| | | 3 | 44 | −243.73 | 719.269 | −1892.00 |

| Secretion | Antigen | Group | Q1 | Median | Q3 | Max | P-value |
|---|---|---|---|---|---|---|---|
| CD40L/IFN-γ/ | Peptide | 1 | 0.00 | 0.00 | 37.50 | 430.00 | 0.0765 |
| TNF-α in CD4 | Influenza | 2 | −15.00 | 0.00 | 26.00 | 514.00 | |
| | | 3 | −37.00 | 0.00 | 0.00 | 212.00 | |
| | Split | 1 | 1888.00 | 3396.00 | 6634.00 | 19555.00 | <0.0001 |
| | Influenza | 2 | 699.00 | 1490.00 | 2573.00 | 15169.00 | |
| | | 3 | 466.00 | 1183.50 | 2186.50 | 7851.00 | |
| | Whole | 1 | 2170.00 | 4009.00 | 11681.00 | 25570.00 | 0.0003 |
| | Influenza | 2 | 1246.00 | 2382.00 | 3992.00 | 33801.00 | |
| | | 3 | 503.00 | 1382.50 | 3300.50 | 19337.00 | |
| CD40L/IFN-γ/ | Peptide | 1 | −106.00 | 0.00 | 81.00 | 655.00 | 0.0932 |
| TNF-α in CD8 | Influenza | 2 | −58.00 | 13.00 | 202.00 | 703.00 | |
| | | 3 | −160.50 | 0.00 | 53.00 | 567.00 | |
| | Split | 1 | −122.00 | 35.50 | 221.00 | 1387.00 | 0.2121 |
| | Influenza | 2 | −64.50 | 0.00 | 160.50 | 1252.00 | |
| | | 3 | −99.00 | 0.00 | 76.00 | 1060.00 | |
| | Whole | 1 | −420.00 | 49.00 | 591.00 | 5045.00 | 0.0851 |
| | Influenza | 2 | −1016.00 | −263.50 | 180.00 | 3743.00 | |
| | | 3 | −651.00 | −86.50 | 180.00 | 1011.00 | |

TABLE 15
Antigen-specific CD4 T-cell responses expressed into cells producing
at least CD40L and another cytokine: Descriptive Statistics on
difference between PRE and POST (′Total vaccinated cohort)

| Secretion | Antigen | Group | N | Mean | SD | Min |
|---|---|---|---|---|---|---|
| CD40L in CD4 | Peptide | 1 | 44 | 10.09 | 153.007 | −815.00 |
| | Influenza | 2 | 42 | −29.40 | 316.983 | −1921.00 |
| | | 3 | 45 | −43.73 | 251.146 | −1629.00 |
| | Split | 1 | 46 | 4266.20 | 4470.807 | −8093.00 |
| | Influenza | 2 | 45 | 2026.42 | 3511.508 | −11482.0 |
| | | 3 | 47 | 1512.34 | 1576.133 | −494.00 |
| | Whole | 1 | 47 | 6071.96 | 7118.132 | −11691.0 |
| | Influenza | 2 | 47 | 3764.64 | 5740.762 | −2114.00 |
| | | 3 | 48 | 2544.27 | 3959.879 | −4390.00 |
| CD40 L in CD8 | Peptide | 1 | 44 | −19.41 | 81.675 | −370.00 |
| | Influenza | 2 | 41 | −3.98 | 100.998 | −399.00 |
| | | 3 | 45 | −5.56 | 64.666 | −181.00 |
| | Split | 1 | 43 | 39.53 | 190.122 | −438.00 |
| | Influenza | 2 | 44 | 27.61 | 91.173 | −155.00 |
| | | 3 | 45 | 30.18 | 191.326 | −291.00 |
| | Whole | 1 | 41 | −91.24 | 617.077 | −1779.00 |
| | Influenza | 2 | 44 | −115.91 | 588.424 | −2583.00 |
| | | 3 | 45 | −150.89 | 367.300 | −1239.00 |

| Secretion | Antigen | Group | Q1 | Median | Q3 | Max | P-value |
|---|---|---|---|---|---|---|---|
| CD40L n CD4 | Peptide | 1 | 0.00 | 0.00 | 36.50 | 428.00 | 0.1233 |
| | Influenza | 2 | −8.00 | 0.00 | 27.00 | 494.00 | |
| | | 3 | −35.00 | 0.00 | 3.00 | 230.00 | |
| | Split | 1 | 1799.00 | 3156.50 | 6647.00 | 19480.00 | <0.0001 |
| | Influenza | 2 | 783.00 | 1485.00 | 2546.00 | 15021.00 | |
| | | 3 | 469.00 | 1107.00 | 2035.00 | 7687.00 | |
| | Whole | 1 | 2109.00 | 4048.00 | 11472.00 | 25448.00 | 0.0004 |
| | Influenza | 2 | 1212.00 | 2509.00 | 3957.00 | 33428.00 | |
| | | 3 | 523.00 | 1392.00 | 3261.50 | 19478.00 | |
| CD40L in CD8 | Peptide | 1 | −2.00 | 0.00 | 0.50 | 100.00 | 0.9721 |
| | Influenza | 2 | −28.00 | 0.00 | 24.00 | 231.00 | |
| | | 3 | −13.00 | 0.00 | 3.00 | 176.00 | |
| | Split | 1 | −35.00 | 0.00 | 140.00 | 608.00 | 0.6175 |
| | Influenza | 2 | −18.50 | 0.00 | 77.50 | 326.00 | |
| | | 3 | −9.00 | 0.00 | 28.00 | 1188.00 | |
| | Whole | 1 | −142.00 | −8.00 | 175.00 | 2087.00 | 0.3178 |
| | Influenza | 2 | −195.50 | −34.50 | 150.00 | 1258.00 | |
| | | 3 | −270.00 | −103.00 | 88.00 | 588.00 | |

TABLE 16
Antigen-specific CD4 T-cell responses expressed into cells producing
at least IFNγ and another cytokine: Descriptive Statistics
on difference between PRE and POST (′Total vaccinated cohort)

| | | | | N | | | |
|---|---|---|---|---|---|---|---|
| Secretion | Antigen | Group | N | missing | Mean | SD | Min |
| IFNγ in CD4 | Peptide | 1 | 44 | 5 | 7.50 | 64.539 | −171.00 |
| | Influenza | 2 | 42 | 7 | −30.67 | 277.984 | −1766.00 |
| | | 3 | 45 | 5 | −27.91 | 103.403 | −639.00 |
| | Split | 1 | 46 | 3 | 2712.87 | 2905.629 | −4394.00 |
| | Influenza | 2 | 45 | 4 | 1148.56 | 2526.536 | −10586.0 |
| | | 3 | 47 | 3 | 871.00 | 1016.251 | −764.00 |
| | Whole | 1 | 47 | 2 | 4240.09 | 4811.891 | −8272.00 |
| | Influenza | 2 | 47 | 2 | 2445.38 | 4030.694 | −3018.00 |
| | | 3 | 48 | 2 | 1535.48 | 2456.915 | −3670.00 |
| IFNγ in CD8 | Peptide | 1 | 44 | 5 | 7.75 | 146.412 | −226.00 |
| | Influenza | 2 | 41 | 8 | 10.68 | 176.026 | −420.00 |
| | | 3 | 44 | 6 | −49.80 | 217.214 | −699.00 |
| | Split | 1 | 43 | 6 | 138.58 | 365.565 | −470.00 |
| | Influenza | 2 | 44 | 5 | −112.82 | 793.746 | −4919.00 |
| | | 3 | 44 | 6 | 29.91 | 238.157 | −708.00 |
| | Whole | 1 | 41 | 8 | 6.66 | 1642.577 | −5610.00 |
| | Influenza | 2 | 44 | 5 | −471.55 | 1792.348 | −9586.00 |
| | | 3 | 44 | 6 | −189.05 | 685.291 | −1879.00 |

| Secretion | Antigen | Group | Q1 | Median | Q3 | Max | P-value |
|---|---|---|---|---|---|---|---|
| IFNγ in CD4 | Peptide | | −9.50 | 0.00 | 7.50 | 265.00 | 0.1541 |
| | Influenza | 2 | −5.00 | 0.00 | 24.00 | 222.00 | |
| | | 3 | −20.00 | 0.00 | 0.00 | 51.00 | |
| | Split | 1 | 1273.00 | 1644.00 | 4057.00 | 13296.00 | <0.0001 |
| | Influenza | 2 | 405.00 | 931.00 | 1757.00 | 9426.00 | |
| | | 3 | 283.00 | 624.00 | 1114.00 | 5031.00 | |
| | Whole | 1 | 1610.00 | 2693.00 | 7437.00 | 17489.00 | <0.0001 |
| | Influenza | 2 | 723.00 | 1487.00 | 2983.00 | 21594.00 | |
| | | 3 | 232.50 | 810.00 | 2218.50 | 11319.00 | |
| IFNγ in CD8 | Peptide | 1 | −52.50 | 0.00 | 40.00 | 615.00 | 0.3322 |
| | Influenza | 2 | −1.00 | 0.00 | 72.00 | 610.00 | |
| | | 3 | −172.00 | 0.00 | 90.50 | 424.00 | |
| | Split | 1 | −46.00 | 42.00 | 294.00 | 1549.00 | 0.1257 |
| | Influenza | 2 | −62.00 | 0.00 | 74.00 | 1028.00 | |
| | | 3 | −59.50 | 26.50 | 123.00 | 643.00 | |
| | Whole | 1 | −385.00 | 131.00 | 450.00 | 5068.00 | 0.1179 |
| | Influenza | 2 | −955.50 | −221.00 | 177.00 | 3492.00 | |
| | | 3 | −476.50 | −36.50 | 198.00 | 1299.00 | |

TABLE 17
Antigen-specific CD4 T-cell responses expressed into cells producing
at least IL2 and another cytokine: Descriptive Statistics on difference
between PRE and POST (′Total vaccinated cohort)

| Secretion | Antigen | Group | N | Mean | SD | Min |
|---|---|---|---|---|---|---|
| IL2 in CD4 | Peptide | 1 | 44 | 2.82 | 118.164 | −595.00 |
| | Influenza | 2 | 42 | 0.90 | 84.255 | −167.00 |
| | | 3 | 45 | −28.62 | 191.709 | −1222.00 |
| | Split | 1 | 46 | 3456.15 | 3853.960 | −7009.00 |
| | Influenza | 2 | 45 | 1738.29 | 2406.045 | −451.00 |
| | | 3 | 47 | 1210.02 | 1361.705 | −634.00 |
| | Whole | 1 | 47 | 4839.02 | 5978.277 | −9178.00 |
| | Influenza | 2 | 47 | 2891.00 | 4493.387 | −1370.00 |
| | | 3 | 48 | 2042.50 | 3123.912 | −3179.00 |
| IL2 in CD8 | Peptide | 1 | 42 | −30.60 | 219.777 | −630.00 |
| | Influenza | 2 | 41 | 38.85 | 210.715 | −674.00 |
| | | 3 | 45 | −44.80 | 197.026 | −526.00 |
| | Split | 1 | 41 | 54.85 | 250.817 | −336.00 |
| | Influenza | 2 | 44 | −2.36 | 423.957 | −2272.00 |
| | | 3 | 45 | −26.07 | 244.870 | −1004.00 |
| | Whole | 1 | 39 | 56.21 | 406.262 | −704.00 |
| | Influenza | 2 | 44 | −151.02 | 822.384 | −4304.00 |
| | | 3 | 45 | −63.56 | 359.699 | −1036.00 |

| Secretion | Antigen | Group | Q1 | Median | Q3 | Max | P-value |
|---|---|---|---|---|---|---|---|
| IL2 in CD4 | Peptide | 1 | −1.50 | 0.00 | 31.50 | 324.00 | 0.0806 |
| | Influenza | 2 | −34.00 | 0.00 | 2.00 | 362.00 | |
| | | 3 | −19.00 | 0.00 | 0.00 | 253.00 | |
| | Split | 1 | 1309.00 | 2598.50 | 5926.00 | 16988.00 | <0.0001 |
| | Influenza | 2 | 453.00 | 1113.00 | 2049.00 | 12273.00 | |
| | | 3 | 331.00 | 806.00 | 1596.00 | 6474.00 | |
| | Whole | 1 | 1516.00 | 3341.00 | 8955.00 | 21032.00 | 0.0006 |
| | Influenza | 2 | 995.00 | 1942.00 | 3007.00 | 26358.00 | |
| | | 3 | 371.50 | 1083.50 | 2624.50 | 14057.00 | |
| IL2 in CD8 | Peptide | 1 | −111.00 | 0.00 | 103.00 | 412.00 | 0.1684 |
| | Influenza | 2 | −41.00 | 0.00 | 138.00 | 542.00 | |
| | | 3 | −150.00 | −34.00 | 71.00 | 447.00 | |
| | Split | 1 | −76.00 | 26.00 | 133.00 | 803.00 | 0.2311 |
| | Influenza | 2 | −78.50 | 0.00 | 121.50 | 1064.00 | |
| | | 3 | −93.00 | −1.00 | 30.00 | 705.00 | |
| | Whole | 1 | −167.00 | 63.00 | 261.00 | 1302.00 | 0.4586 |
| | Influenza | 2 | −444.50 | −4.00 | 199.00 | 1398.00 | |
| | | 3 | −198.00 | 9.00 | 131.00 | 838.00 | |

TABLE 18
Antigen-specific CD4 T-cell responses expressed into cells producing
at least TNFα and another cytokine: Descriptive Statistics
on difference between PRE and POST (′Total vaccinated cohort)

| Secretion | Antigen | Group | N | Mean | SD | Min |
|---|---|---|---|---|---|---|
| TNF-α in CD4 | Peptide | 1 | 44 | 9.48 | 92.992 | −466.00 |
| | Influenza | 2 | 42 | −47.71 | 367.624 | −2333.00 |
| | | 3 | 45 | −37.38 | 179.147 | −1169.00 |
| | Split | 1 | 46 | 2343.11 | 2596.177 | −4450.00 |
| | Influenza | 2 | 45 | 703.87 | 2973.241 | −14260.0 |
| | | 3 | 47 | 732.00 | 740.001 | −611.00 |
| | Whole | 1 | 47 | 3103.74 | 4248.997 | −5146.00 |
| | Influenza | 2 | 47 | 1658.38 | 3639.959 | −1393.00 |
| | | 3 | 48 | 1010.15 | 1689.394 | −1482.00 |
| TNF-α in CD8 | Peptide | 1 | 42 | 11.71 | 201.031 | −453.00 |
| | Influenza | 2 | 41 | 37.46 | 245.241 | −612.00 |
| | | 3 | 44 | −42.95 | 210.185 | −645.00 |
| | Split | 1 | 41 | 138.54 | 362.601 | −329.00 |
| | Influenza | 2 | 44 | −70.27 | 790.309 | −4741.00 |
| | | 3 | 44 | −39.75 | 348.803 | −1044.00 |
| | Whole | 1 | 39 | 279.59 | 1048.352 | −1184.00 |
| | Influenza | 2 | 44 | −280.70 | 1562.095 | −9070.00 |
| | | 3 | 44 | −71.57 | 492.135 | −1574.00 |

| Secretion | Antigen | Group | Q1 | Median | Q3 | Max | P-value |
|---|---|---|---|---|---|---|---|
| TNF-α in CD4 | Peptide | 1 | −1.50 | 0.00 | 39.00 | 239.00 | 0.1836 |
| | Influenza | 2 | −4.00 | 0.00 | 12.00 | 277.00 | |
| | | 3 | −26.00 | 0.00 | 5.00 | 53.00 | |
| | Split | 1 | 862.00 | 1466.50 | 3931.00 | 9267.00 | <0.0001 |
| | Influenza | 2 | 251.00 | 698.00 | 1229.00 | 12275.00 | |
| | | 3 | 191.00 | 540.00 | 1010.00 | 3288.00 | |
| | Whole | 1 | 868.00 | 1607.00 | 5266.00 | 17199.00 | 0.0008 |
| | Influenza | 2 | 367.00 | 871.00 | 1584.00 | 23540.00 | |
| | | 3 | 175.00 | 592.00 | 1385.50 | 8760.00 | |
| TNF-α in CD8 | Peptide | 1 | −80.00 | 0.50 | 70.00 | 772.00 | 0.2759 |
| | Influenza | 2 | −81.00 | 0.00 | 155.00 | 791.00 | |
| | | 3 | −179.00 | 0.00 | 39.50 | 566.00 | |
| | Split | 1 | −23.00 | 60.00 | 178.00 | 1468.00 | 0.0790 |
| | Influenza | 2 | −107.00 | 0.00 | 158.00 | 1286.00 | |
| | | 3 | −185.00 | 0.00 | 78.50 | 1021.00 | |
| | Whole | 1 | −250.00 | 108.00 | 399.00 | 4601.00 | 0.1482 |
| | Influenza | 2 | −392.00 | −56.50 | 205.00 | 3258.00 | |
| | | 3 | −233.50 | −54.00 | 160.00 | 1543.00 | |

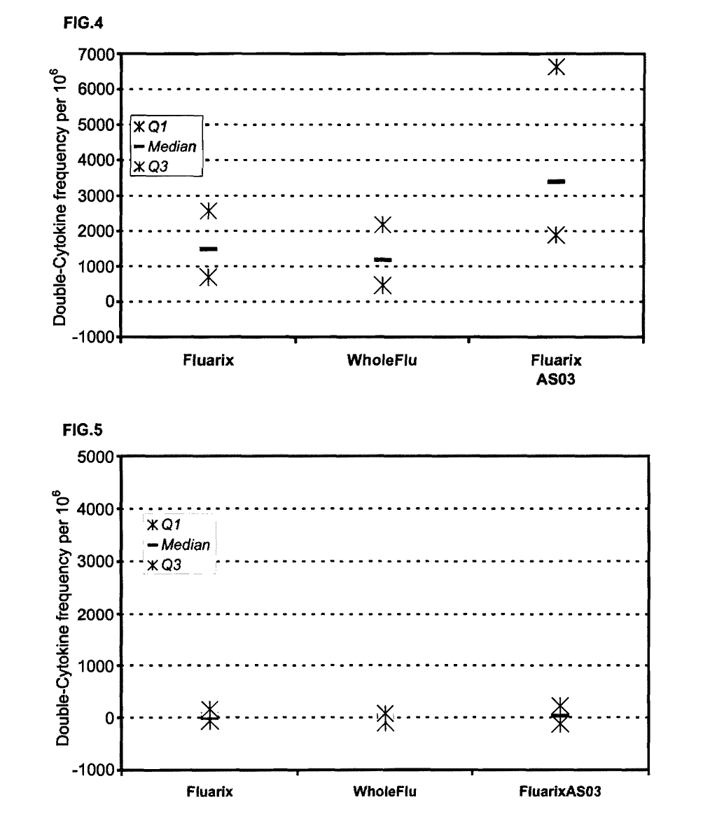

Results were also expressed as a frequency of cytokine(s)-positive CD4 or CD8 T cell within the CD4 or CD8 T cell sub-population and presented in FIG. 4 and FIG. 5.

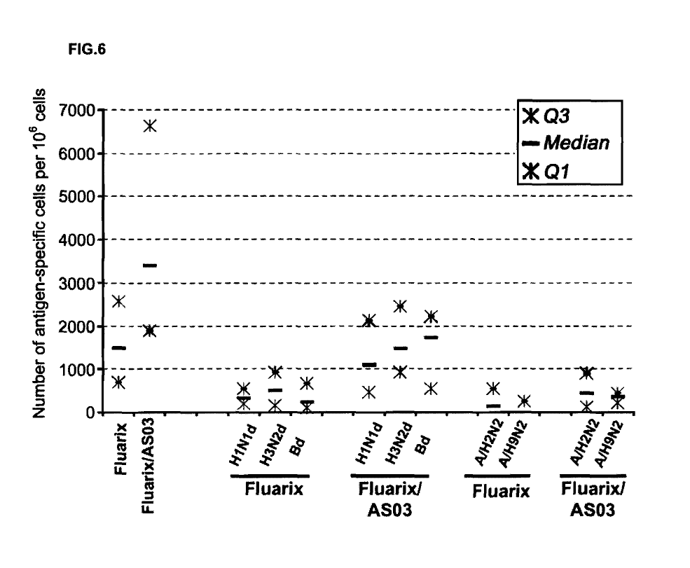

In a similar analysis, the cross-reactive CD4 T-cells response was evaluated using influenza antigen from drifted strains (A/H1N1/Beijing/262/95 (H1N1d), A/H3N2/Sydney/5/97 (H3N2d), B/Yamanashi/166/98 (Bd)) or shift strains (A/Singapore/1/57 (H2N2), A/Hongkong/1073/99 (H9N2)). Results expressed as a frequency of cytokine(s)-positive CD4 T cells are presented in FIG. 6.

The main findings are:
Vaccination with Fluarix or Whole virus slightly boosts the CD4 T-cell response. Vaccination with Flu AS03 induces a strong CD4 T-cell response (FIG. 4), and this is statistically significant. The same conclusion is made after In Vitro stimulation with the split antigen or Whole virus, and this with all cytokines investigated (IL-2, IFNγ, TNFα, and CD40L).
Most individuals have a CD8 T-cell response against the whole flu, however the vaccination has no measurable impact on the CD8 T-cell response (i.e. Pre=post), whatever the group studied (FIG. 5).

Vaccination with Fluarix only induces low levels of cross-reactive CD4 T-cell response (FIG. 6). Vaccination with FluAS03 induces a strong CD4 T-cell response against drifted influenza strains and this is statistically significant (FIG. 6). A little response was detected against shift strains.

### III.5.3. B-Cells Elispot Memory

### III.5.3.1 Objective

In order to better characterise the CMI response induced by the AS03-adjuvanted influenza vaccine, the B-cells Elispot memory response induced to differentiate into plasma cells in vitro using influenza vaccine strains or anti-human immunoglobulin was evaluate in order to enumerate anti-influenza or IgG secreting plasma. The results are described in Table 19 and Table 20 and in FIG. 7.

A subset of 22 first subjects having received one dose of FluAS03 vaccine and 21 first subjects having received one dose of Fluarix vaccine was selected to evaluate the impact of vaccination on influenza-specific memory B-cells using the B-cell memory Elispot technology. The following endpoints were determined
At days 0 and 21: Influenza-specific memory B-cells have been measured by B-cell Elispot in all subjects. Results have been expressed as a frequency of Influenza specific-antibody forming cells per million (10⁶) of antibody forming cells.
Difference between post (day 21) and pre (day 0) vaccination is also expressed as a frequency of Influenza specific-antibody forming cells per million (10⁶) of antibody forming cells.

### III.5.3.2 Statistical Methods

Descriptive statistics for each vaccination group at days 0 and day 21 expressed as a frequency of Influenza specific-antibody forming cells per million (10⁶) of antibody forming cells. Descriptive statistics in individual difference between day 21 and day 0 (Post-Pre) as a frequency of Influenza specific-antibody forming cells per million (10⁶) of antibody forming cells.

A Wilcoxon test was used to compare the location of difference between the two groups and the statistical p-value was calculated for each of 3 strains (A/New Calcdonia, A/Panama and B/Shangdong).

### III.5.3.3 Results

There is a tendency in favour of the influenza adjuvanted AS03 vaccine compared to Fluarix group. For A/New Calcdonia strain, there is a statistical significant difference (p-value=0.021) in favour of FluAS03 compared to Fluarix. No statistical difference between the two groups was observed for A/Panama and B/Shangdong strains.

TABLE 19
B-cells Memory: descriptive statistics on pre (Day 0) and post (Day 21)
and inferential statistics of post (Day 21) frequency of antigen-plasma within
a 10⁶ of IgG-producing plasma cells (subset of subjects)

| | | Time- | | | | |
|---|---|---|---|---|---|---|
| STRAIN | Group | point | N | Mean | SD | Min |
| A/NEW | 1 | Day 0 | 22 | 9751.58 | 6630.335 | 0.00 |
| CALEDONIA | 1 | Day 21 | 22 | 22001.65 | 11308.261 | 3981.90 |
| | 2 | Day 0 | 21 | 9193.61 | 4339.421 | 1300.81 |
| | 2 | Day 21 | 21 | 12263.08 | 7285.698 | 789.47 |
| A/PANAMA | 1 | Day 0 | 22 | 4329.17 | 2923.497 | 0.00 |
| | 1 | Day 21 | 22 | 18066.69 | 14604.842 | 714.29 |
| | 2 | Day 0 | 21 | 4860.41 | 3392.373 | 0.00 |
| | 2 | Day 21 | 21 | 13872.95 | 12052.163 | 0.00 |
| B/SHANDONG | 1 | Day 0 | 22 | 3722.80 | 2347.315 | 0.00 |
| | 1 | Day 21 | 22 | 15949.60 | 12385.965 | 0.00 |
| | 2 | Day 0 | 21 | 3030.39 | 2206.589 | 640.57 |
| | 2 | Day 21 | 21 | 9714.03 | 5656.805 | 0.00 |

| | | | | | | | P-value |
|---|---|---|---|---|---|---|---|
| | | Time- | | | | | (Wilcoxon |
| STRAIN | Gr | point | Q1 | Median | Q3 | Max | test) |
| A/NEW | 1 | Day 0 | 4117.65 | 9606.46 | 13430.66 | 25570.78 | 0.0056 |
| CALEDONIA | 1 | Day 21 | 11052.63 | 20450.55 | 30234.74 | 40526.32 | |
| | 2 | Day 0 | 6363.64 | 9686.41 | 11698.11 | 19164.84 | |
| | 2 | Day 21 | 7741.05 | 9545.45 | 17069.60 | 32000.00 | |
| A/PANAMA | 1 | Day 0 | 2275.45 | 4003.02 | 5764.55 | 10842.49 | 0.1814 |
| | 1 | Day 21 | 9347.37 | 13176.41 | 21471.39 | 54789.92 | |
| | 2 | Day 0 | 2222.22 | 4545.45 | 7495.74 | 11698.11 | |
| | 2 | Day 21 | 6231.88 | 10147.06 | 20540.54 | 52188.84 | |
| B/SHANDONG | 1 | Day 0 | 2058.82 | 2956.78 | 5972.22 | 7832.17 | 0.1483 |
| | 1 | Day 21 | 6860.47 | 12796.90 | 22947.37 | 48947.37 | |
| | 2 | Day 0 | 1290.32 | 2113.82 | 4770.02 | 7783.25 | |
| | 2 | Day 21 | 6590.91 | 9009.01 | 12774.87 | 21201.72 | |

Group 1: Flu vaccine Fluarix ™ + AS03 oil-in-water emulsion adjuvant
Group 2: Flu vaccine Fluarix ™
SD = Standard Deviation
Min, Max = Minimum, Maximum
Q1 = First quartile
Q3 = Third quartile
N = number of subjects with available results
P-value: Wilcoxon Test (Non-parametric procedure) to test location difference (Wilcoxon rank-sum test) between the 2 groups at Day 21.

TABLE 20
B cells Memory: Descriptive and inferential statistics on difference
between POST (Day 21) and PRE (Day 0) frequency of antigen-specific plasma
within a 10 6 of IgG-producing plasma cells (subset of subjects)

| STRAIN | Group | N | Mean | SD | Min |
|---|---|---|---|---|---|
| A/NEW | 1 | 22 | 12250.07 | 12875.755 | −4365.08 |
| CALEDONIA | 2 | 21 | 3069.46 | 7309.731 | −10043.4 |
| A/PANAMA | 1 | 22 | 13737.52 | 13677.942 | −188.29 |
| | 2 | 21 | 9012.54 | 11489.012 | −1551.05 |
| B/SHANDONG | 1 | 22 | 12226.81 | 12243.895 | −2222.22 |
| | 2 | 21 | 6683.64 | 6240.312 | −2113.82 |

| | | | | | | P-value |
|---|---|---|---|---|---|---|
| | | | | | | (Wilcoxon |
| STRAIN | Gr | Q1 | Median | Q3 | Max | test) |
| A/NEW | 1 | 2418.07 | 6776.65 | 26036.01 | 35059.98 | 0.0210 |
| CALEDONIA | 2 | −1762.54 | 1694.51 | 6850.19 | 18579.97 | |
| A/PANAMA | 1 | 4551.30 | 11039.04 | 16614.85 | 49881.94 | 0.1449 |
| | 2 | 1522.85 | 6480.96 | 9214.67 | 47812.47 | |
| B/SHANDONG | 1 | 1788.75 | 9322.70 | 18907.05 | 42134.18 | 0.1895 |
| | 2 | 2117.44 | 5384.41 | 9897.27 | 19801.28 | |

Group 1: Flu vaccine Fluarix ™ + AS03 oil-in-water emulsion adjuvant
Group 2: Flu vaccine Fluarix ™
SD = Standard Deviation
Min, Max = Minimum, Maximum
Q1 = First quartile
Q3 = Third quartile
N = number of subjects with available results
P-value: Wilcoxon Test (Non-parametric procedure) to test location difference (Wilcoxon rank-sum test) between the 2 groups at Day 21.

### III.6. Overall Conclusions

### III.6.1. Reactogenicity and Safety Results

While influenza immunization significantly reduces the risk of pneumonia and associated deaths, vaccination of elderly only affords 23-72% protection against influenza disease. Formulation of vaccine antigen with potent adjuvants is an attractive approach for enhancing immune responses to subunit antigens. This study was designed to evaluate (1) the safety and reactogenicity in healthy elderly of an influenza vaccine adjuvanted with oil in water emulsion, i.e. AS03, (2) the antibody and cell-mediated immune responses. Reactogenicity data show that the influenza vaccine adjuvanted with AS03 induced more local and general symptoms than the two other vaccines. However regarding unsolicited adverse events, no difference was observed between the three vaccines. From these results, it can be concluded that the reactogenicity and safety profile of the candidate vaccines is satisfactory and clinically acceptable.

### III.6.2. Immunogenicity Results

Regarding the immune response, the three vaccines exceeded the requirements of the European authorities for annual registration of split virion influenza vaccines (“Note for Guidance on Harmonisation of Requirements for influenza Vaccines” for the immuno-logical assessment of the annual strain changes -CPMP/BWP/214/96). The three influenza vaccines tested in this study were immunogenic in the healthy elderly, who developed a good antibody response to influenza hemagglutinin and neutralising antigens (Table 21).

TABLE 21

| Variable | EU standard for antibody response | Results |
|---|---|---|
| Conversion factor | >2.0 | >6.1 |
| Seroconversion rate | >30% | >50% |
| Protection rate | >60% | >88% |

Regarding cell-mediated immunity (CMI) response, the influenza vaccine adjuvanted with AS03 induced a significantly stronger CD4 response (included drifted strains) than the two other vaccines (Fluarix and whole influenza virus vaccine). However, vaccination has no measurable impact on the CD8 response.

Regarding the B cell memory response, there is a tendency in favour of the influenza adjuvanted vaccine compared to the un-adjuvanted vaccine.

### Example IV

### Clinical Trial in an Elderly Population Aged Over 65 Years with a Vaccine Containing a Split Influenza Antigen Preparation and AS03 Adjuvant—Explo-Flu-002

A phase I/II, open, controlled study has been conducted in order to evaluate the reactogenicity and the immunogenicity of the GlaxoSmithKline Biologicals influenza candidate vaccine containing the adjuvant AS03, in an elderly population aged over 65 years and previously vaccinated in 2003 with the candidate vaccine in the Explo-Flu-001 clinical trial. For immunogenicity and safety evaluations, Fluarix™ vaccine (known as α-rix™ in Belgium) has been used as reference.

### IV.1. Objective

The humoral immune response (i.e. anti-hemagglutinin antibody titres) and cell mediated immune response (CD4 and/or CD8 T cell responses) and B memory cell response were measured 21 days after intramuscular administration of one dose of an AS03 adjuvanted vaccine. Fluarix™ was used as reference.

The objectives were:

1) to determine if AS03 adjuvanted Flu (40 subjects) versus Fluarix (18 subjects) confirm his strongest immunostimulating activity on CD4- and/or CD8-mediated immunity of individuals vaccinated with influenza antigens;
2) to investigate, using a longitudinal analysis, the influence of AS03 adjuvanted on the immune response in prevaccination 2004 (so response one year after the first vaccination in 2003).

### IV.2. Study Design, Vaccine Composition and End-Points

40 subjects aged>65 years who have previously received one dose of the AS03 adjuvanted influenza vaccine during the Explo-Flu-001 clinical trial in 2003 (FluAS03)
one control group of about 20 subjects aged>65 years who have previously received one dose of Fluarix™ during the Explo-Flu-001 clinical trial in 2003 (Fluarix)

### IV.2.1. Vaccine Composition

The vaccine composition is similar to that used for the study Explo-Flu-001 except for the influenza strains included in the vaccine (year 2004 vaccine). The strains are as follows:
A/New Calcdonia/20/99 (IVR-116) (H1N1)=A/New Calcdonia/(HINI)—like strain
A/Wyoming/3/2003 (X-147) (H3N2)=A/Fujian (H3N2)—like strain
B/Jiangsu/10/2003=B/Shanghai—like strain

### IV.2.2. Immunogenicity (HI) End-Points

GMTs (taking the anti-log of the mean of the log titre transformations)
Conversion factors (the fold increase in serum HI GMTs on day 21 compared to day 0)
Seroconversion rate (the percentage of vaccinees with at least a four-fold increases in HI titers on day 21 compared to day 0, for each vaccine strain)
Protection rate (the percentage of vaccinees with a serum HI ≧1: 40 at day 21)

### IV.2.3. CMI-Endpoints

### Observed Variable:

At days 0 and 21: frequency of cytokine-positive CD4/CD8 cells per 10⁶ into 4 different cytokines. Each test quantifies the response of CD4/CD8 T cell to:
Pool of the 3 following antigens
New Calcdonia antigen
Wyoming antigen
Jiangsu antigen.

### Derived Variables:

Antigen-specific CD4 and CD8-T-cell response expressed into the 5 different tests (cytokines):

1. cells producing at least two different cytokines (CD40L, IL-2, IFNγ, TNFα)
2. cells producing at least CD40L and another cytokine (IL-2, TNFα, IFNγ)
3. cells producing at least IL-2 and another cytokine (CD40L, TNFα, IFNγ)
4. cells producing at least IFNγ and another cytokine (IL-2, TNFα, CD40L)
5. cells producing at least TNFα and another cytokine (IL-2, CD40L, IFNγ)

### IV.2.4. CMI Analysis

The first CMI analysis was based on the Total Vaccinated cohort (N=40 subjects for FluAS03 group and N=18 subjects for Fluarix group).

A longitudinal analysis was based on the Kinetic cohort of the Explo-Flu-001 (split protein) and Explo-Flu-002 (pool flu antigen) studies:
Pre: N=36 subjects for FluAS03 group and N=15 for Fluarix group.
Post-Pre: N=34 subjects for FluAS03 group and N=15 for Fluarix group.
(a) The frequency of CD4/CD8 T-lymphocytes secreting in response was summarised by descriptive statistics for each antigen, for each cytokine, for each vaccine group and at each timepoint (pre- and post-vaccination).
(b) Descriptive statistics in individual difference between timepoints (Post-Pre) responses were tabulated for each antigen, for each cytokine and for each vaccine group.
(c) For the timepoints post and (post-pre) vaccination, non-parametric Wilcoxon's test was used to compare the location differences between the two vaccine groups and to calculate the statistical p-value regarding the 4 different cytokines on:
   CD4 T-cell response to New Calcdonia, Wyoming, Jiangsu and the pool of the 3 strains.
   CD8 T-cell response to New Calcdonia, Wyoming, Jiangsu and the pool of the 3 strains.
(d) Non-parametric test (Wilcoxon-test) was also used:
   To investigate the kinetic of the immune response at Pre (Day 0) in term of frequency of specific CD4 between Explo-Flu-001 and Explo-Flu-002 in each vaccine group
   To investigate the kinetic of the immune response at Pre (Day 0) in term of frequency of specific CD4 between the 2 vaccine groups in each of the study Explo-Flu-001 and Explo-Flu-002
   To investigate the kinetic of the immune response in term of differences (Post-Pre) of frequency of specific CD4 between Explo-Flu-001 and Explo-Flu-002 in each vaccine group.
   To investigate the kinetic of the immune response in term of differences (Post-Pre) of frequency of specific CD4 between the 2 vaccine groups in each of the study Explo-Flu-001 and Explo-Flu-002

All significance tests were two-tailed. P-values less than or equal to 0.05 were considered as statistically significant.

### IV.3. Results

Results were expressed as a frequency of cytokine(s)-positive CD4 or CD8 T cell within the CD4 or CD8 T cell sub-population.

### IV.3.1. Antigen Specific CD4 T-Lymphocytes

The frequency of antigen-specific CD4 T-lymphocytes secreting in response was summarised by descriptive statistics for each antigen, for each cytokine, for each vaccine group and at each timepoint (pre- and post-vaccination).

Descriptive statistics in individual difference between time points (Post-Pre) in CD4 T-lymphocytes responses for each antigen at each 5 different cytokines and for each vaccine group are shown in Table 22.

TABLE 22
Descriptive Statistics on difference between Post-vaccination (at Day 21) and Prevaccination
(at Day 0) for the antigen-specific CD4 T-lymphocyte responses (Total vaccinated cohort)

| | | Vaccine | | | | | | | | |
|---|---|---|---|---|---|---|---|---|---|---|
| Antigen | Cytokine | Group | N | Mean | SD | Min | Q1 | Median | Q3 | Max |
| Pool Flu | All double | Fluarix | 18 | 1268.67 | 1051.744 | 197.00 | 724.00 | 863.00 | 1561.00 | 4676.00 |
| | | FluAS03 | 36 | 1781.31 | 1484.860 | −2379.00 | 929.50 | 1664.50 | 2821.00 | 4669.00 |
| | CD40L | Fluarix | 18 | 1260.11 | 1054.487 | 243.00 | 721.00 | 849.00 | 1602.00 | 4743.00 |
| | | FluAS03 | 36 | 1711.56 | 1433.113 | −2359.00 | 838.00 | 1576.00 | 2759.50 | 4575.00 |
| | IFNγ | Fluarix | 18 | 762.94 | 813.884 | −12.00 | 294.00 | 496.00 | 1061.00 | 3564.00 |
| | | FluAS03 | 36 | 1179.92 | 881.255 | −817.00 | 692.50 | 1180.50 | 1865.50 | 2831.00 |
| | IL2 | Fluarix | 18 | 1019.06 | 917.905 | −258.00 | 544.00 | 702.00 | 1174.00 | 3850.00 |
| | | FluAS03 | 36 | 1423.33 | 1359.471 | −2702.00 | 651.00 | 1260.00 | 2200.50 | 4342.00 |
| | TNFα | Fluarix | 18 | 803.39 | 915.838 | 32.00 | 231.00 | 533.00 | 936.00 | 3892.00 |
| | | FluAS03 | 36 | 1078.28 | 1029.122 | −1816.00 | 446.00 | 983.00 | 1836.00 | 3310.00 |
| A/New | All double | Fluarix | 18 | 481.44 | 381.534 | −241.00 | 282.00 | 448.50 | 598.00 | 1412.00 |
| Caledonia | | FluAS03 | 36 | 812.78 | 749.192 | −828.00 | 215.50 | 911.50 | 1274.50 | 3206.00 |
| | CD40L | Fluarix | 18 | 450.78 | 360.378 | −239.00 | 291.00 | 447.00 | 580.00 | 1248.00 |
| | | FluAS03 | 36 | 783.75 | 711.608 | −760.00 | 242.00 | 808.00 | 1161.00 | 3050.00 |
| | IFNγ | Fluarix | 18 | 316.28 | 279.662 | −165.00 | 175.00 | 259.00 | 387.00 | 1111.00 |
| | | FluAS03 | 36 | 438.22 | 420.770 | −685.00 | 125.00 | 393.00 | 733.50 | 1557.00 |
| | IL2 | Fluarix | 18 | 326.06 | 290.792 | −294.00 | 193.00 | 330.00 | 488.00 | 834.00 |
| | | FluAS03 | 36 | 634.72 | 616.478 | −557.00 | 179.50 | 678.50 | 952.00 | 2602.00 |
| | TNFα | Fluarix | 18 | 316.44 | 372.492 | −140.00 | 50.00 | 278.00 | 542.00 | 1449.00 |
| | | FluAS03 | 36 | 449.17 | 591.796 | −916.00 | 100.50 | 343.50 | 848.00 | 2452.00 |
| A/Wyoming | All double | Fluarix | 18 | 609.56 | 559.396 | −176.00 | 257.00 | 510.50 | 957.00 | 1998.00 |
| | | FluAS03 | 36 | 766.61 | 579.191 | −568.00 | 316.00 | 864.50 | 1221.00 | 1662.00 |
| | CD40L | Fluarix | 18 | 616.33 | 550.853 | −176.00 | 274.00 | 488.00 | 939.00 | 2017.00 |
| | | FluAS03 | 36 | 728.61 | 570.316 | −670.00 | 260.00 | 789.50 | 1216.00 | 1675.00 |
| | IFNγ | Fluarix | 18 | 407.06 | 424.758 | −311.00 | 129.00 | 370.50 | 723.00 | 1372.00 |
| | | FluAS03 | 36 | 526.72 | 443.938 | −770.00 | 219.00 | 556.50 | 776.00 | 1342.00 |
| | IL2 | Fluarix | 18 | 495.83 | 503.805 | −187.00 | 88.00 | 540.50 | 801.00 | 1841.00 |
| | | FluAS03 | 36 | 572.89 | 533.728 | −789.00 | 220.00 | 602.00 | 882.50 | 1512.00 |
| | TNFα | Fluarix | 18 | 424.56 | 485.591 | −260.00 | 110.00 | 359.50 | 461.00 | 1718.00 |
| | | FluAS03 | 36 | 550.58 | 538.461 | −765.00 | 269.50 | 543.50 | 905.50 | 1678.00 |
| B/Jiangsu | All double | Fluarix | 18 | 698.44 | 793.119 | −306.00 | 233.00 | 433.00 | 961.00 | 2822.00 |
| | | FluAS03 | 36 | 861.42 | 688.852 | −223.00 | 339.00 | 745.00 | 1325.50 | 2284.00 |
| | CD40L | Fluarix | 18 | 678.39 | 777.259 | −206.00 | 227.00 | 401.50 | 962.00 | 2878.00 |
| | | FluAS03 | 36 | 825.89 | 674.879 | −223.00 | 305.00 | 722.00 | 1282.00 | 2337.00 |
| | IFNγ | Fluarix | 18 | 431.72 | 489.912 | −95.00 | 191.00 | 272.50 | 382.00 | 1712.00 |
| | | FluAS03 | 36 | 615.94 | 473.543 | −286.00 | 288.50 | 501.50 | 897.50 | 1740.00 |
| | IL2 | Fluarix | 18 | 552.50 | 666.853 | −234.00 | 155.00 | 278.50 | 833.00 | 2386.00 |
| | | FluAS03 | 36 | 696.19 | 622.931 | −359.00 | 207.50 | 540.50 | 1146.50 | 2182.00 |
| | TNFα | Fluarix | 18 | 441.39 | 695.792 | −338.00 | 97.00 | 269.50 | 564.00 | 2440.00 |
| | | FluAS03 | 36 | 500.03 | 448.636 | −166.00 | 107.50 | 436.00 | 745.00 | 1626.00 |

SD = Standard Deviation
Min, Max = Minimum, Maximum
Q1 = First quartile
Q3 = Third quartile
N = number of subjects tested with available results

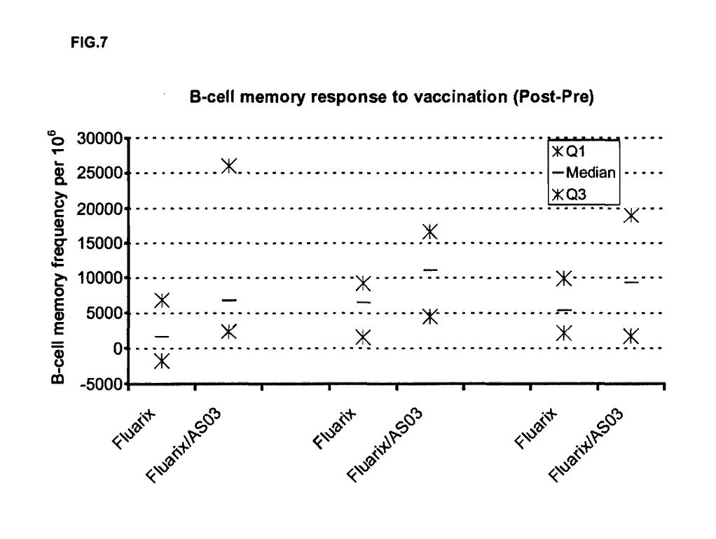

Vaccine-induced CD4 T-cells are shown to be able to persist at least for one year since there is an observable difference in prevaccination levels of CD4 T-cell responses between individuals vaccinated with Fluarix has compared to those vaccinated with Fluarix/AS03 the year before. The results are also shown in FIG. 8, showing the CD4 T-cell response to split Flu antigen before and after revaccination. D0 corresponds to 12 months after first year vaccination and thus indicates persistence.

Comparing the difference in the frequency of antigen-specific CD4 T-lymphocytes between the 2 groups by Wilcoxon test at post-vaccination, almost all p-values were less than 0.05 and were considered as statistically significant (see Table 23) in favour of the FluAS03 group.

TABLE 23
Inferential statistics: p-values from Wilcoxon rank-sum test
between the two vaccine groups at Day 21 for antigen-
specific CD4 T-lymphocyte responses (Total vaccinated cohort)

P-value

| | | New | | |
|---|---|---|---|---|
| Cytokine | Pool | Caledonia | Wyoming | Jiangsu |
| All | 0.0014 | 0.0023 | 0.0286 | 0.0133 |
| double | | | | |
| CD40L | 0.0016 | 0.0014 | 0.0427 | 0.0155 |
| INFγ | 0.0006 | 0.0366 | 0.0400 | 0.0041 |
| IL2 | 0.0037 | 0.0024 | 0.0584 | 0.0162 |
| TNFα | 0.0031 | 0.0103 | 0.0918 | 0.0114 |

P-value: Wilcoxon Test (Non-parametric procedure) to test location difference (Wilcoxon rank-sum test) between the 2 groups at Day 21.

Comparing the difference of the individual difference (Post-Pre) in the frequency of antigen-specific CD4−T-lymphocytes responses between the 2 groups by Wilcoxon test, p-values less than 0.05 and considered as statistically significant occurred for the following antigen-cytokine combinations: pool flu-all double, pool flu-IFNγ and Jiangsu-IFNγ in favour of the FluAS03 group (Table 24).

TABLE 24
Inferential statistics: p-values calculated by Wilcoxon
rank-sum test between the different groups on the
difference between Post-vaccination (at Day 21) and
Prevaccination (at 0) for the antigen-specific CD4 T-
lymphocyte responses (Total vaccinated cohort)

P-value

| | | New | | |
|---|---|---|---|---|
| Cytokine | Pool | Caledonia | Wyoming | Jiangsu |
| All | 0.0435 | 0.1124 | 0.2189 | 0.3085 |
| double | | | | |
| CD40L | 0.0638 | 0.0781 | 0.2831 | 0.2872 |
| INFγ | 0.0290 | 0.3589 | 0.2553 | 0.0435 |
| IL2 | 0.1024 | 0.0563 | 0.3986 | 0.0435 |
| TNFα | 0.0693 | 0.4090 | 0.1232 | 0.3129 |

P-value: Wilcoxon Test (Non-parametric procedure) to test location difference (Wilcoxon rank-sum test) between the 2 groups.

### IV.3.2. Antigen Specific CD8 T-Lymphocytes

The frequency of antigen-specific CD8 T-lymphocytes secreting in response was summarised by descriptive statistics for each antigen, for each cytokine, for each vaccine group and at each timepoint (pre- and post-vaccination), similarly to the procedure followed in respect of CD4 T cell response.

Comparing the difference in the frequency of antigen-specific CD8 T-lymphocytes between the 2 groups by Wilcoxon test at post-vaccination, all p-values were higher than 0.05 and were not considered as statistically significant. Comparing the difference of the individual difference (Post-Pre) in the frequency of antigen-specific CD8-T-lymphocytes responses between the 2 groups by Wilcoxon test, all p-values were higher than 0.05 and were not considered as statistically significant.

IV.3.3. Kinetic Analysis: Immune Response at Prevaccination (One Year after the First Vaccination in 2003)

The frequency of antigen-specific CD4 T-lymphocytes secreting in response at prevaccination was summarised by descriptive statistics for each cytokine and for each vaccine group and for each of the two studies in Table 25, for each of the two studies study and for each vaccine group in Table 27. Inferential statistics are given in Table 26 and Table 28.

TABLE 25
Descriptive Statistics on prevaccination (Day 0) for the
specific CD4 T-lymphocytes response vaccination (Kinetic)

| Cytokine | Group | Study | N | Mean | SD | Min | Q1 | Median | Q3 | Max |
|---|---|---|---|---|---|---|---|---|---|---|
| All double | FluAS03 | EXPLO 001 | 36 | 2000.86 | 1783.474 | 102.00 | 911.50 | 1461.50 | 2791.00 | 9514.00 |
| | | EXPLO 002 | 36 | 2028.28 | 1427.000 | 55.00 | 1190.50 | 1647.50 | 2575.00 | 7214.00 |
| | Fluarix | EXPLO 001 | 15 | 2152.87 | 2162.463 | 747.00 | 930.00 | 1354.00 | 2101.00 | 7868.00 |
| | | EXPLO 002 | 15 | 1587.07 | 2123.841 | 192.00 | 468.00 | 735.00 | 1578.00 | 8536.00 |
| CD40L | FluAS03 | EXPLO 001 | 35 | 1946.66 | 1771.102 | 120.00 | 837.00 | 1340.00 | 2819.00 | 9462.00 |
| | | EXPLO 002 | 35 | 1992.20 | 1440.721 | 77.00 | 1125.00 | 1590.00 | 2587.00 | 7286.00 |
| | Fluarix | EXPLO 001 | 15 | 2094.93 | 2076.632 | 745.00 | 902.00 | 1340.00 | 2077.00 | 7385.00 |
| | | EXPLO 002 | 15 | 1561.73 | 2097.201 | 34.00 | 475.00 | 672.00 | 1579.00 | 8428.00 |
| INFγ | FluAS03 | EXPLO 001 | 35 | 1068.63 | 1030.745 | 91.00 | 448.00 | 790.00 | 1503.00 | 5425.00 |
| | | EXPLO 002 | 35 | 1259.23 | 890.590 | 312.00 | 725.00 | 984.00 | 1354.00 | 4146.00 |
| | Fluarix | EXPLO 001 | 15 | 1248.07 | 1452.459 | 320.00 | 388.00 | 778.00 | 1227.00 | 5431.00 |
| | | EXPLO 002 | 15 | 974.80 | 1394.044 | 52.00 | 252.00 | 337.00 | 1057.00 | 5576.00 |
| IL2 | FluAS03 | EXPLO 001 | 35 | 1690.20 | 1524.689 | 37.00 | 688.00 | 1211.00 | 2416.00 | 8235.00 |
| | | EXPLO 002 | 35 | 1883.60 | 1361.337 | 14.00 | 1068.00 | 1413.00 | 2370.00 | 6891.00 |
| | Fluarix | EXPLO 001 | 15 | 1888.40 | 2085.857 | 568.00 | 715.00 | 1136.00 | 1770.00 | 7403.00 |
| | | EXPLO 002 | 15 | 1493.93 | 2037.139 | 58.00 | 444.00 | 755.00 | 1485.00 | 8193.00 |
| TNFα | FluAS03 | EXPLO 001 | 35 | 1174.74 | 1119.633 | 55.00 | 466.00 | 795.00 | 1720.00 | 5415.00 |
| | | EXPLO 002 | 35 | 1545.40 | 1159.490 | 135.00 | 831.00 | 1203.00 | 1857.00 | 5354.00 |
| | Fluarix | EXPLO 001 | 15 | 1444.20 | 1946.211 | 201.00 | 520.00 | 688.00 | 1254.00 | 7213.00 |
| | | EXPLO 002 | 15 | 1304.73 | 1759.716 | 144.00 | 316.00 | 824.00 | 1171.00 | 7056.00 |

SD = Standard Deviation
Min, Max = Minimum, Maximum
Q1 = First quartile
Q3 = Third quartile
N = number of subjects tested with available results

Comparing the difference in the frequency of antigen-specific CD4 T-lymphocytes between the 2 studies by Wilcoxon test for each vaccine group, p-values less than 0.05 and considered as statistically significant (in favour of Explo-Flu-002) occurred only for FluAS03 group and with TNFα cytokine (see Table 26).

TABLE 26
Inferential statistics: p-values from Wilcoxon rank-sum test
between the different studies at Day 0 for antigen-specific
CD4 T-lymphocyte responses (Kinetic)

| Cytokine | Group | p-value |
|---|---|---|
| ALL | FluAS03 | 0.5209 |
| DOUBLE | Fluarix | 0.0712 |
| CD40L | FluAS03 | 0.4957 |
| | Fluarix | 0.0744 |
| INFγ | FluAS03 | 0.0896 |
| | Fluarix | 0.1103 |
| IL2 | FluAS03 | 0.1903 |
| | Fluarix | 0.1647 |
| TNFα | FluAS03 | 0.0427 |
| | Fluarix | 0.5476 |

P-value: Wilcoxon Test (Non-parametric procedure) to test location difference (Wilcoxon rank-sum test) between the 2 groups at Day 21.

TABLE 27
Descriptive Statistics on Prevaccination (Day 0) for the
specific CD4 T-lymphocytes response vaccination (Kinetic)

| Cytokine | Study | Group | N | Mean | SD | Min | Q1 | Median | Q3 | Max |
|---|---|---|---|---|---|---|---|---|---|---|
| All double | EXPLO 001 | FluAS03 | 36 | 2000.86 | 1783.474 | 102.00 | 911.50 | 1461.50 | 2791.00 | 9514.00 |
| | | Fluarix | 15 | 2152.87 | 2162.463 | 747.00 | 930.00 | 1354.00 | 2101.00 | 7868.00 |
| | EXPLO 002 | FluAS03 | 36 | 2028.28 | 1427.000 | 55.00 | 1190.50 | 1647.50 | 2575.00 | 7214.00 |
| | | Fluarix | 15 | 1587.07 | 2123.841 | 192.00 | 468.00 | 735.00 | 1578.00 | 8536.00 |
| CD40L | EXPLO 001 | FluAS03 | 35 | 1946.66 | 1771.102 | 120.00 | 837.00 | 1340.00 | 2819.00 | 9462.00 |
| | | Fluarix | 15 | 2094.93 | 2076.632 | 745.00 | 902.00 | 1340.00 | 2077.00 | 7385.00 |
| | EXPLO 002 | FluAS03 | 35 | 1992.20 | 1440.721 | 77.00 | 1125.00 | 1590.00 | 2587.00 | 7286.00 |
| | | Fluarix | 15 | 1561.73 | 2097.201 | 34.00 | 475.00 | 672.00 | 1579.00 | 8428.00 |
| INFγ | EXPLO 001 | FluAS03 | 35 | 1068.63 | 1030.745 | 91.00 | 448.00 | 790.00 | 1503.00 | 5425.00 |
| | | Fluarix | 15 | 1248.07 | 1452.459 | 320.00 | 388.00 | 778.00 | 1227.00 | 5431.00 |
| | EXPLO 002 | FluAS03 | 35 | 1259.23 | 890.590 | 312.00 | 725.00 | 984.00 | 1354.00 | 4146.00 |
| | | Fluarix | 15 | 974.80 | 1394.044 | 52.00 | 252.00 | 337.00 | 1057.00 | 5576.00 |
| IL2 | EXPLO 001 | FluAS03 | 35 | 1690.20 | 1524.689 | 37.00 | 688.00 | 1211.00 | 2416.00 | 8235.00 |
| | | Fluarix | 15 | 1888.40 | 2085.857 | 568.00 | 715.00 | 1136.00 | 1770.00 | 7403.00 |
| | EXPLO 002 | FluAS03 | 35 | 1883.60 | 1361.337 | 14.00 | 1068.00 | 1413.00 | 2370.00 | 6891.00 |
| | | Fluarix | 15 | 1493.93 | 2037.139 | 58.00 | 444.00 | 755.00 | 1485.00 | 8193.00 |
| TNFα | EXPLO 001 | FluAS03 | 35 | 1174.74 | 1119.633 | 55.00 | 466.00 | 795.00 | 1720.00 | 5415.00 |
| | | Fluarix | 15 | 1444.20 | 1946.211 | 201.00 | 520.00 | 688.00 | 1254.00 | 7213.00 |
| | EXPLO 002 | FluAS03 | 35 | 1545.40 | 1159.490 | 135.00 | 831.00 | 1203.00 | 1857.00 | 5354.00 |
| | | Fluarix | 15 | 1304.73 | 1759.716 | 144.00 | 316.00 | 824.00 | 1171.00 | 7056.00 |

SD = Standard Deviation
Min, Max = Minimum, Maximum
Q1 = First quartile
Q3 = Third quartile
N = number of subjects tested with available results

Comparing the difference in the frequency of antigen-specific CD4 T-lymphocytes between the 2 vaccine groups by Wilcoxon test for each study, all p-values for Explo-Flu-002 were less than 0.05 and were considered as statistically significant (in favour of FluAS03) (see Table 28).

TABLE 28
Inferential statistics: p-values from Wilcoxon rank-sum test
between the different groups at Day 21 for antigen-specific
CD4 T-lymphocyte responses (Kinetic)

| Cytokine | Study | p-value |
|---|---|---|
| ALL DOUBLE | Explo Flu 001 | 0.9423 |
| | Explo Flu 002 | 0.0300 |
| CD40L | Explo Flu 001 | 0.8989 |
| | Explo Flu 002 | 0.0361 |
| INFγ | Explo Flu 001 | 0.8738 |
| | Explo Flu 002 | 0.0121 |
| IL2 | Explo Flu 001 | 0.9747 |
| | Explo Flu 002 | 0.0216 |
| TNFα | Explo Flu 001 | 0.9916 |
| | Explo Flu 002 | 0.0514 |

P-value: Wilcoxon Test (Non-parametric procedure) to test location difference (Wilcoxon rank-sum test) between the 2 groups at Day 21.

### IV.3.4. Kinetic Analysis: Immune Response at Post Minus Prevaccination

The frequency of antigen-specific CD4 T-lymphocytes secreting in response at (post-pre) timepoint was summarised by descriptive statistics for each cytokine and for each vaccine group and for each study in Table 29, for each study and for each vaccine group in Table 31. Inferential statistics are given in Table 30 and Table 32.

TABLE 29
Descriptive Statistics on the difference between Post-vaccination (Day 21) and Prevaccination
(Day 0) for the specific CD4 T-lymphocytes response vaccination (Kinetic)

| Cytokine | Group | Study | N | Mean | SD | Min | Q1 | Median | Q3 | Max |
|---|---|---|---|---|---|---|---|---|---|---|
| All double | FluAS03 | EXPLO 001 | 34 | 4837.56 | 4476.129 | −609.00 | 1888.00 | 3483.50 | 8148.00 | 19555.00 |
| | | EXPLO 002 | 34 | 1737.79 | 1450.177 | −2379.00 | 936.00 | 1664.50 | 2743.00 | 4669.00 |
| | Fluarix | EXPLO 001 | 15 | 3103.53 | 3726.645 | 436.00 | 800.00 | 2283.00 | 3226.00 | 15169.00 |
| | | EXPLO 002 | 15 | 1369.00 | 1127.784 | 197.00 | 725.00 | 869.00 | 1808.00 | 4676.00 |
| CD40L | FluAS03 | EXPLO 001 | 33 | 4819.06 | 4489.788 | −718.00 | 1799.00 | 3479.00 | 8288.00 | 19480.00 |
| | | EXPLO 002 | 33 | 1694.73 | 1431.082 | −2359.00 | 921.00 | 1659.00 | 2662.00 | 4575.00 |
| | Fluarix | EXPLO 001 | 15 | 3090.00 | 3684.759 | 477.00 | 822.00 | 2189.00 | 3208.00 | 15021.00 |
| | | EXPLO 002 | 15 | 1360.93 | 1131.051 | 243.00 | 725.00 | 860.00 | 1687.00 | 4743.00 |
| IFNγ | FluAS03 | EXPLO 001 | 33 | 3127.09 | 2974.067 | −453.00 | 1325.00 | 1721.00 | 5162.00 | 13296.00 |
| | | EXPLO 002 | 33 | 1167.85 | 893.363 | −817.00 | 633.00 | 1207.00 | 1803.00 | 2831.00 |
| | Fluarix | EXPLO 001 | 15 | 1660.13 | 1834.023 | −84.00 | 480.00 | 1386.00 | 2284.00 | 7120.00 |
| | | EXPLO 002 | 15 | 851.87 | 859.585 | 148.00 | 294.00 | 501.00 | 1222.00 | 3564.00 |
| IL2 | FluAS03 | EXPLO 001 | 33 | 3950.18 | 3878.538 | −358.00 | 1309.00 | 2780.00 | 6635.00 | 16988.00 |
| | | EXPLO 002 | 33 | 1404.67 | 1355.665 | −2702.00 | 719.00 | 1341.00 | 2109.00 | 4342.00 |
| | Fluarix | EXPLO 001 | 15 | 2413.87 | 3027.392 | 263.00 | 674.00 | 1672.00 | 2425.00 | 12273.00 |
| | | EXPLO 002 | 15 | 1117.80 | 975.934 | −258.00 | 575.00 | 714.00 | 1618.00 | 3850.00 |
| TNFα | FluAS03 | EXPLO 001 | 33 | 2627.36 | 2574.458 | −825.00 | 862.00 | 1475.00 | 4764.00 | 9267.00 |
| | | EXPLO 002 | 33 | 1072.36 | 1044.140 | −1816.00 | 447.00 | 1000.00 | 1752.00 | 3310.00 |
| | Fluarix | EXPLO 001 | 15 | 1460.53 | 3115.174 | −1586.00 | 251.00 | 813.00 | 1314.00 | 12275.00 |
| | | EXPLO 002 | 15 | 904.67 | 974.958 | 32.00 | 338.00 | 752.00 | 965.00 | 3892.00 |

SD = Standard Deviation
Min, Max = Minimum, Maximum
Q1 = First quartile
Q3 = Third quartile
N = number of subjects tested with available results

Comparing the difference in the frequency of antigen-specific CD4 T-lymphocytes between the 2 studies by Wilcoxon test for each vaccine group, all p-values for FluAS03 group were less than 0.05 and were considered as statistically significant (in favour of Explo-Flu-001) (see Table 30).

TABLE 30
Inferential statistics on the difference between Post-vaccination
(Day 21) and Prevaccination (Day 0): p-values from Wilcoxon
rank-sum test between the different studies at Day 21 for
antigen-specific CD4 T-lymphocyte responses (Kinetic)

| Cytokine | Group | p-value |
|---|---|---|
| ALL | FluAS03 | 0.0005 |
| DOUBLE | Fluarix | 0.1300 |
| CD40L | FluAS03 | 0.0007 |
| | Fluarix | 0.0890 |
| INFγ | FluAS03 | 0.0012 |
| | Fluarix | 0.1103 |
| IL2 | FluAS03 | 0.0025 |
| | Fluarix | 0.1409 |
| TNFα | FluAS03 | 0.0327 |
| | Fluarix | 0.6936 |

P-value: Wilcoxon Test (Non-parametric procedure) to test location difference (Wilcoxon rank-sum test) between the 2 groups at Day 21.

TABLE 31
Descriptive Statistics on the difference between Post-vaccination (Day 21) and Prevaccination
(Day 0) for the specific CD4 T-lymphocytes response vaccination (Kinetic)

| Cytokine | Study | Group | N | Mean | SD | Min | Q1 | Median | Q3 | Max |
|---|---|---|---|---|---|---|---|---|---|---|
| All double | EXPLO 001 | FluAS03 | 34 | 4837.56 | 4476.129 | −609.00 | 1888.00 | 3483.50 | 8148.00 | 19555.00 |
| | | Fluarix | 15 | 3103.53 | 3726.645 | 436.00 | 800.00 | 2283.00 | 3226.00 | 15169.00 |
| | EXPLO 002 | FluAS03 | 34 | 1737.79 | 1450.177 | −2379.00 | 936.00 | 1664.50 | 2743.00 | 4669.00 |
| | | Fluarix | 15 | 1369.00 | 1127.784 | 197.00 | 725.00 | 869.00 | 1808.00 | 4676.00 |
| CD40L | EXPLO 001 | FluAS03 | 33 | 4819.06 | 4489.788 | −718.00 | 1799.00 | 3479.00 | 8288.00 | 19480.00 |
| | | Fluarix | 15 | 3090.00 | 3684.759 | 477.00 | 822.00 | 2189.00 | 3208.00 | 15021.00 |
| | EXPLO 002 | FluAS03 | 33 | 1694.73 | 1431.082 | −2359.00 | 921.00 | 1659.00 | 2662.00 | 4575.00 |
| | | Fluarix | 15 | 1360.93 | 1131.051 | 243.00 | 725.00 | 860.00 | 1687.00 | 4743.00 |
| IFNγ | EXPLO 001 | FluAS03 | 33 | 3127.09 | 2974.067 | −453.00 | 1325.00 | 1721.00 | 5162.00 | 13296.00 |
| | | Fluarix | 15 | 1660.13 | 1834.023 | −84.00 | 480.00 | 1386.00 | 2284.00 | 7120.00 |
| | EXPLO 002 | FluAS03 | 33 | 1167.85 | 893.363 | −817.00 | 633.00 | 1207.00 | 1803.00 | 2831.00 |
| | | Fluarix | 15 | 851.87 | 859.585 | 148.00 | 294.00 | 501.00 | 1222.00 | 3564.00 |
| IL2 | EXPLO 001 | FluAS03 | 33 | 3950.18 | 3878.538 | −358.00 | 1309.00 | 2780.00 | 6635.00 | 16988.00 |
| | | Fluarix | 15 | 2413.87 | 3027.392 | 263.00 | 674.00 | 1672.00 | 2425.00 | 12273.00 |
| | EXPLO 002 | FluAS03 | 33 | 1404.67 | 1355.665 | −2702.00 | 719.00 | 1341.00 | 2109.00 | 4342.00 |
| | | Fluarix | 15 | 1117.80 | 975.934 | −258.00 | 575.00 | 714.00 | 1618.00 | 3850.00 |
| TFNα | EXPLO 001 | FluAS03 | 33 | 2627.36 | 2574.458 | −825.00 | 862.00 | 1475.00 | 4764.00 | 9267.00 |
| | | Fluarix | 15 | 1460.53 | 3115.174 | −1586.00 | 251.00 | 813.00 | 1314.00 | 12275.00 |
| | EXPLO 002 | FluAS03 | 33 | 1072.36 | 1044.140 | −1816.00 | 447.00 | 1000.00 | 1752.00 | 3310.00 |
| | | Fluarix | 15 | 904.67 | 974.958 | 32.00 | 338.00 | 752.00 | 965.00 | 3892.00 |

SD = Standard Deviation
Min, Max = Minimum, Maximum
Q1 = First quartile
Q3 = Third quartile
N = number of subjects tested with available results

Comparing the difference in the frequency of antigen-specific CD4 T-lymphocytes between the 2 vaccine groups by Wilcoxon test for each study, p-value was less than 0.05 only for Explo-Flu-001 and was considered as statistically significant (in favour of FluAS03) (see Table 32).

TABLE 32
Inferential statistics: p-values from Wilcoxon rank-sum test
between the different groups at Day 21 for antigen-specific
CD4 T-lymphocyte responses (Kinetic)

| Cytokine | Study | p-value |
|---|---|---|
| ALL DOUBLE | Explo Flu 001 | 0.0827 |
| | Explo Flu 002 | 0.0992 |
| CD40L | Explo Flu 001 | 0.0931 |
| | Explo Flu 002 | 0.1391 |
| INFγ | Explo Flu 001 | 0.0543 |
| | Explo Flu 002 | 0.1068 |
| IL2 | Explo Flu 001 | 0.0847 |
| | Explo Flu 002 | 0.2254 |
| TNFα | Explo Flu 001 | 0.0375 |
| | Explo Flu 002 | 0.2009 |

P-value: Wilcoxon Test (Non-parametric procedure) to test location difference (Wilcoxon rank-sum test) between the 2 groups at Day 21.

### IV.4. HI Titers

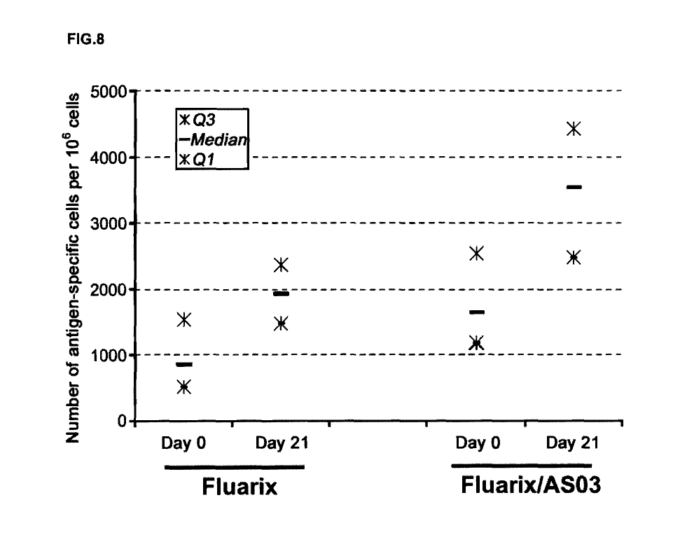

Results are shown in FIG. 9 and in Tables 33 to 36.

TABLE 33
Geometric Mean Titers (GMT) and seropositivity rates of
anti-HI titers (GMTs calculated on vaccinated subjects)

| | | | | | | 95% CI | | | 95% CI | |
|---|---|---|---|---|---|---|---|---|---|---|
| Antibody | Group | Timing | N | S+ | % | L.L. | U.L. | GMT | L.L. | U.L. |
| New | Fluarix | PRE | 18 | 17 | 94.4 | 72.6 | 99.9 | 63.5 | 38.1 | 105.9 |
| Caledonia | | PI(D 21) | 18 | 18 | 100 | 81.5 | 100 | 131.9 | 77.1 | 225.6 |
| | FluAS03 | PRE | 40 | 39 | 97.5 | 86.8 | 99.9 | 70.3 | 50.5 | 97.7 |
| | | PI(D 21) | 40 | 40 | 100 | 91.3 | 100 | 218.6 | 158.2 | 302.0 |
| A/Fujian | Fluarix | PRE | 18 | 18 | 100 | 81.5 | 100 | 95.0 | 51.0 | 176.9 |
| | | PI(D 21) | 18 | 18 | 100 | 81.5 | 100 | 498.3 | 272.1 | 912.7 |
| | FluAS03 | PRE | 40 | 40 | 100 | 91.3 | 100 | 94.3 | 71.4 | 124.6 |
| | | PI(D 21) | 40 | 40 | 100 | 91.3 | 100 | 735.1 | 564.4 | 957.5 |
| B/Shanghai | Fluarix | PRE | 18 | 16 | 88.9 | 65.3 | 98.6 | 23.3 | 15.2 | 35.8 |
| | | PI(D 21) | 18 | 17 | 94.4 | 72.6 | 99.9 | 139.8 | 64.0 | 305.0 |
| | FluAS03 | PRE | 40 | 38 | 95.0 | 83.1 | 99.4 | 58.6 | 43.9 | 78.1 |
| | | PI(D 21) | 40 | 40 | 100 | 91.3 | 100 | 364.4 | 269.7 | 492.4 |

PRE = Prevaccination,
PI(D 21) = day 21 post vaccination
95% CI, LL, and UL = 95% confidence interval, lower and upper limit
S+ = number of seropositive subjects

TABLE 34
Conversion factor of anti-HI titers (All vaccinated subjects)

| | A/N-Caledonia | | A/Fujian | | B/Shanghai | |
|---|---|---|---|---|---|---|
| | | GMR | | GMR | | GMR |
| Group | N | [95% CI] | N | [95% CI] | N | [95% CI] |
| Fluarix | 18 | 2.1 | 18 | 5.2 | 18 | 6.0 |
| | | [1.4; 3.2] | | [3.0; 9.3] | | [3.5; 10.2] |
| FluAS03 | 40 | 3.1 | 40 | 7.8 | 40 | 6.2 |
| | | [2.4; 4.0] | | [5.6; 10.9] | | [4.7; 8.2] |

N = total number of subjects
GMR = Geometric Mean Ratio (antilog of the mean log day 21/day 0 titers ratios)
95% CI = 95% confidence interval

TABLE 35
Seroprotection rates of anti-HI titers (All vaccinated subjects)

| | | | | >=40 | | | |
|---|---|---|---|---|---|---|---|
| Antibody | Group | Timing | N | n | % | 95% CI | |
| A/ | Fluarix | PRE | 18 | 14 | 77.8 | 52.4 | 93.6 |
| New | | PI(D 21) | 18 | 16 | 88.9 | 65.3 | 98.6 |
| Caledonia | FluAS03 | PRE | 40 | 32 | 80 | 64.4 | 90.9 |
| | | PI(D 21) | 40 | 39 | 97.5 | 86.8 | 99.9 |
| A/ | Fluarix | PRE | 18 | 14 | 77.8 | 52.4 | 93.6 |
| Fujian | | PI(D 21) | 18 | 18 | 100 | 81.5 | 100 |
| | FluAS03 | PRE | 40 | 36 | 90 | 76.3 | 97.2 |
| | | PI(D 21) | 40 | 40 | 100 | 91.2 | 100 |
| B/ | Fluarix | PRE | 18 | 6 | 33.3 | 13.3 | 59.0 |
| Shanghai | | PI(D 21) | 18 | 14 | 77.8 | 52.4 | 93.6 |
| | FluAS03 | PRE | 40 | 34 | 85 | 70.2 | 94.3 |
| | | PI(D 21) | 40 | 40 | 100 | 91.2 | 100 |

PRE = Prevaccination,
PI(D 21) = day 21 post vaccination
N = number of subjects with available results.
n = number of subjects with titres within the specified range.
% = percentage of subjects with titres within the specified range

TABLE 36
Seroconversion rates at PI day 21(fold-
increase = 4) (All vaccinated subjects)

| | | | Responders | | | |
|---|---|---|---|---|---|---|
| | | | | | 95% CI | |
| Antibody | Vaccine Group | N | n | % | LL | UL |
| A/ | Fluarix | 18 | 3 | 16.7 | 3.6 | 41.5 |
| New | FluAS03 | 40 | 19 | 47.5 | 31.5 | 63.9 |
| Caledonia | | | | | | |
| A/ | Fluarix | 18 | 13 | 72.2 | 46.5 | 90.3 |
| Fujian | FluAS03 | 40 | 34 | 85.0 | 70.2 | 94.3 |
| B/ | Fluarix | 18 | 12 | 66.7 | 41.0 | 86.7 |
| Shanghai | FluAS03 | 40 | 31 | 77.5 | 61.5 | 89.2 |

N = number of subjects with both pre and post vaccination result available.
n = number of responders.
% = Proportion of responders (n/N × 100).
95% CI = exact 95% confidence interval; LL = lower limit, UL = upper limit

### IV.5. Overall Conclusions

From this clinical study it is confirmed that the adjuvanted vaccine Flu-AS03 is superior to the equivalent unadjuvated vaccine Fluarix in terms of frequency of influenza specific CD4 T cells, and also in terms of persistence of the immune response elicited by the first Flu-AS03 vaccination (primo-vaccination in Explo Flu 001) until D0 of the revaccination study (Explo Flu 002 i.e. +/−1 year later). Furthermore this response is capable to recognise drifted influenza strains present in the new vaccine and to recognise the strains of the 2004 influenza vaccine.

In contrast to first year vaccination, upon revaccination individuals previously vaccinated with the adjuvanted Fluarix™ showed increased HI titer responsiveness as compared to those vaccinated with un-adjuvanted Fluarix™. There is an observable trend for 1.5- to 2-fold increase in HI titer directed against H1N1 and H3N2 strains and a demonstrated statistical increase in HI titer directed against B strain.

### Example V

### Pre-Clinical Evaluation of Adjuvanted and Unadjuvanted Influenza Vaccines in Ferrets

### First Study—Efficacy of New Formulations AS03 and AS03+MPL

### V.1. Rationale and Objectives

Influenza infection in the ferret model closely mimics human influenza, with regards both to the sensitivity to infection and the clinical response.

The ferret is extremely sensitive to infection with both influenza A and B viruses without prior adaptation of viral strains. Therefore, it provides an excellent model system for studies of protection conferred by administered influenza vaccines.

This study investigated the efficacy of various Trivalent Split vaccines, adjuvanted or not, to reduce disease symptoms (body temperature) and viral shedding in nasal secretions of ferrets challenged with homologous strains.

The objective of this experiment was to demonstrate the efficacy of an adjuvanted influenza vaccine compared to the plain (un-adjuvanted) vaccine.

The end-points were:

1) primary end-point: Reduction of viral shedding in nasal washes after homologous challenge:
2) secondary end-points: Analysis of the humoral reponse by IHA and monitoring of the temperature around the priming and the challenge.

### V.2. Experimental Design

### V.2.1. Treatment/Group (Table 37)

Female ferrets (Mustela putorius furo) (6 ferrets/group) aged 14-20 weeks were obtained from MISAY Consultancy (Hampshire, UK). Ferrets were primed on day 0 with heterosubtypic strain H1N1 A/Stockholm/24/90 (4 Log TCID₅₀/ml). On day 21, ferrets were injected intramuscularly with a full human dose (500 μg vaccine dose, 15 μg HA/strain) of a combination of H1N1 A/New Calcdonia/20/99, H3N2 A/Panama/2007/99 and B/Shangdong/7/97. Ferrets were then challenged on day 41 by intranasal route with an homotypic strain H3N2 A/Panama/2007/99 (4.51 Log TCID₅₀/ml).

TABLE 37

| | | | Comments | |
|---|---|---|---|---|
| | Antigen(s) + | Formulation + | (schedule/route/ | |
| Group | dosage | dosage | challenge) | Other treatments |
| 1 | Trivalent | Full HD: 15 μg | IM; Day 21 | Priming H1N1 |
| | Plain | HA/strain | | (A/Stockolm/24/ |
| | | | | 90) Day 0 |
| 2 | Trivalent | Full HD: 15 μg | IM; Day 21 | Priming H1N1 |
| | AS03 | HA/strain | | (A/Stockolm/24/90) |
| | | | | Day 0 |
| 3 | Trivalent | Full HD: 15 μg | IM; Day 21 | Priming H1N1 |
| | AS03 + MPL | HA/strain | | (A/Stockolm/24/ |
| | | | | 90) Day 0 |
| 4 | PBS | | IM; Day 21 | Priming H1N1 |
| | | | | (A/Stockolm/24/ |
| | | | | 90) Day 0 |

### V.2.2. Preparation of the Vaccine Formulations

### Formulation 1: Trivalent Plain (Un-Adjuvanted) Formulation (500 μl):

PBS 10 fold concentrated (pH 7.4 when one fold concentrated) as well as a mixture containing Tween 80, Triton X-100 and VES (quantities taking into account the detergents present in the strains) are added to water for injection. The detergents quantities reached are the following: 750 μg Tween 80, 110 μg Triton X-100 and 100 μg VES per 1 ml. After 5 min stirring, 15 μg of each strain H1N1, H3N2 and 17.5 μg of B strain are added in sequence with 10 min stirring between each addition. The formulation is stirred for 15 minutes at room temperature and stored at 4° C. if not administered directly.

Formulation 2: Trivalent Split Influenza Adjuvanted with AS03 (500 μl):

PBS 10 fold concentrated (pH 7.4 when one fold concentrated) as well as a mixture containing Tween 80, Triton X-100 and VES (quantities taking into account the detergents present in the strains) is added to water for injection. The detergents quantities reached are the following: 750 μg Tween 80, 110 μg Triton X-100 and 100 μg VES per 1 ml. After 5 min stirring, 15 μg of each strain H1N1, H3N2 and 17.5 μg of B strain are added with 10 min stirring between each addition. After 15 min stirring, 250 μl of SB62 emulsion (prepared as in taught in Example II.1) is added. The formulation is stirred for 15 minutes at room temperature and stored at 4° C. if not administered directly.

Formulation 3: Trivalent Split Influenza Adjuvanted with AS03+MPL

PBS 10 fold concentrated (pH 7.4 when one fold concentrated) as well as a mixture containing Tween 80, Triton X-100 and VES (quantities taking into account the detergents present in the strains) is added to water for injection. The detergents quantities reached are the following: 750 μg Tween 80, 110 μg Triton X-100 and 100 μg VES per 1 ml. After 5 min stirring, 15 μg of each strain H1N1, H3N2 and 17.5 μg of B strain are added with 10 min stirring between each addition. After 15 min stirring, 250 μl of SB62 emulsion (prepared as in taught in Example II.1) is added. The mixture is stirred again for 15 min just prior addition of 25 μg of MPL from a suspension prepared as detailed in Example II.3.1. The formulation is stirred for 15 minutes at room temperature and stored at 4° C. if not administered directly.

Remark: In each formulation, PBS 10 fold concentrated is added to reach isotonicity and is 1 fold concentrated in the final volume. H2O volume is calculated to reach the targeted volume.

TABLE 38

| | | | | Analysis |
|---|---|---|---|---|
| Readout | Timepoint | Sample-type | I/P | method |
| Viral | D−1 to D+7 Post priming | Nasal | In | Titration |
| shedding | D−1 to D+5 Post challenge | washes | | |
| T° | D−1 to D+3 Post priming | Implant in | In | Telemetry |
| monitoring | D−2 to D+3 Post challenge | peritoneal | | |
| | | cavity | | |
| IHA | Pre, Post priming, Post | Serum | In | IHA |
| | imm, Post challenge | | | |

In = Individual/Po = Pool

### V.3. Results

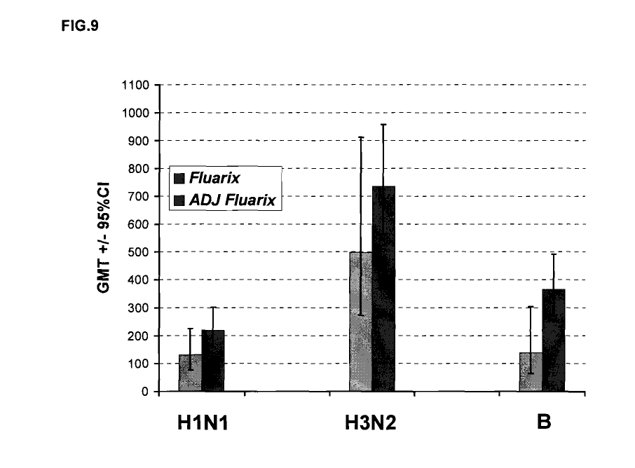

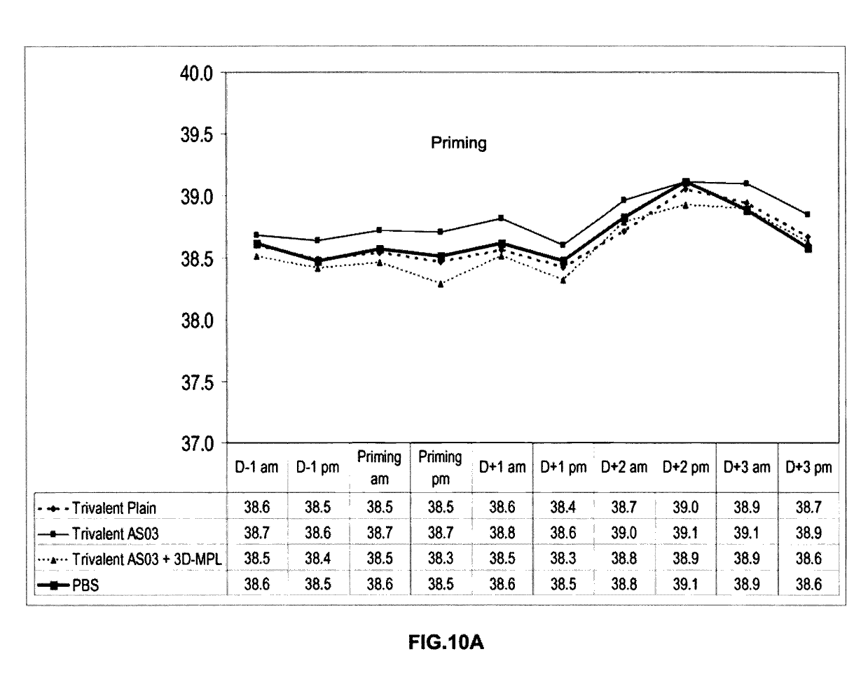

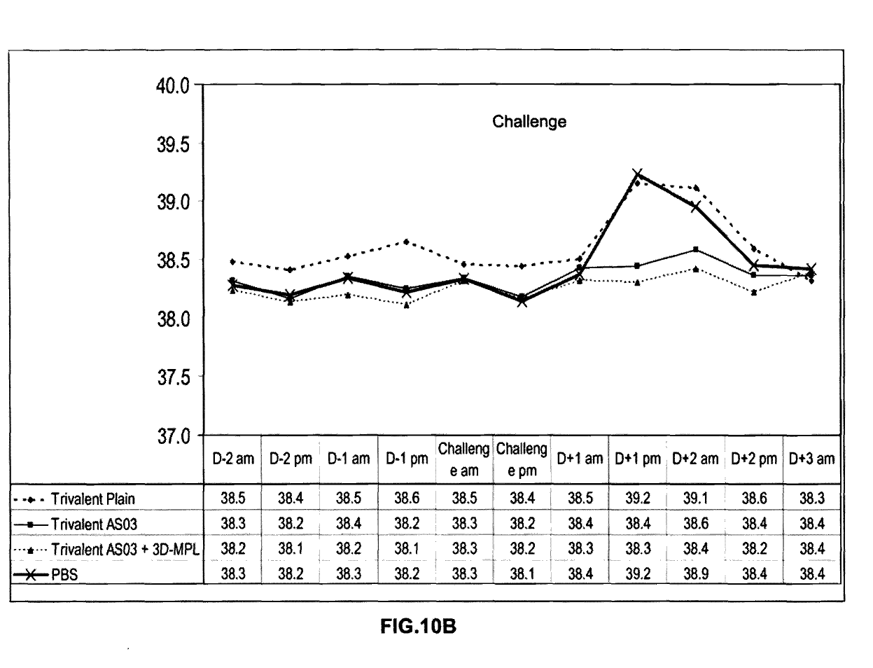

A schematic representation of the results is given in FIG. 10 and FIG. 11.

### V.3.1. Temperature Monitoring

Individual temperature were monitored with the transmitters and by the telemetry recording (according to the procedure detailed under I.2.2). All implants were checked and refurbished and a new calibration was performed by DSI before placement in the intraperitoneal cavity. All animals were individually housed in single cage during these measurements.

Temperature were monitored from 3 days Pre-challenge until 5 days Post challenge every 15 minutes and an average has been calculated by mid-day. Results from baseline to baseline body temperature are shown in FIGS. 10A (results from −1 to +3 days are shown) and 10B (results from −2 to +3 days are shown).

Post-challenge, a peak of body temperature only observed after immunization with trivalent split plain or PBS. No peak observed after immunization with trivalent split adjuvanted with AS03 or AS03+MPL.

### V.3.2. Viral Shedding (FIG. 11)

Viral titration of nasal washes was performed on 6 animals per group.

The nasal washes were performed by administration of 5 ml of PBS in both nostrils in awake animals. The inoculation was collected in a Petri dish and placed into sample containers at −80° C. (dry ice).

All nasal samples were first sterile filtered through Spin X filters (Costar) to remove any bacterial contamination. 50 μl of serial ten-fold dilutions of nasal washes were transferred to microtiter plates containing 50 μl of medium (10 wells/dilution). 100 μl of MDCK cells (2.4×10⁵ cells/ml) were then added to each well and incubated at 35° C. until cell confluence is reached for the control cells, e.g. for 5-7 days. After 6-7 days of incubation, the culture medium is gently removed and 100 μl of a 1/20 WST-1 containing medium is added and incubated for another 18 hrs.

The intensity of the yellow formazan dye produced upon reduction of WST-1 by viable cells is proportional to the number of viable cells present in the well at the end of the viral titration assay and is quantified by measuring the absorbance of each well at the appropriate wavelength (450 nanometers). The cut-off is defined as the OD average of uninfected control cells—0.3 OD (0.3 OD correspond to +/−3 StDev of OD of uninfected control cells). A positive score is defined when OD is <cut-off and in contrast a negative score is defined when OD is >cut-off. Viral shedding titers were determined by “Reed and Muench” and expressed as Log TCID50/ml.

Lower viral shedding was observed Post-challenge with Trivalent Split adjuvanted with AS03 or AS03+MPL compared to Trivalent Split Plain or PBS. The protective effect was slightly better with AS03 compared to AS03+MPL (see Day 2 Post-challenge). Statistical significance could not be determined due to the low number of animals per group.

### V.3.3. Conclusion of the Experiment

Higher humoral responses (HI titers) were observed with Trivalent Split adjuvanted with AS03 or AS03+MPL compared to the Trivalent Split Plain for all 3 strains (at least 2-fold for 2 out of 3 strains, i.e. H3N2 and B strains).

AS03 and AS03+MPL formulations showed added benefit in terms of protective efficacy in ferrets (lower viral shedding and temperature) (FIGS. 10 and 11).

Post-challenge, no boost of the humoral response was observed after immunization with Trivalent Split adjuvanted with AS03 or AS03+MPL.

### Second Study—Heterotypic Challenge Study in Ferrets: Demonstration of Efficacy of New Formulation Tested

### V.4. Rationale and Objectives

This study investigated the efficacy of various Trivalent Split vaccines, adjuvanted or not, by their ability to reduce disease symptoms (body temperature) and their effects on viral shedding in nasal secretions of immunized ferrets after a heterologuous challenge.

### V.5. Experimental Design

Female ferrets (Mustela putorius furo) (6 ferrets/group) aged 14-20 weeks were obtained from MISAY Consultancy (Hampshire, UK). Four groups were tested:
Fluarix
Trivalent Split AS03
Trivalent Split AS03+MPL
PBS

Ferrets were primed on day 0 with heterosubtypic strain H1N1 A/Stockholm/24/90 (4 Log TCID₅₀/ml). On day 21, ferrets were injected intramuscularly with a full human dose (500 μg vaccine dose, 15 μg HA/strain) of a combination of H1N1 A/New Calcdonia/20/99, H3N2 A/Panama/2007/99 and B/Shangdong/7/97 (17.5 μg HA). Ferrets were then challenged on day 43 by intranasal route with an heterosubtypic strain H3N2 A/Wyoming/3/2003 (4.51 Log TCID₅₀/ml).

### V.6. Results

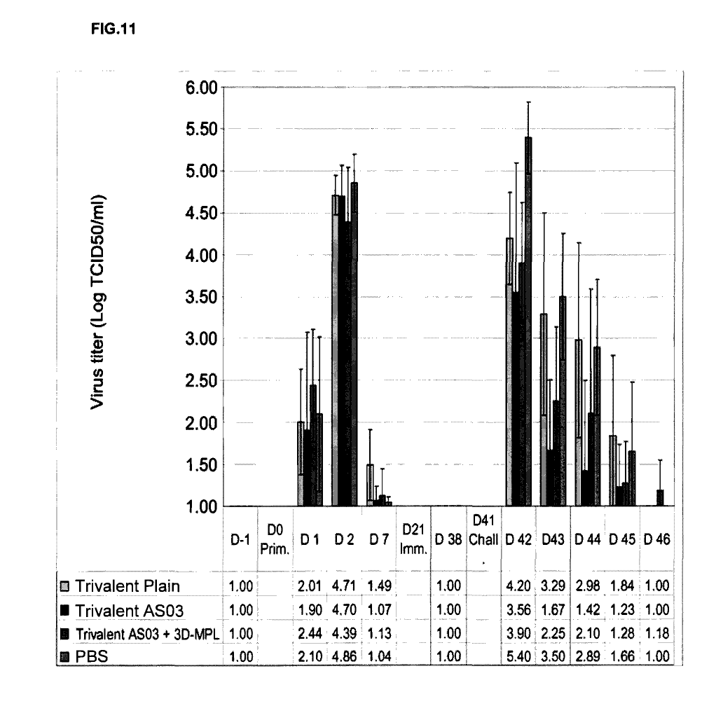

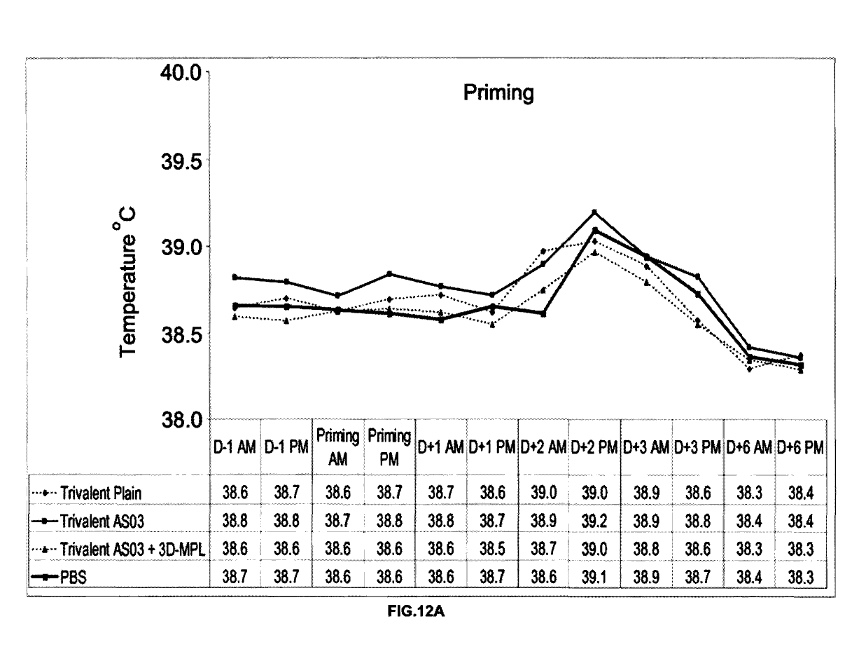

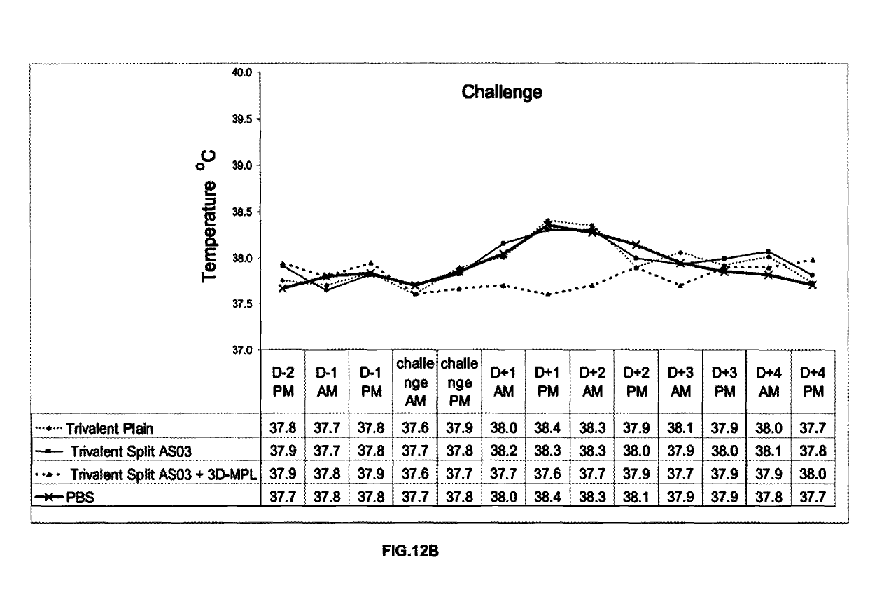

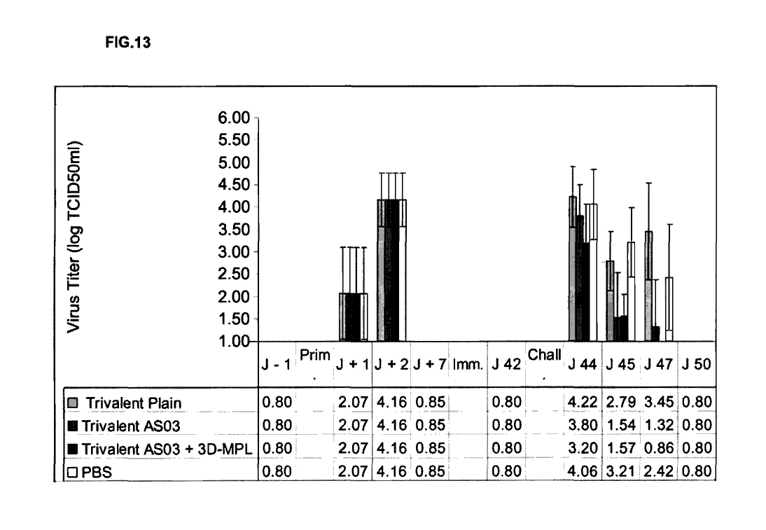

A schematic representation of the results is given in FIG. 12 and in FIG. 13.

### V.6.1. Temperature Monitoring

Individual temperature were monitored with the transmitters and by the telemetry recording. All implants were checked and refurbished and a new calibration was performed by DSI before placement in the intraperitoneal cavity. All animals were individually housed in single cage during these measurements.

The results (FIG. 12) show that:
A high variability from one group to another was observed around the priming. The baseline seemed to be higher before priming than after priming.
Despite the high variability in the body temperature, a peak was only observed Post-challenge in ferrets immunized with PBS (6/6 ferrets), Trivalent Split Plain (5/6 ferrets) and Trivalent Split adjuvanted with AS03 (2/6 ferrets). No peak was observed after immunization with trivalent split adjuvanted with AS03+MPL (0/6 ferrets).
AS03 seemed to be less efficient than AS03+MPL against heterologous strains in terms of fever prevention. We cannot conclude the possibility that difference between adjuvant is due to different level in pre-challenge antibody levels.

### V.6.2. Viral Shedding (FIG. 13)

The nasal washes were performed by administration of 5 ml of PBS in both nostrils in awake animals. The inoculation was collected in a Petri dish and placed into sample containers at −80° C. (dry ice).

All nasal samples were first sterile filtered through Spin X filters (Costar) to remove any bacterial contamination. 50 μl of serial ten-fold dilutions of nasal washes were transferred to microtiter plates containing 50 μl of medium (10 wells/dilution). 100 μl of MDCK cells (2.4×10⁵ cells/ml) were then added to each well and incubated at 35° C. until cell confluence is reached for the control cells, e.g. for 5-7 days. After 6-7 days of incubation, the culture medium is gently removed and 100 μl of a 1/20 WST-1 containing medium is added and incubated for another 18 hrs.

The intensity of the yellow formazan dye produced upon reduction of WST-1 by viable cells is proportional to the number of viable cells present in the well at the end of the viral titration assay and is quantified by measuring the absorbance of each well at the appropriate wavelength (450 nanometers). The cut-off is defined as the OD average of uninfected control cells—0.3 OD (0.3 OD corresponds to +/−3 St Dev of OD of uninfected control cells). A positive score is defined when OD is <cut-off and in contrast a negative score is defined when OD is >cut-off. Viral shedding titers were determined by “Reed and Muench” and expressed as Log TCID50/ml.

Viral Shedding after Priming

Viral shedding was measured for 12 ferrets from Day 1 Pre-priming- to Day 7 Post-priming. Results are expressed in pool.

The viral clearance was observed on Day 7 Post-priming in all ferrets.

Viral Shedding after Challenge

Viral shedding was measured for 6 ferrets/group from Day 1 Pre-challenge to Day 7 Post-challenge.

Two days Post-challenge, statistically significant lower viral titers were observed in ferrets immunized with Trivalent Split adjuvanted with AS03 and AS03+MPL compared to ferrets immunized with Trivalent Split Plain and PBS (difference of 1.25/1.22 log and 1.67/1.64 log with adjuvanted groups AS03/AS03+MPL compared to the Plain vaccine, respectively).

On Day 50, no virus was detected in nasal washes.

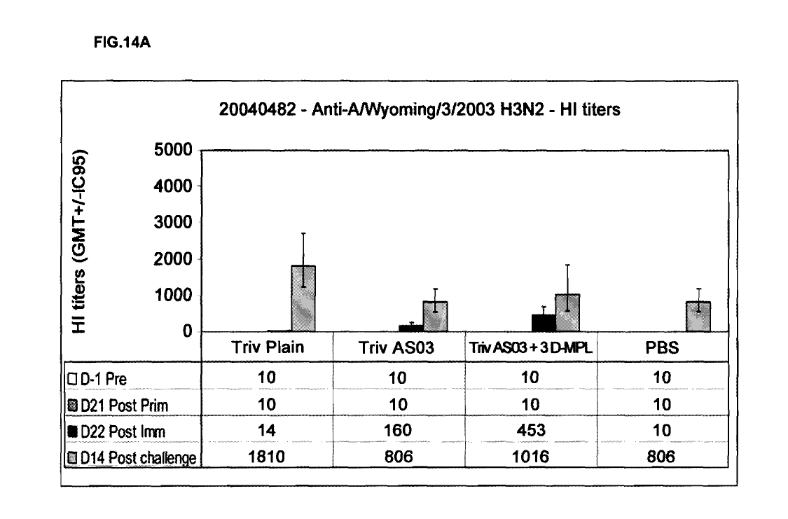

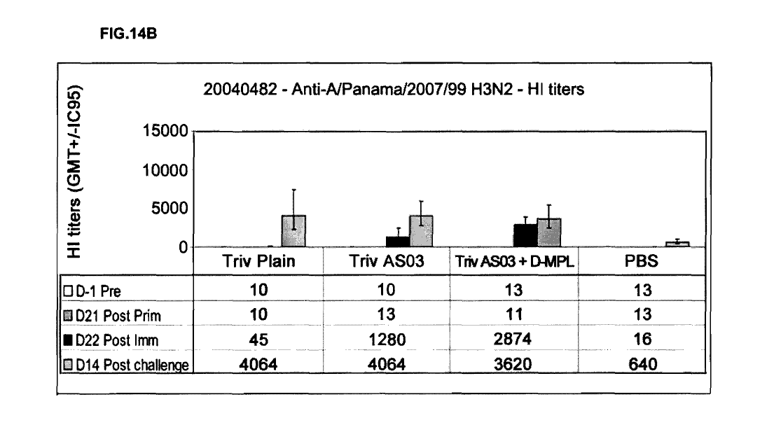

### V.6.3. Hemagglutination Inhibition Test (HI Titers) (FIGS. 14A and B)

Serum samples were collected 1 day before priming, 21 days Post-priming, 22 days post-immunization and 14 days post-challenge.

Anti-Hemagglutinin antibody titers to the H3N2 influenza virus (vaccine and challenge strains) were determined using the hemagglutination inhibition test (HI). The principle of the HI test is based on the ability of specific anti-Influenza antibodies to inhibit hemagglutination of chicken red blood cells (RBC) by influenza virus hemagglutinin (HA). Sera were first treated with a 25% neuraminidase solution (RDE) and were heat-inactivated to remove non-specific inhibitors. After pre-treatment, two-fold dilutions of sera were incubated with 4 hemagglutination units of each influenza strain. Chicken red blood cells were then added and the inhibition of agglutination was scored. The titers were expressed as the reciprocal of the highest dilution of serum that completely inhibited hemagglutination. As the first dilution of sera was 1:10, an undetectable level was scored as a titer equal to 5.

### Results:

Results are shown in FIGS. 14A and 14B. After immunization with H3N2 A/Panama, higher humoral responses (HI titers) were observed in ferrets immunized with the trivalent split vaccine adjuvanted with AS03 or AS03+MPL, as compared to the humoral response observed after immunization of ferrets with the un-adjuvanted (plain) trivalent split vaccine (Fluarix™).

Similar HI titers were observed in ferrets immunized with H3N2 A/Panama adjuvanted with AS03 or AS03+MPL.

Cross-reactive HI titers to the heterologous strain A/Wyoming H3N2 was only observed after immunization with A/Panama H3N2 strain containing vaccine adjuvanted with AS03 or AS03+MPL (not observed after immunization with Trivalent Split Plain).

A boost of A/Wyoming-specific HI titers was observed in ferrets immunized with the heterologous strain A/Panama H3N2 and challenged with A/Wyoming H3N2. As expected and contrary to the homologous challenge, the heterologous challenge resulted in an increase of A/Panama-specific HI titers in ferrets immunized with A/Panama H3N2 adjuvanted with AS03 and AS03+MPL.

V.6.4. Conclusion of this Experiment

As expected, a boost of anti-H3N2 HI titers was observed after heterologous challenge compared to the situation after homologous challenge (no boost).

However, similar protection (viral shedding) was observed after heterologous and homologous challenge.

### Example VI

### Pre-Clinical Evaluation of Adjuvanted and Unadjuvanted Influenza Vaccines in C57BI/6 Primed Mice

### VI.1. Experimental Design and Objective

Significant higher CD4 T cell responses were observed, in Explo-Flu-001 clinical study (see Example III), for Trivalent Flu Split AS03 compared to Fluarix Plain (un-adjuvanted). No difference was observed for both CD8 T cell and humoral responses between these two groups.

The purpose was to select readouts to induce in mice similar CMI responses than observed in humans. Particularly, the purpose was to show higher CMI responses in mice by using Split AS03 or split AS03+MPL compared to Split plain.

### VI.1.1. Treatment/Group

Female C57BI/6 mice (15 mice/group) aged 6-8 weeks were obtained from Harlan Horst, Netherland. The groups tested were:
Trivalent Split Plain
Trivalent Split AS03
Trivalent Split AS03+MPL
PBS

Mice were primed on day 0 with heterosubtypic strains (5 μg HA whole inactivated H1N1 A/Johnannesburg/82/96, H3N2 A/Sydney/5/97, B/Harbin/7/94). On day 28, mice were injected intramuscularly with 1.5 μg HA Trivalent split (A/New Calcdonia/20/99, A/Panama/2007/99, B/Shangdong/7/97) plain or adjuvanted (see groups below).

### VI.1.2. Preparation of the Vaccine Formulations

In each formulation, PBS 10 fold concentrated is added to reach isotonicity and is 1 fold concentrated in the final volume. H₂O volume is calculated to reach the targeted volume.

### Split Trivalent Plain (Un-Adjuvanted):

Formulation 1 (for 500 μl): PBS 10 fold concentrated (pH 7.4 when one fold concentrated) as well as a mixture containing Tween 80, Triton X-100 and VES (quantities taking into account the detergents present in the strains) are added to water for injection. The detergents quantities reached are the following: 750 μg Tween 80, 110 μg Triton X-100 and 100 μg VES per 1 ml After 5 min stirring, 15 μg of each strain H1N1, H3N2 and B are added with 10 min stirring between each addition. The formulation is stirred for 15 minutes at room temperature and stored at 4° C. if not administered directly.

Split Trivalent Adjuvanted with the Oil-in-Water Emulsion Adjuvant AS03:

PBS 10 fold concentrated (pH 7.4 when one fold concentrated) as well as a mixture containing Tween 80, Triton X-100 and VES (quantities taking into account the detergents present in the strains) is added to water for injection. The detergents quantities reached are the following: 750 μg Tween 80, 110 μg Triton X-100 and 100 μg VES per 1 ml. After 5 min stirring, 15 μg of each strain H1N1, H3N2 and B are added with 10 min stirring between each addition. After 15 min stirring, 250 μl of SB62 emulsion (prepared as taught in Example II.1) is added. The formulation is stirred for 15 minutes at room temperature and stored at 4° C. if not administered directly.

Split Trivalent Adjuvanted with AS03+MPL:

PBS 10 fold concentrated (pH 7.4 when one fold concentrated) as well as a mixture containing Tween 80, Triton X-100 and VES (quantities taking into account the detergents present in the strains) is added to water for injection. The detergents quantities reached are the following: 750 μg Tween 80, 110 μg Triton X-100 and 100 μg VES per 1 ml After 5 min stirring, 15 μg of each strain H1N1, H3N2 and B are added with 10 min stirring between each addition. After 15 min stirring, 250 μl of SB62 emulsion (prepared as taught in Example II.1) is added. The mixture is stirred again for 15 min just prior addition of 25 μg of MPL. The formulation is stirred for 15 minutes at room temperature and stored at 4° C. if not administered directly.

### VI.1.3. Read-Outs

### CMI Analysis (ICS: CD4/CD8, IL-2/IFNg Staining)

PBMCs from primed mice were harvested 7 days post-immunization. They were tested in pools/group.

### VI.2. Results

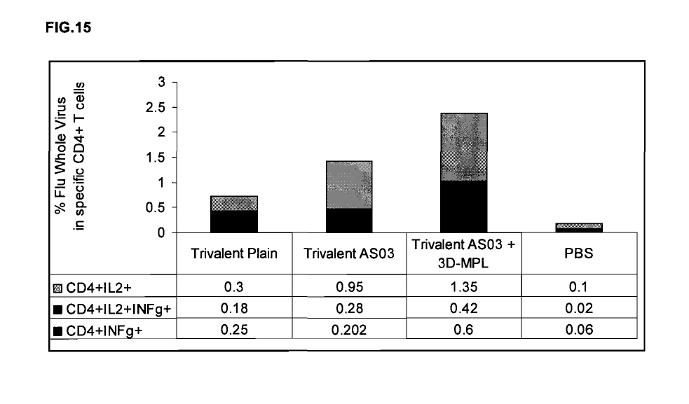

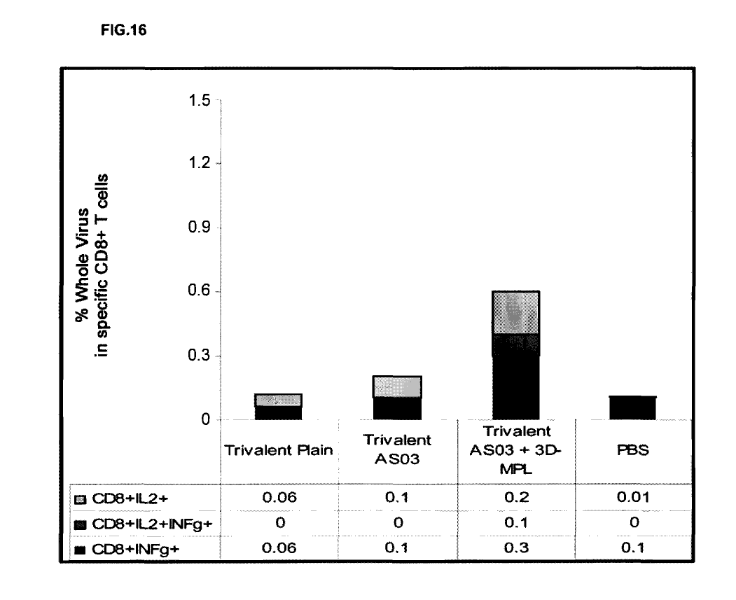

Conditions that showed higher frequencies of CD4 and CD8+ T cells, as well as lower background, were determined by using C57BI/6 primed mice and whole inactivated virus 1 μg/ml as re-stimulating antigen. Results are shown in FIG. 15 (CD4 T-cell responses) and in FIG. 16 (CD8 T-cell response).

With these conditions, it was possible to induce:
Higher CD4 T cell responses for Split AS03 compared to Split Plain, as observed in humans.
Higher CD4 T cell responses for Split AS03+MPL compared to Split Plain.
Similar CD8 T cell responses between Split Plain and Split AS03, as observed in humans.
Trend for higher CD8 T cell responses for AS03+MPL compared to Split AS03 or Split Plain

### Example VII

### Pre-Clinical Evaluation of Adjuvanted and Unadjuvanted Split and Sub-Unit Influenza Vaccines in C57BI/6 Mice Primed with Heterologous Strains

### VII.1. Experimental Design and Objective

Significant higher CD4 T cell responses were observed, in Explo-Flu-001 clinical study (see Example III), for Trivalent Flu Split AS03 compared to Fluarix Plain (un-adjuvanted). No difference was observed for both CD8 T cell and humoral responses between these two groups.

An animal model reproducing similar immune profiles than observed in humans was developed by using C57BI/6 mice primed with heterologous strains. For ICS (intracellular cytokine staining), the re-stimulation is performed with an inactivated whole virus.

The purpose was to compare the CMI response induced by a GlaxoSmithKline commercially available split vaccine (Fluarix™) versus a subunit vaccine (Chiron's vaccine Fluad™) as well as the CMI response obtained with these vaccines adjuvanted with AS03, or AS03+MPL or another oil-in-water emulsion adjuvant (OW).

### VII.1.1. Treatment/Group

Female C57BI/6 mice (24 mice/group) aged 6-8 weeks were obtained from Harlan Horst, Netherland. Mice were primed intranasally on day 0 with heterosubtypic strains (5 μg HA whole formaldehyde inactivated H1N1 A/Johnannesburg/82/96, H3N2 A/Sydney/5/97, B/Harbin/7/94). On day 29, mice were injected intramuscularly with 1.5 μg HA Trivalent split (A/New Calcdonia/20/99, A/Wyoming/3/2003, B/Jiangsu/10/2003) plain or adjuvanted (see groups in Table 39 below).

TABLE 39

| Gr | Antigen/Formulation | Other treatment |
|---|---|---|
| 1 | Trivalent split*/Plain (un-adjuvanted) = | Heterologous priming D0 |
| | Fluarix ™ | |
| 2 | Trivalent split*/OW | Heterologous priming D0 |
| 3 | Trivalent split*/AS03 | Heterologous priming D0 |
| 4 | Trivalent split*/AS03 + MPL | Heterologous priming D0 |
| | (2.5 μg per dose) | |
| 5 | Gripguard (= Fluad ™) = sub-unit in an | Heterologous priming D0 |
| | oil-in-water emulsion | |
| 6 | Aggripal ™ (sub-unit)/AS03 | Heterologous priming D0 |
| 7 | Aggripal ™ (sub-unit)/AS03 + MPL | Heterologous priming D0 |
| | (2.5 μg per dose) | |
| 8 | Aggripal ™ (sub-unit)/OW** | Heterologous priming D0 |
| 9 | Aggripal ™ (sub-unit) | Heterologous priming D0 |
| 10 | PBS | Heterologous priming D0 |

*Fluarix ™
**OW produced as explained in the section below

### VII.1.2. Preparation of the Vaccine Formulations

### Preparation of OW

An oil-in-water emulsion called OW is prepared following the recipe published in the instruction booklet contained in Chiron Behring FluAd vaccine.

Water for injection, 36.67 mg of Citric acid and 627.4 mg of Na Citrate.2H2O are mixed together and the volume is adjusted to 200 ml. 470 mg of Tween 80 is mixed with 94.47 ml of this buffer and this mixture is called “solution A”. The oil mixture is prepared by mixing 3.9 g of squalene and 470 mg of Span 85 under magnetic stirring. Solution A is then added to the oil mixture and the final volume obtained is 100 ml. The mixture is then first passed trough a 18Gx 1½ needle and is then put in the M110S microfluidiser (from Microfluidics) in two samples to reduce the size of the oil dropplets. When a particle size around 150 nm is obtained for each, the 2 samples are pooled and filtrated on 0.2 μm filter. A z average mean of 143 nm with a polydispersity of 0.10 is obtained for the pooled sample at TO and of 145 nm with a polydispersity of 0.06 after 4 months storage at 4° C. This size is obtained using the Zetasizer 3000HS (from Malvern), under the following technical conditions:
laser wavelength: 532 nm (Zeta3000HS).
laser power: 50 mW (Zeta3000HS).
scattered light detected at 90° (Zeta3000HS).
temperature: 25° C.,
duration: automatic determination by the soft,
number: 3 consecutive measurements,
z-average diameter: by cumulants analysis

### Formulation for Group 1 (for 1 ml):

PBS 10 fold concentrated (pH 7.4 when one fold concentrated) as well as a mixture containing Tween 80, Triton X-100 and VES (quantities taking into account the detergents present in the strains) to reach a final concentration of 375 μg/ml Tween 80, 55 μg/ml Triton X-100 and 50 μg/ml VES, are added to water for injection. After 5 min stirring, 15 μg of each strain H1N1, H3N2 and B are added with 10 min stirring between each addition. The formulation is stirred for 15 minutes and stored at 4° C. if not administered directly.

### Formulation for Group 2 (for 1 ml):

PBS 10 fold concentrated (pH 7.4 when one fold concentrated) as well as a mixture containing Tween 80, Triton X-100 and VES (quantities taking into account the detergents present in the strains) to reach a final concentration of 375 μg/ml Tween 80, 55 μg/ml Triton X-100 and 50 μg/ml VES, is added to water for injection. After 5 min stirring, 15 μg of each strain H1N1, H3N2 and B are added with 10 min stirring between each addition. After 15 min stirring, 250 μl of OW emulsion is added. The formulation is stirred for 15 minutes and stored at 4° C. if not administered directly.

### Formulation for Group 3: for 1 ml:

PBS 10 fold concentrated (pH 7.4 when one fold concentrated) as well as a mixture containing Tween 80, Triton X-100 and VES (quantities taking into account the detergents present in the strains) to reach a final concentration of 375 μg/ml Tween 80, 55 μg/ml Triton X-100 and 50 μg/ml VES, is added to water for injection. After 5 min stirring, 15 μg of each strain H1N1, H3N2 and B are added with 10 min stirring between each addition. After 15 min stirring, 250 μl of SB62 emulsion is added. The formulation is stirred for 15 minutes and stored at 4° C. if not administered directly.

### Formulation for Group 4: for 1 ml:

PBS 10 fold concentrated (pH 7.4 when one fold concentrated) as well as a mixture containing Tween 80, Triton X-100 and VES (quantities taking into account the detergents present in the strains) to reach a final concentration of 375 μg/ml Tween 80, 55 μg/ml Triton X-100 and 50 μg/ml VES, is added to water for injection. After 5 min stirring, 15 μg of each strain H1N1, H3N2 and B are added with 10 min stirring between each addition. After 15 min stirring, 250 μl of SB62 emulsion is added. The mixture is stirred again for 15 min just prior addition of 25 μg of MPL. The formulation is stirred for 15 minutes and stored at 4° C. if not administered directly.

### Formulation for Group 5: for 1 ml:

Equal volume of PBS and FluAd™/Gripguard™ (commercial vaccine) vaccine are mixed.

The formulation is stirred for 15 minutes and stored at 4° C. if not administered directly.

### Formulation for Group 6: for 1 ml:

250 μl of PBS mod pH 7.4 are added to a 500 μl dose of Aggripa™ (commercial vaccine). After 15 min stirring, 250 μl of SB62 is added (prepared according to the methodoly detailed for the scaled-up production). The formulation is stirred for 15 minutes and stored at 4° C. if not administered directly.

### Formulation for Group 7: for 1 ml:

PBS mod pH 7.4 (to reach a final volume of 1 ml) is added to a 500 μl dose of Aggripal™ (commercial vaccine). After 15 min stirring, 250 μl of SB62 is added (prepared according to the methodoly detailed for the scaled-up production). 25 μg of MPL are then added. The formulation is stirred for 15 minutes and stored at 4° C. if not administered directly.

### Formulation for Group 8: for 1 ml:

250 μl of PBS mod pH 7.4 are added to a 500 μl dose of Aggripal. After 15 min stirring, 250 μl of OW as prepared for group 2 is added and the formulation is stirred 15 min and stored at 4° C. if not administered directly.

### Formulation for Group 9: for 1 ml:

Equal volume of PBS mod pH 7.4 and Aggripal are mixed. The formulation is stirred for 15 minutes and stored at 4° C. if not administered directly.

### VII.1.3. Read-Outs (Table 40)

CMI (ICS): 7 Days Post-immunization.

IHA/neutralization assay: 21 Days Post-immunization.

TABLE 40

| | | | | Analysis |
|---|---|---|---|---|
| Read-out | Timepoint | Sample type | I/P | method |
| ICS | D35 | PBLs | Po | FACS analysis |
| (CD4, CD8, IL- | | | | |
| 2, IFN-γ) | | | | |
| Humoral | D14, D44 | Sera | In | IHA, neutra |
| response | | | | |

In = Individual/Po = Pool

### CMI Analysis (ICS: CD4/CD8; IL-2/IFN-Gamma Staining)

PBMCs from 24 mice/group were harvested 7 days post-immunization and tested in pools/group.

### VII.2. Results

### VII.2.1. Humoral Immunity

Haemagglutination inhibition activity against the 3 vaccine strains was detected in sera from 24 animals per group at Day 14 after intranasal heterologous priming and at Day 16 Post-immunization.

For the 3 strains and for all groups, a boost of HI titers was observed after immunization.
For a same adjuvant and for the 3 strains, similar HI titers were induced by the subunit vaccine and the Split vaccine.
Similar HI titers were observed for Fluad compared to Aggripal OW for the 3 strains
No difference was observed between Fluarix and Aggripal for H1N1 and B strains.
For the 3 strains, statistically significant higher HI titers were observed when the Flu vaccine (Split or subunit) was adjuvanted with AS03 with or without MPL compared to the plain Flu vaccine.
HI titers were statistically significant higher for the Flu vaccine (Split or subunit) adjuvanted with OW compared to the Flu vaccine plain only for the A/Wyoming strain.

### VII.2.2. Cell-Mediated Immune Response (ICS at day 7 Post Immunization)

### CD4 T Cell Responses—FIG. 17 Upper Part

PBMCs from 24 mice per group were harvested at Day 7 Post-immunization and tested in one pool/group. Inactivated trivalent whole viruses (1 μg/ml) were used as re-stimulating antigen. Results are shown in FIG. 17 upper part.

In terms of Flu whole virus-specific CD4+ T cells expressing IL-2, IFN-γ or both cytokines (FIG. 17 upper part):
1. GSK adjuvants showed the same trend as previously observed (Example VI): AS03+MPL was superior to AS03 which was in turn superior to the result obtained with the plain vaccine. This trend was observed both for the split or the subunit vaccine.
2. Whatever the formulation (Plain, AS03 or AS03+MPL), the split vaccine induced a higher CD4+ T cell responses than the subunit vaccine.
3. Fluad (subunit+oil-in-water emulsion OW—see preparation section) seemed to induce similar frequencies than Fluarix Plain.
4. Formulations Trivalent Split/AS03 or Trivalent Split/AS03+MPL induced higher CD4+T cell responses than the formulation subunit/oil-in-water emulsion OW.

### CD8 T Cell Responses—FIG. 17 Lower Part

PBMCs from 24 mice per group were harvested at Day 7 Post-immunization and tested in one pool/group. Inactivated trivalent whole viruses (1 μg/ml) were used as re-stimulating antigen.

In terms of Flu whole virus-specific CD8+ T cells expressing IL-2, IFN-γ or both cytokines (FIG. 17 lower part):
The cut-off of this experiment was relatively high due to the high background observed for the PBS negative control group.
However higher specific CD8 T cell responses were observed for mice immunized with Trivalent Split/AS03+MPL compared to other vaccine formulations.

### VII.3. Summary of Results and Conclusions

The following results were obtained:

1) Flu-specific CD4+ T cells obtained by ICS at Day 7 post-immunization showed:
1. Similar responses were obtained for Fluad compared to Fluarix.
2. The adjuvanted formulation induced higher immune response compared to the un-adjuvanted vaccine, both for the split influenza vaccine (as observed in humans) and for the subunit (Aggripal) vaccine (not assessed in humans). The oil-in-water emulsion adjuvant AS03 supplemented with MPL (groups 4 and 9) gave higher responses than the oil-in-water emulsion adjuvant AS03 (groups 3 and 8).
3. There is a trend of a higher CD4 responses with Split/AS03+MPL compared to Split/AS03 (FIG. 17).
4. The responses induced by the split vaccine were superior to the responses obtained with the subunit vaccine (compare groups 1 to 4 and groups 5 to 9).
5. The split vaccine, whether adjuvanted with AS03 with or without MPL (groups 3 and 4) performed showed higher CD4+ T cell responses than the sub-unit vaccine, either Fluad (group 5) or Aggripal+OW (group 7).
   2) Flu-specific CD8+ T cells obtained by ICS at Day 7 post-immunization showed no differences are observed between Split/AS3 and Split Plain (as observed in humans). There was a trend for a higher CD8+ T cell response by using Split/AS03+MPL compared to Split/AS03 or Split Plain.
   3) For a same adjuvant and for the 3 strains, similar HI titers were induced by the subunit vaccine and the split vaccine. For the 3 strains, statistically significant higher titers were observed when the Flu vaccine (subunit or split) was adjuvanted with AS03 or AS03+MPL compared to the Flu vaccine plain (Flu vaccine OW>Flu vaccine Plain only for the A/Wyoming strain).

### Example VIII

### Clinical Trial in an Elderly Population Aged Over 65 Years with a Vaccine Containing a Split Influenza Antigen Preparation and AS03 with or without MPL Adjuvant

### VIII.1. Study Design

A phase I, open, randomised, controlled study in an elderly population aged over 65 years (≧65 years-old) in order to evaluate the reactogenicity and the immunogenicity of GlaxoSmithKline Biologicals influenza candidate vaccines containing the adjuvant AS03 or AS03+MPL, administered intramuscularly as compared to Fluarix™ vaccine (known as α-Rix™ in Belgium).

Three parallel groups were assessed:
one group of 50 subjects receiving one dose of the reconstituted and AS03 adjuvanted SV influenza vaccine (Flu AS03)
one group of 50 subjects receiving one dose of the reconstituted and Flu AS03+MPL adjuvanted SV influenza vaccine (Flu AS03+MPL)
one control group of 50 subjects receiving one dose of Fluarix™ (Fluarix)

### VIII.2. Vaccine Composition and Administration

The strains used in the three vaccines were the ones that had been recommended by the WHO for the 2004-2005 Northern Hemisphere season, i.e. A/New Calcdonia/20/99 (H1N1), A/New California/3/2003 (H3N2) and B/Jiangsu/10/2003. Like Fluarix™/α-Rix™, the commercially available vaccine used as a comparator, the adjuvanted vaccines (AS03, or AS03+MPL) contain 15 μg haemagglutinin (HA) of each influenza virus strain per dose.

The adjuvanted influenza candidate vaccines are 2 component vaccines consisting of a concentrated trivalent inactivated split virion antigens presented in a type I glass vial and of a pre-filled type I glass syringe containing the adjuvant (AS03 or AS03+MPL). They have been prepared as detailed in Example II. The three inactivated split virion antigens (monovalent bulks) used in formulation of the adjuvanted influenza candidate vaccines, are exactly the same as the active ingredients used in formulation of the commercial Fluarix™/α-Rix.

### AS03 Adjuvanted Vaccine:

The AS03-adjuvanted influenza candidate vaccine is a 2 components vaccine consisting of a concentrated trivalent inactivated split virion antigens presented in a type I glass vial (335 μl) (antigen container) and of a pre-filled type I glass syringe containing the SB62 emulsion (335 μl) (adjuvant container). Description and composition of the AS03 candidate vaccine is explained in Example III.

### AS03+MPL Adjuvanted Vaccine:

Briefly, the AS03+MPL-adjuvanted influenza candidate vaccine is a 2 components vaccine consisting of a concentrated trivalent inactivated split virion antigens presented in a type I glass vial (335 μl) (antigen container) and of a pre-filled type I glass syringe containing the AS03+MPL adjuvant (360 μl) (adjuvant container). At the time of injection, the content of the antigen container is removed from the vial by using the syringe containing the AS03+MPL adjuvant, followed by gently mixing of the syringe. Prior to injection, the used needle is replaced by an intramuscular needle and the volume is corrected to 530 μl. One dose of the reconstituted the AS03+MPL-adjuvanted influenza candidate vaccine corresponds to 530 μl. To obtain the 15 μg HA for each influenza strain at reconstitution of the AS03+MPL adjuvanted vaccine, the inactivated split virion antigen are concentrated two-fold in the antigen container (i.e. 60 μg HA/ml) as compared to Fluarix™ (i.e. 30 μg HA/ml).

The composition of one dose of the reconstituted adjuvanted influenza vaccine is identical to that reported in Table 45 (see Example XI) except for the influenza strains. Both vaccines were given intramuscularly.

### VIII.3. CMI Objective, End-Points and Results

The CMI objectives were to determine which immunogenic composition between the formulation adjuvanted with AS03, or AS03+MPL versus the composition without any adjuvant has the strongest immunostimulating activity on CD4− and CD8− mediated immunity of individuals vaccinated with influenza antigens.

### VIII.3.1. CMI End Points and Results

### Observed Variable

At days 0 and 21: frequency of cytokine-positive CD4/CD8 cells per 10⁶ into 5 different cytokines. Each test quantifies the response of CD4/CD8 T cell to:
Pool of the 3 following antigens
New Calcdonia antigen
Wyoming antigen
Jiangsu antigen.

### Derived Variables:

Antigen-specific CD4 and CD8−T-cell response expressed into the 5 different tests:

(a) cells producing at least two different cytokines (CD40L, IL-2, IFNγ, TNFα)
(b) cells producing at least CD40L and another cytokine (IL-2, TNFα, IFNγ)
(c) cells producing at least IL-2 and another cytokine (CD40L, TNFα, IFNγ)
(d) cells producing at least IFNγ and another cytokine (IL-2, TNFα, CD40L)
(e) cells producing at least TNFα and another cytokine (IL-2, CD40L, IFNγ)

### Analysis of the CMI Response:

The CMI analysis was based on the Total vaccinated cohort.
(a) For each treatment group, the frequency of CD4/CD8 T-lymphocytes secreting in response was determined for each vaccination group, at each timepoint (Day 0, Day 21) and for each antigen: New Calcdonia, Wyoming and Jiangsu and the pooled of the 3 different strains.
(b) Descriptive statistics in individual difference between timepoint (POST-PRE) responses for each vaccination group and each antigen at each 5 different cytokines.
(c) Comparison of the 3 groups regarding the 5 different cytokines on:
   CD4 T-cell response to New Calcdonia, Wyoming, Jiangsu and the pool of the 3 strains
   CD8 T-cell response to New Calcdonia, Wyoming, Jiangsu and the pool of the 3 strains
(d) A non-parametric test (Kruskall-Wallis test) was used to compare the location differences between the 3 groups and the statistical p-value was calculated for each antigen at each 5 different cytokines.
(e) A Wilcoxon test were use to test pairwise comparison of 2 groups respectively between Flu AS03+MPL versus Fluarix, Flu AS03+MPL versus Flu AS03 and Flu AS03 versus Fluarix
(f) All significance tests were two-tailed. P-values less than or equal to 0.05 were considered as statistically significant.

### VIII.3.2. CMI Results

Results were expressed as a frequency of cytokine(s)-positive CD4 or CD8 T cell within the CD4 or CD8 T cell sub-population.

### Frequency of Antigen Specific CD4 T-Lymphocytes

(a) The frequency of antigen-specific CD4 T-lymphocytes secreting in response was determined for each vaccination group, at each time point (Day 0, Day 21) and for each antigen (Pool, New Calcdonia, Wyoming and Jiangsu), similarly to that performed in Example III.
(b) Comparing the difference in the frequency of antigen-specific CD4 T-lymphocytes between the 3 groups by Kruskall-Wallis test, all p-values were less than 0.05 and were considered as statistically significant.
(c) Comparing the difference in the frequency of antigen-specific CD4 T-lymphocytes between Flu AS03+MPL and Fluarix groups by the Wilcoxon test, all p-values were less than 0.05 and were considered as statistically significant.
(d) Comparing the difference in the frequency antigen-specific of CD4 T-lymphocytes between Flu AS03 and Fluarix groups by the Wilcoxon test, all p-values were less than 0.05 and were considered as statistically significant.
(e) Comparing the difference in the frequency of antigen-specific CD4 T-lymphocytes between Flu AS03 and Flu AS03+MPL groups by the Wilcoxon test, all p-values were more than 0.05 and were considered as no statistically significant.

### Individual Difference Between Time Point (Post-Pre) in CD4 T-Lymphocytes

(a) Descriptive statistics in individual difference between time point (POST-PRE) in CD4 T-lymphocytes responses was calculated for each vaccination group and for each antigen at each 5 different cytokines, similarly to what has been done in Example III.
(b) Comparing the individual difference POST-PRE in the antigen-specific CD4−T-lymphocytes responses between the 3 groups by Kruskall-Wallis test, all p-values were less than to 0.001 and were considered as highly statistically significant.
(c) Comparing the individual difference POST-PRE in the antigen-specific CD4−T-lymphocytes responses between Flu AS03+MPL and Fluarix using Wilcoxon test, all p-values were less than to 0.05 and were considered as statistically significant.
(d) Comparing the individual difference POST-PRE in the antigen-specific CD4−T-lymphocytes responses between Flu AS03 and Fluarix using Wilcoxon test, all p-values were less than to 0.001 and were considered as highly statistically significant.
(e) Comparing the individual difference POST-PRE in the antigen-specific CD4−T-lymphocytes responses between Flu AS03+MPL and Flu AS03 using Wilcoxon test, all p-values were more than 0.05 and were considered as no statistically significant.

### VIII.4. B Cell Memory Response Objective, End-Points and Results

The objective of the study was to investigate whether the frequency of memory B cell specific to Flu Antigen are significantly induced upon one intramuscular vaccination with the Flu candidate vaccine containing the Adjuvant AS03+MPL or AS03, as compared to Fluarix in elderly population. The frequency of memory B cell has been assessed by B cell Elispot assay.

### VIII.4.1. B Cell Memory Response End-Points

The end points are:
(a) At days 0, 21: cells generated in vitro cultivated memory B-cells measured by B-cell ELISPOST in all subjects in term of frequency of specific-antigen plasma within a million (10⁶) of IgG producing plasma cells.
(b) Difference between post (day 21) and pre (day 0) vaccination are also expressed as a frequency of Influenza specific-antibody forming cells per million (10⁶) of antibody forming cells.

### VIII.4.2. B Cell Memory Response Results

The frequency of Influenza-specific antibody forming cells per million (10⁶) of antibody forming cells were determined. The results showed that the frequency of memory B cell specific to Flu antigen between Flu AS03+MPL and Fluarix groups by the Wilcoxon test was significantly (p<0.05) higher for B/Jiangsu strain, whilst not for the other two strains (A strains New Calcdonia and Wyoming).

The individual difference between time point (post-pre) in memory B cell specific to Flu antigen was also determined. The results showed that individual difference between time point (post-pre) in the frequency of memory B cell specific to Flu antigen between Flu AS03+MPL and Fluarix groups by the by the Kruskall-Wallis test was significantly (p<0.05) higher for B/Jiangsu strain, whilst not for the other two strains (A strains New Calcdonia and Wyoming).

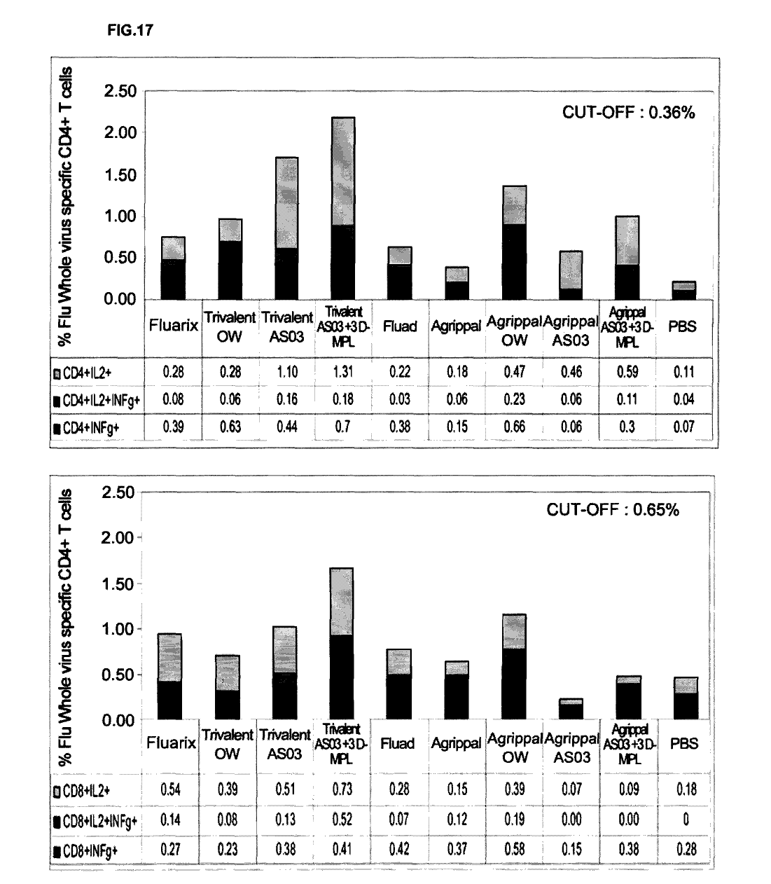

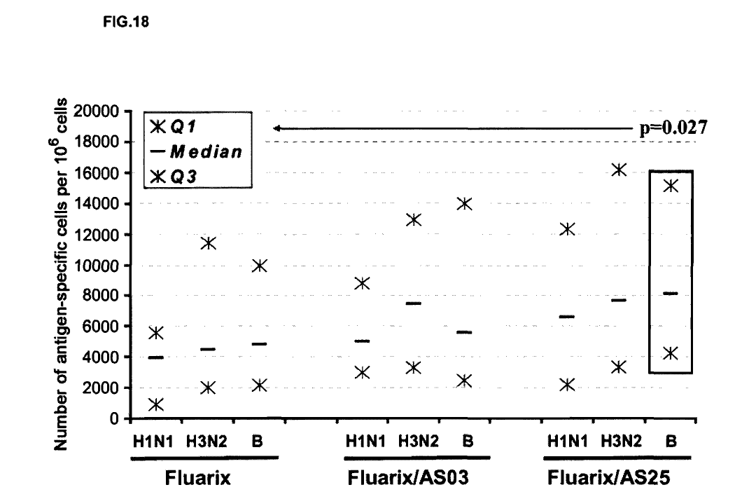

The results are shown in FIG. 18.

### Example IX

### Pre-Clinical Evaluation of Adjuvanted and Unadjuvanted Influenza Vaccines in Ferrets (Study III)

### IX.1. Rationale and Objectives

This study compared GSK commercial influenza trivalent split vaccine, either un-adjuvanted (Fluarix™) or adjuvanted with AS03+MPL, with two other commercially available sub-unit vaccines:
Fluad™, Chiron's adjuvanted subunit vaccine (the adjuvant is Chiron's MF59 adjuvant),
Agrippal™, Chiron un-adjuvanted commercial sub-unit vaccine, which was in the present study adjuvanted with AS03 adjuvant.

The objective of this experiment was to evaluate the ability of these vaccines to reduce disease symptoms (body temperature and viral shedding) in nasal secretions of ferrets challenged with heterologous strains.

The end-points were:

1) Primary end-point: reduction of viral shedding in nasal washes after heterologous challenge:
2) Secondary end-points: analysis of the humoral response by IHA and monitoring of the temperature around the priming and the heterologous challenge.

### IX.2. Experimental Design

### IX.2.1. Treatment/Group

Female ferrets (Mustela putorius furo) aged 14-20 weeks were obtained from MISAY Consultancy (Hampshire, UK). Ferrets were primed intranasally on day 0 with the heterosubtypic strain H1N1 A/Stockholm/24/90 (4 Log TCID₅₀/ml). On day 21, ferrets were injected intramuscularly with a full human dose (1 ml vaccine dose, 15 μg HA/strain) of a combination of H1N1 A/New Calcdonia/20/99, H3N2 A/Wyoming/3/2003 and B/Jiangsu/10/2003. Ferrets were then challenged on day 42 by intranasal route with a heterotypic strain H3N2 A/Panama/2007/99 (4.51 Log TCID₅₀/ml). The groups (6 ferrets/group) are illustrated in Table 41. The read-out that were performed are detailed in Table 42.

TABLE 41

| | | | Comments | |
|---|---|---|---|---|
| | Antigen(s) + | Formulation + | (ex: schedule/ | |
| Group | dosage | dosage | route/challenge) | Other treatments |
| 1 | Trivalent plain | Full HD: 15 μg | IM; Day 21 | Priming H1N1 |
| | (Fluarix ™) | HA/strain | | (A/Stockolm/24/ |
| | | | | 90) Day 0 |
| 2 | Trivalent | Full HD: 15 μg | IM; Day 21 | Priming H1N1 |
| | AS03 + MPL | HA/strain | | (A/Stockolm/24/90) |
| | | | | Day 0 |
| 3 | Fluad ™ | Full HD: 15 μg | IM; Day 21 | Priming H1N1 |
| | | HA/strain | | (A/Stockolm/24/ |
| | | | | 90) Day 0 |
| 4 | Agrippal ™ | Full HD: 15 μg | IM; Day 21 | Priming H1N1 |
| | AS03 | HA/strain | | (A/Stockolm/24/ |
| | | | | 90) Day 0 |

### IX.2.2. Preparation of the Vaccine Formulations

### Split Trivalent Plain (Un-Adjuvanted): Formulation for 1 ml:

PBS 10 fold concentrated (pH 7.4 when one fold concentrated) as well as a mixture containing Tween 80, Triton X-100 and VES (quantities taking into account the detergents present in the strains) are added to water for injection. The detergents quantities reached are the following: 375 μg Tween 80, 55 μg Triton X-100 and 50 μg VES per 1 ml. After 5 min stirring, 15 μg of each strain H1N1, H3N2 and 17.5 μg of B strain are added with 10 min stirring between each addition. The formulation is stirred for 15 minutes at room temperature and stored at 4° C. if not administered directly.

Split Trivalent Adjuvanted with AS03+MPL: Formulation for 1 ml:

PBS 10 fold concentrated (pH 7.4 when one fold concentrated) as well as a mixture containing Tween 80, Triton X-100 and VES (quantities taking into account the detergents present in the strains) is added to water for injection. The detergents quantities reached are the following: 375 μg Tween 80, 55 μg Triton X-100 and 50 μg VES per 1 ml. After 5 min stirring, 15 μg of each strain H1N1, H3N2 and B are added with 10 min stirring between each addition. After 15 min stirring, 250 μl of SB62 emulsion (prepared as detailed in Example II.1) is added. The mixture is stirred again for 15 minutes just prior addition of 25 μg of MPL. The formulation is stirred for 15 minutes at room temperature and stored at 4° C. if not administered directly.

### FluAd™ Formulation: Formulation for 1 ml:

A 2 fold dilution of FluAd™ vaccine is made in PBS buffer pH 7.4.

### Agrippal™ AS03 Formulation: Formulation for 1 ml:

250 μl of PBS buffer pH 7.4 is added to one dose of Aggripal™. After mixing, 250 μl of SB62 emulsion (prepared as detailed in Example II.1) is added. The mixture is stirred at room temperature.

### IX.2.2. Read-Outs

TABLE 42

| | | Sample- | | Analysis |
|---|---|---|---|---|
| Readout | Timepoint | type | I/Po | method |
| Viral | D−3 to D+7 Post priming | Nasal | In | Titration |
| shedding | D+1 to D+5 Post challenge | washes | | |
| T° | D−3 to D+4 Post priming | Implant in | In | Telemetry |
| monitoring | D−2 to D+4 Post challenge | peritoneal | | |
| | | cavity | | |
| IHA | Pre, Post priming, Post | Serum | In | IHA |
| | imm, Post challenge | | | |

In = Individual/Po = Pool

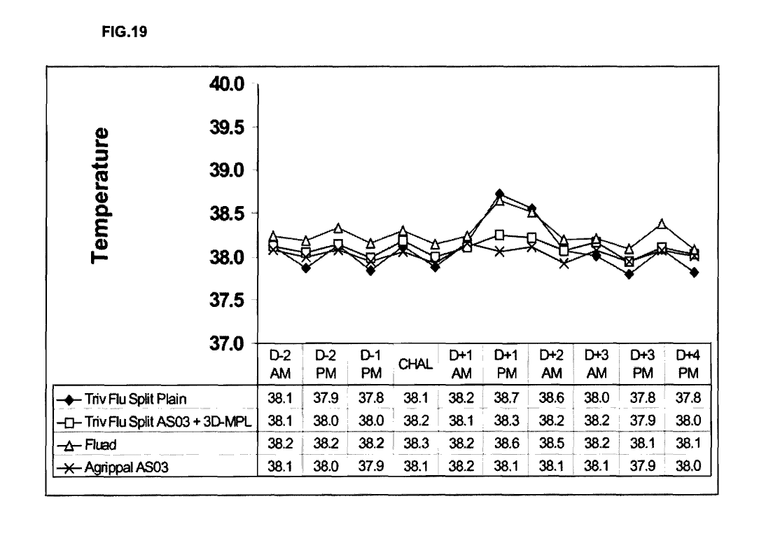

### IX.3. Results (FIGS. 19 to 22)

### IX.3.1. Temperature Monitoring

Individual temperatures were monitored with the transmitters and by the telemetry recording. All implants were checked and refurbished and a new calibration was performed by DSI before placement in the intraperitoneal cavity. All animals were individually housed in single cage during these measurements. Temperature was monitored from 2 days Pre-challenge until 4 days Post challenge every 15 minutes and an average temperature calculated by mid-day. Results are shown in FIG. 19.

### Results:

Post-challenge, a peak of body temperature was observed after immunization of ferrets with the un-adjuvanted (plain) trivalent split (Fluarix™) or the sub-unit vaccine Fluad T (which contains MF59 oil-in-water emulsion). No peak was observed after immunization of ferrets with the trivalent split vaccine adjuvanted neither with AS03+MPL nor with sub-unit Agrippal™ adjuvanted with AS03. In conclusion, an added value of the AS03-containing vaccines in the prevention of body temperature rise after challenge was shown for both the split and sub-unit tested vaccines, by contrast to the inability of the MF59—containing vaccines to prevent this temperature rise in ferrets after challenge.

### IX.3.2. Viral Shedding

Viral titration of nasal washes was performed on 6 animals per group. The nasal washes were performed by the administration of 5 ml of PBS in both nostrils in awake animals. The inoculation was collected in a Petri dish and placed into sample containers on dry ice (−80° C.).

All nasal samples were first sterile filtered through Spin X filters (Costar) to remove any bacterial contamination. 50 μl of serial ten-fold dilutions of nasal washes were transferred to microtiter plates containing 50 μl of medium (10 wells/dilution). 100 μl of MDCK cells (2.4×10⁵ cells/ml) were then added to each well and incubated at 35° C. for 5-7 days. After 5-7 days of incubation, the culture medium is gently removed and 100 μl of a 1/20 WST-1 containing medium is added and incubated for another 18 hrs.

The intensity of the yellow formazan dye produced upon reduction of WST-1 by viable cells is proportional to the number of viable cells present in the well at the end of the viral titration assay and is quantified by measuring the absorbance of each well at the appropriate wavelength (450 nanometers). The cut-off is defined as the OD average of uninfected control cells—0.3 OD (0.3 OD corresponds to +/−3 St Dev of OD of uninfected control cells). A positive score is defined when OD is <cut-off and in contrast a negative score is defined when OD is >cut-off. Viral shedding titers were determined by “Reed and Muench” and expressed as Log TCID50/ml.

### Results:

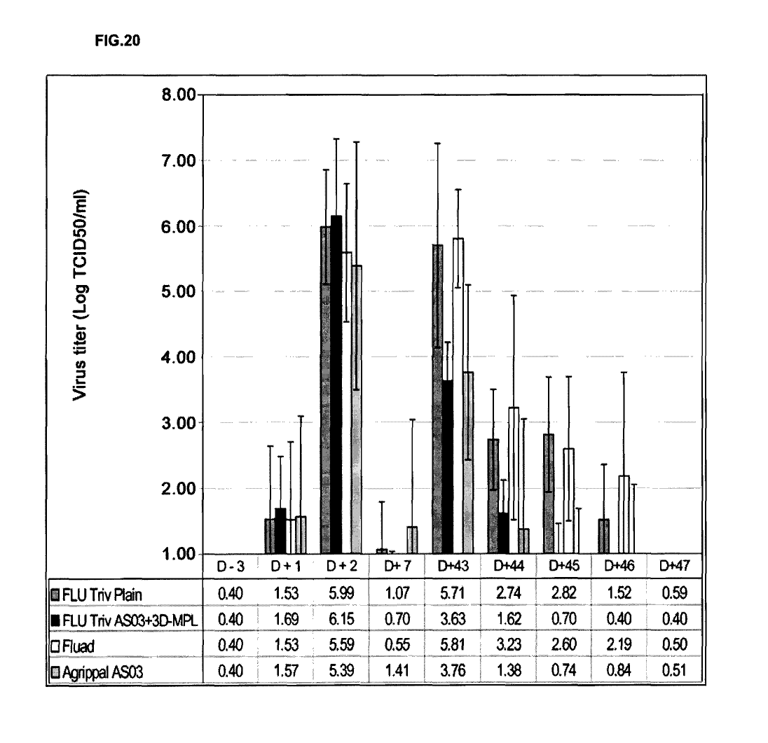

Results are shown in FIG. 20. Lower viral shedding was observed post-challenge with the trivalent split vaccine adjuvanted with AS03+MPL, or with the Agrippal™ sub-unit vaccine adjuvanted with AS03, as compared to the very low viral shedding reduction observed after immunization of ferrets with the un-adjuvanted (plain) trivalent split vaccine (Fluarix™) or with Fluad™ sub-unit vaccine.

Similarly to what was discussed in respect of body temperature rise, an added value of the AS03-containing vaccines was observed compared to the MF59-containing vaccines.

IX.3.3. HI titers

Anti-Hemagglutinin antibody titers to the H3N2 influenza virus strains were determined using the hemagglutination inhibition test (HI). The principle of the HI test is based on the ability of specific anti-Influenza antibodies to inhibit hemagglutination of chicken red blood cells (RBC) by influenza virus hemagglutinin (HA). Sera were first treated with a 25% neuraminidase solution (RDE) and were heat-inactivated to remove non-specific inhibitors. After pre-treatment, two-fold dilutions of sera were incubated with 4 hemagglutination units of each influenza strain. Chicken red blood cells were then added and the inhibition of agglutination was scored. The titers were expressed as the reciprocal of the highest dilution of serum that completely inhibited hemagglutination. As the first dilution of sera was 1:10, an undetectable level was scored as a titer equal to 5.

### Results:

After immunization with H3N2 A/Wyoming, higher humoral responses (HI titers) were observed in ferrets immunized with the trivalent split vaccine adjuvanted with AS03+MPL or with the Agrippal™ sub-unit vaccine adjuvanted with AS03, as compared to the humoral response observed after immunization of ferrets with the un-adjuvanted (plain) trivalent split vaccine (Fluarix™) or with Fluad™ sub-unit vaccine (FIG. 21).

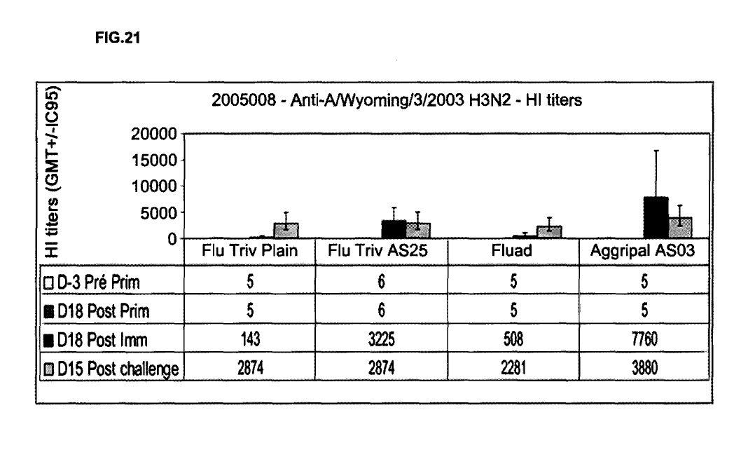

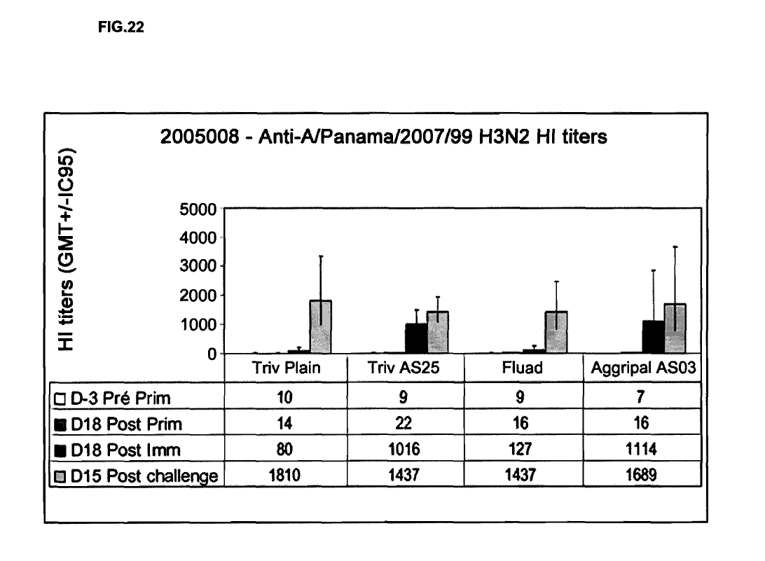

After immunization with H3N2 A/Wyoming, higher humoral responses (HI titers) were also observed against the drift strain H3N2 A/Panama, used as the challenge strain, in ferrets immunized with Trivalent Split adjuvanted with AS03+MPL or Agrippal™ adjuvanted with AS03 compared to ferrets immunized with Trivalent Split Plain or Fluad (FIG. 22).

This cross-reaction observed with our adjuvant (AS03 or AS03+MPL) against a heterologous strain correlated with the protection observed in ferrets immunized with the trivalent split vaccine adjuvanted with AS03+MPL or with the Agrippal™ sub-unit vaccine adjuvanted with AS03, and then challenged with this heterologous strain. This cross-reactivity to heterologous strain induced by AS03-containing vaccines was not induced by the MF59's adjuvanted vaccines (FluAd™).

### Example X

### Clinical Trial in an Elderly Population Aged Over 65 Years with a Vaccine Containing a Split Influenza Antigen Preparation and AS03 with or without MPL Adjuvant: Immunogenicity Persistence Data at Day 90 and 180

### X.1. Study Design

A phase I, open, randomised, controlled study in an elderly population aged over 65 years (≧65 years-old) in order to evaluate the reactogenicity and the immunogenicity of GlaxoSmithKline Biologicals influenza candidate vaccines containing the adjuvant AS03 or AS03+MPL, administered intramuscularly as compared to Fluarix™ vaccine (known as α-Rix™ in Belgium). This study follows that reported in Example VIII.

Three parallel groups were assessed:
one group of 50 subjects receiving one dose of the reconstituted and AS03 adjuvanted SV influenza vaccine (Flu AS03)
one group of 50 subjects receiving one dose of the reconstituted and Flu AS03+MPL adjuvanted SV influenza vaccine (Flu AS03+MPL)
one control group of 50 subjects receiving one dose of Fluarix™ (Fluarix)

### X.2. Immunogenicity Results

### X.2.1. Humoral Immune Response Endpoints and Results

In order to evaluate the humoral immune response induced by the AS03 and AS03+MPL adjuvanted vaccines and its persistence, the following parameters were calculated for each treatment group.

At Days 0, 21, 90 and 180: serum haemagglutination-inhibition (HI) antibody titres, tested separately against each of the three influenza virus strains represented in the vaccine (anti-H1N1, anti-H3N2 & anti-B-antibodies).
Serum HI antibody GMTs' with 95% CI at Days 0, 21, 90 and 180
Seroconversion rates with 95% CI at Days 21, 90 and 180
Conversion factors with 95% CI at Day 21
Seroprotection rates with 95% CI at Days 0, 21, 90 and 180

### Results

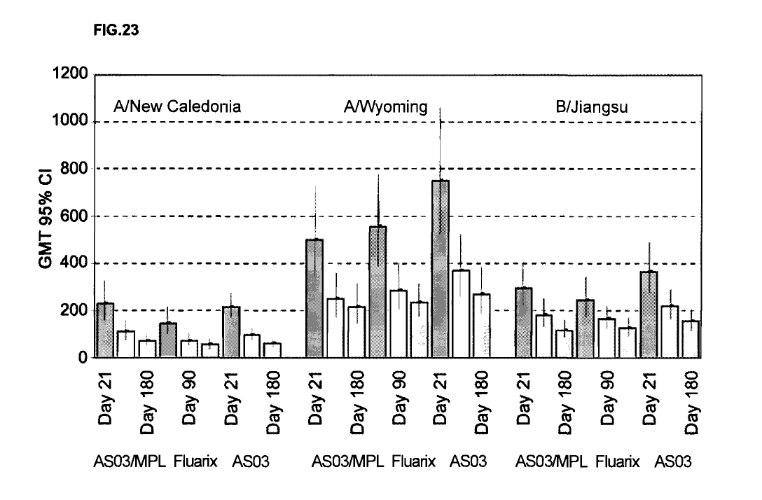

The GMTs for HI antibodies with 95% CI are shown in FIG. 23. Pre-vaccination GMTs of antibodies for all 3 vaccine-strains were within the same range in the 3 groups. After vaccinations, anti-haemagglutinin antibody levels increased significantly. Post-vaccination GMTs of antibodies for the 3 vaccine strains remained however within the same ranges for all vaccines. On Day 21, a slight tendency in favour of the 2 adjuvanted vaccines compared to Fluarix was noted for the A/New Calcdonia and the B/Jiangsu strains and among the two adjuvanted vaccines, the higher GMTs were observed with FLU AS03 for the A/Wyoming and B/Jiangsu strains.

The same trends were observed at Day 90. On Day 180, GMTs of antibodies for the 3 vaccine strains were within the same ranges for the 3 vaccines.

All influenza vaccines fulfilled the requirements of the European authorities for annual registration of influenza inactivated vaccines [“Note for Guidance on Harmonisation of Requirements for Influenza Vaccines for the immunological assessment of the annual strain changes” (CPMP/BWP/214/96)] in subjects aged over 60 years.

Three months (90 days) and 6 months (180 days) after vaccination, the seroprotection rates were still higher than the minimum rate of 60% required by the European Authorities whatever the study group considered. On Day 90, the minimum seroconversion rate of 30% required by the European Authorities was still achieved for all vaccines strains in the 3 vaccine groups except with Fluarix for the A/New Calcdonia strain. On Day 180, it was still achieved for the A/Wyoming and B/Jiangsu strains with the 3 vaccines but not for the A/New Calcdonia strain (Table 43 and Table 44).

TABLE 43
Seroprotection rates as the percentage of vaccinees
with a serum haemagglutination inhibition titre superior
or equal to 1:40 (ATP cohort for immunogenicity)

| | | | | ≧1:40 | | 95% CI | |
|---|---|---|---|---|---|---|---|
| Antibody | Group | Timing | N | n | % | LL | UL |
| A/ | Flu AS03 + | PRE | 50 | 28 | 56.0 | 41.3 | 70.0 |
| New | MPL | PI(D 21) | 50 | 46 | 92.0 | 80.8 | 97.8 |
| Caledonia | | PI(D 90) | 50 | 43 | 86.0 | 73.3 | 94.2 |
| | | PI(D 180) | 50 | 39 | 78.0 | 64.0 | 88.5 |
| | Fluarix | PRE | 50 | 26 | 52.0 | 37.4 | 66.3 |
| | | PI(D 21) | 50 | 46 | 92.0 | 80.8 | 97.8 |
| | | PI(D 90) | 50 | 38 | 76.0 | 61.8 | 86.9 |
| | | PI(D 180) | 50 | 34 | 68.0 | 53.3 | 80.5 |
| | FluAS03 | PRE | 49 | 28 | 57.1 | 42.2 | 71.2 |
| | | PI(D 21) | 49 | 48 | 98.0 | 89.1 | 99.9 |
| | | PI(D 90) | 49 | 45 | 91.8 | 80.4 | 97.7 |
| | | PI(D 180) | 49 | 38 | 77.6 | 63.4 | 88.2 |
| A/ | Flu AS03 + | PRE | 50 | 33 | 66.0 | 51.2 | 78.8 |
| Wyoming | MPL | PI(D 21) | 50 | 47 | 94.0 | 83.5 | 98.7 |
| | | PI(D 90) | 50 | 46 | 92.0 | 80.8 | 97.8 |
| | | PI(D 180) | 50 | 45 | 90.0 | 78.2 | 96.7 |
| | Fluarix | PRE | 50 | 32 | 64.0 | 49.2 | 77.1 |
| | | PI(D 21) | 50 | 50 | 100 | 92.9 | 100.0 |
| | | PI(D 90) | 50 | 49 | 98.0 | 89.4 | 99.9 |
| | | PI(D 180) | 50 | 50 | 100 | 92.9 | 100.0 |
| | FluAS03 | PRE | 49 | 34 | 69.4 | 54.6 | 81.7 |
| | | PI(D 21) | 49 | 48 | 98.0 | 89.1 | 99.9 |
| | | PI(D 90) | 49 | 46 | 93.9 | 83.1 | 98.7 |
| | | PI(D 180) | 49 | 47 | 95.9 | 86.0 | 99.5 |
| B/ | Flu AS03 + | PRE | 50 | 19 | 38.0 | 24.7 | 52.8 |
| Jiangsu | MPL | PI(D 21) | 50 | 50 | 100 | 92.9 | 100.0 |
| | | PI(D 90) | 50 | 47 | 94.0 | 83.5 | 98.7 |
| | | PI(D 180) | 50 | 46 | 92.0 | 80.8 | 97.8 |
| | Fluarix | PRE | 50 | 17 | 34.0 | 21.2 | 48.8 |
| | | PI(D 21) | 50 | 48 | 96.0 | 86.3 | 99.5 |
| | | PI(D 90) | 50 | 47 | 94.0 | 83.5 | 98.7 |
| | | PI(D 180) | 50 | 47 | 94.0 | 83.5 | 98.7 |
| | FluAS03 | PRE | 49 | 25 | 51.0 | 36.3 | 65.6 |
| | | PI(D 21) | 49 | 49 | 100 | 92.7 | 100.0 |
| | | PI(D 90) | 49 | 47 | 95.9 | 86.0 | 99.5 |
| | | PI(D 180) | 49 | 46 | 93.9 | 83.1 | 98.7 |

N = number of subjects with available results
n/% = number/percentage of subjects with titre within the specified range
PRE = pre-vaccination titre
PI (D 21) = post-vaccination blood sampling at Day 21
PI (D 90) = post-vaccination blood sampling at Day 90
PI (D 180) = post-vaccination blood sampling at Day 180

TABLE 44
Seroconversion rate for haemagglutination inhibition (HI) antibody
titres defined as the percentage of vaccinees who have at least
a 4-fold increase in serum HI titre at each post-vaccination time
point compared to Day 0 (ATP cohort for immunogenicity)

| | | | | 4-fold | | | |
|---|---|---|---|---|---|---|---|
| Vaccine | | | | | | 95% CI | |
| strain | Timing | Group | N | n | % | LL | UL |
| A/ | Day 21 | Flu AS03 + MPL | 50 | 30 | 60.0 | 45.2 | 73.6 |
| NEW | | Fluarix | 50 | 25 | 50.0 | 35.5 | 64.5 |
| CALEDONIA | | Flu AS03 | 49 | 31 | 63.3 | 48.3 | 76.6 |
| | Day 90 | Flu AS03 + MPL | 50 | 19 | 38.0 | 24.7 | 52.8 |
| | | Fluarix | 50 | 14 | 28.0 | 16.2 | 42.5 |
| | | Flu AS03 | 49 | 17 | 34.7 | 21.7 | 49.6 |
| | Day 180 | Flu AS03 + MPL | 50 | 12 | 24.0 | 13.1 | 38.2 |
| | | Fluarix | 50 | 11 | 22.0 | 11.5 | 36.0 |
| | | Flu AS03 | 49 | 10 | 20.4 | 10.2 | 34.3 |
| A/ | Day 21 | Flu AS03 + MPL | 50 | 46 | 92.0 | 80.8 | 97.8 |
| WYOMING | | Fluarix | 50 | 38 | 76.0 | 61.8 | 86.9 |
| | | Flu AS03 | 49 | 40 | 81.6 | 68.0 | 91.2 |
| | Day 90 | Flu AS03 + MPL | 50 | 33 | 66.0 | 51.2 | 78.8 |
| | | Fluarix | 50 | 33 | 66.0 | 51.2 | 78.8 |
| | | Flu AS03 | 49 | 31 | 63.3 | 48.3 | 76.6 |
| | Day 180 | Flu AS03 + MPL | 50 | 27 | 54.0 | 39.3 | 68.2 |
| | | Fluarix | 50 | 23 | 46.0 | 31.8 | 60.7 |
| | | Flu AS03 | 49 | 26 | 53.1 | 38.3 | 67.5 |
| B/ | Day 21 | Flu AS03 + MPL | 50 | 44 | 88.0 | 75.7 | 95.5 |
| JIANGSU | | Fluarix | 50 | 38 | 76.0 | 61.8 | 86.9 |
| | | Flu AS03 | 49 | 43 | 87.8 | 75.2 | 95.4 |
| | Day 90 | Flu AS03 + MPL | 50 | 37 | 74.0 | 59.7 | 85.4 |
| | | Fluarix | 50 | 36 | 72.0 | 57.5 | 83.8 |
| | | Flu AS03 | 49 | 37 | 75.5 | 61.1 | 86.7 |
| | Day 180 | Flu AS03 + MPL | 50 | 32 | 64.0 | 49.2 | 77.1 |
| | | Fluarix | 50 | 29 | 58.0 | 43.2 | 71.8 |
| | | Flu AS03 | 49 | 31 | 63.3 | 48.3 | 76.6 |

N = number of subjects with both pre- and post-vaccination results available
n/% = number/percentage of subjects with at least a 4-fold increase
95% CI = exact 95% confidence interval; LL = lower limit, UL = upper limit

### X.2.2. CMI Response Endpoints and Results

In order to evaluate the cellular immune response induced by the adjuvanted vaccines and its persistence, the following parameters were calculated for each treatment group:

At each time point (Days 0, 21, 90 and 180): frequency of cytokine-positive CD4/CD8 cells per 10⁶ in different tests (New Calcdonia, Wyoming and Jiangsu antigens considered separately as well as pooled at Days 0 and 21; New Calcdonia, Wyoming, Jiangsu and New York antigens considered separately as well as pooled at Days 90 and 180)
All double: cells producing at least two different cytokines (CD40L, IFN-γ, IL-2, TN F-α).
CD40L: cells producing at least CD40L and another cytokine (IFN-γ, IL-2, TNF-α).
IFN-γ: cells producing at least IFN-γ and another cytokine (CD40L, IL-2, TNF-α).
IL-2: cells producing at least IL-2 and another cytokine (CD40L, IFN-γ, TNF-α).
TNF-α: cells producing at least TNF-α and another cytokine (CD40L, IFN-γ, IL-2).

### Results

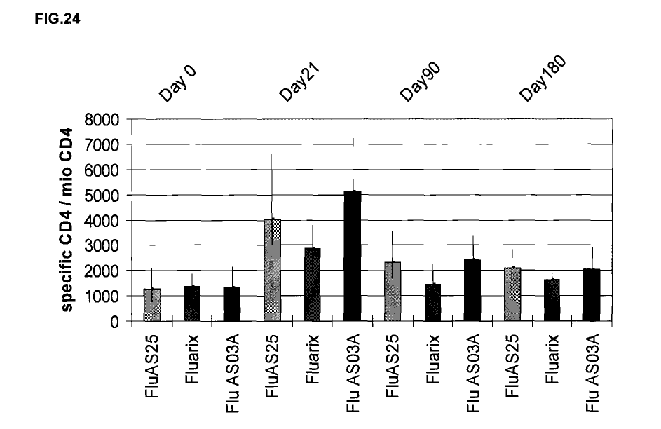

The main findings were (FIG. 24):
(a) Twenty-one days after the vaccination, the frequency of cytokine-positive CD4 T cells (IL-2, CD40L, TNF-α and IFN-γ) was significantly higher in the two adjuvanted vaccine groups compared to the Fluarix group. No significant difference was however detected between the two adjuvants.
(b) All statistical differences between adjuvanted vaccines and Fluarix were maintained up to Day 90 and Day 180 with the following exceptions at Day 180:
   No statistically significant difference was found between FluAS03/MPL and Fluarix for all double, CD40L, IFN-γ and IL2 (Wyoming strain only) and for all double, CD40L and TNF-α (New York strain only)
   No statistically significant difference was found between FluAS03 and Fluarix for IL2 (Jiangsu strain only)
(c) The absence of statistically significant difference between the two adjuvanted vaccines was confirmed up to Day 90 and Day 180.
(d) The difference between pre and post-vaccination (Day 21) in CD4 T-lymphocytes responses for all cytokines investigated (IL-2, CD40L, TNF-α and IFN-γ) was significantly higher with the two adjuvanted vaccines compared to Fluarix™. No significant difference was however detected between both adjuvants.
(e) The vaccination had no measurable impact on the CD8 response whatever the treatment group.

### Example XI

### Clinical Trial in an Elderly Population Aged Over 65 Years with a Vaccine Containing a Split Influenza Antigen Preparation and AS03 with MPL Adjuvant

### XI.1. Study Design and Objectives

A phase I/II, open, controlled study was conducted in order to evaluate the reactogenicity and the immunogenicity of GlaxoSmithKline Biologicals influenza candidate vaccine containing the AS03+MPL adjuvant in an elderly population aged over 65 years (>65 years-old) previously vaccinated in 2004 with the same candidate vaccine. For immunogenicity and safety evaluations, Fluarix™ (known as α-Rix™ in Belgium) vaccine was used as reference.

Two parallel groups were assessed:
One group of about 50 subjects who had previously received one dose of the reconstituted adjuvanted influenza vaccine during the previous clinical trial
One control group (Fluarix) of about 50 subjects who had previously received one dose of Fluarix™ during the previous clinical trial

One objective of this study was to evaluate the humoral immune response (anti-haemagglutinin and anti-MPL titres) of the revaccination with the adjuvanted influenza vaccine Flu AS03+MPL administered about one year after administration of the first dose. For comparison purposes, subjects who had already received Fluarix™ in the previous trial received a dose of commercial vaccine and formed the control group of this trial.

### XI.2. Vaccine Composition and Administration

The strains used in the three vaccines were the ones that had been recommended by the WHO for the 2005-2006 Northern Hemisphere season, i.e. A/New Calcdonia/20/99 (H1N1), A/New California/7/2004 (H3N2) and B/Jiangsu/10/2003. Like Fluarix™/α-Rix™, the commercially available vaccine used as a comparator, the (AS03+MPL—adjuvanted vaccine, hereinafter in short “the adjuvanted vaccine”) contains 15 μg haemagglutinin (HA) of each influenza virus strain per dose.

The adjuvanted influenza candidate vaccine is a 2 component vaccine consisting of a concentrated trivalent inactivated split virion antigens presented in a type I glass vial and of a pre-filled type I glass syringe containing the AS03+MPL adjuvant. It has been prepared according the method detailed in Example II.

At the time of injection, the content of the prefilled syringe containing the adjuvant is injected into the vial that contains the concentrated trivalent inactivated split virion antigens. After mixing the content is withdrawn into the syringe and the needle is replaced by an intramuscular needle. One dose of the reconstituted the adjuvanted influenza candidate vaccine corresponds to 0.7 mL. The adjuvanted influenza candidate vaccine is a preservative-free vaccine.

The composition of one dose of the reconstituted adjuvanted influenza vaccine is given in Table 45. Both vaccines were given intramuscularly.

TABLE 45
Composition of the reconstituted vaccine adjuvanted
(AS03 + MPL) influenza candidate vaccine

| Component | Quantity per dose | |
|---|---|---|
| Inactivated split virions | | |
| A/New Caledonia/20/99 (H1N1) | 15 | μg HA |
| A/New California/7/2004 (H3N2) | 15 | μg HA |
| B/Jiangsu/10/2003 | 15 | μg HA |
| Adjuvant | | |
| SB62 emulsion | | |
| (squalene) | 10.68 | mg |
| (DL-alpha-tocopherol) | 11.86 | mg |
| (polysorbate 80 - Tween 80) | 4.85 | mg |
| MPL | 25 | μg |

### XI.3. Immunogenicity Results

### XI.3.1. Anti-HA Humoral Immune Response Endpoints and Results

### Observed Variables:

At days 0 and 21: serum haemagglutination-inhibition (HI) antibody titres, tested separately against each of the three influenza virus strains represented in the vaccine (anti-H1N1, anti-H3N2 & anti-B-antibodies).

Derived Variables (with 95% Confidence Intervals):
(f) Geometric mean titres (GMTs) of serum HI antibodies with 95% confidence intervals (95% CI) pre and post-vaccination
(g) Seroconversion rates* with 95% CI at day 21
(h) Seroconversion factors** with 95% CI at day 21
(i) Seroprotection rates*** with 95% CI at day 21 * Seroconversion rate defined as the percentage of vaccinees with either a pre-vaccination HI titre<1:10 and a post-vaccination titre≧1:40, or a pre-vaccination titre≧1:10 and a minimum 4-fold increase at post-vaccination titre, for each vaccine strain.**Seroconversion factor defined as the fold increase in serum HI GMTs on day 21 compared to day 0, for each vaccine strain.***Protection rate defined as the percentage of vaccinees with a serum HI titre≧40 after vaccination (for each vaccine strain) that usually is accepted as indicating protection.

### Results

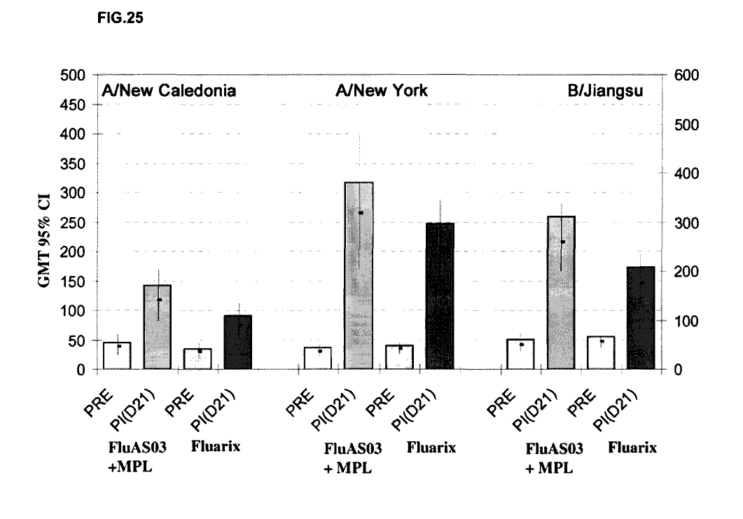

As expected, the vast majority of subjects were already seropositive for the three strains in both groups before vaccination. Pre-vaccination GMTs for all 3 vaccine strains were within the same range in the 2 groups. There was a trend for higher GMTs at post-vaccination for all 3 vaccine strains in the Flu AS03+MPL group compared to the Fluarix group, although 95% CI were overlapping (FIG. 25).

The two influenza vaccines fulfilled the requirements of the European authorities for annual registration of influenza inactivated vaccines [“Note for Guidance on Harmonisation of Requirements for Influenza Vaccines for the immunological assessment of the annual strain changes” (CPMP/BWP/214/96)] in subjects aged over 60 years (Table 46).

TABLE 46
Seroprotection rates seroconversion rates and conversion
factors at day 21 (ATP cohort for immunogenicity)

| | | | | Seroconversion rate | Seroconversion |
|---|---|---|---|---|---|
| | | | Seroprotection rate | (≧4-fold increase) | factor |
| Strains | Group | N | (HI titre ≧ 40) % | [95% CI] % | [95% CI] % |
| EU standard (>60 | | | >60% | >30% | >2.0 |
| A/New Caledonia | Flu + MPL-AS03 | 38 | 89.5 [75.20-97.06] | 31.6 [17.5-48.7] | 3.1 [2.2-4.4] |
| | Fluarix | 45 | 82.2 [67.95-92.00] | 31.1 [18.2-46.6] | 2.5 [1.8-3.5] |
| A/New York (H3N2) | Flu + MPL-AS03 | 38 | 92.1 [78.62-98.34] | 78.9 [62.7-90.4] | 8.8 [6.1-12.5] |
| | Fluarix | 45 | 95.6 [84.85-99.46] | 68.9 [53.4-818] | 6.0 [4.4-8.3] |
| B/Jiangsu (B) | Flu + MPL-AS03 | 38 | 100 [90.75-100] | 57.9 [40.8-73.7] | 5.1 [3.7-7.0] |
| | Fluarix | 45 | 100 [92.13-100] | 37.8 [23.8-53.5] | 3.1 [2.4-4.0] |

N = total number of subject;
% = Percentage of subjects with titre at day 21 within the specified range;
CI = confidence interval

### Example XII

### Clinical Trial in an Elderly Population Aged Over 65 Years with a Vaccine Containing a Split Influenza Antigen Preparation Adjuvanted with AS03 and MPL at Two Different Concentrations

### XII.1. Study Design and Objectives

An open, randomized phase I/II study to demonstrate the non inferiority in term of cellular mediated immune response of GlaxoSmithKline Biologicals influenza candidate vaccines containing various adjuvants administered in elderly population (aged 65 years and older) as compared to Fluarix™ (known as α-Rix™ in Belgium) administered in adults (18-40 years)

Four parallel groups were assigned:
(a) 75 adults (aged 18-40 years) in one control group receiving one dose of Fluarix™ (Fluarix group)
(b) 200 elderly subjects (aged 65 years and older) randomized 3:3:2 into three groups:
   one group with 75 subjects receiving influenza vaccine adjuvanted with AS03+MPL (concentration 1-25 μg)
   One group with 75 subjects receiving influenza vaccine adjuvanted with AS03+MPL (concentration 2-50 μg)
   Reference Flu group with 50 subjects receiving one dose of Fluarix™

### Primary Objective

The primary objective is to demonstrate the non inferiority 21 days post-vaccination of the influenza adjuvanted vaccines administered in elderly subjects (aged 65 years and older) as compared to Fluarix™ administered in adults (aged 18-40 years) in terms of frequency of influenza-specific CD4 T-lymphocytes producing at least two different cytokines (CD40L, IL-2, TNF-α, IFN-γ).

### Secondary Objectives

The secondary objectives are
(a) To evaluate the safety and reactogenicity of vaccination with candidate influenza vaccines adjuvanted during 21 days following the intramuscular administration of the vaccine in elderly subjects (aged 65 years and older). Fluarix™ is used as reference.
(b) To evaluate the humoral immune response (anti-haemagglutinin titre) 21, 90 and 180 days after vaccination with influenza candidate vaccines adjuvanted. Fluarix™ is used as reference.

### Tertiary Objective

The tertiary objective is to evaluate the cell mediated immune response (production of IFN-γ, IL-2, CD40L, and TNF-α and memory B-cell response) 21, 90 and 180 days after vaccination with adjuvanted influenza-vaccines. Fluarix™ is used as reference.

### XII.2. Vaccine Composition and Administration

The influenza vaccine adjuvanted with AS03+MPL (25 μg per dose) system is also used in study illustrated in Example XI. The influenza vaccine adjuvanted with AS03+MPL (50 μg per dose) system is of identical composition except that the concentration of MPL is doubled. The process is the same as the one described in Example VIII for the influenza vaccine adjuvanted with AS03+MPL, with as only difference that the concentration of MPL is doubled.
Control: full dose of Fluarix™ by IM administration.
Four scheduled visits per subject: at days 0, 21, 90 and 180 with blood sample collected at each visit to evaluate immunogenicity.
Vaccination schedule: one injection of influenza vaccine at day 0

### XII.3. Immunogenicity Results

### XII.3.1. CMI Endpoints and Results

### Evaluation of the Primary Endpoint.

At day 21: CMI response in all subjects in terms of frequency of influenza-specific CD4 T-lymphocyte per 10⁶ in tests producing at least two different cytokines (IL-2, IFN-γ, TNF-α and CD40L)

For evaluation of CMI response, frequency of influenza-specific CD4 are analysed as follows:

The GM ratio in term of influenza-specific CD4 frequency between groups vaccinated with adjuvanted vaccines and Flu YNG is obtained using an ANCOVA model on the logarithm-transformed titres. The ANCOVA model includes the vaccine group as fixed effect and the pre-vaccination log-transformed titre as regressor. The GM ratio and their 98.75% CI are derived as exponential-transformation of the corresponding group contrast in the model. The 98.75% CI for the adjusted GM is obtained by exponential-transformation of the 98.75% CI for the group least square mean of the above ANCOVA model.

### Results—Inferential Analysis (Table 47)

The adjusted GM and GM ratios (with their 98.75% CI) of influenza-specific CD4 T-lymphocyte producing at least two cytokines (IL-2, IFN-γ, TNF-α and CD40L) at day 21, after in vitro restimulation with “pooled antigens II”, are presented in Table 47. For each adjuvanted influenza vaccine, the upper limit of two-sided 98.75% CI of GM ratio is far below the clinical limit of 2.0. This shows the non-inferiority of both adjuvanted influenza vaccines administered to elderly subjects compared to the Fluarix™ vaccine administered in adults aged between 18 and 40 years in term of post-vaccination frequency of influenza-specific CD4.

TABLE 47
Adjusted GM ratio of influenza-specific CD4 producing at least
two cytokines, Day 21 (ATP cohort for immunogenicity)

| | | | | Adjusted GM ratio | | |
|---|---|---|---|---|---|---|
| Flu YNG | | AS03 + MPL (conc. 1) | | (Flu YNG/AS03 + MPL (conc. 1) | | |
| | Adjusted | | Adjusted | | 98.8% CI | |
| N | GM | N | GM | Value | LL | UL |
| 70 | 1995.3 | 72 | 2430.0 | 0.82 | 0.65 | 1.04 |
| | | | | Adjusted GM ratio | | |
| Flu YNG | | AS03 + MPL (conc. 2) | | (Flu YNG/AS03 + MPL (conc. 2) | | |
| | Adjusted | | Adjusted | | 98.8% CI | |
| N | GM | N | GM | Value | LL | UL |
| 70 | 1979.4 | 72 | 2603.8 | 0.76 | 0.59 | 0.98 |

Adjusted GM = geometric mean antibody adjusted for baseline titre; N = Number of subjects with both pre- and post-vaccination results available; 98.8% CI = 98.8% confidence interval for the adjusted GM ratio (Ancova model: adjustment for baseline); LL = lower limit, UL = upper limit

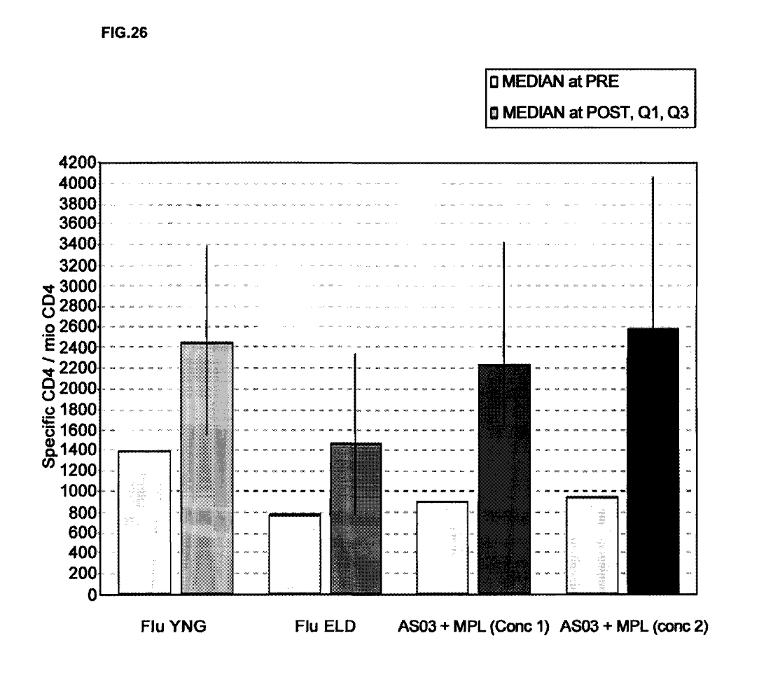

### Results—Descriptive Analysis (FIG. 26)

The main findings were:
Before vaccination the CMI response if higher in young adults than in elderly
After vaccination,
   there was a booster effect of the influenza vaccine on the CMI response in young adults (18-40 years)
   CMI response in the elderly having received adjuvanted influenza vaccine is comparable to the CMI response of young adults.
The difference between pre and post-vaccination in CD4 T-lymphocytes responses for all cytokines investigated (IL-2, CD40L, TNF-α and IFN-γ) was significantly higher with the adjuvanted vaccines compared to Fluarix™ (18-40 years) for all tests excepted for IFNγ when we compare Fluarix (18-40 years) and Flu/AS03+MPL (conc. 1).

It should be noted that the in vitro stimulation was performed with the Flu strains (i) B/Jiangsu, (ii) A/H3N2/New-York and (iii) A/H3N2/Wyoming instead of A/H1N1/New-Calcdonia included in the vaccine. However, preliminary data including the A/H1N1/New Calcdonia vaccine strain from subsets of subjects indicate that the results will be similar.

### Results—Evaluation of the Tertiary End-Point (Table 48)

In order to evaluate the tertiary end point, the frequency of influenza-specific CD4/CD8 T-lymphocytes and memory B-cells were measured at days 0, 21, 90 and 180.

The frequency of influenza-specific cytokine-positive CD4/CD8 T-lymphocytes was summarised (descriptive statistics) for each vaccination group at days 0 and 21, for each antigen.

A Non-parametric test (Wilcoxon test) was used to compare the location of difference between the two groups (influenza adjuvanted vaccine versus Fluarix™) and the statistical p-value is calculated for each antigen at each different test.

Descriptive statistics in individual difference between day 21/day 0 (Post-/Pre-vaccination) responses is calculated for each vaccination group and each antigen at each different test.

A Non-parametric test (Wilcoxon test) is used to compare the individual difference Post-/Pre-vaccination) and the statistical p-value will be calculated for each antigen at each different test.

The p-values from Wilcoxon test used to compare the difference in the frequency of influenza-specific CD4 T-lymphocytes are presented in Table 48.

TABLE 48
Inferential statistics: p-values from Kruskal-Wallis Tests for
CD4 T cells at each time point (ATP Cohort for immunogenicity)

| | p-value | | | | | | | |
|---|---|---|---|---|---|---|---|---|
| | Group 1 | | Group 2 | | Group 1 | | Group 2 | |
| | and Flu ELD | | and Flu ELD | | and Flu YNG | | and Flu YNG | |
| | day 0 | day 21 | day 0 | day 21 | day 0 | day 21 | day 0 | day 21 |
| ALL DOUBLES | 0.4380 | 0.0003 | 0.4380 | 0.0003 | 0.0000 | 0.9014 | 0.0005 | 0.4889 |
| CD4OL | 0.3194 | 0.0002 | 0.3194 | 0.0002 | 0.0000 | 0.9841 | 0.0003 | 0.5412 |
| IFNγ | 0.5450 | 0.0004 | 0.5450 | 0.0004 | 0.0000 | 0.5397 | 0.0001 | 0.7895 |
| IL2 | 0.3701 | 0.0008 | 0.3701 | 0.0008 | 0.0003 | 0.8557 | 0.0022 | 0.4766 |
| TFNα | 0.3716 | 0.0004 | 0.3716 | 0.0004 | 0.0000 | 0.8730 | 0.0013 | 0.2114 |

Group 1: Influenza vaccine adjuvanted with AS03 + MPL (conc. 1)
Group 2: Influenza vaccine adjuvanted with AS03 + MPL (conc. 2)

The main conclusions are:
(a) Pre-vaccination GM frequencies of influenza-specific CD4 were similar in all groups of elderly subjects but superior in the adults aged between 18 and 40 years.
(b) Post-vaccination (day 21) frequency of influenza-specific CD4 T lymphocytes was similar in elderly subjects vaccinated with adjuvanted vaccines and in adults aged between 18 and 40 years vaccinated with Fluarix™.
(c) In elderly subjects, post-vaccination (day 21) frequency of influenza-specific CD4 T lymphocytes was significantly higher after vaccination with adjuvanted vaccines than with Fluarix™.
(d) Pre-vaccination and post vaccination GM frequency of influenza-specific CD8 T cell was essentially similar in all groups.

### Results—Evaluation of the Humoral Immune Response Endpoints

### Observed Variables:

At days 0, 21, 90 and 180: serum haemagglutination-inhibition (HI) antibody titres, tested separately against each of the three influenza virus strains represented in the vaccine (anti-H1N1, anti-H3N2 & anti-B-antibodies).

The cut-off value for HI antibody against all vaccine antigens was defined by the laboratory before the analysis (and equals 1:10). A seronegative subject is a subject whose antibody titre is below the cut-off value. A seropositive subject is a subject whose antibody titre is greater than or equal to the cut-off value. Antibody titre below the cut-off of the assay is given an arbitrary value of half the cut-off.

Based on the HI antibody titres, the following parameters are calculated:
(j) Geometric mean titres (GMTs) of HI antibody at days 0 and 21, calculated by taking the anti-log of the mean of the log titre transformations.
(k) Seroconversion factors (SF) at day 21 defined as the fold increase in serum HI GMTs on day 21 compared to day 0.
(l) Seroconversion rates (SC) at day 21 defined as the percentage of vaccinees with either a pre-vaccination HI titre<1:10 and a post-vaccination titre≧1:40, or a pre-vaccination titre≧1:10 and a minimum 4-fold increase at post-vaccination titre.
(m) Seroprotection rates (SPR) at day 21 defined as the percentage of vaccinees with a serum HI titre≧1:40.

The 95% CI for GM is obtained within each group separately. The 95% CI for the mean of log-transformed titre is first obtained assuming that log-transformed titres are normally distributed with unknown variance. The 95% CI for the GM is then obtained by exponential-transformation of the 95% CI for the mean of log-transformed titre.

Missing serological result for a particular antibody measurement is not replaced. Therefore a subject without serological result at a given time point do not contribute to the analysis of the assay for that time point.

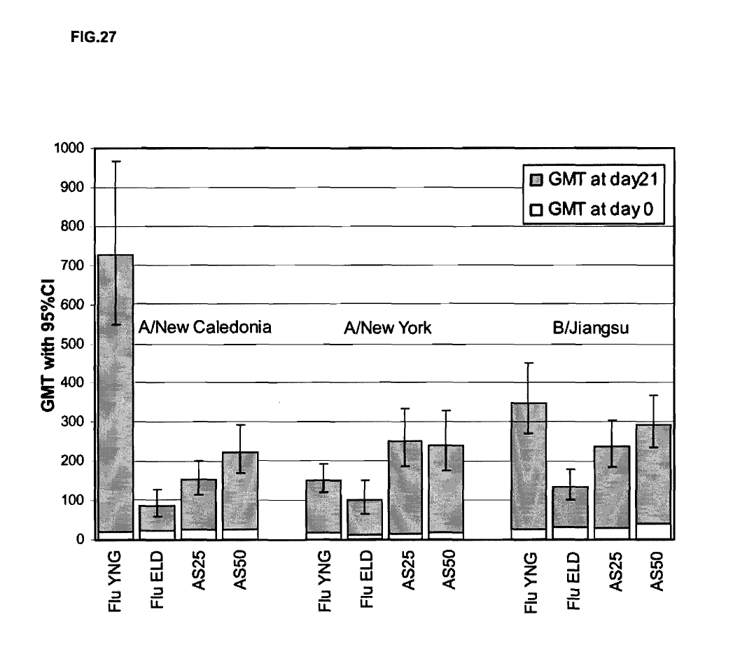

### Humoral Immune Response Results (FIG. 27 and Table 49)

Pre-vaccination GMTs of HI antibodies for all 3 vaccine strains were within the same range in the 4 treatment groups. After vaccination, there is clear impact of the 2 adjuvants which increase the humoral response in elderly, compared to standard Fluarix in the same population.

GMTs are
significantly higher for H1N1 for AS03+MPL (conc. 2),
significantly higher for H3N2 and for B for both adjuvants, Twenty one days after vaccination, the subjects of Fluarix (18-40 years) had a higher HI response for New Calcdonia and B/Jangsu strains.

As shown in Table 49, the adjuvanted influenza vaccines exceeded the requirements of the European authorities for annual registration of split virion influenza vaccines [“Note for Guidance on Harmonization of Requirements for Influenza Vaccines for the immunological assessment of the annual strain changes” (CPMP/BWP/214/96)] in subjects aged over 60 years.

After vaccination, there was a statistically difference in terms of seroprotection rates of HI antibodies between Fluarix (≧65 years) group and
Flu AS03+MPL (conc 2) for A/New Calcdonia strain

For each vaccine strain, the seroprotection rates for the 2 influenza adjuvanted vaccine groups are in the same range compared to Fluarix (18-40 years) group.

There was a statistically difference in terms of seroconversion rates of HI antibodies between Fluarix (≧65 years) group and
Flu AS03+MPL (conc 2) for A/New Calcdonia strain
Flu AS03+MPL (conc 1) for B/Jiangsu strain

For each vaccine strain, the seroconversion rates for the 2 influenza adjuvanted vaccine groups are in the same range compared to Fluarix (18-40 years) group excepted for New Calcdonia strain.

TABLE 49
Seroprotection rates seroconversion rates and conversion
factors at day 21 (ATP cohort for immunogenicity)

| | | | Seroprotection rate | Seroconversion rate | Conversion factor |
|---|---|---|---|---|---|
| | | | (HI titre ≧40) | (>4-fold increase) | [95% CI] |
| Strains | Group | N | % | [95% CI] | % |
| EU standard (>60 years) | | | >60% | >30% | >2.0 |
| EU standard (<60 years) | | | >70% | >40% | >2.5 |
| A/New | Flu Yng | 75 | 100 [95.20-100] | 77.3 [66.2-86.2] | 35.1 \hat{ }21.9-56.4] |
| Caledonia | Flu Elderly | 49 | 71.4 [56.74-83.42] | 30.6 [18.3-45.4] | 3.7 [2.4-5.7] |
| (H1N1) | FluAS03 + MPL (conc. 1) | 75 | 90.5 [81.48-96.11] | 55.4 [43.4-67.0] | 6.4 [4.5-9.0] |
| | FluAS03 + MPL (conc. 2) | 75 | 98.7 [92.79-99.97] | 74.7 [63.3-84.0] | 9.2 [6.4-13.3] |
| A/New York | Flu Yng | 75 | 93.3 [85.12-97.80] | 76.0 [64.7-85.1] | 9.2 [7.1-11.8] |
| (H3N2) | Flu Elderly | 49 | 81.6 [67.98-91.24] | 69.4 [54.6-81.7] | 8.2 [5.7-11.8] |
| | FluAS03 + MPL (conc. 1) | 75 | 94.6 [86.73-98.51] | 90.5 [81.5-96.1] | 19.2 [14.6-25.3] |
| | FluAS03 + MPL (conc. 2) | 75 | 93.3 [85.12-97.80] | 82.7 [72.2-90.4] | 15.0 [11.2-20.2] |
| B/Jiangsu | Flu Yng | 75 | 100 [95.20-100] | 80.0 [69.2-88.4] | 13.9 [10.1-19.1] |
| (B) | Flu Elderly | 49 | 93.9 [83.13-98.72] | 81.3 [70.7-89.4] | 4.3 [3.0-6.1] |
| | FluAS03 + MPL (conc. 1) | 75 | 95.9 [88.61-99.16] | 73.0 [61.4-82.6] | 8.5 [6.5-11.2] |
| | FluAS03 + MPL (conc. 2) | 75 | 98.7 [92.79-99.97] | 66.7 [54.8-77.1] | 7.6 [5.6-10.2] |

N = total number of subject; % = Percentage of subjects with titre at day 21 within the specified range; CI = confidence interval

### XII.3.2. Immunogenicity Conclusions

(a) Pre-vaccination frequency of influenza-specific CD4 was significantly inferior in elderly adults compared to adults aged between 18 and 40 years. After vaccination with Fluarix™, post-vaccination frequency (day 21) remained inferior in elderly adults compared to younger ones. On the contrary, the non-inferiority in term of frequency of post-vaccination frequency of influenza-specific CD4 after vaccination with adjuvanted vaccines of elderly subjects was demonstrated compared to vaccination with Fluarix™ in adults aged between 18 and 40 years.
(b) Regarding the humoral immune response in term of HI antibody response, all influenza vaccines fulfilled the requirements of the European authorities for annual registration of influenza inactivated vaccines [“Note for Guidance on Harmonisation of Requirements for Influenza Vaccines for the immunological assessment of the annual strain changes” (CPMP/BWP/214/96)]. In elderly adults, adjuvanted vaccines mediated at least a trend for a higher humoral immune response to influenza haemagglutinin than Fluarix™. Significant difference between the humoral immune response against each vaccine strain mediated in elderly subjects by adjuvanted vaccines compared to Fluarix™ are summarised in Table 50. Compared to adults aged between 18 and 40 years vaccinated with Fluarix™, elderly subjects vaccinated with the adjuvanted vaccines showed a trend for higher post-vaccination GMTs and seroconversion factor at day 21 against the A/New York strain.

TABLE 50
Significant difference in humoral immune response between
adjuvanted vaccines and Fluarix in elderly subjects

| | | Seroconversion | Seroprotection | Seroconversion |
|---|---|---|---|---|
| | Post-vacc GMT | Factor | rate | Rate |
| Flu AS03 + MPL | A/New York | A/New York | — | B/Jiangsu |
| (conc. 1) | B/Jiangsu | | | |
| Flu AS03 + MPL | A/New York | A/New Caledonia | A/New Caledonia | A/New Caledonia |
| (conc. 2) | B/Jiangsu | | | |
| | A/New | | | |
| | Caledonia | | | |

### XII.4. Reactogenicity Results

### XII.4.1. Recording of Adverse Events (AE)

Solicited symptoms (see Table 51) occurring during a 7-day follow-up period (day of vaccination and 6 subsequent days) were recorded. Unsolicited symptoms occurring during a 21-day follow-up period (day of vaccination and 20+3 subsequent days) were also recorded. Intensity of the following AEs was assessed as described in Table 52.

TABLE 51
Solicited local/general adverse events

| Solicited local AEs | Solicited general AEs |
|---|---|
| Pain at the injection site | Fatigue |
| Redness at the injection site | Fever |
| Swelling at the injection site | Headache |
| Haematoma | Muscle ache |
| | Shivering |
| | Joint pain in the arm of the injection |
| | Joint pain at other locations |

N.B. Temperature was recorded in the evening. Should additional temperature measurements performed at other times of day, the highest temperature was recorded.

TABLE 52
Intensity scales for solicited symptoms in adults

| | Intensity | |
|---|---|---|
| Adverse Event | grade | Parameter |
| Pain at injection site | 0 | Absent |
| | 1 | on touch |
| | 2 | when limb is moved |
| | 3 | prevents normal activity |
| Redness at injection site | | Record greatest surface diameter |
| | | in mm |
| Swelling at injection site | | Record greatest surface diameter |
| | | in mm |
| Haematoma at injection site | | Record greatest surface diameter |
| | | in mm |
| Fever* | | Record temperature in ° C./° F. |
| Headache | 0 | Absent |
| | 1 | is easily tolerated |
| | 2 | interferes with normal activity |
| | 3 | prevents normal activity |
| Fatigue | 0 | Absent |
| | 1 | is easily tolerated |
| | 2 | interferes with normal activity |
| | 3 | prevents normal activity |
| Joint pain at the injection | 0 | Absent |
| site and other locations | 1 | is easily tolerated |
| | 2 | interferes with normal activity |
| | 3 | prevents normal activity |
| Muscle ache | 0 | Absent |
| | 1 | is easily tolerated |
| | 2 | interferes with normal activity |
| | 3 | prevents normal activity |
| Shivering | 0 | Absent |
| | 1 | is easily tolerated |
| | 2 | interferes with normal activity |
| | 3 | prevents normal activity |

*Fever is defined as axillary temperature ≧37.5° C. (99.5° F.)

The maximum intensity of local injection site redness/swelling is scored as follows:

0 is 0 mm; 1 is >0-≦20 mm; 2 is >20-≦50 mm; 3 is >50 mm.

The maximum intensity of fever is scored as follows:

### 1 is >37.5-≦38.0° C.; 2 is >38.0-≦39.0° C.; 3 is >39.0

The investigator makes an assessment of intensity for all other AEs, i.e. unsolicited symptoms, including SAEs reported during the study. The assessment is based on the investigator's clinical judgement. The intensity of each AE recorded is assigned to one of the following categories:

1 (mild)=An AE which is easily tolerated by the subject, causing minimal discomfort and not interfering with everyday activities;
2 (moderate)=An AE which is sufficiently discomforting to interfere with normal everyday activities;
3 (severe)=An AE which prevents normal, everyday activities (In adults/adolescents, such an AE would, for example, prevent attendance at work/school and would necessitate the administration of corrective therapy).

### XII.4.2. Recording of Adverse Events (AE)

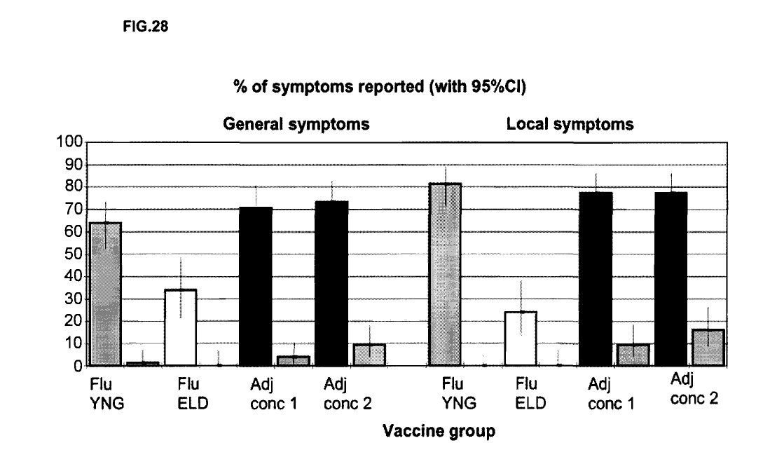

The reactogenicity observed in elderly subjects with adjuvanted vaccines, in terms of both local and general symptoms, was found to be higher than with Fluarix™ in the same population. However, it was shown to be similar to the level seen in the adult population. The incidence and the intensity of symptoms was increased after vaccination with adjuvanted vaccines compared to the reactogenity seen in elderly subjects with Fluarix™ (FIG. 28). In all cases, symptoms resolved rapidly.

Grade 3 symptoms showed a trend to be higher in the group who received the vaccine adjuvanted with the highest MPL concentration compared to the group who received the adjuvanted vaccine wherein the MPL is at a lower concentration. In all cases, symptoms however resolved rapidly.

### Example XIII

### Preparation of the Oil-in-Water Emulsion and Adjuvant Formulations

Unless otherwise stated, the oil/water emulsion used in the subsequent examples is composed an organic phase made of 2 oils (alpha-tocopherol and squalene), and an aqueous phase of PBS containing Tween 80™ or Polysorbate 80™ as emulsifying agent. Unless otherwise stated, the oil-in-water emulsion adjuvant formulations used in the subsequent examples were made comprising the following oil-in-water emulsion component (final concentrations given): 2.5% squalene (v/v), 2.5% alpha-tocopherol (v/v), 0.9% polyoxyethylene sorbitan monooleate (v/v) (Tween 80), see WO 95/17210. This emulsion, termed AS03 in the subsequent examples, was prepared as followed as a two-fold concentrate.

The preparation of the emulsion is made by mixing under strong agitation of an oil phase composed of hydrophobic components (DL-α-tocopherol and squalene) and an aqueous phase containing the water soluble components (the anionic detergent Tween 80 and PBS mod (modified), pH 6.8). While stirring, the oil phase ( 1/10 total volume) is transferred to the aqueous phase ( 9/10 total volume), and the mixture is stirred for 15 minutes at room temperature. The resulting mixture then subjected to shear, impact and cavitation forces in the interaction chamber of a microfluidizer (15000 PSI—8 cycles, or 3 cycles in the adjuvant used in the clinical trial reported in Example III and Example IV) to produce submicron droplets (distribution between 100 and 200 nm). The resulting pH is between 6.8±0.1. The SB62 emulsion is then sterilised by filtration through a 0.22 μm membrane and the sterile bulk emulsion is stored refrigerated in Cupac containers at 2 to 8° C. Sterile inert gas (nitrogen or argon) is flushed into the dead volume of the SB62 emulsion final bulk container for at least 15 seconds.

The final composition of the emulsion is as described in Examples XIV and XV.

### Example XIV

### Clinical Trial in a Population Aged 18-60 Years with a Vaccine Containing an Adjuvanted Influenza Split Virus Antigen Preparation According to Different Vaccination Schedules

### XIV.1. Introduction

A phase II, open, randomized study in adults aged between 18 and 60 years designed to evaluate the reactogenicity and immunogenicity of a 1- and 2-dose prime-boost concept of pandemic monovalent (H5N1) influenza vaccine (split virus formulation) adjuvanted with AS03, administered according to different vaccination schedules.

### XIV.2. Eight Study Groups of 63 Subjects (Planned) in Parallel

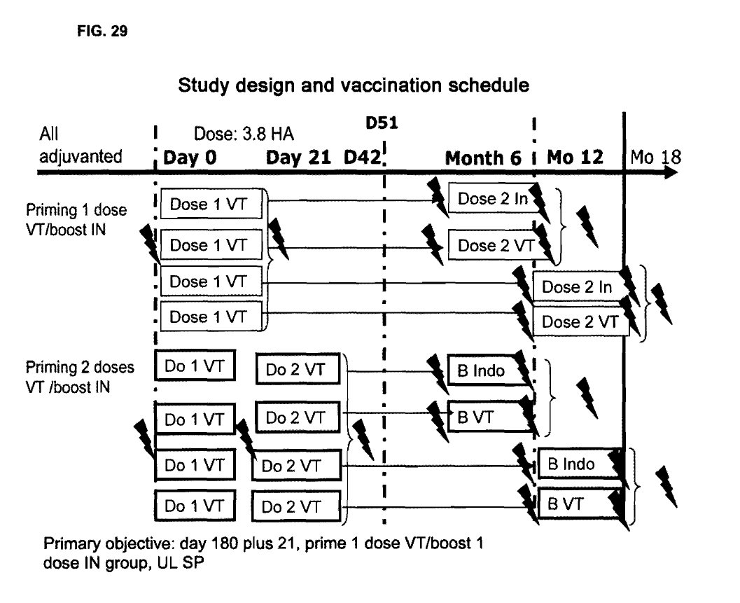

Healthy male of female between the ages of 18-60 years at the time of first vaccination. The injections were administered in the non-dominant arm according to the following schedule (FIG. 29):
VT/VT/6Mo (66 subjects): two administrations of the pandemic influenza candidate vaccine containing the Vietnam (VT) strain at Day 0 and Month 6.
VT/VT/12Mo (64 subjects): two administrations of the pandemic influenza candidate vaccine containing the VT strain at Day 0 and Month 12.
VT/IN/6Mo (63 subjects): one administration of the pandemic influenza candidate vaccine containing the VT strain at Day 0 and one administration of the pandemic vaccine containing the Indonesia (IN) strain at Month 6.
VT/IN/12Mo (64 subjects): one administration of the pandemic influenza candidate vaccine containing the VT strain at Day 0 and one administration of the pandemic vaccine containing the IN strain at Month 12.
2VT/VT/6Mo (63 subjects): two administrations of the pandemic influenza candidate vaccine containing the VT strain at Day 0 and Day 21 and a third dose of vaccine containing the VT strain at Month 6.
2VT/VT/12Mo (63 subjects): two administrations of the pandemic influenza candidate vaccine containing the VT strain at Day 0 and Day 21 and a third dose of vaccine containing the VT strain at Month 12.
2VT/IN/6Mo (64 subjects): two administrations of the pandemic influenza candidate vaccine containing the VT strain at Day 0 and Day 21 and a third dose of vaccine containing the IN strain at Month 6;
2VT/IN/12Mo (65 subjects): two administrations of the pandemic influenza candidate vaccine containing the VT strain at Day 0 and Day 21 and a third dose of vaccine containing the IN strain at Month 12.

Subjects in each group were stratified by age: between 18 and 30 years and above 30 years (ratio 1:1).

### XIV.2. Immunogenicity Objectives of the Study

Immunogenicity and reactogenicity/safety parameters have been assessed. Only the immunogenicity parameters are reported.

### XIV.2.1. Immunogenicity Objectives:

The humoral immune response (in terms of HI antibody and neutralizing antibody titres) and cell mediated immune response induced by a booster dose of the vaccine given 6 months after priming vaccination with a single dose of the vaccine formulated from a heterologous strain are assessed.

Humoral Immune Response (in Terms of HI and Neutralizing Antibody Titres)

(i) 21 days after the vaccination(s) for the priming administration(s) and
(ii) 7 and 21 days after vaccination for the booster administration;
(iii) persistence 6 and 12 months after the priming administration(s) of the vaccines;
(iv) persistence 6 or 12 months after the booster administration of the vaccines;
(v) comparison of the humoral immune response induced by a booster dose of the vaccine formulated from the homologous strain (with respect to the strain used for priming), given following a priming with either one or two doses of the vaccine;
(vi) comparison of the humoral immune response induced by a booster dose of the vaccine formulated from the heterologous strain (with respect to the strain used for priming) given following a priming with either one or two doses of the vaccine;

Cell Mediated (CMI) Immune Response

(i) Before and after the booster administration of the vaccines in terms of the Th1-specific cytokine resp. activation marker expression (CD40L, IL-2, TNF-α and IFN-γ) after in vitro re-stimulation of influenza-specific CD4/CD8 T-cells.

### XIV.2.2. Evaluation Methods

In Order to Evaluate the Humoral Response in Terms of HI Antibodies, the Following Parameters are Calculated with 95% Confidence Intervals:
Seropositivity and GMTs of H5N1 antibody titres at Day 0, Day 21, Day 42, Month 6/12, Month 6/12+7 days, Month 6/12+21 days, Month 18.
Seroconversion rates (SCR; defined as the percentage of vaccinees that had either a pre-vaccination titre<1:10 and a post-vaccination titre≧1:40 or a pre-vaccination titre≧1:10 and at least a 4-fold increase in post-vaccination titre, using Day 0 as reference) at Day 21, Day 42, Month 6/12, Month 0.6/12+7 days, Month 6/12+21 days, Month 18.
Seroconversion factors (SCF; defined as the fold increase in serum HI antibody GMTs post-vaccination compared to Day 0) at Month 6/12, Month 6/12+7 days, Month 6/12+21 days, Month 18.
Seroprotection rates (SPR; defined as the percentage of vaccinees with a serum HI antibody titre≧1:40) at Day 0, Month 6, Month 6+7 days, Month 6+21 days.
Booster Response (BR; defined as at least a 4-fold increase in serum HI antibodies between pre-booster vaccination and post-booster vaccination) at Month 6+7 days, Month 6+21 days.
Booster Factor (BF; defined as the fold-increase HI antibody GMT between pre-booster vaccination and post-booster vaccination) at Month 6+7 days, Month 6+21 days.

For the humoral immune response in terms of neutralizing antibodies, the following parameters (with 95% confidence intervals) are calculated:
Seropositivity and Geometric mean titres (GMTs) of HI antibody titres at Day 0, Day 21, Day 42, Month 6/12, Month 6/12+7 days, Month 6/12+21 days, Month 18.
Seroconversion rates (SCR; for HI antibody response, is defined as the percentage of vaccinees who have either a pre-vaccination titre<1:10 and a post-vaccination titre≧1:40 or a pre-vaccination titre≧1:10 and at least a 4-fold increase in post-vaccination titre and for neutralizing antibody response is defined as the percentage of vaccinees with a minimum 4-fold increase in neutralizing antibody titre at post-vaccination, using Day 0 as reference) at Day 21, Day 42, Month 6/12, Month 0.6/12+7 days, Month 6/12+21 days, Month 18.

For the CMI response, the following parameters (with 95% CIs) are calculated at Day 0, Month 6/12, Month 6/12+7 days, Month 6/12+21 days and Month 18 in each group:
Frequency of influenza-specific CD4/CD8 T-cells per 10⁶ in tests producing at least two out of four different Th1-specific activation markers (CD40L, IL-2, TNF-α, IFN-γ).

Safety/reactogenicity: The following parameters were recorded:
Percentage, intensity and relationship to vaccination of solicited local and general signs and symptoms during a 7-day follow-up period (i.e. day of vaccination and 6 subsequent days) after each vaccination and overall.
Percentage, intensity and relationship to vaccination of unsolicited local and general signs and symptoms during a 30-day follow-up period after priming vaccination(s) and booster vaccination, and overall.
Occurrence of serious adverse events during the entire study.

### Statistical Methods for Immunogenicity

Humoral immune response: For the comparisons between groups (VT/VT/6Mo versus VT/VT/12Mo, 2VT/VT/6Mo versus 2VT/VT/12 Mo, VT/IN/6Mo versus VT/IN/12Mo, 2VT/IN/6Mo versus 2VT/IN/12Mo, 95% CI adjusted GMT ratio between vaccine groups are computed using a one-way ANCOVA model on the logarithm 10 transformed titres. The ANCOVA model included the vaccine group effect and pre-vaccination as regressor. The GMT ratio was derived from the contrast of the vaccine group effect.

Cell-mediated immune response: A non-parametric test (Wilcoxon Test) is used to compare the location of the difference between groups and the statistical p-value was calculated at each different test and each appropriate time-point. Wilcoxon test is also used to compare the individual difference (Post-Pre-vaccination) and the statistical p-value is calculated at each different test.

### XIV.3. Vaccine Composition and Administration

XIV.3.1. Vaccine composition: Monovalent, split virus, influenza pandemic candidate vaccine formulated from either the A/Vietnam/1194/2004 (H5N1) or the A/Indonesia/5/2005 (H5N1) strains, adjuvanted with AS03. The total injected volume was 0.5 ml.

The manufacturing process for the monovalent bulks of split, inactivated influenza H5N1 strain is identical to the manufacturing process for the monovalent bulks of GSK Biologicals licensed interpandemic influenza vaccine Fluarix™/α-Rix® (WO02/097072 and WO2008/009309). For the purpose of this clinical trial the virus strains used to manufacture the clinical lots is the H5N1 vaccine strain A/Vietnam/1194/04 NIBRG-14 recombinant H5N1 prototype vaccine strain derived from the A/Vietnam/1194/04 strain (VT strain) and the A/Indonesia/05/2005 (H5N1)/PR8-IBCDC-RG2 strain (IN strain). The VT strain has been developed by NIBSC using reverse genetics (a suitable reference is Nicolson et al. 2005, Vaccine, 23, 2943-2952)). The IN strain has been developed by CDC using reverse genetics. Each reassortant strain combines the H5 and N1 segments to the A/PR/8/34 strain backbone, and the H5 was engineered to eliminate the polybasic stretch of amino-acids at the HA cleavage site that is responsible for high virulence of the original strains. The active substance of the pandemic influenza vaccine candidates is a formaldehyde inactivated split virus antigen.

The AS03 adjuvanted inactivated split virus influenza vaccines are 2 component vaccines consisting of concentrated inactivated split virion (H5N1) antigens presented in a type I glass vial and of the AS03 adjuvant contained in a pre-filled type I glass syringe. One adult dose of reconstituted AS03-adjuvanted vaccine corresponds to 0.5 ml. Their composition is given in Table 53.

TABLE 53
Composition of the reconstituted AS03 adjuvanted
influenza candidate vaccines

| | | |
|---|---|---|
| Component | Quantity per dose VIET | Quantity per dose INDO |

ACTIVE INGREDIENTS

| | | | | |
|---|---|---|---|---|
| Inactivated split virions (H5N1) | 3.75 | μg HA | 3.75 | μg HA |

AS03 ADJUVANT

| | | | | |
|---|---|---|---|---|
| o/w emulsion | | | | |
| squalene | 10.68 | mg | 10.68 | mg |
| DL-α-tocopherol | 11.86 | mg | 11.86 | mg |
| Polysorbate 80 (Tween 80) | 4.86 | mg | 4.86 | mg |

EXCIPIENTS

| | | | | |
|---|---|---|---|---|
| Polysorbate 80 (Tween 80) | Not less than 28.75 | μg | Not less than 28.75 | μg |
| Octoxynol 10 (Triton X-100)² | 3.75 | μg | 3.75 | μg |
| Thiomersal | 5 | μg | 5 | μg |
| Sodium chloride | 3.7 | mg | 3.7 | mg |
| Disodium hydrogen phosphate | 510 | μg | 510 | μg |
| Potassium dihydrogen phosphate | 130 | μg | 130 | μg |
| Potassium chloride | 90 | μg | 90 | μg |
| Magnesium chloride hexahydrate | 12 | μg | 12 | μg |
| Water for injections q.s. ad. | 0.5 | ml | 0.5 | ml |

For this study, 0.25 ml each of the content of the prefilled syringe containing the adjuvant and 0.25 ml each of the content of the vial containing monovalent split influenza virus antigen was used. After extemporaneous mixing of the contents, a 0.5 ml dose is withdrawn into the syringe and injected intramuscularly. At the time of injection, the content of the prefilled syringe containing the adjuvant is injected into the vial that contains the concentrated inactivated split virion antigens. One dose of the reconstituted the AS03-adjuvanted influenza candidate vaccine corresponds to 0.5 ml, containing 3.75 μg haemagglutinin (HA). If necessary, the formulation process was adapted to ensure that the same amounts of antigen and adjuvants are present in the final vaccine. Thiomersal is added as a preservative at a concentration of 10 μg/ml (5 μg per dose).

### XIV.4. Immunogenicity Results

### XIV.4.1 Humoral Response to the Booster Administration

The humoral response to the booster administration was evaluated based on the HI antibody titres with 95% CIs against the A/Vietnam/1194/2004 and A/Indonesia/5/2005 strains and measured at 7 days and 21 days after booster vaccination for the subjects who received a booster vaccination at Month 6.

### XIV. 4.1.1. Booster Administration: HI Response Against the A/Vietnam/1194/2004 and A/Indonesia/5/2005 Strains

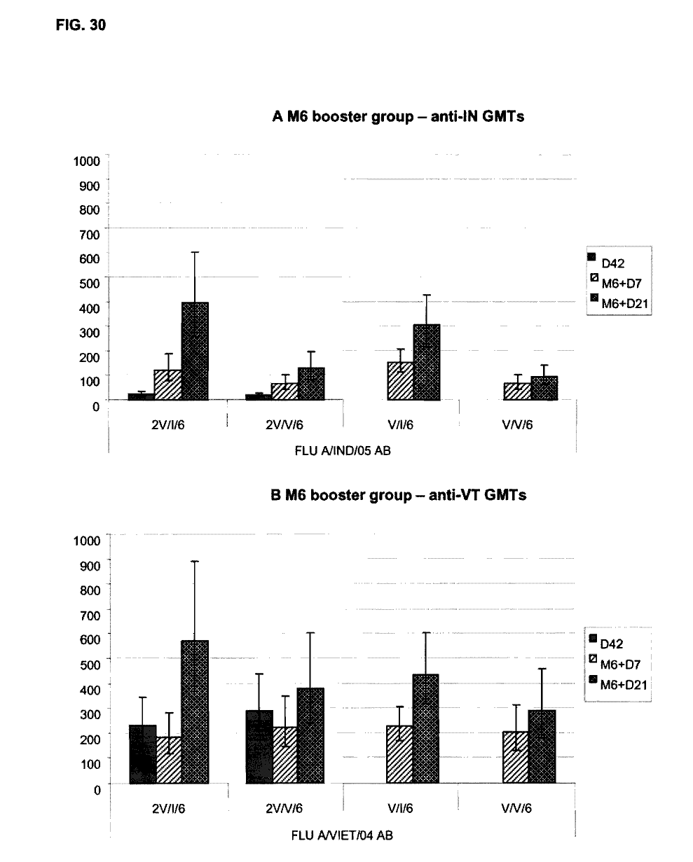

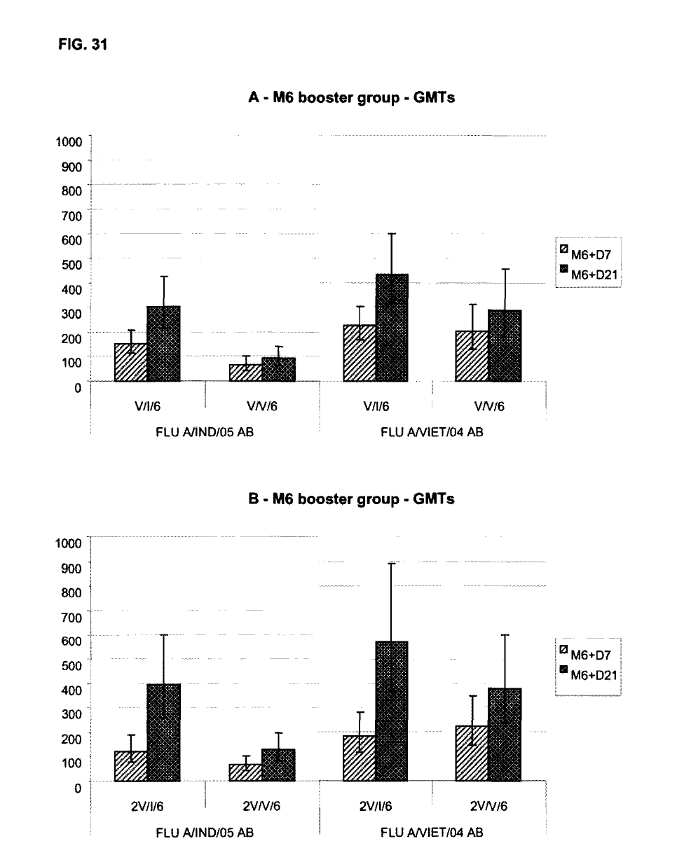

The GMTs and seropositivity rates measured at Month 6, Month 6+7 days and Month 6+21 days for HI antibodies against the A/Vietnam/1194/2004 and A/Indonesia/5/2005 strains (Table 54 and FIGS. 30 and 31).

TABLE 54
Seropositivity rates and GMTs of HI antibodies against the VT or IN strains
up to Month 6 + 21 days, for adults who received the booster dose at Month 6

| | | | | >=10 1/DIL | | | | GMT | | | | |
|---|---|---|---|---|---|---|---|---|---|---|---|---|
| Antibodies | | | | | | 95% CI | | | 95% CI | | | |
| against | Group | Timing | N | n | % | LL | UL | value | LL | UL | Min | Max |
| A/ | VT/IN/6Mo | PRE | 56 | 0 | 0.0 | 0.0 | 6.4 | 5.0 | 5.0 | 5.0 | <10.0 | <10.0 |
| Indonesia | | PI(D 21) | 56 | 8 | 14.3 | 6.4 | 26.2 | 6.3 | 5.4 | 7.4 | <10.0 | 57.0 |
| | | PI(M 6) | 55 | 4 | 7.3 | 2.0 | 17.6 | 5.5 | 5.0 | 6.0 | <10.0 | 28.0 |
| | | PII(M 6 + D 7) | 53 | 52 | 98.1 | 89.9 | 100 | 152.9 | 112.2 | 208.2 | <10.0 | 1280.0 |
| | | PII(M 6 + D 21) | 52 | 51 | 98.1 | 89.7 | 100 | 303.4 | 215.7 | 426.6 | <10.0 | 3620.0 |
| | VT/VT/6Mo | PRE | 55 | 0 | 0.0 | 0.0 | 6.5 | 5.0 | 5.0 | 5.0 | <10.0 | <10.0 |
| | | PI(D 21) | 55 | 7 | 12.7 | 5.3 | 24.5 | 5.6 | 5.1 | 6.0 | <10.0 | 14.0 |
| | | PI(M 6) | 49 | 2 | 4.1 | 0.5 | 14.0 | 5.3 | 4.9 | 5.7 | <10.0 | 28.0 |
| | | PII(M 6 + D 7) | 47 | 37 | 78.7 | 64.3 | 89.3 | 64.6 | 41.3 | 101.1 | <10.0 | 640.0 |
| | | PII(M 6 + D 21) | 48 | 41 | 85.4 | 72.2 | 93.9 | 92.4 | 61.1 | 139.9 | <10.0 | 1280.0 |
| | 2VT/IN/6Mo | PRE | 50 | 0 | 0.0 | 0.0 | 7.1 | 5.0 | 5.0 | 5.0 | <10.0 | <10.0 |
| | | PI(D 21) | 50 | 6 | 12.0 | 4.5 | 24.3 | 6.2 | 5.2 | 7.4 | <10.0 | 80.0 |
| | | PII(D 42) | 44 | 27 | 61.4 | 45.5 | 75.6 | 23.6 | 15.6 | 35.7 | <10.0 | 320.0 |
| | | PII(M 6) | 49 | 18 | 36.7 | 23.4 | 51.7 | 8.6 | 6.6 | 11.3 | <10.0 | 640.0 |
| | | PIII(M 6 + D 7) | 47 | 41 | 87.2 | 74.3 | 95.2 | 120.0 | 76.8 | 187.6 | <10.0 | 1280.0 |
| | | PIII(M 6 + D 21) | 49 | 47 | 95.9 | 86.0 | 99.5 | 392.9 | 256.7 | 601.4 | <10.0 | 5120.0 |
| | 2VT/VT/6Mo | PRE | 48 | 0 | 0.0 | 0.0 | 7.4 | 5.0 | 5.0 | 5.0 | <10.0 | <10.0 |
| | | PI(D 21) | 48 | 5 | 10.4 | 3.5 | 22.7 | 5.5 | 5.0 | 6.0 | <10.0 | 20.0 |
| | | PII(D 42) | 41 | 22 | 53.7 | 37.4 | 69.3 | 18.4 | 11.9 | 28.3 | <10.0 | 226.0 |
| | | PII(M 6) | 48 | 13 | 27.1 | 15.3 | 41.8 | 7.2 | 6.0 | 8.6 | <10.0 | 40.0 |
| | | PIII(M 6 + D 7) | 45 | 37 | 82.2 | 67.9 | 92.0 | 65.0 | 42.1 | 100.2 | <10.0 | 640.0 |
| | | PIII(M 6 + D 21) | 46 | 41 | 89.1 | 76.4 | 96.4 | 127.7 | 83.8 | 194.6 | <10.0 | 1810.0 |
| A/ | VT/IN/6Mo | PRE | 56 | 0 | 0.0 | 0.0 | 6.4 | 5.0 | 5.0 | 5.0 | <10.0 | <10.0 |
| Vietnam | | PI(D 21) | 56 | 31 | 55.4 | 41.5 | 68.7 | 20.9 | 14.1 | 30.9 | <10.0 | 453.0 |
| | | PI(M 6) | 55 | 25 | 45.5 | 32.0 | 59.4 | 12.0 | 8.9 | 16.1 | <10.0 | 160.0 |
| | | PII(M 6 + D 7) | 53 | 52 | 98.1 | 89.9 | 100 | 226.3 | 168.3 | 304.3 | <10.0 | 1280.0 |
| | | PII(M 6 + D 21) | 52 | 51 | 98.1 | 89.7 | 100 | 434.7 | 314.8 | 600.5 | <10.0 | 5120.0 |
| | VT/VT/6Mo | PRE | 55 | 2 | 3.6 | 0.4 | 12.5 | 5.3 | 4.9 | 5.7 | <10.0 | 28.0 |
| | | PI(D 21) | 55 | 28 | 50.9 | 37.1 | 64.6 | 16.3 | 11.5 | 23.2 | <10.0 | 160.0 |
| | | PI(M 6) | 49 | 18 | 36.7 | 23.4 | 51.7 | 9.7 | 7.4 | 12.7 | <10.0 | 80.0 |
| | | PII(M 6 + D 7) | 47 | 42 | 89.4 | 76.9 | 96.5 | 202.6 | 130.7 | 314.1 | <10.0 | 1280.0 |
| | | PII(M 6 + D 21) | 48 | 43 | 89.6 | 77.3 | 96.5 | 287.2 | 180.3 | 457.5 | <10.0 | 5120.0 |
| | 2VT/IN/6Mo | PRE | 50 | 1 | 2.0 | 0.1 | 10.6 | 5.2 | 4.8 | 5.7 | <10.0 | 40.0 |
| | | PI(D 21) | 50 | 33 | 66.0 | 51.2 | 78.8 | 33.6 | 21.4 | 52.8 | <10.0 | 905.0 |
| | | PII(D 42) | 44 | 41 | 93.2 | 81.3 | 98.6 | 229.8 | 153.6 | 344.0 | <10.0 | 1810.0 |
| | | PII(M 6) | 49 | 35 | 71.4 | 56.7 | 83.4 | 29.9 | 20.1 | 44.4 | <10.0 | 1280.0 |
| | | PIII(M 6 + D 7) | 47 | 43 | 91.5 | 79.6 | 97.6 | 182.7 | 118.5 | 281.7 | <10.0 | 2560.0 |
| | | PIII(M 6 + D 21) | 49 | 47 | 95.9 | 86.0 | 99.5 | 571.4 | 366.3 | 891.3 | <10.0 | 7240.0 |
| | 2VT/VT/6Mo | PRE | 48 | 2 | 4.2 | 0.5 | 14.3 | 5.7 | 4.7 | 7.0 | <10.0 | 226.0 |
| | | PI(D 21) | 48 | 29 | 60.4 | 45.3 | 74.2 | 29.3 | 17.9 | 48.1 | <10.0 | 1280.0 |
| | | PII(D 42) | 41 | 39 | 95.1 | 83.5 | 99.4 | 289.1 | 190.3 | 439.2 | <10.0 | 3620.0 |
| | | PII(M 6) | 48 | 34 | 70.8 | 55.9 | 83.0 | 32.0 | 21.3 | 48.0 | <10.0 | 640.0 |
| | | PIII(M 6 + D 7) | 45 | 41 | 91.1 | 78.8 | 97.5 | 224.5 | 144.9 | 347.7 | <10.0 | 1280.0 |
| | | PIII(M 6 + D 21) | 46 | 43 | 93.5 | 82.1 | 98.6 | 380.5 | 240.2 | 602.9 | <10.0 | 5120.0 |

N = Number of subjects with available results; n/% = number/% of seropositive subjects (HI titre >=1:10); 95% CI = 95% confidence interval, LL = Lower Limit, UL = Upper Limit; MIN/MAX = Minimum/Maximum; MIN/MAX = Minimum/Maximum; PRE = Pre-vacc dose 1 at Day 0; PI(D 21) = Post-vacc dose 1 at Day 21; PI(M 6) = Post-vacc dose 1 at M 6; PII(D 42) = Post-vacc dose 2 at Day 42; PII(M 6) = Post-vacc dose 2 at M 6; PII(M 6 + D 7) = Post-vacc dose 2 at Day 7 after M 6; PII(M 6 + D 21) = Post-vacc dose 2 at Day 21 after M 6; PIII(M 6 + D 7) = Post-vacc dose 3 at Day 7 after M 6; PIII(M 6 + D 21) = Post-vacc dose 3 at Day 21 after M 6

All groups who received a booster dose at Month 6 had a significant increase in GMTs for HI antibodies against the A/Indonesia/5/2005 and the A/Vietnam/1194/2004 strains at both Month 6+7 days and Month 6+21 days, compared to the Month 6 pre-booster time-point.
The response against the A/Indonesia/5/2005 strain for the four groups who received the booster vaccination at Month 6 was slightly higher at Month 6+7 days for groups boosted with the A/Indonesia/5/2005 strain (120.0 for the group primed with two doses and 152.9 for the group primed with one dose) than for groups boosted with the A/Vietnam/1194/2004 strain (64.6 for the group primed with one dose and 65.0 for the group primed with two doses). The response increased further for the Month 6+21 day time-point and same observation applies.
At Month 6+7 days, the HI GMTs had increased significantly for the four groups who received the booster vaccination at Month 6 compared to the pre-booster time-point. The response against the A/Vietnam/1194/2004 strain was not significantly different between groups, though it tended to be higher for the 2VT/IN/6Mo group.
The two groups boosted with A/Vietnam/1194/2004 had a significant increase in GMTs for HI antibodies against the heterologous A/Indonesia/5/2005 strain at Month 6+7 days compared to the Month 6 values.
At Month 6+21 days, the HI response against the A/Indonesia/5/2005 strain was significantly higher for the two groups boosted with the A/Indonesia/5/2005 strain (303.4-392.9) than with the A/Vietnam/1194/2004 strain (92.7-127.7).
At Month 6+21 days, GMTs had increased further compared to the Month 6+7 day time-point. The HI response against the A/Vietnam/1194/2004 strain was not significantly different between groups. However, groups boosted with the A/Indonesia/5/2005 strain tended to have higher GMTs than groups boosted with the A/Vietnam/1194/2004 strain.
At the Month 6+7 days time-point, seropositivity levels had increased significantly compared to the Month 6 time-point for all groups and ranged between 78.7-98.1%.
At the Month 6+21 days time-point, seropositivity levels showed only a marginal increase for all groups compared to the previous time-point at Month 6+7 days (85.4%-98.1%).

The seroconversion rates (SCR, defined as the percentage of vaccinees with either a pre-vaccination titre<1:10 and a post-vaccination titre≧1:40 or a pre-vaccination titre≧1:10 and at least a 4-fold increase in post-vaccination titre) using Day 0 as a reference, are shown in Table 55.

TABLE 55
SCR using Day 0 as a reference, of HI antibodies against the VT or INstrains
up to Month 6 + 21 days, for adults who received the booster dose at Month 6

| | | | | SCR | | | |
|---|---|---|---|---|---|---|---|
| Antibodies | | | | | | 95% CI | |
| against | Group | Timing | N | n | % | LL | UL |
| A/ | VT/IN/6Mo | PI(D 21) | 56 | 3 | 5.4 | 1.1 | 14.9 |
| Indonesia | | PI(M 6) | 55 | 0 | 0.0 | 0.0 | 6.5 |
| | | PII(M 6 + D 7) | 53 | 49 | 92.5 | 81.8 | 97.9 |
| | | PII(M 6 + D 21) | 52 | 51 | 98.1 | 89.7 | 100 |
| | VT/VT/6Mo | PI(D 21) | 55 | 0 | 0.0 | 0.0 | 6.5 |
| | | PI(M 6) | 49 | 0 | 0.0 | 0.0 | 7.3 |
| | | PII(M 6 + D 7) | 47 | 35 | 74.5 | 59.7 | 86.1 |
| | | PII(M 6 + D 21) | 48 | 40 | 83.3 | 69.8 | 92.5 |
| | 2VT/IN/6Mo | PI(D 21) | 50 | 3 | 6.0 | 1.3 | 16.5 |
| | | PII(D 42) | 44 | 24 | 54.5 | 38.8 | 69.6 |
| | | PII(M 6) | 49 | 5 | 10.2 | 3.4 | 22.2 |
| | | PIII(M 6 + D 7) | 47 | 40 | 85.1 | 71.7 | 93.8 |
| | | PIII(M 6 + D 21) | 49 | 46 | 93.9 | 83.1 | 98.7 |
| | 2VT/VT/6Mo | PI(D 21) | 48 | 0 | 0.0 | 0.0 | 7.4 |
| | | PII(D 42) | 41 | 17 | 41.5 | 26.3 | 57.9 |
| | | PII(M 6) | 48 | 2 | 4.2 | 0.5 | 14.3 |
| | | PIII(M 6 + D 7) | 45 | 33 | 73.3 | 58.1 | 85.4 |
| | | PIII(M 6 + D 21) | 46 | 40 | 87.0 | 73.7 | 95.1 |
| A/ | VT/IN/6Mo | PI(D 21) | 56 | 25 | 44.6 | 31.3 | 58.5 |
| Vietnam | | PI(M 6) | 55 | 14 | 25.5 | 14.7 | 39.0 |
| | | PII(M 6 + D 7) | 53 | 52 | 98.1 | 89.9 | 100 |
| | | PII(M 6 + D 21) | 52 | 51 | 98.1 | 89.7 | 100 |
| | VT/VT/6Mo | PI(D 21) | 55 | 21 | 38.2 | 25.4 | 52.3 |
| | | PI(M 6) | 49 | 6 | 12.2 | 4.6 | 24.8 |
| | | PII(M 6 + D 7) | 47 | 42 | 89.4 | 76.9 | 96.5 |
| | | PII(M 6 + D 21) | 48 | 43 | 89.6 | 77.3 | 96.5 |
| | 2VT/IN/6Mo | PI(D 21) | 50 | 30 | 60.0 | 45.2 | 73.6 |
| | | PII(D 42) | 44 | 41 | 93.2 | 81.3 | 98.6 |
| | | PII(M 6) | 49 | 26 | 53.1 | 38.3 | 67.5 |
| | | PIII(M 6 + D 7) | 47 | 42 | 89.4 | 76.9 | 96.5 |
| | | PIII(M 6 + D 21) | 49 | 46 | 93.9 | 83.1 | 98.7 |
| | 2VT/VT/6Mo | PI(D 21) | 48 | 24 | 50.0 | 35.2 | 64.8 |
| | | PII(D 42) | 41 | 38 | 92.7 | 80.1 | 98.5 |
| | | PII(M 6) | 48 | 27 | 56.3 | 41.2 | 70.5 |
| | | PIII(M 6 + D 7) | 45 | 39 | 86.7 | 73.2 | 94.9 |
| | | PIII(M 6 + D 21) | 46 | 42 | 91.3 | 79.2 | 97.6 |

Seroconversion defined as: For initially seronegative subjects (at day 0), antibody titre >=40 1/DIL after vaccination; For initially seropositive subjects (at day 0), antibody titre after vaccination >=4 fold the; pre-vaccination antibody titre; N = Number of subjects with pre- and post-vaccination results available; n/% = Number/percentage of seroconverted subjects; 95% CI = 95% confidence interval, LL = Lower Limit, UL = Upper Limit; PI(D 21) = Post-vaccination dose 1 at Day 21; PI(M 6) = Post-vaccination dose 1 at Month 6; PII(D 42) = Post-vaccination dose 2 at Day 42; PII(M 6) = Post-vaccination dose 2 at Month 6; PII(M 6 + D 7) = Post-vaccination dose 2 at Day 7 after Month 6; PII(M 6 + D 21) = Post-vaccination dose 2 at Day 21 after Month 6; PIII(M 6 + D 7) = Post-vaccination dose 3 at Day 7 after Month 6; PIII(M 6 + D 21) = Post-vaccination dose 3 at Day 21 after Month 6

The ≧40% SCR threshold required by the European Committee for Medicinal Products for Human Use (CHMP) for adults aged 18-60 years was exceeded at Month 6+7 days for HI antibodies against both the A/Vietnam/1194/2004 and A/Indonesia/5/2005 strains (73.3%-98.1%), for all groups boosted at Month 6 and remained high for both strains at Month 6+21 days (83.3%-98.1%).
The FDA Guidance for Industry for pandemic vaccines requires that ≧40% of subjects meet the lower limit of the 95% confidence interval for seroconversion. This threshold was also exceeded at Month 6+7 days for both strains, for all groups boosted at Month 6 and remained high for both strains at Month 6+21 days.
Before the booster administration at Month 6, the four groups show appreciable persistence of seroconversion rates for the HI antibodies against the A/Vietnam/1194/2004 strain (12.2%-56.3%) and some degree of seroconversion rates for the HI antibodies against the A/Indonesia/5/2005 strain (0%-10.2%), with no significant difference observed between the groups. The study was not powered to demonstrate such a difference.

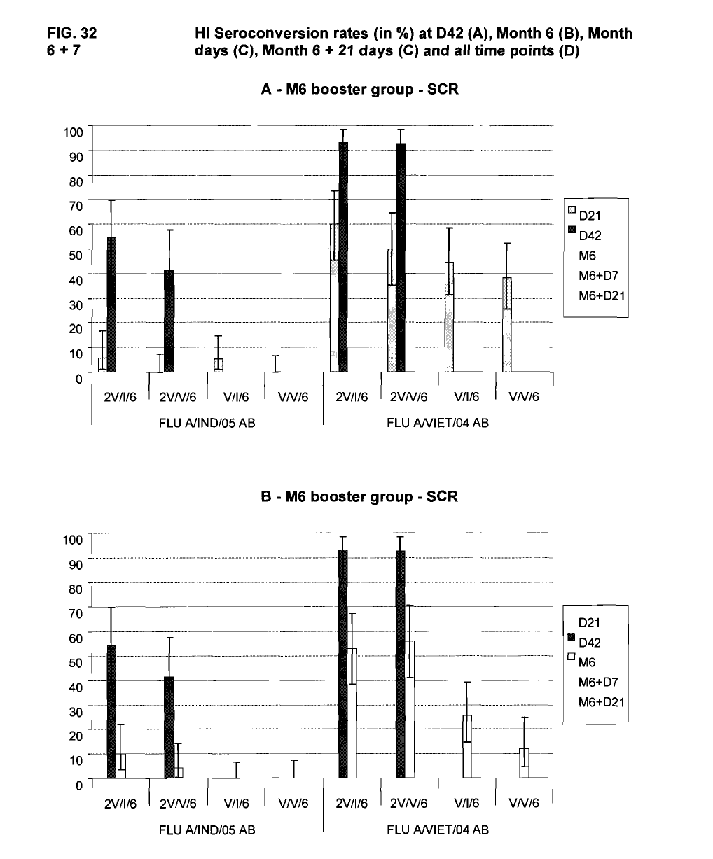

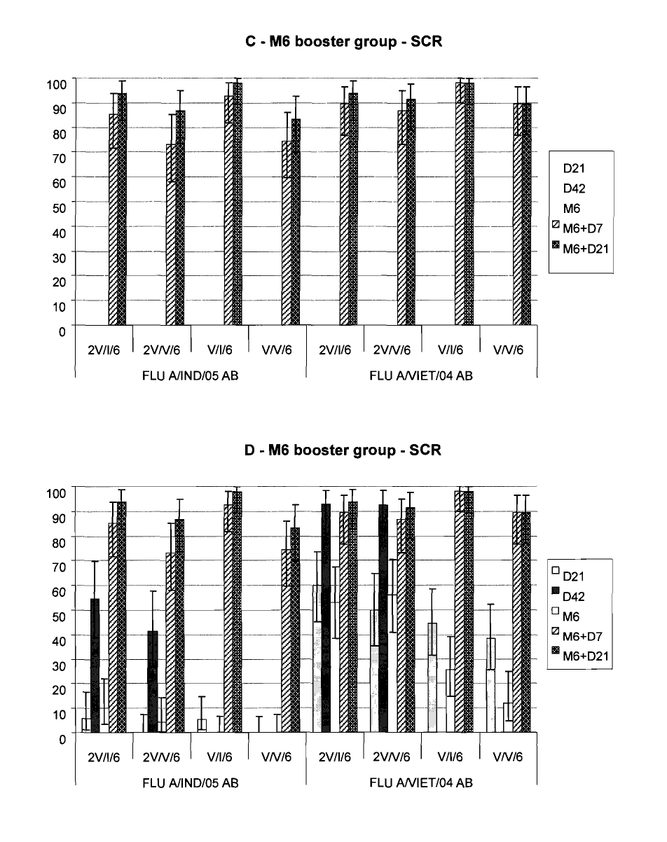

These data are summarised in FIG. 32A-D.

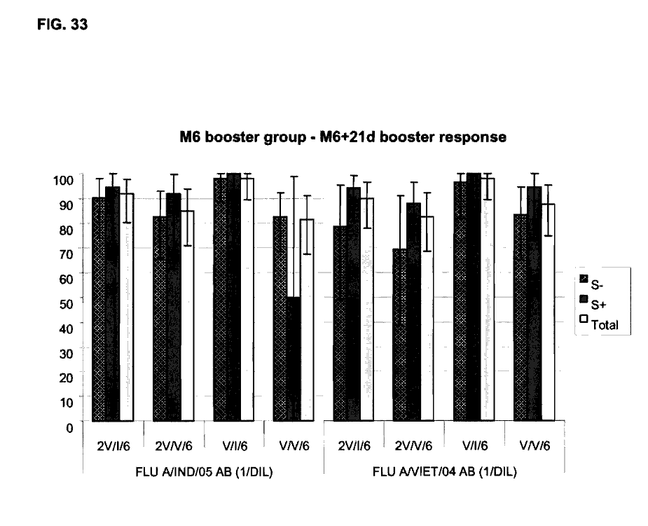

The booster seroconversion rate (same calculation as that performed for the SCR, but using the Month 6 HI value as the pre-vaccination value, i.e. pre-booster) is shown in Table 56 and in FIG. 33. The same analysis at Month 6+7 days is shown in Table 57.

TABLE 56
Booster Response (BR = Seroconversion rate using Month 6
as a ref) of HI antibodies against the VT or INstrains at Month
6 + 21 days for adults who received the booster dose at Month 6

| | | Pre- | | | | | |
|---|---|---|---|---|---|---|---|
| | | vaccination | | SCR booster | | | |
| Antibodies | | status | | | | 95% CI | |
| against | Group | (M 6) | N | n | % | LL | UL |
| A/ | VT/IN/6Mo | S− | 48 | 47 | 97.9 | 88.9 | 99.9 |
| Indonesia | | S+ | 4 | 4 | 100 | 39.8 | 100 |
| | | Total | 52 | 51 | 98.1 | 89.7 | 100 |
| | VT/VT/6Mo | S− | 46 | 38 | 82.6 | 68.6 | 92.2 |
| | | S+ | 2 | 1 | 50.0 | 1.3 | 98.7 |
| | | Total | 48 | 39 | 81.3 | 67.4 | 91.1 |
| | 2VT/IN/6Mo | S− | 31 | 28 | 90.3 | 74.2 | 98.0 |
| | | S+ | 18 | 17 | 94.4 | 72.7 | 99.9 |
| | | Total | 49 | 45 | 91.8 | 80.4 | 97.7 |
| | 2VT/VT/6Mo | S− | 34 | 28 | 82.4 | 65.5 | 93.2 |
| | | S+ | 12 | 11 | 91.7 | 61.5 | 99.8 |
| | | Total | 46 | 39 | 84.8 | 71.1 | 93.7 |
| A/ | VT/IN/6Mo | S− | 28 | 27 | 96.4 | 81.7 | 99.9 |
| Vietnam | | S+ | 24 | 24 | 100 | 85.8 | 100 |
| | | Total | 52 | 51 | 98.1 | 89.7 | 100 |
| | VT/VT/6Mo | S− | 30 | 25 | 83.3 | 65.3 | 94.4 |
| | | S+ | 18 | 17 | 94.4 | 72.7 | 99.9 |
| | | Total | 48 | 42 | 87.5 | 74.8 | 95.3 |
| | 2VT/IN/6Mo | S− | 14 | 11 | 78.6 | 49.2 | 95.3 |
| | | S+ | 35 | 33 | 94.3 | 80.8 | 99.3 |
| | | Total | 49 | 44 | 89.8 | 77.8 | 96.6 |
| | 2VT/VT/6Mo | S− | 13 | 9 | 69.2 | 38.6 | 90.9 |
| | | S+ | 33 | 29 | 87.9 | 71.8 | 96.6 |
| | | Total | 46 | 38 | 82.6 | 68.6 | 92.2 |

S− = seronegative subjects (antibody titre <10 1/DIL) prior to booster vaccination at month 6; S+ = seropositive subjects (antibody titre >=10 1/DIL) prior to booster vaccination at month 6; Total = subjects either seropositive or seronegative at pre-vaccination; Seroconversion rate Booster defined as: For seronegative subjects at pre-booster (Month 6), antibody titre >=40 1/DIL at Month 6 + 21 days; For seropositive subjects at pre-booster (Month 6), antibody titre at Month 6 + 21 days >=4 fold the pre-vaccination antibody titre at month 6; N = number of subjects with both pre- and post- booster vaccination results available; n/% = number/percentage of responders; 95% CI = exact 95% confidence interval; LL = lower limit, UL = upper limit

TABLE 57
Booster Response (BR = Seroconversion rate using Month 6
as a ref) of HI antibodies against the VT or INstrains at Month
6 + 7 days, for adults who received the booster dose at Month 6

| | | Pre- | | | | | |
|---|---|---|---|---|---|---|---|
| | | vaccination | | SCR booster | | | |
| Antibodies | | status | | | | 95% CI | |
| against | Group | (M 6) | N | n | % | LL | UL |
| A/ | VT/IN/6Mo | S− | 49 | 45 | 91.8 | 80.4 | 97.7 |
| Indonesia | | S+ | 4 | 3 | 75.0 | 19.4 | 99.4 |
| | | Total | 53 | 48 | 90.6 | 79.3 | 96.9 |
| | VT/VT/6Mo | S− | 45 | 33 | 73.3 | 58.1 | 85.4 |
| | | S+ | 2 | 2 | 100 | 15.8 | 100 |
| | | Total | 47 | 35 | 74.5 | 59.7 | 86.1 |
| | 2VT/IN/6Mo | S− | 31 | 25 | 80.6 | 62.5 | 92.5 |
| | | S+ | 16 | 15 | 93.8 | 69.8 | 99.8 |
| | | Total | 47 | 40 | 85.1 | 71.7 | 93.8 |
| | 2VT/VT/6Mo | S− | 34 | 22 | 64.7 | 46.5 | 80.3 |
| | | S+ | 11 | 9 | 81.8 | 48.2 | 97.7 |
| | | Total | 45 | 31 | 68.9 | 53.4 | 81.8 |
| A/ | VT/IN/6Mo | S− | 28 | 27 | 96.4 | 81.7 | 99.9 |
| Vietnam | | S+ | 25 | 20 | 80.0 | 59.3 | 93.2 |
| | | Total | 53 | 47 | 88.7 | 77.0 | 95.7 |
| | VT/VT/6Mo | S− | 29 | 24 | 82.8 | 64.2 | 94.2 |
| | | S+ | 18 | 16 | 88.9 | 65.3 | 98.6 |
| | | Total | 47 | 40 | 85.1 | 71.7 | 93.8 |
| | 2VT/IN/6Mo | S− | 14 | 9 | 64.3 | 35.1 | 87.2 |
| | | S+ | 33 | 22 | 66.7 | 48.2 | 82.0 |
| | | Total | 47 | 31 | 66.0 | 50.7 | 79.1 |
| | 2VT/VT/6Mo | S− | 13 | 8 | 61.5 | 31.6 | 86.1 |
| | | S+ | 32 | 22 | 68.8 | 50.0 | 83.9 |
| | | Total | 45 | 30 | 66.7 | 51.0 | 80.0 |

S− = seronegative subjects (antibody titre <10 1/DIL) prior to booster vaccination at month 6; S+ = seropositive subjects (antibody titre >=10 1/DIL) prior to booster vaccination at month 6; Total = subjects either seropositive or seronegative at pre-vaccination; Seroconversion rate Booster defined as: For seronegative subjects at pre-booster (Month 6), antibody titre >=40 1/DIL at Month 6 + 7 days; For seropositive subjects at pre-booster (Month 6), antibody titre at Month 6 + 7 days >=4 fold the pre-vaccination antibody titre at month 6; N = number of subjects with both pre- and post- booster vaccination results available; n/% = number/percentage of responders; 95% CI = exact 95% confidence interval; LL = lower limit, UL = upper limit

A ≧40% booster SCR value for HI antibody responses against the A/Vietnam/1194/2004 (61.5%-96.4%) and the A/Indonesia/5/2005 (64.7%-100.0%) strains at Month 6+7 days (i.e. 7 days post-booster). was already reached at Month 6+7 days (i.e. 7 days post-booster) for all groups receiving the booster vaccination at Month 6. This observation is valid regardless of the immune status (seropositive or seronegative) at Month 6 (persistence) At Month 6+21 days, the booster SCR against either the A/Vietnam/1194/2004 (69.2%-100.0%) or A/Indonesia/5/2005 (50.0%-100.0%) strains showed a slight further increase compared to the previous time-point.

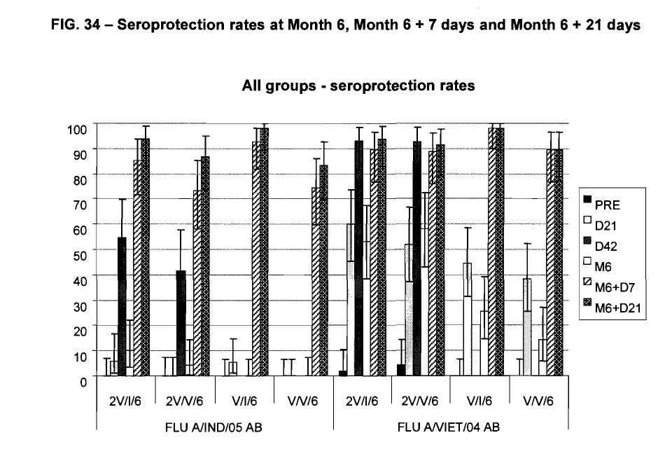

The seroprotection rates (SPR, defined as the percentage of vaccinees with a serum HI antibody titre≧1:40) at Month 6, Month 6+7 days and Month 6+21 days are shown in Table 58 and in FIG. 34.

TABLE 58
SPR of HI antibodies against the VT or INstrains up to Month 6 +
21 days, for adults who received the booster dose at Month 6

| | | | | SPR | | | |
|---|---|---|---|---|---|---|---|
| Antibodies | | | | | | 95% CI | |
| against | Group | Timing | N | n | % | LL | UL |
| A/ | VT/IN/6Mo | PRE | 56 | 0 | 0.0 | 0.0 | 6.4 |
| Indonesia | | PI(D 21) | 56 | 3 | 5.4 | 1.1 | 14.9 |
| | | PI(M 6) | 55 | 0 | 0.0 | 0.0 | 6.5 |
| | | PII(M 6 + D 7) | 53 | 49 | 92.5 | 81.8 | 97.9 |
| | | PII(M 6 + D 21) | 52 | 51 | 98.1 | 89.7 | 100 |
| | VT/VT/6Mo | PRE | 55 | 0 | 0.0 | 0.0 | 6.5 |
| | | PI(D 21) | 55 | 0 | 0.0 | 0.0 | 6.5 |
| | | PI(M 6) | 49 | 0 | 0.0 | 0.0 | 7.3 |
| | | PII(M 6 + D 7) | 47 | 35 | 74.5 | 59.7 | 86.1 |
| | | PII(M 6 + D 21) | 48 | 40 | 83.3 | 69.8 | 92.5 |
| | 2VT/IN/6Mo | PRE | 50 | 0 | 0.0 | 0.0 | 7.1 |
| | | PI(D 21) | 50 | 3 | 6.0 | 1.3 | 16.5 |
| | | PII(D 42) | 44 | 24 | 54.5 | 38.8 | 69.6 |
| | | PII(M 6) | 49 | 5 | 10.2 | 3.4 | 22.2 |
| | | PIII(M 6 + D 7) | 47 | 40 | 85.1 | 71.7 | 93.8 |
| | | PIII(M 6 + D 21) | 49 | 46 | 93.9 | 83.1 | 98.7 |
| | 2VT/VT/6Mo | PRE | 48 | 0 | 0.0 | 0.0 | 7.4 |
| | | PI(D 21) | 48 | 0 | 0.0 | 0.0 | 7.4 |
| | | PII(D 42) | 41 | 17 | 41.5 | 26.3 | 57.9 |
| | | PII(M 6) | 48 | 2 | 4.2 | 0.5 | 14.3 |
| | | PIII(M 6 + D 7) | 45 | 33 | 73.3 | 58.1 | 85.4 |
| | | PIII(M 6 + D 21) | 46 | 40 | 87.0 | 73.7 | 95.1 |
| A/ | VT/IN/6Mo | PRE | 56 | 0 | 0.0 | 0.0 | 6.4 |
| Vietnam | | PI(D 21) | 56 | 25 | 44.6 | 31.3 | 58.5 |
| | | PI(M 6) | 55 | 14 | 25.5 | 14.7 | 39.0 |
| | | PII(M 6 + D 7) | 53 | 52 | 98.1 | 89.9 | 100 |
| | | PII(M 6 + D 21) | 52 | 51 | 98.1 | 89.7 | 100 |
| | VT/VT/6Mo | PRE | 55 | 0 | 0.0 | 0.0 | 6.5 |
| | | PI(D 21) | 55 | 21 | 38.2 | 25.4 | 52.3 |
| | | PI(M 6) | 49 | 7 | 14.3 | 5.9 | 27.2 |
| | | PII(M 6 + D 7) | 47 | 42 | 89.4 | 76.9 | 96.5 |
| | | PII(M 6 + D 21) | 48 | 43 | 89.6 | 77.3 | 96.5 |
| | 2VT/IN/6Mo | PRE | 50 | 1 | 2.0 | 0.1 | 10.6 |
| | | PI(D 21) | 50 | 30 | 60.0 | 45.2 | 73.6 |
| | | PII(D 42) | 44 | 41 | 93.2 | 81.3 | 98.6 |
| | | PII(M 6) | 49 | 26 | 53.1 | 38.3 | 67.5 |
| | | PIII(M 6 + D 7) | 47 | 42 | 89.4 | 76.9 | 96.5 |
| | | PIII(M 6 + D 21) | 49 | 46 | 93.9 | 83.1 | 98.7 |
| | 2VT/VT/6Mo | PRE | 48 | 2 | 4.2 | 0.5 | 14.3 |
| | | PI(D 21) | 48 | 25 | 52.1 | 37.2 | 66.7 |
| | | PII(D 42) | 41 | 38 | 92.7 | 80.1 | 98.5 |
| | | PII(M 6) | 48 | 28 | 58.3 | 43.2 | 72.4 |
| | | PIII(M 6 + D 7) | 45 | 40 | 88.9 | 75.9 | 96.3 |
| | | PIII(M 6 + D 21) | 46 | 42 | 91.3 | 79.2 | 97.6 |

N = Number of subjects with available results; n/% = Number/percentage of seroprotected subjects (HI titre >=40 1/DIL); 95% CI = 95% confidence interval, LL = Lower Limit, UL = Upper Limit; PRE = Pre-vacc. dose 1 at D 0; PI(D 21) = Post-vacc. dose 1 at D 21; PI(M 6) = Post-vacc. dose 1 at M 6; PII(D 42) = Post-vacc. dose 2 at D 42; PII(M 6) = Post-vacc. dose 2 at M 6; PII(M 6 + D 7) = Post-vacc. dose 2 at D 7 after M 6; PII(M 6 + D 21) = Post-vacc. dose 2 at D 21 after M 6; PIII(M 6 + D 7) = Post-vacc. dose 3 at D 7 after M 6; PIII(M 6 + D 21) = Post-vacc. dose 3 at D 21 after M 6

The ≧70% SPR threshold of HI antibodies required by the CHMP for both the A/Vietnam/1194/2004 strain and the A/Indonesia/5/2005 strain was reached by all groups by Month 6+7 days (73.3%-98.1%) and rose slightly further at Month 6+21 days (83.3%-98.1%).
The FDA Guidance for Industry for pandemic vaccines requires that the lower limit of the 95% confidence interval for seroprotection should be reached by □70% of subjects for an HI antibody titre≧1:40. This threshold was met by all groups at Month 6+7 days, except groups VT/VT/6 and 2VT/VT/6 against A/Indonesia/5/05, and by all groups at and Month 6+21 days.
Following the booster administration, all groups show high seroprotection rates with no significant difference observed against either the A/Indonesia/5/2005 or the A/Vietnam/1194/2004 strains between the four groups who received the booster vaccination at Month 6. The study was not powered to demonstrate such a difference.

The seroconversion factor up to Month 6+21 days is shown in Table 59.

TABLE 59
SCF using Day 0 as a reference, of HI antibodies
against the A/Vietnam/1194/2004 or A/Indonesia/5/2005
strains up to Month 6 + 21 days, for adults
who received the booster dose at Month 6

| | | | | SCF | | |
|---|---|---|---|---|---|---|
| Antibodies | | | | | 95% CI | |
| against | Group | Timing | N | Value | LL | UL |
| A/ | VT/IN/6Mo | PI(D 21) | 56 | 1.3 | 1.1 | 1.5 |
| Indonesia | | PI(M 6) | 55 | 1.1 | 1.0 | 1.2 |
| | | PII(M 6 + D 7) | 53 | 30.6 | 22.4 | 41.6 |
| | | PII(M 6 + D 21) | 52 | 60.7 | 43.1 | 85.3 |
| | VT/VT/6Mo | PI(D 21) | 55 | 1.1 | 1.0 | 1.2 |
| | | PI(M 6) | 49 | 1.1 | 1.0 | 1.1 |
| | | PII(M 6 + D 7) | 47 | 12.9 | 8.3 | 20.2 |
| | | PII(M 6 + D 21) | 48 | 18.5 | 12.2 | 28.0 |
| | 2VT/IN/6Mo | PI(D 21) | 50 | 1.2 | 1.0 | 1.5 |
| | | PII(D 42) | 44 | 4.7 | 3.1 | 7.1 |
| | | PII(M 6) | 49 | 1.7 | 1.3 | 2.3 |
| | | PIII(M 6 + D 7) | 47 | 24.0 | 15.4 | 37.5 |
| | | PIII(M 6 + D 21) | 49 | 78.6 | 51.3 | 120.3 |
| | 2VT/VT/6Mo | PI(D 21) | 48 | 1.1 | 1.0 | 5.7 |
| | | PII(D 42) | 41 | 3.7 | 2.4 | 1.7 |
| | | PII(M 6) | 48 | 1.4 | 1.2 | 1.7 |
| | | PIII(M 6 + D 7) | 45 | 13.0 | 8.4 | 20.0 |
| | | PIII(M 6 + D 21) | 46 | 25.5 | 16.8 | 38.9 |
| A/ | VT/IN/6Mo | PI(D 21) | 56 | 4.2 | 2.8 | 6.2 |
| Vietnam | | PI(M 6) | 55 | 2.4 | 1.8 | 3.2 |
| | | PII(M 6 + D 7) | 53 | 45.3 | 33.7 | 60.9 |
| | | PII(M 6 + D 21) | 52 | 86.9 | 63.0 | 120.1 |
| | VT/VT/6Mo | PI(D 21) | 55 | 3.1 | 2.2 | 4.3 |
| | | PI(M 6) | 49 | 1.8 | 1.4 | 2.4 |
| | | PII(M 6 + D 7) | 47 | 38.2 | 24.5 | 59.7 |
| | | PII(M 6 + D 21) | 48 | 54.2 | 33.8 | 87.1 |
| | 2VT/IN/6Mo | PI(D 21) | 50 | 6.5 | 4.2 | 10.0 |
| | | PII(D 42) | 44 | 43.8 | 29.3 | 65.6 |
| | | PII(M 6) | 49 | 6.0 | 4.0 | 8.9 |
| | | PIII(M 6 + D 7) | 47 | 36.5 | 23.7 | 56.3 |
| | | PIII(M 6 + D 21) | 49 | 114.3 | 73.3 | 178.3 |
| | 2VT/VT/6Mo | PI(D 21) | 48 | 5.1 | 3.3 | 8.0 |
| | | PII(D 42) | 41 | 49.2 | 32.6 | 74.5 |
| | | PII(M 6) | 48 | 5.6 | 3.8 | 8.2 |
| | | PIII(M 6 + D 7) | 45 | 38.8 | 24.7 | 60.9 |
| | | PIII(M 6 + D 21) | 46 | 66.0 | 41.2 | 105.7 |

N = Number of subjects with pre- and post-vaccination results available; SCF = Seroconversion Factor or geometric mean ratio (mean[log10(POST/D 0)]); 95% CI = 95% confidence interval, LL = Lower Limit, UL = Upper Limit; PI(M 6) = Post-vaccination dose 1 at Month 6; PII(M 6) = Post-vaccination dose 2 at Month 6; PII(M 6 + D 7) = Post-vaccination dose 2 at Day 7 after Month 6; PII(M 6 + D 21) = Post-vaccination dose 2 at Day 21 after Month 6; PIII(M 6 + D 7) = Post-vaccination dose 3 at Day 7 after Month 6; PIII(M 6 + D 21) = Post-vaccination dose 3 at Day 21 after Month 6

≧2.5 SCF threshold required by the CHMP was reached by all groups for both the A/Vietnam/1194/2004 and the A/Indonesia/5/2005 strains at Month 6+7 days and rose further at Month 6+21 days.

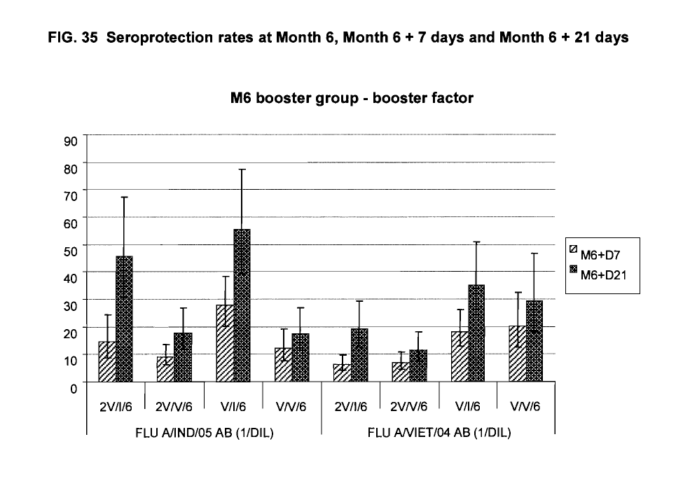

The booster factor (same calculation as that performed for the SCF, but using the Month 6 HI value as the pre-vaccination value, i.e. pre-booster) at Month 6+7 days is shown in Table 60 and in FIG. 35. Table 61 shows the BF at Month 6+21 days.

TABLE 60
Booster factor (BF) using Month 6 as a reference, of HI
antibodies against the VT or IN strains at Month 6 +
7 days for adults who received the booster dose at Month 6

| | | | | BF | | |
|---|---|---|---|---|---|---|
| Antibodies | | | | | 95% CI | |
| against | Group | Timing | N | Value | LL | UL |
| A/ | VT/IN/6Mo | PII(M 6 + D 7) | 53 | 27.9 | 20.4 | 38.3 |
| Indonesia | VT/VT/6Mo | PII(M 6 + D 7) | 47 | 12.3 | 7.8 | 19.2 |
| | 2VT/IN/6Mo | PIII(M 6 + D 7) | 47 | 14.5 | 8.7 | 24.3 |
| | 2VT/VT/6Mo | PIII(M 6 + D 7) | 45 | 9.2 | 6.2 | 13.7 |
| A/ | VT/IN/6Mo | PII(M 6 + D 7) | 53 | 18.2 | 12.8 | 26.0 |
| Vietnam | VT/VT/6Mo | PII(M 6 + D 7) | 47 | 20.3 | 12.7 | 32.5 |
| | 2VT/IN/6Mo | PIII(M 6 + D 7) | 47 | 6.3 | 4.1 | 9.7 |
| | 2VT/VT/6Mo | PIII(M 6 + D 7) | 45 | 6.9 | 4.5 | 10.7 |

N = Number of subjects with pre- and post-vaccination results available; Booster factor (BF) = Seroconversion Factor booster (mean[log10(POST/M 6)]); 95% CI = 95% confidence interval, LL = Lower Limit, UL = Upper Limit; PII(M 6 + D 7) = Post-vaccination dose 2 at Day 7 after Month 6; PIII(M 6 + D 7) = Post-vaccination dose 3 at Day 7 after Month 6

TABLE 61
Booster factor (BF) using Month 6 as a reference, of HI
antibodies against the VT or IN strains at Month 6 +
21 days, for adults who received the booster dose at Month 6

| | | | | BF | | |
|---|---|---|---|---|---|---|
| Antibodies | | | | | 95% CI | |
| against | Group | Timing | N | Value | LL | UL |
| A/ | VT/IN/6Mo | PII(M 6 + D 21) | 52 | 55.3 | 39.5 | 77.4 |
| Indonesia | VT/VT/6Mo | PII(M 6 + D 21) | 48 | 17.6 | 11.5 | 26.8 |
| | 2VT/IN/6Mo | PIII(M 6 + D 21) | 49 | 45.6 | 30.8 | 67.4 |
| | 2VT/VT/6Mo | PIII(M 6 + D 21) | 46 | 17.9 | 11.9 | 27.0 |
| A/ | VT/IN/6Mo | PII(M 6 + D 21) | 52 | 35.4 | 24.5 | 51.1 |
| Vietnam | VT/VT/6Mo | PII(M 6 + D 21) | 48 | 29.2 | 18.2 | 46.6 |
| | 2VT/IN/6Mo | PIII(M 6 + D 21) | 49 | 19.1 | 12.4 | 29.4 |
| | 2VT/VT/6Mo | PIII(M 6 + D 21) | 46 | 11.5 | 7.3 | 18.0 |

N = Number of subjects with pre- and post-vaccination results available; Booster factor (BF) = Seroconversion Factor booster (mean[log10(POST/M 6)]); 95% CI = 95% confidence interval, LL = Lower Limit, UL = Upper Limit; PII(M 6 + D 21) = Post-vaccination dose 2 at Day 21 after Month 6; PIII(M 6 + D 21) = Post-vaccination dose 3 at Day 21 after Month 6

The booster factor is confounded by the higher persistence levels of HI antibodies at Month 6 for the groups who received 2 primary vaccination doses as opposed to the groups who received a single priming dose.
The BF at Month 6+7 days against the A/Vietnam/1194/2004 strain is higher for the one-dose priming schedule (18.2-20.3) than for the two-dose priming schedule (6.3-6.9). The same observation applies at Month 6+21 days.
The BF at Month 6+21 days against the A/Indonesia/5/2005 strain shows a trend towards being higher for the groups boosted with A/Indonesia/5/2005 (45.6-55.3) than those boosted with A/Vietnam/1194/2004 (17.6-17.9), the A/Indonesia/5/2005 strain being homologous and heterologous to the booster strains, respectively.

The booster response (>4-fold increase in serum HI antibody titre between pre-booster vaccination and post-booster vaccination) at Month 6+7 days and Month 6+21 days is shown in Table 62.

TABLE 62
Booster response (BR = Seroconversion rate using Month 6 as a ref)
of HI antibodies against the VT or IN strains at Month 6 + 7 days
and Month 6 + 21 days, for adults who received the booster dose at Month 6

| | | | | BR | | | |
|---|---|---|---|---|---|---|---|
| Antibodies | | | | | | 95% CI | |
| against | Group | Timing | N | n | % | LL | UL |
| A/ | VT/IN/6Mo | PII(M 6 + D 7) | 53 | 51 | 96.2 | 87.0 | 99.5 |
| Indonesia | | PII(M 6 + D 21) | 52 | 51 | 98.1 | 89.7 | 100 |
| | VT/VT/6Mo | PII(M 6 + D 7) | 47 | 37 | 78.7 | 64.3 | 89.3 |
| | | PII(M 6 + D 21) | 48 | 40 | 83.3 | 69.8 | 92.5 |
| | 2VT/IN/6Mo | PIII(M 6 + D 7) | 47 | 41 | 87.2 | 74.3 | 95.2 |
| | | PIII(M 6 + D 21) | 49 | 46 | 93.9 | 83.1 | 98.7 |
| | 2VT/VT/6Mo | PIII(M 6 + D 7) | 45 | 34 | 75.6 | 60.5 | 87.1 |
| | | PIII(M 6 + D 21) | 46 | 40 | 87.0 | 73.7 | 95.1 |
| A/ | VT/IN/6Mo | PII(M 6 + D 7) | 53 | 47 | 88.7 | 77.0 | 95.7 |
| Vietnam | | PII(M 6 + D 21) | 52 | 51 | 98.1 | 89.7 | 100 |
| | VT/VT/6Mo | PII(M 6 + D 7) | 47 | 40 | 85.1 | 71.7 | 93.8 |
| | | PII(M 6 + D 21) | 48 | 42 | 87.5 | 74.8 | 95.3 |
| | 2VT/IN/6Mo | PIII(M 6 + D 7) | 47 | 32 | 68.1 | 52.9 | 80.9 |
| | | PIII(M 6 + D 21) | 49 | 45 | 91.8 | 80.4 | 97.7 |
| | 2VT/VT/6Mo | PIII(M 6 + D 7) | 45 | 31 | 68.9 | 53.4 | 81.8 |
| | | PIII(M 6 + D 21) | 46 | 39 | 84.8 | 71.1 | 93.7 |

Booster response defined as: antibody titre after booster vaccination >=4 fold the pre-booster vaccination antibody titre at Month 6; N = Number of subjects with pre- and post-vaccination results available; n/% = Number/percentage of seroconverted subjects; 95% CI = 95% confidence interval, LL = Lower Limit, UL = Upper Limit; PII(M 6 + D 7) = Post-vaccination dose 2 at Day 7 after Month 6; PII(M 6 + D 21) = Post-vaccination dose 2 at Day 21 after Month 6; PIII(M 6 + D 7) = Post-vaccination dose 3 at Day 7 after Month 6; PIII(M 6 + D 21) = Post-vaccination dose 3 at Day 21 after Month 6

The values at Month 6+7 days for the BR against the A/Vietnam/1194/2004 strain and the A/Indonesia/5/2005 strain were within the same range (61.8%-88.7% and 75.6%-96.2%, respectively).
The values at Month 6+21 days for the BR against the A/Vietnam/1194/2004 strain and the A/Indonesia/5/2005 strain were within the same range (84.8%-98.1% and 83.3%-98.1%, respectively).

### XIV.5. Overall Conclusions

### XIV.5.1 Response to the Booster Administration

A one-dose priming administration followed 6 months later by a booster dose containing a heterologous vaccine strain meets or exceeds all CHMP and FDA criteria set for the (pre-)pandemic candidate vaccine.
GMTs against the A/Vietnam/1194/2004 strain had increased significantly at the post-booster time-points regardless of the priming vaccination schedule (one or two doses) or booster strain received (homologous or heterologous to the priming vaccination strain).
GMTs against the A/Indonesia/5/2005 strain were higher for groups which had received the A/Indonesia/5/2005 strain as a booster (homologous response).
Regardless of the priming vaccination schedule (one or two doses) and regardless of the booster strain received (homologous or heterologous) the three criteria required by the CHMP (SCR, SPR and SCF) were already reached 7 days after the booster dose for both HI antibodies against the A/Vietnam/1194/2004 and A/Indonesia/5/2005 strains for all groups.
A homologous or heterologous booster administration six months after one or two priming doses induces a broad cross-reactive humoral immune response in terms of HI antibodies.
A ≧40% booster SCR threshold was already reached 7 days after the booster dose for HI antibodies against both the A/Vietnam/1194/2004 and A/Indonesia/5/2005 strains for all groups.
The booster factor against the A/Vietnam/1194/2004 strain is higher for the one-dose priming schedule than the two-dose priming schedule at both post-booster time-points, though this is probably confounded by the lower persistence observed following the one-dose priming schedule compared to the two-dose priming schedule.
The booster factor against the A/Indonesia/5/2005 strain shows a trend towards a better response if the booster strain was A/Indonesia/5/2005.
The booster response against both the A/Indonesia/5/2005 and the A/Vietnam/1194/2004 strains were within the same range for the four groups boosted at Month 6.

### XIV.5.2 Response to the Primary Vaccine Administration(s) and Persistence

The three criteria required by the CHMP (SCR, SPR and SCF) for HI antibodies against the A/Vietnam/1194/2004 strain at Day 42 were reached for groups receiving two priming doses.
The two-dose priming schedule induced a marked response against the A/Indonesia/5/2005 strain (heterologous response).
GMTs against both the A/Indonesia/5/2005 and the A/Vietnam/1194/2004 strains at Month 6 remained highest in the groups having received two priming doses.

### Example XV

### Phase II Clinical Trial in a Population Aged 19-61 Years with a Vaccine Containing an Adjuvanted Influenza Split Virus Antigen Preparation According to Different Vaccination Schedules

### XV.1. Introduction

A phase II clinical study has been performed to evaluate the reactogenicity and immunogenicity of one or two booster administrations of an influenza pandemic candidate vaccine in adults aged between 19 and 61 years, previously (about 14-months earlier) vaccinated with 2 doses of a pandemic candidate vaccine H5N1 A/Vietnam/1194/2004 containing 3.8, 7.5, 15 or 30 μg HA, adjuvanted or not with AS03. This study mimics a pandemic being declared after individuals have been primed twice with a pandemic vaccine formulated from a H5N1 strain heterologous to the emerging pandemic strain.

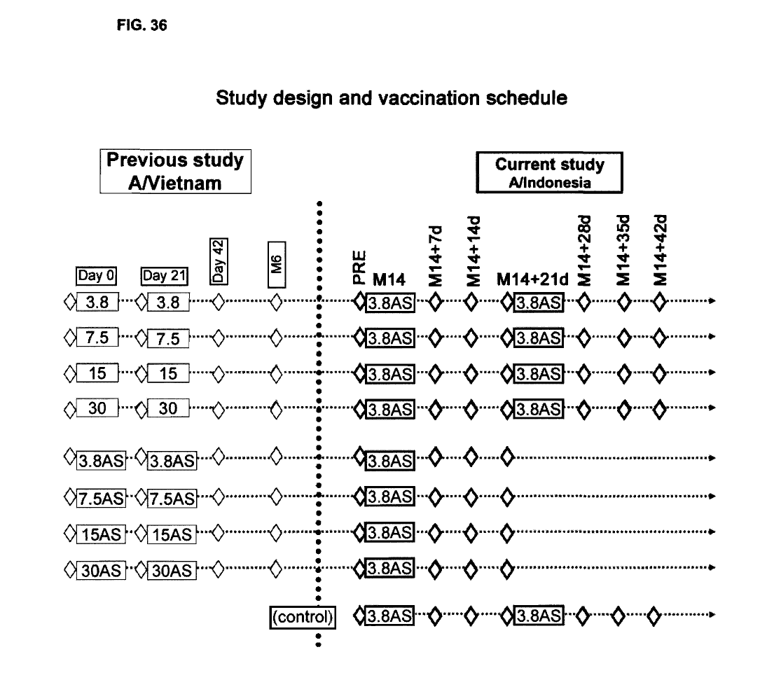

### XV.2. Study Design (FIG. 36)

Subjects who were vaccinated with the candidate vaccines formulated from the A/Vietnam/1194/2004 strain during the primary vaccination dose-range study done before have been revaccinated during the current study with a candidate vaccine formulated from the A/Indonesia/5/05 strain. Preliminary results from that previous study have shown that 2 administrations of non-adjuvanted vaccine is not sufficient to elicit an immune response considered as protective according to currently used regulatory criteria (from the CHMP and CBER). However, the adjuvanted vaccines (see section below for details) were able to induce an immune response exceeding all three criteria mentioned above. In this context subjects who have received non-adjuvanted vaccines during the previous trial were scheduled to receive two administrations of the adjuvanted candidate vaccine during the current booster trial. Subjects already primed with the adjuvanted vaccines during H5N1-007 have received one booster dose of the candidate vaccine during the current trial. In addition, a control group of unprimed subjects have received two doses of the candidate vaccine.

Thus 9 groups of subjects aged between 19-61 years were studied in parallel. All subjects have been vaccinated at Day 0 of the current trial (corresponding to Month 14 after the primary vaccination). In addition, subjects from non-adjuvanted vaccine groups (3.8, 7.5, 15 and 30 μg HA) and the Control group received a second administration at Day 21.
(H5N1_{—}3.8) Group of approximately 50 subjects who have received two doses of the candidate vaccine containing 3.8 μg HA (A/Vietnam/1194/2004 (H5N1)) during the previous trial.
(H5N1_{—}7.5) Group of approximately 50 subjects who have received two doses of the candidate vaccine containing 7.5 μg HA (A/Vietnam/1194/2004 (H5N1)) during the previous trial.
(H5N1_{—}5) Group of approximately 50 subjects who have received two doses of the candidate vaccine containing 15 μg HA (A/Vietnam/1194/2004 (H5N1)) during the previous trial.
(H5N1_{—}30) Group of approximately 50 subjects who have received two doses of the candidate vaccine containing 30 μg HA (A/Vietnam/1194/2004 (H5N1)) during the previous trial.
(H5N1_AS03_{—}3.8) Group of approximately 50 subjects who have received two doses of the candidate vaccine containing 3.8 μg HA (A/Vietnam/1194/2004 (H5N1)) adjuvanted with AS03 during the previous trial.
(H5N1_AS03_{—}7.5) Group of approximately 50 subjects who have received two doses of the candidate vaccine containing 7.5 μg HA (A/Vietnam/1194/2004 (H5N1)) adjuvanted with AS03 during the previous trial.
(H5N1_AS03_{—}15) Group of approximately 50 subjects who have received two doses of the candidate vaccine containing 15 μg HA (A/Vietnam/1194/2004 (H5N1)) adjuvanted with AS03 during the previous trial.
(H5N1 AS03_{—}30) Group of approximately 50 subjects who have received two doses of the candidate vaccine containing 30 μg HA (A/Vietnam/1194/2004 (H5N1)) adjuvanted with AS03 during the previous trial.
(H5N1_Cont) Group of 50 unprimed subjects as a control group.

Subjects from the previous trial's non-adjuvanted vaccine groups (3.8, 7.5, 15 and 30 μg HA) and the control group received two booster administrations (at the selected 3.8 μg adult dose/AS03) of the candidate vaccine at Day 0 and Day 21.

Subjects from the previous trial's AS03-adjuvanted vaccine groups (3.8, 7.5, 15 and 30 μg HA/AS03) received one booster dose (at the selected adult dose containing 3.8 μg HA/AS03) of the candidate vaccine at Day 0.

### XV.2. Study Objectives

Immunogenicity and reactogenicity/safety parameters were assessed. Only immunogenicity date are reported.

### XV.2.1. Immunogenicity Objectives:

The humoral immune response (in terms of HI and neutralizing antibody titres) and cell mediated immune response induced by a booster dose of the vaccine given 6 months after priming vaccination with a single dose of the vaccine formulated from a heterologous strain have been assessed.

Objectives: to Assess
for Humoral immune response (in terms of HI and neutralizing antibody titres)
   (i) if the humoral immune response induced 21 days after one booster administration of the candidate pandemic influenza vaccine fulfils the CHMP criteria in subjects primed approximately 14 months earlier with two administrations (21 days apart) of the candidate vaccine formulated from an heterologous strain and adjuvanted with AS03;
   (ii) the persistence of the humoral immune response in terms of haemagglutination-inhibiting (HI) and neutralizing antibody titers approximately 14 months after priming;
   (iii) the humoral immune response in terms of HI and neutralizing antibody titers 7, 14 and 21 days after each administration of the candidate vaccine;
   (iv) the persistence of the humoral immune response in terms of HI and neutralizing antibody titers approximately 6, 12, 18 and 24 months after the administration(s) of booster dose 1 of the candidate vaccine;
Cell mediated (CMI) immune response
   (i) the in vitro cell-mediated immune response at 0 and 21 days, and 6, 12, 18 and 24 months after the administration(s) of booster dose 1 of the candidate vaccine in terms of CD4/CD8 T cells expressing immune markers (CD40L, IL-2, TNF-α and IFN-γ) after in vitro stimulation of influenza-specific CD4/CD8 T-cells for groups 3.8 μg HA, 3.8 μg HA/AS03 and control;
   (ii) the impact of vaccination on influenza specific memory B-cells using the Elispot technology at 0 and 21 days after the administration(s) of the candidate vaccine;
   (iii) In vitro evaluation of T-Cell cross-reactivity to heterologous Flu strains.

### XV.2.2. Safety/Reactogenicity Objectives: Solicited Local and General Symptoms, Unsolicited Symptoms and Serious Adverse Events.

### XV.2.3. Evaluation Methods

In order to evaluate the humoral response in terms of HI antibodies and neutralizing antibodies, the following parameters were calculated with 95% confidence intervals at various time points:
GMTs of HI antibody titres;
Seroconversion rates (SCR) for the neutralising antibody response (defined as the percentage of vaccinees with a minimum 4-fold increase in neutralising antibody titre post vaccination) and for the HI antibody response (defined as the percentage of vaccinees that had either a pre-vaccination titre<1:10 and a post-vaccination titre≧1:40 or a pre-vaccination titre≧1:10 and at least a 4-fold increase in post-vaccination titre);
Seroconversion factors (SCF) defined as the fold increase in serum HI antibody GMTs post-vaccination compared to Day 0);
Seroprotection rates (SPR; defined as the percentage of vaccinees with a serum HI antibody titre≧1:40);

For the CMI response, the following parameters (with 95% CIs) were calculated at different time points in each group:
Frequency of influenza-specific CD4/CD8 T-cells per 10⁶ in tests producing at least two out of four different Th1-specific activation markers (CD40L, IL-2, TNF-α, IFN-γ).

### Statistical Methods for Immunogenicity

Humoral immune response: For the comparisons between groups, 95% CI adjusted GMT ratio between vaccine groups were computed using a one-way ANOVA model on the logarithm10 transformed titres.
Cell-mediated immune response: A non-parametric test (Wilcoxon Test) was used to compare the location of the difference between groups and the statistical p-value was calculated at each different test and each appropriate time-point. Wilcoxon test was also used to compare the individual difference (Post-Pre-vaccination) and the statistical p-value was calculated at each different test.

### XV.3. Vaccine Composition and Administration

One dose of vaccine was administered intramuscularly (IM) in the deltoid of the non-dominant arm.

XV.3.1. Vaccine composition: Monovalent, split virus, influenza pandemic candidate vaccine formulated from the A/Indonesia/5/2005 (H5N1) strain adjuvanted with AS03. The total injected volume was 0.5 ml.

The manufacturing process for the monovalent bulks of split, inactivated influenza H5N1 strain can be considered as identical to the manufacturing process for the monovalent bulks of GSK Biologicals licensed interpandemic influenza vaccine Fluarix™/α-Rix® (WO02/097072 and WO2008/009309). For the purpose of this clinical trial the virus strain used to manufacture the clinical lots is the H5N1 vaccine strain derived from the A/Indonesia/05/2005 (H5N1)/PR8-IBCDC-RG2 (IN strain), developed by CDC using reverse genetics. The reassortant strain combines the H5 and N1 segments to the A/PR/8134 strain backbone, and the H5 was engineered to eliminate the polybasic stretch of amino-acids at the HA cleavage site that is responsible for high virulence of the original strains. The active substance of the pandemic influenza vaccine candidate is a formaldehyde inactivated split virus antigen. The strain used for the previous study (the primary vaccination study) was the H5N1 vaccine strain A/Vietnam/1194/04 NIBRG-14 recombinant H5N1 prototype vaccine strain derived from the highly pathogenic A/Vietnam/1194/04 (VT strain) and developed by NIBSC using reverse genetics. Its manufacturing details are disclosed in Section XV.4 and in particular Table 7 of WO2008/009309.

The AS03 adjuvanted inactivated split virus influenza vaccines are 2 component vaccines consisting of concentrated inactivated split virion (H5N1) antigens presented in a type I glass vial and of the AS03 adjuvant contained in a pre-filled type I glass syringe. One adult dose of reconstituted AS03-adjuvanted vaccine corresponds to 0.5 ml. Their composition is given in Table 63.

TABLE 63
Composition of the reconstituted AS03 adjuvanted
influenza candidate vaccine

| | |
|---|---|
| Component | Quantity per dose |

ACTIVE INGREDIENTS

| | | |
|---|---|---|
| Purified antigen fractions of Inactivated | 3.75 | μg HA |
| split virions A/Indonesia/05/2005 | | |
| PR8-IBCDC-RG2 (H5N1) | | |

AS03 ADJUVANT

| | | |
|---|---|---|
| o/w emulsion | | |
| squalene | 10.68 | mg |
| DL-α-tocopherol | 11.86 | mg |
| Polysorbate 80 (Tween 80) | 4.86 | mg |

EXCIPIENTS

| | | |
|---|---|---|
| Polysorbate 80 (Tween 80) | Not less than 28.75 | μg |
| Octoxynol 10 (Triton X-100)² | 3.75 | μg |
| Thiomersal | 5 | μg |
| Sodium chloride | 3.7 | mg |
| Disodium hydrogen phosphate | 510 | μg |
| Potassium dihydrogen phosphate | 130 | μg |
| Potassium chloride | 90 | μg |
| Magnesium chloride hexahydrate | 12 | μg |
| Water for injections q.s. ad. | 0.5 | ml |

For this study, 0.25 ml of the content of the prefilled syringe containing the adjuvant and 0.25 ml of the content of the vial containing monovalent split antigen was used. After extemporaneous mixing of the contents, a 0.5 ml dose is withdrawn into the syringe and injected intramuscularly. At the time of injection, the content of the prefilled syringe containing the adjuvant is injected into the vial that contains the concentrated inactivated split virion antigens. One dose of the reconstituted the AS03-adjuvanted influenza candidate vaccine corresponds to 0.5 ml, containing 3.75 μg haemagglutinin (HA). Thiomersal was added as a preservative at a concentration of 10 μg/ml (5 μg per dose).

### XV.4. Immunogenicity Results

### XV.4.1 Humoral Response

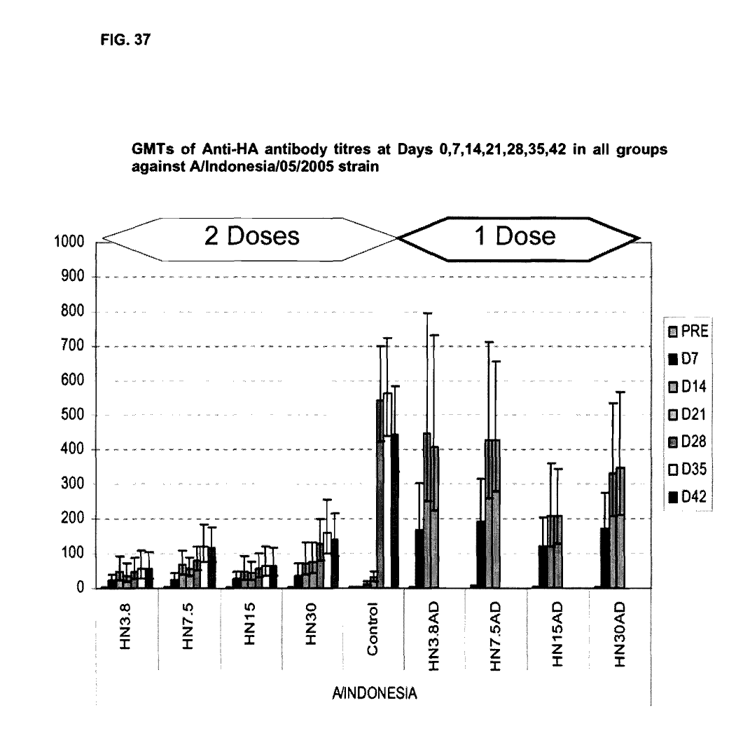

The GMTs and seropositivity rates were measured against the A/Indonesia/05/2005 vaccine strain at Days 0, 7, 14, 21, 28, 35, 42 in the non-adjuvanted and control groups (Table 64 and FIG. 37) and at Days 0, 7, 14, 21 in the adjuvanted vaccine groups (Table 65 and FIG. 37).

TABLE 64
Seropositivity rates and GMTs of HI antibody titres at Days 0, 7, 14, 21, 28, 35, 42 in the non-
adjuvanted and control groups against A/Indonesia/05/2005 strain (ATP cohort for immunogenicity)

| | | | | ≧10 1/DIL | | | | GMT | | | | |
|---|---|---|---|---|---|---|---|---|---|---|---|---|
| | | | | | | 95% CI | | | 95% CI | | | |
| Antibody | Group | Timing | N | n | % | LL | UL | value | LL | UL | Min | Max |
| A/ | HN3_8 | PRE | 34 | 1 | 2.9 | 0.1 | 15.3 | 5.1 | 4.9 | 5.3 | <10.0 | 10.0 |
| INDO | | PI(D 7) | 34 | 17 | 50.0 | 32.4 | 67.6 | 22.1 | 11.7 | 41.8 | <10.0 | 1280.0 |
| | | PI(D 14) | 34 | 22 | 64.7 | 46.5 | 80.3 | 46.6 | 24.0 | 90.7 | <10.0 | 2560.0 |
| | | PI(D 21) | 34 | 22 | 64.7 | 46.5 | 80.3 | 38.0 | 20.4 | 70.6 | <10.0 | 1280.0 |
| | | PII(D 28) | 34 | 25 | 73.5 | 55.6 | 87.1 | 48.1 | 26.1 | 88.7 | <10.0 | 1280.0 |
| | | PII(D 35) | 33 | 23 | 69.7 | 51.3 | 84.4 | 56.0 | 28.6 | 109.8 | <10.0 | 1280.0 |
| | | PII(D 42) | 34 | 23 | 67.6 | 49.5 | 82.6 | 54.3 | 27.9 | 105.8 | <10.0 | 1810.0 |
| | HN7_5 | PRE | 38 | 0 | 0.0 | 0.0 | 9.3 | 5.0 | 5.0 | 5.0 | <10.0 | <10.0 |
| | | PI(D 7) | 38 | 22 | 57.9 | 40.8 | 73.7 | 25.3 | 14.5 | 44.4 | <10.0 | 2560.0 |
| | | PI(D 14) | 38 | 31 | 81.6 | 65.7 | 92.3 | 66.1 | 39.7 | 109.9 | <10.0 | 2560.0 |
| | | PI(D 21) | 38 | 32 | 84.2 | 68.7 | 94.0 | 57.6 | 36.9 | 90.0 | <10.0 | 905.0 |
| | | PII(D 28) | 38 | 35 | 92.1 | 78.6 | 98.3 | 79.3 | 52.7 | 119.3 | <10.0 | 1280.0 |
| | | PII(D 35) | 36 | 33 | 91.7 | 77.5 | 98.2 | 119.9 | 77.5 | 185.5 | <10.0 | 1280.0 |
| | | PII(D 42) | 38 | 35 | 92.1 | 78.6 | 98.3 | 116.3 | 77.2 | 175.4 | <10.0 | 905.0 |
| | HN_15 | PRE | 33 | 0 | 0.0 | 0.0 | 10.6 | 5.0 | 5.0 | 5.0 | <10.0 | <10.0 |
| | | PI(D 7) | 33 | 20 | 60.6 | 42.1 | 77.1 | 27.7 | 16.1 | 47.6 | <10.0 | 226.0 |
| | | PI(D 14) | 32 | 22 | 68.8 | 50.0 | 83.9 | 49.1 | 25.7 | 93.8 | <10.0 | 640.0 |
| | | PI(D 21) | 32 | 22 | 68.8 | 50.0 | 83.9 | 42.2 | 23.0 | 77.4 | <10.0 | 453.0 |
| | | PII(D 28) | 32 | 24 | 75.0 | 56.6 | 88.5 | 56.0 | 31.2 | 100.4 | <10.0 | 640.0 |
| | | PII(D 35) | 32 | 25 | 78.1 | 60.0 | 90.7 | 65.8 | 36.1 | 120.2 | <10.0 | 640.0 |
| | | PII(D 42) | 32 | 24 | 75.0 | 56.6 | 88.5 | 63.7 | 34.5 | 117.9 | <10.0 | 1280.0 |
| | HN_30 | PRE | 28 | 0 | 0.0 | 0.0 | 12.3 | 5.0 | 5.0 | 5.0 | <10.0 | <10.0 |
| | | PI(D 7) | 28 | 18 | 64.3 | 44.1 | 81.4 | 37.6 | 19.3 | 73.4 | <10.0 | 640.0 |
| | | PI(D 14) | 28 | 23 | 82.1 | 63.1 | 93.9 | 71.5 | 39.0 | 131.2 | <10.0 | 640.0 |
| | | PI(D 21) | 28 | 24 | 85.7 | 67.3 | 96.0 | 77.1 | 45.1 | 131.7 | <10.0 | 640.0 |
| | | PII(D 28) | 28 | 27 | 96.4 | 81.7 | 99.9 | 128.0 | 81.8 | 200.4 | <10.0 | 1280.0 |
| | | PII(D 35) | 28 | 27 | 96.4 | 81.7 | 99.9 | 160.0 | 100.8 | 254.2 | <10.0 | 1810.0 |
| | | PII(D 42) | 28 | 27 | 96.4 | 81.7 | 99.9 | 141.4 | 92.3 | 216.7 | <10.0 | 1280.0 |
| | Control | PRE | 49 | 0 | 0.0 | 0.0 | 7.3 | 5.0 | 5.0 | 5.0 | <10.0 | <10.0 |
| | | PI(D 7) | 49 | 0 | 0.0 | 0.0 | 7.3 | 5.0 | 5.0 | 5.0 | <10.0 | <10.0 |
| | | PI(D 14) | 49 | 23 | 46.9 | 32.5 | 61.7 | 13.7 | 9.6 | 19.5 | <10.0 | 453.0 |
| | | PI(D 21) | 49 | 35 | 71.4 | 56.7 | 83.4 | 31.0 | 20.8 | 46.3 | <10.0 | 453.0 |
| | | PII(D 28) | 49 | 49 | 100 | 92.7 | 100 | 543.9 | 423.3 | 698.8 | 80.0 | 7240.0 |
| | | PII(D 35) | 49 | 49 | 100 | 92.7 | 100 | 563.6 | 438.5 | 724.4 | 57.0 | 7240.0 |
| | | PII(D 42) | 49 | 49 | 100 | 92.7 | 100 | 443.0 | 335.6 | 584.7 | 28.0 | 5120.0 |

N = Number of subjects with available results; n/% = number/percentage of seropositive subjects (HI titer >=1:10); 95% CI = 95% confidence interval, LL = Lower Limit, UL = Upper Limit; MIN/MAX = Minimum/Maximum
PRE = Pre-vaccination at Day 0; PI(D 7) = Post-vaccination one at Day 7; PI(D 14) = Post-vaccination one at Day 14; PI(D 21) = Post-vaccination one at Day 21; PII(D 28) = Post-vaccination two at Day 28; PII(D 35) = Post-vaccination two at Day 35; PII(D 42) = Post-vaccination two at Day 42

TABLE 65
Seropositivity rates and geometric means titres (GMTs) of HI antibody titres
at Days 0, 7, 14, 21, 28, 35 and 42 in the non-adjuvanted and control adjuvanted
groups against A/Indonesia/05/2005 strain (ATP cohort for immunogenicity)

| | | | | ≧10 1/DIL | | | | GMT | | | | |
|---|---|---|---|---|---|---|---|---|---|---|---|---|
| | | | | | | 95% CI | | | 95% CI | | | |
| Antibody | Group | Timing | N | n | % | LL | UL | value | LL | UL | Min | Max |
| A/ | HN3_8AD | PRE | 39 | 1 | 2.6 | 0.1 | 13.5 | 5.1 | 4.9 | 5.3 | <10.0 | 10.0 |
| INDONESIA | | PI(D 7) | 39 | 34 | 87.2 | 72.6 | 95.7 | 174.9 | 98.9 | 309.2 | <10.0 | 1280.0 |
| | | PI(D 14) | 40 | 37 | 92.5 | 79.6 | 98.4 | 460.7 | 263.4 | 806.0 | <10.0 | 5120.0 |
| | | PI(D 21) | 40 | 37 | 92.5 | 79.6 | 98.4 | 422.3 | 236.9 | 752.7 | <10.0 | 7240.0 |
| | HN7_5AD | PRE | 34 | 3 | 8.8 | 1.9 | 23.7 | 5.6 | 4.9 | 6.4 | <10.0 | 40.0 |
| | | PI(D 7) | 35 | 32 | 91.4 | 76.9 | 98.2 | 173.2 | 104.7 | 286.6 | <10.0 | 2560.0 |
| | | PI(D 14) | 35 | 33 | 94.3 | 80.8 | 99.3 | 378.6 | 220.4 | 650.6 | <10.0 | 7240.0 |
| | | PI(D 21) | 35 | 35 | 100 | 90.0 | 100 | 422.3 | 283.1 | 629.9 | 57.0 | 5120.0 |
| | | PII(D 28) | 1 | 1 | 100 | 2.5 | 100 | 905.0 | — | — | 905.0 | 905.0 |
| | | PII(D 35) | 1 | 1 | 100 | 2.5 | 100 | 905.0 | — | — | 905.0 | 905.0 |
| | | PII(D 42) | 1 | 1 | 100 | 2.5 | 100 | 640.0 | — | — | 640.0 | 640.0 |
| | HN15AD | PRE | 41 | 2 | 4.9 | 0.6 | 16.5 | 5.3 | 4.9 | 5.8 | <10.0 | 20.0 |
| | | PI(D 7) | 41 | 34 | 82.9 | 67.9 | 92.8 | 116.1 | 68.6 | 196.5 | <10.0 | 3620.0 |
| | | PI(D 14) | 41 | 36 | 87.8 | 73.8 | 95.9 | 213.3 | 125.3 | 363.0 | <10.0 | 5120.0 |
| | | PI(D 21) | 41 | 38 | 92.7 | 80.1 | 98.5 | 216.9 | 135.0 | 348.4 | <10.0 | 3620.0 |
| | | PII(D 28) | 1 | 1 | 100 | 2.5 | 100 | 1810.0 | — | — | 1810.0 | 1810.0 |
| | | PII(D 35) | 1 | 1 | 100 | 2.5 | 100 | 1810.0 | — | — | 1810.0 | 1810.0 |
| | | PII(D 42) | 1 | 1 | 100 | 2.5 | 100 | 1810.0 | — | — | 1810.0 | 1810.0 |
| | HN30AD | PRE | 34 | 1 | 2.9 | 0.1 | 15.3 | 5.2 | 4.8 | 5.5 | <10.0 | 14.0 |
| | | PI(D 7) | 35 | 32 | 91.4 | 76.9 | 98.2 | 156.8 | 97.1 | 253.4 | <10.0 | 1280.0 |
| | | PI(D 14) | 34 | 32 | 94.1 | 80.3 | 99.3 | 295.0 | 174.6 | 498.2 | <10.0 | 2560.0 |
| | | PI(D 21) | 35 | 33 | 94.3 | 80.8 | 99.3 | 307.5 | 182.7 | 517.5 | <10.0 | 5120.0 |
| | | PII(D 28) | 1 | 0 | 0.0 | 0.0 | 97.5 | 5.0 | — | — | <10.0 | <10.0 |
| | | PII(D 35) | 1 | 0 | 0.0 | 0.0 | 97.5 | 5.0 | — | — | <10.0 | <10.0 |
| | | PII(D 42) | 1 | 0 | 0.0 | 0.0 | 97.5 | 5.0 | — | — | <10.0 | <10.0 |

N = Number of subjects with available results; n/% = number/percentage of seropositive subjects (HI titer >=1:10); 95% CI = 95% confidence interval, LL = Lower Limit, UL = Upper Limit; MIN/MAX = Minimum/Maximum
PRE = Pre vaccination (Day 0); PI(D 7) = Post vaccination 1(Day 7); PI(D 14) = Post vaccination (Day 14); PI(D 21) = Post vaccination 1 (Day 21); PII(D 28) = Post-vaccination 2 (Day 28); PII(D 35) = Post vaccination 2 (Day 35); PII(D 42) = Post-vaccination 2 (Day 42)

### Intermediate Conclusion

A proportion of subjects who were previously vaccinated in the previous study about 14 months ago (whether AS03 was used or not during this primary vaccination series), became seropositive 7 days after one dose of vaccine in the current trial (PI(D7)), as opposed to the control subjects. Seropositivity rates were higher 7 days after one dose though, in subjects from the adjuvanted vaccine groups, as compared to subjects from the unadjuvanted vaccine groups.
21 days after the last vaccination, a majority of subjects became seropositive, irrespective of the group investigated.
The HI GMTs were significantly higher after the first revaccination dose in subjects from the previous trial's adjuvanted vaccine groups (HN_{—}3.8AD to HN_{—}30AD), as compared to subjects from the previous trial's unadjuvanted vaccine groups (HN_{—}3.8 to HN_{—}30).
The HI GMTs were significantly higher after the second revaccination dose in subjects from the control group, as compared to subjects from the previous trial's unadjuvanted vaccine groups (HN_{—}3.8 to HN_{—}30).

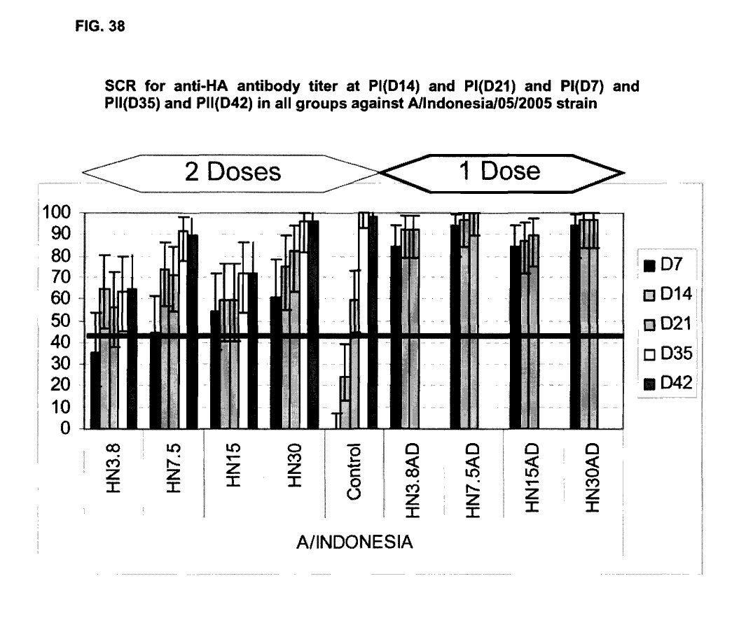

The seroconversion rates (SCR, defined as the percentage of vaccinees with either a pre-vaccination titre<1:10 and a post-vaccination titre≧1:40 or a pre-vaccination titre≧1:10 and at least a 4-fold increase in post-vaccination titre) are shown in Tables 66 and 67 for the previous trial's non-adjuvanted and control groups, and for the previous trial's adjuvanted groups, respectively. Data are also shown in FIG. 38.

TABLE 66
SCR for HI antibody titer at PI(D 14) and PI(D 21) and
PI(D 7) and PII(D 35) and PII(D 42) in the previous trial's
non-adjuvanted and control groups against A/Indonesia/05/2005
strain (ATP cohort for immunogenicity)

| | | | | SCR | | | |
|---|---|---|---|---|---|---|---|
| Vaccine | | | | | | 95% CI | |
| strain | Group | Timing | N | n | % | LL | UL |
| A/ | HN3_8 | PI(D 7) | 34 | 12 | 35.3 | 19.7 | 53.5 |
| INDONESIA | | PI(D 14) | 34 | 22 | 64.7 | 46.5 | 80.3 |
| | | PI(D 21) | 34 | 19 | 55.9 | 37.9 | 72.8 |
| | | PII(D 35) | 33 | 21 | 63.6 | 45.1 | 79.6 |
| | | PII(D 42) | 34 | 22 | 64.7 | 46.5 | 80.3 |
| | HN7_5 | PI(D 7) | 38 | 17 | 44.7 | 28.6 | 61.7 |
| | | PI(D 14) | 38 | 28 | 73.7 | 56.9 | 86.6 |
| | | PI(D 21) | 38 | 27 | 71.1 | 54.1 | 84.6 |
| | | PII(D 35) | 36 | 33 | 91.7 | 77.5 | 98.2 |
| | | PII(D 42) | 38 | 34 | 89.5 | 75.2 | 97.1 |
| | HN15 | PI(D 7) | 33 | 18 | 54.5 | 36.4 | 71.9 |
| | | PI(D 14) | 32 | 19 | 59.4 | 40.6 | 76.3 |
| | | PI(D 21) | 32 | 19 | 59.4 | 40.6 | 76.3 |
| | | PII(D 35) | 32 | 23 | 71.9 | 53.3 | 86.3 |
| | | PII(D 42) | 32 | 23 | 71.9 | 53.3 | 86.3 |
| | HN30 | PI(D 7) | 28 | 17 | 60.7 | 40.6 | 78.5 |
| | | PI(D 14) | 28 | 21 | 75.0 | 55.1 | 89.3 |
| | | PI(D 21) | 28 | 23 | 82.1 | 63.1 | 93.9 |
| | | PII(D 35) | 28 | 27 | 96.4 | 81.7 | 99.9 |
| | | PII(D 42) | 28 | 27 | 96.4 | 81.7 | 99.9 |
| | Control | PI(D 7) | 49 | 0 | 0.0 | 0.0 | 7.3 |
| | | PI(D 14) | 49 | 12 | 24.5 | 13.3 | 38.9 |
| | | PI(D 21) | 49 | 29 | 59.2 | 44.2 | 73.0 |
| | | PII(D 35) | 49 | 49 | 100 | 92.7 | 100 |
| | | PII(D 42) | 49 | 48 | 98.0 | 89.1 | 99.9 |

Seroconversion defined as:
For initially seronegative subjects, antibody titre >=40 1/DIL after vaccination
For initially seropositive subjects, antibody titre after vaccination >=4 fold the pre-vaccination antibody titre
N = Number of subjects with pre- and post-vaccination results available
n/% = Number/percentage of seroconverted subjects
95% CI = 95% confidence interval, LL = Lower Limit, UL = Upper Limit
PI(D 7) = Post vaccination 1 (Day 7); PI(D 14) = Post vaccination 1 (Day 14); PI(D 21) = Post vaccination 1 (Day 21); PII(D 35) = Post vaccination 2 (Day 3); PII(D 42) = Post vaccination 2 (Day 42)

TABLE 67
SCR for HI antibody titer at PI (D 14) and PI(D 21)
and PI(D 7) and PII(D 35) and PII(D 42) in the previous
trial's adjuvanted vaccine groups against A/Indonesia/05/2005
strain (ATP cohort for immunogenicity)

| | | | | SCR | | | |
|---|---|---|---|---|---|---|---|
| Vaccine | | | | | | 95% CI | |
| strain | Group | Timing | N | n | % | LL | UL |
| A/ | HN3_8AD | PI(D 7) | 38 | 32 | 84.2 | 68.7 | 94.0 |
| INDONESIA | | PI(D 14) | 39 | 36 | 92.3 | 79.1 | 98.4 |
| | | PI(D 21) | 39 | 36 | 92.3 | 79.1 | 98.4 |
| | HN7_5AD | PI(D 7) | 33 | 31 | 93.9 | 79.8 | 99.3 |
| | | PI(D 14) | 33 | 32 | 97.0 | 84.2 | 99.9 |
| | | PI(D 21) | 33 | 33 | 100 | 89.4 | 100 |
| | HN15AD | PI(D 7) | 38 | 32 | 84.2 | 68.7 | 94.0 |
| | | PI(D 14) | 38 | 33 | 86.8 | 71.9 | 95.6 |
| | | PI(D 21) | 38 | 34 | 89.5 | 75.2 | 97.1 |
| | HN30AD | PI(D 7) | 32 | 30 | 93.8 | 79.2 | 99.2 |
| | | PI(D 14) | 32 | 31 | 96.9 | 83.8 | 99.9 |
| | | PI(D 21) | 32 | 31 | 96.9 | 83.8 | 99.9 |

Seroconversion defined as:
For initially seronegative subjects, antibody titre >=40 1/DIL after vaccination
For initially seropositive subjects, antibody titre after vaccination >=4 fold the pre-vaccination antibody titre
N = Number of subjects with pre- and post-vaccination results available
n/% = Number/percentage of seroconverted subjects
95% CI = 95% confidence interval, LL = Lower Limit, UL = Upper Limit
PI(D 7) = Post vaccination 1 (Day 7); PI(D 14) = Post vaccination 1 (Day 14); PI(D 21) = Post vaccination 1 (Day 21)

### Intermediate Conclusion

The ≧40% SCR threshold required by the European Committee for Medicinal Products for Human Use (CHMP) for adults aged 18-60 years was exceeded 7 days after the vaccination in subjects from the previous trial's adjuvanted groups and SCR remained high 21 days after vaccination.
The ≧40% SCR threshold was exceeded 21 days after the first vaccination dose; SCR significantly increased after the second vaccination dose and remained high 21 days after the second vaccination in subjects from the control group.
In subjects from the previous trial's unadjuvanted groups, the ≧40% SCR threshold was also exceeded, at the selected 7.5, 15 and 30 μg adult doses, 7 days after the first vaccination (although the lower limit of the 95% confidence interval for seroconversion was inferior to the threshold for the 7.5 and 15 μg groups). Although there was a trend for an increase in SCR after the second vaccination dose, this was not significant, as compared as to after the first dose.

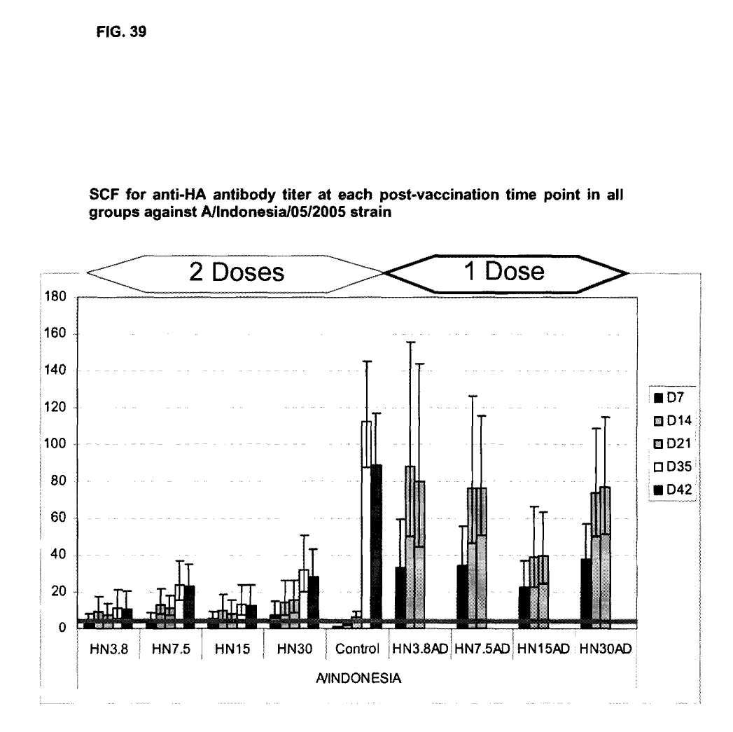

The seroconversion factor (SCF) for HI antibody titer against A/Indonesia/05/2005 strain was measured at each post-vaccination time point in the previous trial's non-adjuvanted and control groups (Table 68) and in the previous trial's adjuvanted groups (Table 69). Data are also shown in FIG. 39.

TABLE 68
SCF for HI antibody titer at each post-vaccination time point
in the previous trial's non-adjuvanted and control groups against
A/Indonesia/05/2005 strain (ATP cohort for immunogenicity)

| | | | | SCF | | |
|---|---|---|---|---|---|---|
| Vaccine | | | | | 95% CI | |
| strain | Group | Timing | N | Value | LL | UL |
| FLU A/IND/05 AB | HN3_8 | PI(D 7) | 34 | 4.3 | 2.3 | 8.2 |
| (1/DIL) | | PI(D 14) | 34 | 9.1 | 4.7 | 17.7 |
| | | PI(D 21) | 34 | 7.4 | 4.0 | 13.8 |
| | | PII(D 35) | 33 | 11.0 | 5.6 | 21.4 |
| | | PII(D 42) | 34 | 10.6 | 5.5 | 20.7 |
| | HN7_5 | PI(D 7) | 38 | 5.1 | 2.9 | 8.9 |
| | | PI(D 14) | 38 | 13.2 | 7.9 | 22.0 |
| | | PI(D 21) | 38 | 11.5 | 7.4 | 18.0 |
| | | PII(D 35) | 36 | 24.0 | 15.5 | 37.1 |
| | | PII(D 42) | 38 | 23.3 | 15.4 | 35.1 |
| | HN15 | PI(D 7) | 33 | 5.5 | 3.2 | 9.5 |
| | | PI(D 14) | 32 | 9.8 | 5.1 | 18.8 |
| | | PI(D 21) | 32 | 8.4 | 4.6 | 15.5 |
| | | PII(D 35) | 32 | 13.2 | 7.2 | 24.0 |
| | | PII(D 42) | 32 | 12.7 | 6.9 | 23.6 |
| | HN30 | PI(D 7) | 28 | 7.5 | 3.9 | 14.7 |
| | | PI(D 14) | 28 | 14.3 | 7.8 | 26.2 |
| | | PI(D 21) | 28 | 15.4 | 9.0 | 26.3 |
| | | PII(D 35) | 28 | 32.0 | 20.2 | 50.8 |
| | | PII(D 42) | 28 | 28.3 | 18.5 | 43.3 |
| | Control | PI(D 7) | 49 | 1.0 | 1.0 | 1.0 |
| | | PI(D 14) | 49 | 2.7 | 1.9 | 3.9 |
| | | PI(D 21) | 49 | 6.2 | 4.2 | 9.3 |
| | | PII(D 35) | 49 | 112.7 | 87.7 | 144.9 |
| | | PII(D 42) | 49 | 88.6 | 67.1 | 116.9 |

N = Number of subjects with pre- and post-vaccination results available
SCF = Seroconversion Factor or geometric mean ratio (mean[log10(POST/PRE)])
95% CI = 95% confidence interval, LL = Lower Limit, UL = Upper Limit
PI(D 7) = Post vaccination 1 (Day 7); PI(D 14) = Post vaccination 1 (Day 14); PI(D 21) = Post vaccination 1 (Day 21); PII(D 35) = Post vaccination 2 (Day 3); PII(D 42) = Post vaccination 2 (Day 42)

TABLE 69
SCF for HI antibody titer at each post-vaccination time point
in the previous trial's adjuvanted groups against A/Indonesia/05/2005
strain (ATP cohort for immunogenicity)

| | | | | SCF | | |
|---|---|---|---|---|---|---|
| Vaccine | | | | | 95% CI | |
| strain | Group | Timing | N | Value | LL | UL |
| FLU A/IND/05 AB | HN3_8AD | PI(D 7) | 38 | 33.2 | 18.6 | 59.3 |
| (1/DIL) | | PI(D 14) | 39 | 88.2 | 49.9 | 155.8 |
| | | PI(D 21) | 39 | 79.9 | 44.5 | 143.6 |
| | HN7_5AD | PI(D 7) | 33 | 34.5 | 21.2 | 55.9 |
| | | PI(D 14) | 33 | 76.5 | 46.4 | 126.2 |
| | | PI(D 21) | 33 | 76.5 | 50.7 | 115.6 |
| | HN15AD | PI(D 7) | 38 | 22.2 | 13.3 | 37.0 |
| | | PI(D 14) | 38 | 38.8 | 22.8 | 66.0 |
| | | PI(D 21) | 38 | 39.1 | 24.2 | 63.1 |
| | HN30AD | PI(D 7) | 32 | 37.6 | 25.0 | 56.6 |
| | | PI(D 14) | 32 | 73.7 | 49.9 | 108.8 |
| | | PI(D 21) | 32 | 76.9 | 51.5 | 115.0 |

N = Number of subjects with pre- and post-vaccination results available
SCF = Seroconversion Factor or geometric mean ratio (mean[log10(POST/PRE)])
95% CI = 95% confidence interval, LL = Lower Limit, UL = Upper Limit
PI(D 7) = Post vaccination 1 (Day 7); PI(D 14) = Post vaccination 1 (Day 14); PI(D 21) = Post vaccination 1 (Day 21)

### Intermediate Conclusion

The SCFs were significantly higher after the first vaccination dose in subjects from the previous trial's adjuvanted vaccine groups (HN_{—}3.8AD to HN_{—}30AD), as compared to subjects from the previous trial's unadjuvanted vaccine groups (HN_{—}3.8 to HN_{—}30).
The SCFs were significantly higher after the second vaccination dose in subjects from the control groups, as compared to subjects from the previous trial's unadjuvanted vaccine groups.

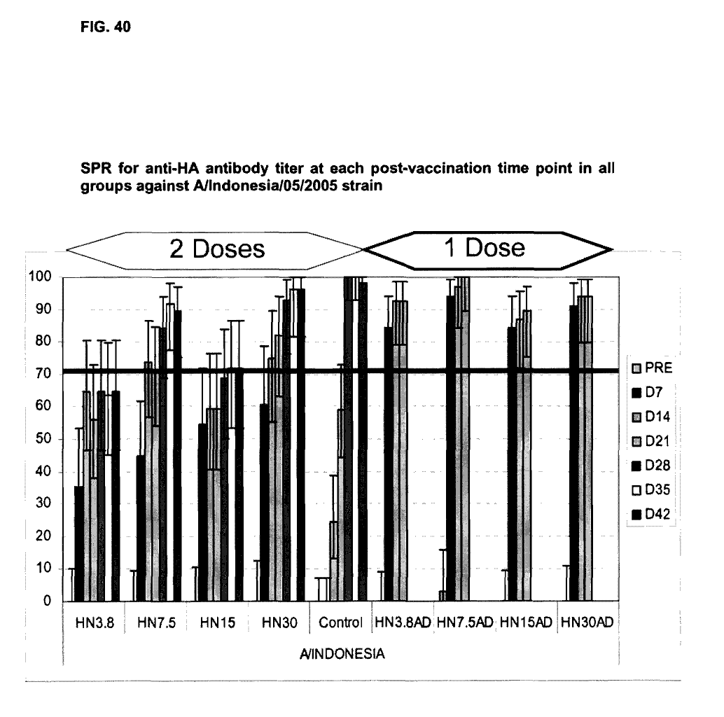

The seroprotection rates (SPR, defined as the percentage of vaccinees with a serum HI antibody titre≧1:40 that is usually accepted as indicating protection) for HI antibody titer against A/Indonesia/05/strain was measured at each time point in the previous trial's non-adjuvanted and control groups (Table 70) and in the previous trial's adjuvanted groups (Table 71). Data are also shown in FIG. 40.

TABLE 70
SPR for HI antibody titer at each time point in the previous
trial's non-adjuvanted and control groups against A/Indonesia/05/strain
(ATP cohort for immunogenicity)

| | | | | SPR | | | |
|---|---|---|---|---|---|---|---|
| Vaccine | | | | | | 95% CI | |
| strain | Group | Timing | N | n | % | LL | UL |
| A/ | HN3_8 | PRE | 34 | 0 | 0.0 | 0.0 | 10.3 |
| INDONESIA | | PI(D 7) | 34 | 12 | 35.3 | 19.7 | 53.5 |
| | | PI(D 14) | 34 | 22 | 64.7 | 46.5 | 80.3 |
| | | PI(D 21) | 34 | 19 | 55.9 | 37.9 | 72.8 |
| | | PII(D 28) | 34 | 22 | 64.7 | 46.5 | 80.3 |
| | | PII(D 35) | 33 | 21 | 63.6 | 45.1 | 79.6 |
| | | PII(D 42) | 34 | 22 | 64.7 | 46.5 | 80.3 |
| | HN7_5 | PRE | 38 | 0 | 0.0 | 0.0 | 9.3 |
| | | PI(D 7) | 38 | 17 | 44.7 | 28.6 | 61.7 |
| | | PI(D 14) | 38 | 28 | 73.7 | 56.9 | 86.6 |
| | | PI(D 21) | 38 | 27 | 71.1 | 54.1 | 84.6 |
| | | PII(D 28) | 38 | 32 | 84.2 | 68.7 | 94.0 |
| | | PII(D 35) | 36 | 33 | 91.7 | 77.5 | 98.2 |
| | | PII(D 42) | 38 | 34 | 89.5 | 75.2 | 97.1 |
| | HN15 | PRE | 33 | 0 | 0.0 | 0.0 | 10.6 |
| | | PI(D 7) | 33 | 18 | 54.5 | 36.4 | 71.9 |
| | | PI(D 14) | 32 | 19 | 59.4 | 40.6 | 76.3 |
| | | PI(D 21) | 32 | 19 | 59.4 | 40.6 | 76.3 |
| | | PII(D 28) | 32 | 22 | 68.8 | 50.0 | 83.9 |
| | | PII(D 35) | 32 | 23 | 71.9 | 53.3 | 86.3 |
| | | PII(D 42) | 32 | 23 | 71.9 | 53.3 | 86.3 |
| | HN30 | PRE | 28 | 0 | 0.0 | 0.0 | 12.3 |
| | | PI(D 7) | 28 | 17 | 60.7 | 40.6 | 78.5 |
| | | PI(D 14) | 28 | 21 | 75.0 | 55.1 | 89.3 |
| | | PI(D 21) | 28 | 23 | 82.1 | 63.1 | 93.9 |
| | | PII(D 28) | 28 | 26 | 92.9 | 76.5 | 99.1 |
| | | PII(D 35) | 28 | 27 | 96.4 | 81.7 | 99.9 |
| | | PII(D 42) | 28 | 27 | 96.4 | 81.7 | 99.9 |
| | Control | PRE | 49 | 0 | 0.0 | 0.0 | 7.3 |
| | | PI(D 7) | 49 | 0 | 0.0 | 0.0 | 7.3 |
| | | PI(D 14) | 49 | 12 | 24.5 | 13.3 | 38.9 |
| | | PI(D 21) | 49 | 29 | 59.2 | 44.2 | 73.0 |
| | | PII(D 28) | 49 | 49 | 100 | 92.7 | 100 |
| | | PII(D 35) | 49 | 49 | 100 | 92.7 | 100 |
| | | PII(D 42) | 49 | 48 | 98.0 | 89.1 | 99.9 |

N = Number of subjects with available results
n/% = Number/percentage of seroprotected subjects (HI titer >=40 1/DIL)
95% CI = 95% confidence interval, LL = Lower Limit, UL = Upper Limit
PI(D 7) = Post vaccination 1 (Day 7); PI(D 14) = Post vaccination 1(Day 14); PI(D 21) = Post vaccination 1 (Day 21); PII(D 35) = Post vaccination 2 (Day 3); PII(D 42) = Post vaccination 2 (Day 42)

TABLE 71
SPR for HI antibody titer at each time point at Day 7, 14 and
21 in the previous trial's adjuvanted vaccine groups against
A/Indonesia/05/2005 strain (ATP cohort for immunogenicity)

| | | | | SPR | | | |
|---|---|---|---|---|---|---|---|
| Vaccine | | | | | | 95% CI | |
| strain | Group | Timing | N | n | % | LL | UL |
| A/ | HN3_8AD | PRE | 39 | 0 | 0.0 | 0.0 | 9.0 |
| INDONESIA | | PI(D 7) | 38 | 32 | 84.2 | 68.7 | 94.0 |
| | | PI(D 14) | 39 | 36 | 92.3 | 79.1 | 98.4 |
| | | PI(D 21) | 39 | 36 | 92.3 | 79.1 | 98.4 |
| | HN7_5AD | PRE | 33 | 1 | 3.0 | 0.1 | 15.8 |
| | | PI(D 7) | 33 | 31 | 93.9 | 79.8 | 99.3 |
| | | PI(D 14) | 33 | 32 | 97.0 | 84.2 | 99.9 |
| | | PI(D 21) | 33 | 33 | 100 | 89.4 | 100 |
| | HN15AD | PRE | 38 | 0 | 0.0 | 0.0 | 9.3 |
| | | PI(D 7) | 38 | 32 | 84.2 | 68.7 | 94.0 |
| | | PI(D 14) | 38 | 33 | 86.8 | 71.9 | 95.6 |
| | | PI(D 21) | 38 | 34 | 89.5 | 75.2 | 97.1 |
| | HN30AD | PRE | 32 | 0 | 0.0 | 0.0 | 10.9 |
| | | PI(D 7) | 33 | 30 | 90.9 | 75.7 | 98.1 |
| | | PI(D 14) | 33 | 31 | 93.9 | 79.8 | 99.3 |
| | | PI(D 21) | 33 | 31 | 93.9 | 79.8 | 99.3 |

N = Number of subjects with available results
n/% = Number/percentage of seroprotected subjects (HI titer >=40 1/DIL)
95% CI = 95% confidence interval, LL = Lower Limit, UL = Upper Limit
PI(D 7) = Post vaccination 1 (Day 7); PI(D 14) = Post vaccination 1 (Day 14); PI(D 21) = Post vaccination 1 (Day 21)

### Intermediate Conclusion

The ≧70% SPR threshold of HI antibodies required by the CHMP was reached by subjects in all previous trial's adjuvanted groups 7 days after vaccination (although the lower limit of the 95% confidence interval for seroprotection was slightly inferior to the threshold for the 3.8 and 15 μg groups) and SPR remained high 21 days after vaccination.
The ≧70% SPR threshold of HI antibodies required by the CHMP was reached by subjects of the control group 7 days after the second vaccination. SPR remained high 21 days after the second vaccination.
In subjects from the previous trial's unadjuvanted groups, the ≧70% SPR threshold was not reached at the selected 3.8 and 15 μg adult doses. The threshold was reached only 7 days after the second vaccination dose for the 7.5 μg group and only 21 days after the first vaccination dose for the 30 μg group (although the lower limit of the 95% confidence interval for seroprotection were inferior to the threshold in both cases).

### General Conclusion for the Humoral Response

When previously vaccinated with an adjuvanted vaccine, formulated with a strain different from the drifted vaccine strain used for the boosting, subjects develop adequate seroprotective humoral response directed against the boosting strain.
The capacity to rapidly (as soon as within 7 days) develop a seroprotective response against a strain drifted from the strain used in the primary vaccination is sustained as long as 14 months, when subjects have been primed with an adjuvanted vaccine and are boosted with adjuvanted vaccine.
HI antibodies levels obtained after boosting with an adjuvanted vaccine at either 6 or 14 months after primary vaccination with an adjuvanted vaccine are similar, when using a vaccine strain drifted from the primary strain for boosting and when testing against the strain used for boosting.
When previously vaccinated with an unadjuvanted vaccine which has been formulated with a strain different from the drifted vaccine strain used for boosting with adjuvanted vaccine, subjects develop a significantly lower humoral response directed against the boosting strain, after the first boosting vaccination dose, when compared to subjects primed with an adjuvanted vaccine.
When previously vaccinated with an unadjuvanted vaccine, formulated with a strain different from the drifted vaccine strain used for boosting with adjuvanted vaccine, subjects develop a significantly lower humoral response directed against the boosting strain, after the second boosting vaccination dose, when compared to subjects assumed to be naïve from H5N1 influenza.

### XV.4.2 Cell-Mediated Immune Response

One of the important features of an adjuvant in enhancing the immunogenicity of a vaccine is the ability to stimulate the cell-mediated immunity (CMI). In this trial, the in vitro specific T-cell responses boosted about 14 months after a primary vaccination course with 2 vaccine doses 21 days apart by a vaccination with 1 dose of an heterologous H5N1 influenza vaccine strain (3.8 μg HA+AS03, 3.8 μg HA and control group, resp.) was investigated in adults aged 18-60 years. The in vitro specific T-cell response is assessed using 6-color IntraCellular Cytokine Stainings.

### Antigens

Split H5N1 Viet: A/Vietnam/1194/2004; Split H5N1 Indo: A/Indonesia/5/05; Pools of peptides covering H5 from A/Vietnam/1194/2004; Pools of peptides covering H5 from A/Indonesia/5/05.

### Intracellular Cytokine Staining

The 6-color Cytokine flow cytometry assays: (CD4, CD8, CD40L, IL-2, TNFα and IFNγ have been used to characterize the specific cellular immune response induced by candidate vaccines.

### Samples and Stimulation Condition

PBMC samples obtained at Day 0 and Day 21 have been stimulated in vitro with:
1. None—subtracted in the clinical data file
2. Split H5N1 Viet: split A/Vietnam/1194/2004
3. Split H5N1 Indo: split A/Indonesia/5/05
4. Pools of peptides covering H5 from A/Vietnam/1194/2004=peptide Viet tot
5. Pools of peptides covering H5 from A/Indonesia/5/05=peptide Indo tot

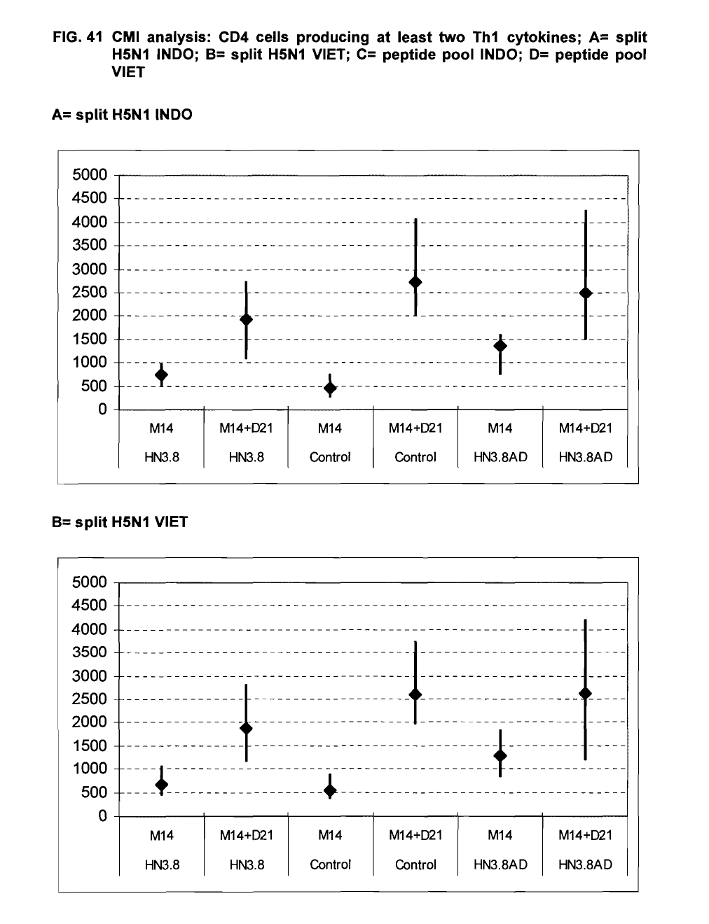

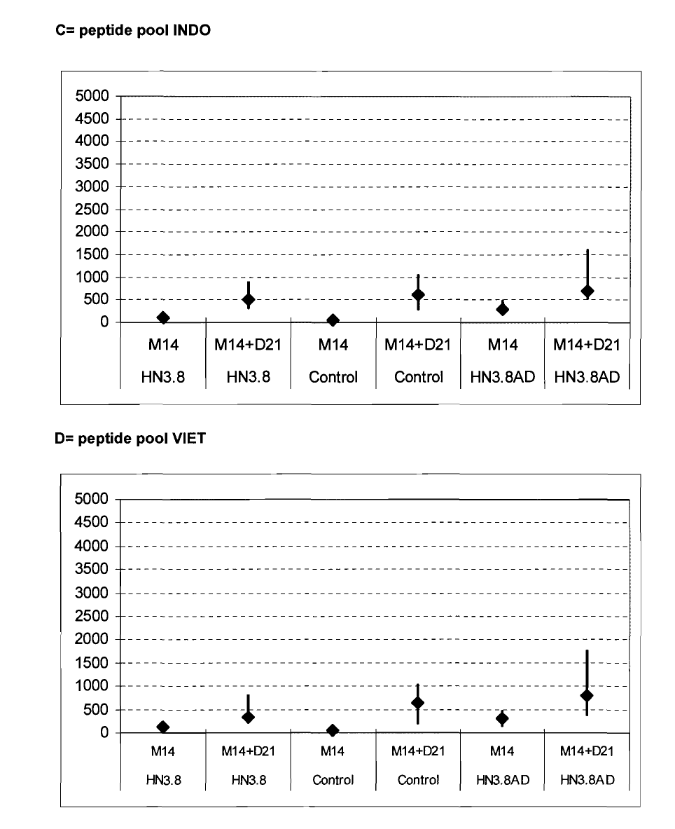

Each result is expressed as a frequency of CD4 T-cells (or CD8 T-cells) expressing at least two cytokines per 10⁶ CD4 T-cells (or CD8 T-cells) in response to antigen stimulation. CMI results are expressed as a frequency of cytokine(s)-positive CD4 T-cells. Results for the “All doubles” i.e. cells producing at least two different cytokines (CD40L, IL-2, TNFα, IFNγ) are presented in Table 72 and in FIG. 41 A-D.

TABLE 72
Descriptive Statistics on the frequency cytokine-positive T-cells (per
million T-cells) for CD4. ALL DOUBLES (ATP cohort for immunogenicity)

| Test | Stimulating | | | | | | | | | | | |
|---|---|---|---|---|---|---|---|---|---|---|---|---|
| description | Ag | Group | Timing | N | Nmiss | GM | SD | Min | Q1 | Median | Q3 | Max |
| CD4. ALL | Split | HN3_8 | PRE | 32 | 2 | 660.79 | 496.52 | 80.00 | 524.00 | 743.00 | 991.00 | 2169.00 |
| DOUBLES | H5N1 Indo | | PI(D 21) | 32 | 2 | 1802.97 | 1208.54 | 680.00 | 1104.50 | 1945.50 | 2737.50 | 5835.00 |
| | | HN3_8AD | PRE | 38 | 1 | 1078.64 | 654.49 | 160.00 | 776.00 | 1370.00 | 1599.00 | 3310.00 |
| | | | PI(D 21) | 38 | 1 | 2556.66 | 3390.98 | 297.00 | 1515.00 | 2504.00 | 4252.00 | 17171.00 |
| | | Control | PRE | 48 | 1 | 432.89 | 377.17 | 13.00 | 277.50 | 473.50 | 757.00 | 1416.00 |
| | | | PI(D 21) | 46 | 3 | 2754.54 | 1581.91 | 1053.00 | 2040.00 | 2721.00 | 4072.00 | 8134.00 |
| | Split | HN3_8 | PRE | 32 | 2 | 644.16 | 574.82 | 27.00 | 475.00 | 682.50 | 1044.50 | 2499.00 |
| | H5N1 Viet | | PI(D 21) | 32 | 2 | 1765.43 | 1194.64 | 614.00 | 1179.50 | 1870.00 | 2806.50 | 5237.00 |
| | | HN3_8AD | PRE | 38 | 1 | 1175.60 | 680.15 | 146.00 | 839.00 | 1284.50 | 1831.00 | 3183.00 |
| | | | PI(D 21) | 38 | 1 | 2416.47 | 3305.79 | 243.00 | 1217.00 | 2640.50 | 4211.00 | 16838.00 |
| | | Control | PRE | 48 | 1 | 432.02 | 415.83 | 1.00 | 379.00 | 540.00 | 873.50 | 1907.00 |
| | | | PI(D 21) | 46 | 3 | 2646.63 | 1477.24 | 826.00 | 1973.00 | 2607.00 | 3738.00 | 8024.00 |
| | Peptide | HN3_8 | PRE | 32 | 2 | 56.83 | 86.23 | 1.00 | 39.50 | 100.50 | 167.50 | 319.00 |
| | Indo tot | | PI(D 21) | 32 | 2 | 470.01 | 523.47 | 18.00 | 331.50 | 499.00 | 873.00 | 2711.00 |
| | | HN3_8AD | PRE | 38 | 1 | 284.85 | 277.26 | 14.00 | 217.00 | 303.00 | 491.00 | 1373.00 |
| | | | PI(D 21) | 38 | 1 | 702.82 | 1264.58 | 1.00 | 545.00 | 702.50 | 1609.00 | 5282.00 |
| | | Control | PRE | 48 | 1 | 26.87 | 89.90 | 1.00 | 1.00 | 64.50 | 131.00 | 374.00 |
| | | | PI(D 21) | 46 | 3 | 520.06 | 937.89 | 1.00 | 307.00 | 613.50 | 1035.00 | 3936.00 |
| | Peptide | HN3_8 | PRE | 32 | 2 | 63.44 | 94.69 | 1.00 | 40.00 | 126.00 | 172.50 | 343.00 |
| | Viet tot | | PI(D 21) | 32 | 2 | 392.98 | 597.93 | 80.00 | 245.50 | 336.00 | 809.00 | 3019.00 |
| | | HN3_8AD | PRE | 38 | 1 | 301.63 | 303.79 | 40.00 | 160.00 | 297.00 | 452.00 | 1506.00 |
| | | | PI(D 21) | 37 | 2 | 799.11 | 1187.29 | 98.00 | 386.00 | 792.00 | 1748.00 | 4868.00 |
| | | Control | PRE | 48 | 1 | 21.12 | 121.94 | 1.00 | 1.00 | 53.00 | 161.00 | 600.00 |
| | | | PI(D 21) | 46 | 3 | 456.67 | 897.42 | 1.00 | 213.00 | 632.00 | 1025.00 | 3790.00 |

HN3_8 = H5N1 3.8 μg
HN3_8AD = H5N1 3.8 μg + AS03
Control = Control
N = number of subjects with available results; Nmiss = number of subjects with missing results
GM = Geometric Mean; SD = Standard Deviation; Q1, Q3 = First and third quartiles
Min/Max = Minimum/Maximum

### Conclusion

The number of specific CD4-positive cells producing at least two Th1 cytokines after in vitro restimulation with Split H5N1 Viet and Split H5N1 Indo antigens was higher in subjects from the adjuvanted groups (HN_{≦}3.8AD and HN_{—}8AD), as compared to subjects from the unadjuvanted vaccine groups (HN_{≦}3.8 and HN_{—}8) (and to subjects from the control group, which represents the baseline level), before revaccination.
The number of specific CD4-positive cells producing at least two Th1 cytokines after in vitro restimulation with Split H5N1 Viet and Split H5N1 Indo antigens was similar in subjects from the adjuvanted groups (HN_{≦}3.8AD and HN_{—}8AD) and in subjects from the control group, and higher as compared to subjects from the unadjuvanted vaccine groups (HN_{—}3.8 and HN_{—}8), 21 days after the first revaccination dose.
The number of specific CD4-positive cells producing at least two Th1 cytokines after in vitro restimulation with pools of peptides covering H5 from A/Vietnam/1194/2004 and A/Indonesia/5/05 viruses was slightly higher in subjects from the adjuvanted groups (HN_{—}3.8AD and HN_{—}8AD), as compared to subjects from the unadjuvanted vaccine groups (HN_{—}3.8 and HN_{—}8) (and to subjects from the control group, which represents the baseline level), before revaccination. The number and increase of CD4-positive cells was not as high as observed after restimulation with Split H5N1 Viet and Split H5N1 Indo antigens, however.
The number of specific CD4-positive cells producing at least two Th1 cytokines after in vitro restimulation with Split H5N1 Viet and Split H5N1 Indo antigens was similarly and slightly increased in subjects from every group, 21 days after the first revaccination dose. The number and increase of CD4-positive cells was not as high as observed after restimulation with Split H5N1 Viet and Split H5N1 Indo antigens, however.

### XV.4.2 B Cell Memory Response

In this trial, the in vitro specific B cell memory responses persistent about 14 months after a primary vaccination with 2 vaccine doses 21 days apart and boosted after vaccination with one dose of an heterologous H5N1 influenza vaccine strain (3.8 μg HA+AS03, 3.8 μg HA and control group, resp.) was investigated in adults aged 18-60 years. In this trial, the in vitro specific B cell memory responses are assessed using the ELISPOT technology.

### ELISPOT

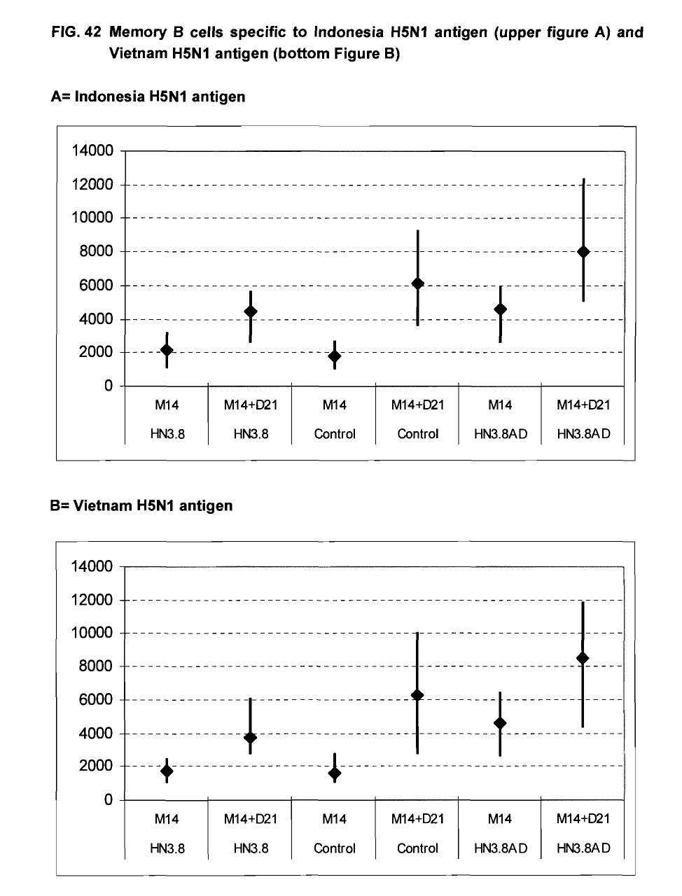

The ELISPOT technology allows the quantification of memory B cells specific to a given antigen. Memory B-cells can be induced to differentiate into plasma cells in vitro following cultivation with CpG for 5 days. In vitro generated antigen-specific plasma cells can therefore be enumerated using the ELISPOT assay. Briefly, in vitro generated plasma cells are incubated in culture plates coated with antigen. Antigen-specific plasma cells form antibody/antigen spots, which can be detected by conventional immunoenzymatic procedure. The stimulating antigens used in vitro are the H5N1 strains: A/Vietnam/1194/2004 and A/Indonesia/5/05. Results have been expressed as a frequency of influenza-specific antibody secreting plasma cells within the IgG-producing plasma cells. Results are presented in Table 73 and in FIG. 42 A-D.

TABLE 73
Descriptive Statistics on the frequency memory B-cell specific to H5N1 antigen
(per million memory B-cells) for IgG (ATP cohort for immunogenicity)

| Test | Stimu- | | | | | | | | | | | | |
|---|---|---|---|---|---|---|---|---|---|---|---|---|---|
| descrip- | lating | Measurement | | | | | | | | | | | |
| tion | Ag | method used | Group | Timing | N | Nmiss | GM | SD | Min | Q1 | Median | Q3 | Max |
| IgG | H5N1- | ELISPOT. B | HN3_8 | PRE | 33 | 1 | 1919.42 | 1322.45 | 0.00 | 1141.00 | 2173.00 | 3204.00 | 5006.00 |
| | Indonesia | | | PI(D 21) | 33 | 1 | 3688.89 | 2486.01 | 311.00 | 2652.00 | 4461.00 | 5657.00 | 11837.00 |
| | | | HN3_8AD | PRE | 35 | 4 | 4076.04 | 2156.34 | 1069.00 | 2659.00 | 4606.00 | 5937.00 | 9803.00 |
| | | | | PI(D 21) | 38 | 1 | 6613.44 | 4910.25 | 206.00 | 5136.00 | 8019.00 | 12350.00 | 20248.00 |
| | | | Control | PRE | 49 | 0 | 1676.82 | 1512.86 | 298.00 | 1047.00 | 1832.00 | 2701.00 | 7721.00 |
| | | | | PI(D 21) | 46 | 3 | 6008.44 | 5858.49 | 763.00 | 3674.00 | 6101.50 | 9238.00 | 27022.00 |
| | H5N1- | ELISPOT. B | HN3_8 | PRE | 33 | 1 | 1516.24 | 1439.33 | 146.00 | 1089.00 | 1714.00 | 2428.00 | 5961.00 |
| | Vietnam | | | PI(D 21) | 33 | 1 | 3624.25 | 2725.49 | 110.00 | 2836.00 | 3746.00 | 6054.00 | 11901.00 |
| | | | HN3_8AD | PRE | 35 | 4 | 3983.35 | 2491.70 | 834.00 | 2647.00 | 4632.00 | 6435.00 | 9476.00 |
| | | | | PI(D 21) | 38 | 1 | 6422.42 | 5149.93 | 53.00 | 4394.00 | 8521.00 | 11849.00 | 19836.00 |
| | | | Control | PRE | 49 | 0 | 1479.00 | 1350.25 | 27.00 | 1113.00 | 1586.00 | 2765.00 | 6060.00 |
| | | | | PI(D 21) | 46 | 3 | 5407.03 | 5005.05 | 694.00 | 2830.00 | 6280.00 | 10004.00 | 23534.00 |

HN3_8 = H5N1 3.8 μg
HN3_8AD = H5N1 3.8 μg + AS03
Control = Control
N = number of subjects with available results; Nmiss = number of subjects with missing results
GM = Geometric Mean; SD = Standard Deviation; Q1, Q3 = First and third quartiles; Min/Max = Minimum/Maximum

### Conclusion

The frequency of H5N1-specific antibody secreting plasma cells after in vitro restimulation with H5N1 Viet and H5N1 Indo antigens was higher in subjects from the previous trial's adjuvanted groups (HN_{—}3.8AD and HN_{—}8AD), as compared to subjects from the previous trial's unadjuvanted vaccine groups (HN_{—}3.8 and HN_{—}8) (and to subjects from the control group, which represents the baseline level), before revaccination.
The frequency of H5N1-specific antibody secreting plasma cells after in vitro restimulation with H5N1 Viet and H5N1 Indo antigens was grossly similar in subjects from the previous trial's adjuvanted groups (HN_{—}3.8AD and HN_{—}8AD) and in subjects from the control group, and tended to be higher as compared to subjects from the previous trial's unadjuvanted vaccine groups (HN_{—}3.8 and HN_{—}8), 21 days after the first revaccination dose.

### XV.5. Overall Conclusions

Persistent, cross-clade CD4-positive and memory B cell responses were detected 14 months after primary vaccination with 2 vaccine doses 21 days apart in subjects from the previous trial's adjuvanted groups (HN_{≦}3.8AD and HN_{—}8AD), that were higher as compared to subjects from the previous trial's unadjuvanted groups (HN_{—}3.8 and HN_{≦}8).
Increases in cross-clade CD4-positive and memory B cell responses were observed in all groups, but were higher in subjects from the previous trial's adjuvanted groups (HN_{—}3.8AD and HN_{—}8AD) and from the control group, 21 days after one dose of revaccination, as compared to subjects from the previous trial's unadjuvanted groups.

1. A method of protecting a subject against a strain of influenza, the method comprising:
administering to a subject a composition comprising an antigenic preparation of a first strain of influenza in combination with an oil and water emulsion adjuvant; and
administering to the subject a composition comprising an antigenic preparation of a second strain of influenza,
wherein the second strain of influenza is a drift variant of the first strain of influenza.

2. The method of claim 1, wherein the humoral immune response to the second strain of influenza is enhanced relative to a humoral immune response in a subject who received a first administration of a composition comprising an antigenic preparation of the first strain in the absence of an oil and water emulsion adjuvant.

3. The method of claim 1, wherein the composition comprising the antigenic composition comprising the second strain of influenza comprises an adjuvant.

4. The method of claim 3, wherein the adjuvant is an oil and water emulsion adjuvant.

5. The method of claim 1, wherein the subject is naïve with respect to the first and second strains of influenza.

6. The method of claim 1, wherein the first and second strains of influenza are selected from a subtype of influenza that is associated with or has the potential to be associated with a pandemic.

7. The method of claim 6, wherein the first and second strains of influenza are of a subtype selected from the group of: H1, H2, H5, H3, H7, H9, H10 influenza virus.

8. The method of claim 7, wherein the first and second strains of influenza are selected from the H5 subtype.

9. The method of claim 8, wherein the first and second strains of influenza are of the H5N1 subtype.

10. The method of claim 1, wherein the antigenic preparation comprises haemagglutinin (HA).

11. The method of claim 10, wherein the composition comprises less than 15 pg of haemagglutinin (HA) per strain per dose.

12. The method of claim 11, wherein the composition comprises less than or equal to about 7.5 pg of HA per strain per dose.

13. The method of claim 11, wherein the composition comprises less than or equal to about 5 pg of HA per strain per dose.

14. The method of claim 1, wherein the subject is selected from the group of: children of between 1 months and 6 months, children below the age of 36 months, children of between 6 and 12 years, children below the age of 18, young adults (18-49 years), adults of between 18-64, elderly over the age of 65.
